(19) United States
(12) Patent Application Publication
(10) Pub. No.: US 2026/0199272 A1
(43) Pub. Date: Jul. 16, 2026

(54) D-BHB (D-BETA-HYDROXYBUTYRATE) FOR TREATMENT OF LONG CHAIN FATTY ACID OXYDATION DEFICIENCY (LC-FAOD)

(71) Applicant: Société des Produits Nestlé S.A., Vevey (CH)

(72) Inventors: Steve LASSUEUR, Brent (CH); Joy RICHARD, Corcelles Le Jorat (CH); Philippe DELERIVE, Lausanne (CH)

(21) Appl. No.: 19/397,378

(22) Filed: Nov. 21, 2025

Related U.S. Application Data
(60) Provisional application No. 63/724,190, filed on Nov. 22, 2024.

(51) Int. Cl. A61K 31/191; A61K 31/198; A61K 31/225; A61K 31/706; A61P 3/00

(52) U.S. Cl. CPC A61K 31/191; A61K 31/198; A61K 31/225; A61K 31/706; A61P 3/00

(57) ABSTRACT

The present invention relates to the use of D-beta-hydroxybutyrate (D-BHB) in the treatment or dietary management of a Long-Chain Fatty Acid Oxidation Deficiency (LC-FAOD), for example, in the prevention and/or amelioration of one or more symptoms associated with LC-FOAD. Additionally, the invention provides compositions comprising D-BHB as the active ingredient. These compositions may be formulated as pharmaceutical compositions or nutritional compositions, such as Foods for Special Medical Purposes (FSMPs).

The present invention relates to the use of D-beta-hydroxybutyrate (D-BHB) in the treatment or dietary management of Long-Chain Fatty Acid Oxidation Deficiency (LC-FAOD), for example, in the prevention and/or amelioration of one or more symptoms associated with LC-FOAD. Additionally, the invention provides compositions comprising D-BHB as the active ingredient. These compositions may be formulated as pharmaceutical compositions or nutritional compositions, including Foods for Special Medical Purposes (FSMPs).

### BACKGROUND TO THE INVENTION

Long-chain fatty acid oxidation disorders (LC-FAODs) represent a group of rare, life-threatening, inborn errors of metabolism that prevent the body from converting long-chain fatty acids (LC-FAs) into energy during periods of fasting and physiological stress leading to episodes of hypoglycemia, rhabdomyolysis, hepatotoxicity and development of cardiomyopathy, nerve conduction abnormalities and retinal degeneration. LC-FAODs are caused by mutations in genes encoding mitochondrial enzymes involved in the carnitine shuttle (for transport of LC-FAs into the mitochondria) or β-oxidation (for conversion of LC-FAs into energy) and include carnitine palmitoyl transferase (CPT) I or II deficiency, very long-chain acyl-CoA dehydrogenase (VLCAD) deficiency (the most common LC-FAOD), long-chain 3-hydroxy-acyl-CoA dehydrogenase (LCHAD) deficiency, carnitine acyl-carnitine translocase (CACT) and trifunctional protein (TFP) deficiency (including long-chain 3 ketoacyl-CoA thiolase [thiolase (LCKAT]) deficiency). Deficiencies in these crucial mitochondrial enzymes lead to partial or incomplete oxidation of LC-FAs, compromises energy homeostasis and causes the accumulation of toxic fatty acid intermediates (acylcarnitines) in the blood and organs, thereby causing detrimental effects on multiple organs like heart, brain, muscle, and liver.

LC-FAODs patients may present with rhabdomyolysis induced by exercise, fasting or illness; hepatic dysfunction, including severe hypoglycemia and hyperammonemia; and cardiomyopathy. These clinical manifestations can lead to frequent hospitalizations and premature death. However, there is great heterogeneity even within the same disorder causing problems with management and prognostic prediction. Early onset disease is also generally associated with more severe disease and poor outcomes, with hepatic dysfunction, hypoglycemia, hyperammonemia more commonly observed before the age of 6 years, whereas cardiomyopathy is linked to disorders that manifest shortly after birth or get diagnosed only in the adulthood. Adults with previously undiagnosed LC-FAOD frequently present with episodic rhabdomyolysis, which is usually precipitated by intense exercise or extreme periods of fasting.

Standard of care varies, but in general, consists of the use of an emergency plan during times of physiological stress. This tends to be a high calorie carbohydrate drink followed by hospital admission and intravenous energy support if the oral route fails. Carnitine supplementation and a low-fat diet is also used in some patients and can be with or without medium-chain triglyceride oil to support the energy demands continuously or only during exercise. Despite these measures, patients continue to require frequent hospital admissions and are at risk of developing cardiomyopathy, peripheral neuropathy, and progressive retinopathy, all of which represent unmet medical needs for these patients.

Triheptanoin (DOJOLVI™) is a triglyceride of medium-chain, odd-carbon of 7 chain length fatty acids with anaplerotic properties that has been approved in the USA as a source of calories and fatty acids for the treatment of pediatric and adult patients with molecularly confirmed LC-FAODs following confirmatory studies in patients (Vockley et al. 2019). Long-term follow-up of patients treated with triheptanoin shows that there is still a significant unmet medical need, particularly with regards to the muscular components of these disorders. Patients continued to experience episodes of rhabdomyolysis, and although there was improvement in cardiac function, it did not reach statistical significance. No beneficial effects were reported in patients with peripheral neuropathy or retinopathy (Vockley et al. 2023).

Thus, there continues to be an unmet medical need for new treatments to offer therapeutic options for patients affected by LC-FAOD, as the current standard of care and approved therapies are unable to address the disease manifestations.

Accordingly, one objective of the present invention is the provision of improved therapies for the treatment of LC-FAODs or the symptoms thereof.

The compound D-beta-hydroxybutyrate (D-BHB), which may also be referred to as R-beta-hydroxybutyrate or (R)-3-hydroxybutrate, is the conjugate base of D-beta-hydroxybutyric acid (shown below).

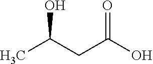

D-BHB is of interest as a ketone body that has been associated with a variety of health benefits. Herein, the present inventors surprisingly identified that D-BHB can also be utilized in the treatment or dietary management of LC-FAODs. The treatment and dietary management are, in non-limiting examples, of particular use in the prevention or amelioration of one or more symptoms associated with LC-FOADs

Due to the high doses of D-BHB that may be required to achieve a therapeutic benefit, there is a concern about high mineral intake from a safety/compliance perspective. Moreover, compositions comprising salts of D-BHB are prone to stability issues and low solubility. Therefore, there is also a need for compositions comprising D-BHB that are both chemically and physically stable, have good solubility in water and address these safety and compliance concerns.

Accordingly, another objective of the present invention is the provision of improved compositions comprising D-BHB as an active ingredient.

### SUMMARY OF THE INVENTION

The present invention is characterized in the herein provided embodiments and claims. In particular, the present invention relates, inter alia, to the following embodiments:
1. A composition comprising D-β-hydroxybutyrate (D-BHB) for use in the treatment and/or dietary management of Long-chain Fatty Acid Oxidation Deficiency (LC-FAOD).
2. The composition for use according to embodiment 1, wherein said treatment or management is the prevention or amelioration of one or more symptoms of said LC-FAOD.
3. The composition for use according to embodiment 1 or 2, wherein the LC-FAOD is caused by a mutation in one or more genes encoding the following mitochondrial enzymes: carnitine palmitoyl transferase I (CPT-I), carnitine palmitoyl transferase (CPT-II), very long-chain acyl-CoA dehydrogenase (VLCAD), long-chain 3-hydroxy-acyl-CoA dehydrogenase (LCHAD), carnitine acyl-carnitine translocase (CACT), trifunctional protein (TFP), and/or long-chain 3 ketoacyl-CoA thiolase (LCKAT).
4. The composition for use according to any one of embodiments 1 to 3, wherein the subject is a pediatric subject or an adult subject.
5. The composition for use according to any one of embodiments 1 to 4, wherein the composition comprises one or more salts of D-β-hydroxybutyrate (D-BHB).
6. The composition for use according to embodiment 5, wherein the one or more salts of D-BHB is selected from a sodium salt, a calcium salt, and/or a magnesium salt.
7. The composition for use according to embodiment 5, wherein the one or more salts of D-BHB is selected from a sodium salt, an L-arginine salt, an-L-lysine salt and/or an erbumine salt.
8. The composition for use according to embodiment 7, wherein the composition comprises a sodium salt of D-BHB (Na-D-BHB) and an L-arginine salt of D-BHB (L-Arg-D-BHB).
9. The composition for use according to embodiment 7 or embodiment 8, wherein the sodium salt of D-BHB (Na-D-BHB) comprises salt form C.
10. The composition for use according to any one of embodiments 7 to 9, wherein the L-arginine salt of D-BHB (L-Arg-D-BHB) comprises salt form B.
11. The composition for use according to any one of embodiments 8 to 10, wherein the molar ratio between the sodium salt of D-BHB (Na-D-BHB) and the L-arginine salt of D-BHB (L-Arg-D-BHB) is between 1:10 and 10:1, preferably between 1:5 and 5:1, more preferably between 1:2 and 2:1, most preferably wherein the molar ratio between the sodium salt of D-BHB (Na-D-BHB) and the L-arginine salt of D-BHB (L-Arg-D-BHB) is about 1:1.
12. The composition for use according to any one of embodiments 1 to 11, wherein the composition is free or essentially free of L-β-hydroxybutyrate (L-BHB).
13. The composition for use according to any one of embodiments 1 to 12, wherein the composition is administered enterally, preferably orally or gastrically, including nasogastrically or by gastrostomy.
14. The composition for use according to any one of embodiments 1 to 12, wherein the composition is administered parentally, preferably intravenously.
15. The composition for use according to any one of embodiments 1 to 13, wherein an active dose of 25 to 1000 mg D-BHB per kg body weight per day (25 to 1000 mg/kg/d) is administered to the subject, preferably as two doses per day, three doses per day, or four doses per day.
16. The composition for use according to embodiment 15, wherein the active dose is adjusted, preferably increased, during the treatment or dietary management.
17. The composition for use according to any one of embodiments 1 to 16, wherein the composition is administered daily, preferably for an indefinite number of days.
18. The composition for use according to any one of embodiments 1 to 17, wherein the composition is co-administered with a medium-chain triglyceride, in particular wherein the medium-chain triglyceride is triheptanoin.
19. A method for restoring or promoting normative cellular energetics, preferably by providing an alternative energy source and/or bypassing mitochondrial fatty acid oxidation, in a subject, comprising administering to the subject a composition comprising D-β-hydroxybutyrate (D-BHB).
20. A method for the treatment or dietary management of a Long-chain Fatty Acid Oxidation Deficiency (LC-FAOD) in a subject in need thereof, comprising administering to the subject a composition comprising D-β-hydroxybutyrate (D-BHB).
21. The method according to embodiment 20, wherein the treatment or dietary management is the prevention or amelioration of one or more symptoms of said LC-FAOD.
22. The method according to embodiment 20 or 21, wherein the LC-FAOD is caused by a mutation in one or more genes encoding the following mitochondrial enzymes: carnitine palmitoyl transferase I (CPT-I), carnitine palmitoyl transferase (CPT-II), very long-chain acyl-CoA dehydrogenase (VLCAD), long-chain 3-hydroxy-acyl-CoA dehydrogenase (LCHAD), carnitine acyl-carnitine translocase (CACT), trifunctional protein (TFP), and/or long-chain 3 ketoacyl-CoA thiolase (LCKAT).
23. The method according to any one of embodiments 19 to 22, wherein the subject is a pediatric subject or an adult subject.
24. The method according to any one of embodiments 19 to 23, wherein the composition comprises one or more salts of D-β-hydroxybutyrate (D-BHB).
25. The method according to embodiment 24, wherein the one or more salts of D-BHB is selected from a sodium salt, a calcium salt, and/or a magnesium salt.
26. The method according to embodiment 24, wherein the one or more salts of D-BHB is selected from a sodium salt, an L-arginine salt, an-L-lysine salt and/or an erbumine salt.
27. The method according to embodiment 26, wherein the composition comprises a sodium salt of D-BHB (Na-D-BHB) and an L-arginine salt of D-BHB (L-Arg-D-BHB).
28. The method according to embodiment 26 or 27, wherein the sodium salt of D-BHB (Na-D-BHB) comprises salt form C.
29. The method according to any one of embodiments 26 to 28, wherein the L-arginine salt of D-BHB (L-Arg-D-BHB) comprises salt form B.
29. The method according to any one of embodiments 27 to 29, wherein the molar ratio between the sodium salt of D-BHB (Na-D-BHB) and the L-arginine salt of D-BHB (L-Arg-D-BHB) is between 1:10 and 10:1, preferably between 1:5 and 5:1, more preferably between 1:2 and 2:1, most preferably wherein the molar ratio between the sodium salt of D-BHB (Na-D-BHB) and the L-arginine salt of D-BHB (L-Arg-D-BHB) is about 1:1.
30. The method according to any one of embodiments 19 to 29, wherein the composition is free or essentially free of L-β-hydroxybutyrate (L-BHB).
31. The method according to any one of embodiments 19 to 30, wherein the composition is administered enterally, preferably orally or gastrically, including nasogastrically or by gastrostomy.
32. The method according to any one of embodiments 19 to 31, wherein the composition is administered parentally, preferably intravenously.
33. The method according to any one of embodiments 19 to 32, wherein an active dose of 25 to 1000 mg D-BHB per kg body weight per day (25 to 1000 mg/kg/d) is administered to the subject, preferably as two doses per day, three doses per day, or four doses per day.
34. The method according to embodiment 33, wherein the active dose is adjusted, preferably increased, during the treatment or dietary management.
35. The method according to any one of embodiments 19 to 34, wherein the composition is administered daily, preferably for an indefinite number of days.
36. The method according to any one of embodiments 19 to 35, wherein the composition is co-administered with a medium-chain triglyceride, in particular wherein the medium-chain triglyceride is triheptanoin.
37. A composition comprising two or more salts of D-β-hydroxybutyrate (D-BHB), wherein at least one of the two or more salts is selected from a sodium salt, an L-arginine salt, an-L-lysine salt and/or an erbumine salt.
38. The composition according to embodiment 37, wherein the composition comprises at least one amino acid salt of D-BHB and one mineral salt of D-BHB, preferably wherein the mineral salt of D-BHB is a sodium salt of D-BHB (Na-D-BHB).
39. The composition according to embodiment 37 or embodiment 38, wherein the composition comprises a sodium salt of D-BHB (Na-D-BHB) and an L-arginine salt of D-BHB (L-Arg-D-BHB).
40. The composition according to any one of embodiments 37 to 39, wherein the sodium salt of D-BHB (Na-D-BHB) comprises salt form C.
41. The composition according to any one of embodiments 37 to 40, wherein the L-arginine salt of D-BHB (L-Arg-D-BHB) comprises salt form B.
42. The composition according to any one of embodiments 39 to 41, wherein the molar ratio between the sodium salt of D-BHB (Na-D-BHB) and the L-arginine salt of D-BHB (L-Arg-D-BHB) is between 1:10 and 10:1, preferably between 1:5 and 5:1, more preferably between 1:2 and 2:1, most preferably wherein the molar ratio between the sodium salt of D-BHB (Na-D-BHB) and the L-arginine salt of D-BHB (L-Arg-D-BHB) is about 1:1.
43. The composition according to embodiment 37, wherein the two or more salts of D-BHB are selected from a sodium salt, a calcium salt, and/or a magnesium salt, preferably wherein the composition comprises all three of a sodium salt, a calcium salt, and/or a magnesium salt of D-BHB.
44. The composition according to embodiment 43, wherein the molar ratio between (i) the sodium salt and (ii) the calcium salt and/or the magnesium salt ranges from 1:1 to 1:10, preferably wherein the molar ratio between the sodium salt, the calcium salt and the magnesium salt (Na:Ca:Mg) is about 1:2:2.
45. The composition according to embodiment 43 or embodiment 44, wherein the composition further comprises D-BHB in its free form.
46. The composition according to embodiment 45, wherein the ratio between the D-BHB salts and D-BHB in its free form ranges from 1:10 to 10:1, preferably 1:5 to 5:1, more preferably 1:2 to 2:1, even more preferably 1:1.5 to 1.5:1, most preferably about 1:1.
47. The composition according to any one of embodiments 43 to 46, wherein the composition further comprises nicotinamide riboside.
48. The composition according to any one of embodiments 37 to 47, wherein the composition is free or essentially free of L-β-hydroxybutyrate (L-BHB).
49. The composition according to any one of embodiments 37 to 48, wherein the composition is formulated as a powder, preferably a reconstitutable powder.
50. The composition according to any one of embodiments 37 to 49, wherein the composition is a nutritional composition, preferably a food for special medical purpose (FSMP) and preferably comprising at least one macronutrient selected from the group consisting of a protein, a carbohydrate, a lipid, and combinations thereof.
51. The composition according to any one of embodiments 37 to 49, wherein the composition is a pharmaceutical composition further comprising a pharmaceutically acceptable carrier.
52. The composition according to any one of embodiments 37 to 51, wherein the composition further comprises a diluent, a filler, a flow aid/glidant, a lubricant and/or a flavoring agent.
53. The composition according to any one of embodiments 37 to 52, wherein the composition is formulated for enteral administration, preferably oral administration or gastric administration, including nasogastric administration or administration by gastrostomy, or for parental administration, preferably intravenous administration.

Accordingly, in one aspect, the invention relates to a composition comprising D-β-hydroxybutyrate (D-BHB) for use in the treatment and/or dietary management of a Long-chain Fatty Acid Oxidation Deficiency (LC-FAOD). In particular, the treatment and/or dietary management disclosed herein are of use, for example, in the prevention or amelioration of one or more symptom of LC-FAOD.

LC-FAODs are a group of inherited metabolic disorders characterized by the body's inability to break down long-chain fatty acids into energy through the process of fatty acid oxidation. The inability to properly metabolize long-chain fatty acids leads to a lack of energy (ATP) production, which is critical for cellular function and overall metabolism. Specifically, this defect prevents fatty acids from entering the tricarboxylic acid (TCA) cycle, also known as the Krebs cycle, which is essential for generating ATP from fatty acids. The accumulation of toxic fatty acid intermediates further exacerbates the condition, causing cellular damage and contributing to the severity of symptoms.

The inventors have surprisingly found that delivering D-β-hydroxybutyrate (D-BHB), in particular substantially enantiomerically pure D-BHB or enantiomerically pure B-BHB, as an alternative energy source can restore cellular energetic status in cells that are defective in long-chain fatty acid metabolism. For instance, as demonstrated in Example 17, cells lacking functional genes involved in long-chain fatty acid metabolism exhibited reduced basal respiration compared to wild-type cells and severely impaired respiration in response to palmitate. However, it was shown that the respiration of these cells markedly improved when D-BHB was provided as an alternative fuel source. This data suggests that D-BHB can effectively bypass the genetic defects inherent in these disorders, allowing cells to produce energy despite the metabolic blockages.

Of note, the role of ketone body metabolism is not limited to its involvement in energy metabolism since D-BHB ketone bodies also function as lipogenic and sterol biosynthetic substrates in a variety of tissues including brain, liver, and heart (Morris, 2005, Journal of Inherited Metabolic Disease, 28 (2), 109-121). The mode of signaling can be either direct on specific receptors or indirect due to their effects on mitochondrial energy production. Some of these effects are direct actions of D-BHB itself. Some are indirect effects governed by downstream metabolites into which D-BHB is converted, such as acetyl-CoA (Nelson et al, 2023, Nature Metabolism, 5, 2062-2074). Among these various pathways modulated by D-BHB, three of them appear to be particularly relevant in the context of LC-FAOD. First, inhibition of class I histone deacetylases by D-BHB is associated with global changes in transcription, including that of the genes encoding oxidative stress resistance resulting ultimately in suppression of oxidative stress (Shimazu et al, 2013, Science, 339 (6116), 211-214). Second, D-BHB is the only known endogenous ligand for HCAR2 with an EC50 around 0.7 mM (Taggart et al, 2005, The Journal of Biological Chemistry, 280 (29), 26649-26652). HCAR2 (also known as HCA2, PUMA-G, and Gpr109) is a G-protein-coupled receptor that was first identified as a nicotinic acid receptor activated by D-BHB leading to reduction in lipolysis in adipocytes (Taggart et al., 2005, The Journal of Biological Chemistry, 280 (29), 26649-26652). In addition, HCAR2 activation has been linked to anti-inflammatory properties in various diseases such as obesity, atherosclerosis, and inflammatory bowel disease (see Graff et al, 2016, Metabolism: Clinical and Experimental, 65 (2), 102-113, for review). Last but not least, D-BHB has been shown to block the NOD-like receptor protein 3 inflammasome-mediated inflammatory disease (Youm et al, 2015, Nature Medicine, 21 (3), 263-269). In conclusion, in addition to its energetic properties, D-BHB supplementation may provide via these non-canonical signaling pathways both anti-inflammatory and antioxidant properties. This is particularly relevant in the context of LC-FAOD where mitochondrial alterations lead to the increase in toxic metabolites such as LC-FAs, long-chain acylcarnitines and reactive oxygen species which are key pathophysiological drivers in tissues such as heart and skeletal muscle.

The present invention is directed to the use of D-BHB in the treatment or dietary management of LC-FAODs. The treatments and dietary managements disclosed herein are of particular use in the prevention and amelioration of symptoms of LC-FAODs. Reported symptoms of or associated with LC-FAODs are wide ranging, due to the body's inability to properly metabolize long-chain fatty acids. These symptoms may include, without limitation, cardiac involvement (cardiomyopathy), brain involvement, hyperammonemia, hypoglycemia, liver-hepatic dysfunction, muscular involvement (including rhabdomyolysis), neuropathy and retinopathy. Cardiac involvement (cardiomyopathy) refers to a condition where the heart muscle becomes diseased, leading to impaired heart function and potentially heart failure.

Brain involvement encompasses neurological symptoms that can affect cognitive and motor functions, potentially leading to developmental delays, seizures, or other brain-related issues. Hyperammonemia is characterized by elevated levels of ammonia in the blood, which can be toxic and lead to neurological disturbances and other health issues.

Hypoglycemia involves dangerously low blood sugar levels, which can cause symptoms such as dizziness, confusion, seizures, and loss of consciousness.

Liver-hepatic dysfunction refers to damage to the liver, impairing its ability to perform essential metabolic processes and detoxify the body.

Muscular involvement (including rhabdomyolysis) involves the breakdown of muscle tissue, leading to muscle pain, weakness, and potential kidney damage due to the release of muscle breakdown products into the bloodstream.

Neuropathy is the damage to the nerves, causing symptoms such as pain, tingling, numbness, and muscle weakness.

Retinopathy refers to damage to the retina of the eye, which can lead to vision problems and potentially blindness.

It is to be understood that the interventions disclosed herein cannot cure the underlying cause of an LC-FAOD, i.e., the genetic defect causing the disease. Thus, the term “treat,” “treatment,” and variations thereof as used herein refer to interventions taken to prevent, manage, ameliorate and/or alleviate one or more symptoms associated with LC-FAODs, such as any one of the symptoms disclosed herein. Accordingly, the invention is also directed to the treatment, management, prevention or amelioration of one or more symptoms of an LC-FAOD. Such treatment, management, prevention or amelioration may include, in non-limiting examples, restoring and maintaining cellular energy production by providing D-BHB as an alternative energy source, which can bypass the defective metabolic pathways. The goal of treatment and management is to prevent symptoms from occurring, reduce the frequency of symptom appearance, reduce the severity of symptoms, improve the patient's overall health and well-being, and/or enhance their quality of life by effectively managing the disorder.

The term “prevent,” “prevention,” or variations thereof as used herein refers to strategies and interventions aimed at reducing the risk, severity, or frequency of one or more existing or expected symptoms associated with long-chain fatty acid oxidation disorders (LC-FAODs), such as any one of the symptoms disclosed herein. Accordingly, as used herein, “prevention” and analogous terms are not understood to reference an intervention necessarily completely eliminating the recurrence of symptoms or the chance of recurrence of symptoms. As used herein, “prevention” and analogous terms also include measures to avert the onset of symptoms. The goal of prevention is to minimize the occurrence of symptoms, stabilize the condition, and improve the overall quality of life for those affected by these disorders.

The intervention may include the dietary management of LC-FAOD, which includes the management of symptoms thereof. As used herein, dietary management is understood to mean the exclusive or partial feeding of a subject who because of a disease, disorder or medical condition has limited or impaired capacity to digest, absorb or otherwise metabolize certain foods or certain nutrients contained therein; or has determined nutrient requirements. Thus, it is understood that dietary management as used herein provides D-BHB to a subject deemed to be in need thereof, e.g. as a component of a nutritional composition. The dietary management of LC-FAOD is understood to include the minimization or prevention of one or more existing or expected symptoms of LC-FAOD, such as any of the symptoms disclosed herein. D-BHB for the dietary management according to the methods and uses of the invention may be formulated according to well-known and standard practices in the art and may be any suitable nutritional composition known in the art, including formulation as a food product, food supplement, a functional beverage product, nutritional supplement, dietary supplement, over-the-counter (OCT) supplement, medical food, Enteral Formula for Special Medical Use, Food for Specified Health Uses, Food for Special Medical Purposes (FSMP), Food for Special Dietary Use (FSDU), and a Medical Food. In certain embodiments, D-BHB is used in the preparation of the nutritional composition for the uses and methods according to the invention. In certain embodiments, D-BHB is the only active ingredient or agent in the nutritional composition for the uses and methods according to the invention. However, encompassed herein are also nutritional compositions comprising further active ingredients, as are specified elsewhere herein.

A subject may be determined to be in need of an intervention according to the invention if the subject has been diagnosed with, or is suspected to have, an LC-FAOD. Preferably, a subject in need of an intervention is one that has been diagnosed with an LC-FAOD, preferably through genetic testing. Consequently, the subject may or may not exhibit one or more symptoms of an LC-FAOD at the time the intervention is initiated. In particular, a subject diagnosed with an LC-FAOD may receive an intervention prior to the onset of any symptoms. This proactive approach aims to manage the disorder effectively and prevent the development of symptoms, thereby improving the subject's overall health and quality of life.

Suitable subjects of the present invention include mammalian subjects, but are preferably human or companion animals (including without limitation, dogs and cats). The mammalian subject according to the present invention includes, but is not limited to a human, canine, feline, lagomorph, mustelid, rodent, bovine, caprine, equine, camelid, ovine, porcine, primate, and a simian. The subject according to the present invention may be avian.

Most commonly, LC-FAOD is caused by mutations in one or more of the following genes:
ACADVL: Encodes the enzyme very-long-chain acyl-CoA dehydrogenase (VLCAD), which is involved in the initial steps of long-chain fatty acid oxidation.
HADHA: Encodes the alpha subunit of the mitochondrial trifunctional protein (MTP), which has both long-chain 3-hydroxyacyl-CoA dehydrogenase and enoyl-CoA hydratase activities.
HADHB: Encodes the beta subunit of the mitochondrial trifunctional protein (MTP), which has long-chain 3-ketoacyl-CoA thiolase activity.
CPT1A: Encodes carnitine palmitoyltransferase I (CPT I), which is involved in the transport of long-chain fatty acids into the mitochondria.
CPT2: Encodes carnitine palmitoyltransferase II (CPT II), which is involved in the final steps of transporting long-chain fatty acids into the mitochondrial matrix.
SLC25A20: Encodes carnitine-acylcarnitine translocase (CACT), which is responsible for transporting acylcarnitines into the mitochondria for fatty acid oxidation.

Mutations in these genes can lead to various forms of LC-FAODs, each with distinct clinical presentations and severity. By providing D-BHB, an alternative fuel source, the treatment can circumvent these genetic defects and restore energy production in affected cells.

Mutations in these genes can be identified through genetic testing, including newborn screening, which allows for early diagnosis and intervention.

The prevalence of the different types of LC-FAODs and the associated symptoms are listed in Table below:

| | | Conditions affected by age category | | |
|---|---|---|---|---|
| Disorder (Gene) | Approximate prevalence | Neonatal | Childhood | Adult |
| VLCADD (ACADVL) | 1:85,000 | C, HG, L | C, M, HG | M, C |
| LCHADD (HADHA) | 1:250,000 to 1:750,000 | C, L, HG | M, C, N, R, HG | M, C, N, R |
| TFPD (HADHA, HADHB) | — | C, L, HG | L, HG | M, N, R |
| CPT-IAD (CPT1) | 1:750,000 to 1:2,000,000 | L, HG, B | M | — |
| CPT-IID (CPT2) | — | L, HG, C, B | HG, C | M, C |
| CACTD (SLC25A20) | — | C, HG, L, HA | HG | M, C |

Approximate prevalence data taken from Lindner et al. 2010 (Journal of Inherited Metabolic Disease, 33(5), 521-526) and Merritt et al., 2020 (Reviews in Endocrine Metabolic Disorders, 21(4), 479-493).
B = brain involvement; C = cardiac involvement (cardiomyopathy); CACTD = carnitine acyl-carnitine translocase deficiency; CPT-IAD/CPT-IID = carnitine palmitoyl transferase I/II deficiency; HA = hyperammonemia; HG = hypoglycemia; L = liver-hepatic dysfunction; LCHADD = long-chain L-3 hydroxyacyl-CoA dehydrogenase deficiency; M = muscular involvement including rhabdomyolysis; N = neuropathy; R = retinopathy TFPD = tri-functional protein deficiency; VLCADD = very long-chain acyl-CoA dehydrogenase deficiency.

That is, in certain embodiments, the LC-FAOD is very long-chain acyl-CoA dehydrogenase deficiency (VLCADD). In certain embodiments, VLCADD is neonatal VLCADD and the associated symptoms involve one or more of cardiac involvement (cardiomyopathy), hypoglycemia and/or liver-hepatic dysfunction. In certain embodiments, VLCADD is childhood VLCADD and the associated symptoms involve one or more of cardiac involvement (cardiomyopathy), muscular involvement including rhabdomyolysis and/or hypoglycemia. In certain embodiments, VLCADD is adult VLCADD and the associated symptoms involve one or more of cardiac involvement (cardiomyopathy) and/or muscular involvement including rhabdomyolysis.

In certain embodiments, the LC-FAOD is long-chain L-3 hydroxyacyl-CoA dehydrogenase deficiency (LCHADD). In certain embodiments, LCHADD is neonatal LCHADD and the associated symptoms involve one or more of cardiac involvement (cardiomyopathy), hypoglycemia and/or liver-hepatic dysfunction. In certain embodiments, LCHADD is childhood LCHADD and the associated symptoms involve one or more of cardiac involvement (cardiomyopathy), muscular involvement including rhabdomyolysis, neuropathy, retinopathy and/or hypoglycemia. In certain embodiments, LCHADD is adult LCHADD and the associated symptoms involve one or more of cardiac involvement (cardiomyopathy), neuropathy, retinopathy and/or muscular involvement including rhabdomyolysis.

In certain embodiments, the LC-FAOD is tri-functional protein deficiency (TFPD). In certain embodiments, TFPD is neonatal TFPD and the associated symptoms involve one or more of cardiac involvement (cardiomyopathy), hypoglycemia and/or liver-hepatic dysfunction. In certain embodiments, TFPD is childhood TFPD and the associated symptoms involve one or more of liver-hepatic dysfunction and/or hypoglycemia. In certain embodiments, TFPD is adult TFPD and the associated symptoms involve one or more of neuropathy, retinopathy and/or muscular involvement including rhabdomyolysis.

In certain embodiments, the LC-FAOD is carnitine palmitoyl transferase I deficiency (CPT-IAD). In certain embodiments, CPT-IAD is neonatal CPT-IAD and the associated symptoms involve one or more of brain involvement, hypoglycemia and/or liver-hepatic dysfunction. In certain embodiments, CPT-IAD is childhood CPT-IAD and the associated symptoms involve muscular involvement including rhabdomyolysis.

In certain embodiments, the LC-FAOD is carnitine palmitoyl transferase II deficiency (CPT-IID). In certain embodiments, CPT-IID is neonatal CPT-IID and the associated symptoms involve one or more of cardiac involvement (cardiomyopathy), hypoglycemia, brain involvement and/or liver-hepatic dysfunction. In certain embodiments, CPT-IID is childhood CPT-IID and the associated symptoms involve one or more of cardiac involvement (cardiomyopathy) and/or hypoglycemia. In certain embodiments, CPT-IID is adult CPT-IID and the associated symptoms involve one or more of cardiac involvement (cardiomyopathy) and/or muscular involvement including rhabdomyolysis.

In certain embodiments, the LC-FAOD is carnitine acyl-carnitine translocase deficiency (CACTD). In certain embodiments, CACTD is neonatal CACTD and the associated symptoms involve one or more of cardiac involvement (cardiomyopathy), hypoglycemia, hyperammonemia and/or liver-hepatic dysfunction. In certain embodiments, CACTD is childhood CACTD and the associated symptoms involve hypoglycemia. In certain embodiments, CACTD is adult CACTD and the associated symptoms involve one or more of cardiac involvement (cardiomyopathy) and/or muscular involvement including rhabdomyolysis.

The subject suffering from a Long-chain Fatty Acid Oxidation Deficiency (LC-FAOD) may be any subject, including newborns, children and adults. In a particular embodiment, the invention relates to the composition for use according to the invention, wherein the subject is a pediatric subject. In other embodiments, the invention relates to the composition for use according to the invention in an elderly adult. An elderly adult may be 60 years or older, 65 years or older, 70 years or older, 75 years or older, or 80 years or older.

The unmet medical needs in the population of LC-FAOD patients continue to be very significant, especially in pediatric populations. Pediatric presentations represent the most severe end of the disease spectrum, resulting in frequent hospital admissions and emergency room visits due to very short fasting tolerance, episodes of hypoglycemia, and multi-organ involvement. Cardiomyopathy is often lethal in cases of very early onset disease, while those with a slower rate of progression still heavily depend on supportive care to manage arrhythmias, cardiac failure, and debilitating episodes of rhabdomyolysis. A pediatric patient is defined as an individual who is in the developmental stages of life, from birth through adolescence. This typically includes newborns, infants, children, and adolescents up to the age of 18 years. Pediatric patients are characterized by their unique physiological, developmental, and psychological needs, which can differ significantly from those of adults.

In certain embodiments, the subject is a neonatal subject. That is, in certain embodiments, the composition according to the invention is administered to a pediatric subject during the neonatal stage, i.e., during the first 28 days of life. Newborns are typically tested for LC-FAODs during their first few days of life through newborn screening. If an LC-FAOD is detected in a newborn, treatment with the composition according to the invention can be initiated immediately to control the symptoms and manage the disorder effectively. If treatment is initiated at the neonatal stage, it is preferably continued throughout the individual's life to ensure ongoing management of the LC-FAOD. In certain embodiments, the subject suffering from an LC-FAOD is an adult subject, i.e., a subject aged 18 years or older.

Adult subjects include elderly subjects (which may be used interchangeably with the term geriatric subject), i.e., subjects aged 60 years or older, 65 years or older, 70 years or older, 75 years or older, or 80 years or older.

In another aspect, the invention relates to a method for the treatment or dietary management of a Long-chain Fatty Acid Oxidation Deficiency (LC-FAOD) in a subject in need thereof, comprising administering to the subject a composition comprising D-β-hydroxybutyrate (D-BHB); preferably wherein the treatment or dietary management is the prevention or amelioration of one or more symptoms of said LC-FAOD. The same principles and applications described herein above for the use of the composition according to the invention apply to such methods.

In a further aspect, the invention relates to a method for restoring or promoting normative cellular energetics, preferably by providing an alternative energy source and/or bypassing mitochondrial fatty acid oxidation, in a subject, comprising administering to the subject a composition comprising D-β-hydroxybutyrate (D-BHB).

It has been surprisingly found herein that D-BHB can be used to restore or promote normative cellular energetics. Without being bound by theory, utilizing D-BHB as an alternative fuel source may restore cellular energetics, and/or reduce the likelihood of metabolic dysfunction in subjects suffering from LC-FAOD. As an alternative fuel source, D-BHB may effectively bypass the genetic defect in LC-FAOD subjects, resulting in improved mitochondrial function and/or cellular energetics.

The term “cellular energetics” refers to the processes by which cells generate, store, and utilize energy, primarily in the form of adenosine triphosphate (ATP), through metabolic pathways such as glycolysis, the citric acid cycle, and oxidative phosphorylation. In this context, “normative” denotes the standard or typical state of these processes under healthy conditions, where energy production and utilization function optimally to meet cellular demands and maintain metabolic balance. Restoring or promoting normative cellular energetics involves re-establishing or enhancing this normal, healthy state to ensure proper cellular function and overall metabolic homeostasis.

Normative cellular energetics may be restored or promoted by providing an alternative fuel source that can be converted into energy, effectively bypassing impaired metabolic pathways responsible for abnormal cellular energetics. In particular, D-β-hydroxybutyrate (D-BHB) can serve as such an alternative energy source, allowing cells to produce ATP even when traditional pathways, such as mitochondrial fatty acid oxidation pathways, are compromised.

In certain embodiments, D-BHB is used to restore or promote normal cellular energetics in a subject experiencing abnormal or reduced cellular energetics. These abnormalities or reductions in cellular energetics can have various causes. However, in preferred embodiments, the abnormal or reduced cellular energetics are caused by a genetic defect, particularly in genes involved in mitochondrial fatty acid oxidation. Such genes include, but are not limited to, ACADVL, HADHA, CPT1A, CPT2, SLC22A5, and SLC25A20, as disclosed herein.

Further encompassed herein are methods for promoting cellular energetics in a healthy subject, i.e., in a subject with normal cellular energetics.

It is to be understood that any composition comprising D-BHB may be used in the treatment or dietary management of Long-chain Fatty Acid Oxidation Deficiencies (LC-FAODs); or the treatment, dietary management, prevention or amelioration of one or more symptoms associated with an LC-FAOD; or for restoring or promoting normative cellular energetics in a subject. That is, the composition for use in the treatment or dietary management of Long-chain Fatty Acid Oxidation Deficiencies (LC-FAODs), or for restoring or promoting normative cellular energetics may comprise D-BHB in any form.

In a particular embodiment, the invention relates to the composition for use or the method according to the invention, wherein the composition comprises one or more salts of D-β-hydroxybutyrate (D-BHB). The salts may be any salts, including, without limitation, one or more salts of alkali metals, alkaline earth metals, transition metals, amino acids, or metabolites of amino acids. Examples include, without limitation, lithium salts, sodium salts, potassium salts, magnesium salts, calcium salts, zinc salts, iron salts (as iron II and/or iron III), chromium salts, manganese salts, cobalt salts, copper salts, molybdenum salts, selenium salts, arginine salts, lysine salts, leucine salts, isoleucine salts, histidine salts, ornithine salts, citrulline salts, glutamine salts, and creatine salts.

Alternative or in addition, the D-BHB may be provided as one or more esters, such as mono-, di-, tri-, oligo-, and polyesters. Examples include mono-ester of ethanol, mono-ester of 1-propanol, mono-ester of 1,2-propanediol, di-ester of 1,2-propanediol, mono-ester of 1,3-propanediol, di-ester of 1,3-propanediol, mono-ester of S-,R-, or S-R-1,3-butanediol, di-ester of S-,R-, or S-R-1,3-butanediol, mono-ester of glycerin, (3S)-hydroxybutyl (3S)-hydroxy butyrate mono-ester, (3R)-hydroxybutyl (3-S)-hydroxy butyrate, mono-ester, di-ester of glycerin, tri-ester of glycerin, ester of acetoacetate, dimers, trimers, oligomers, and polyesters containing repeating units of beta-hydroxybutyrate, and complex oligomers or polymers of beta-hydroxybutyrate and one or more other hydroxy-carboxylic acids, such as lactic acid, citric acid, acetoacetic acid, quinic acid, shikimic acid, salicylic acid, tartaric acid, and malic acid, and/or beta-hydroxybutyrate and or one or more diols, such as 1,3-propanediol and 1,3-butanediol, and one or more polyacids, such as tartaric acid, citric acid, malic acid, succinic acid, and fumaric acid.

In certain embodiments, the composition for use according to the invention may comprise a free form of D-BHB, such as a crystalline free base form of D-BHB. Crystalline free base Form I can be characterized by XRPD pattern, obtained as set forth in the Examples, having peaks at 10.93, 12.11, 15.16, 17.53, and 18.88±0.2° 2θ using Cu Kα radiation. Form I can be further characterized by having an XRPD pattern having additional peaks at 22.75, 24.30, 26.14, 29.10, and 29.95±0.2° 2θ using Cu Kα radiation. Form I can be further characterized by having an XRPD pattern having additional peaks at 30.48, 31.60, 32.96, 33.98, 36.80, and 38.37±0.2° 2θ using Cu Kα radiation. In some embodiments, Form I has an XRPD pattern substantially as shown in FIG. 1A, wherein by “substantially” is meant that the reported peaks can vary by about #0.2°. It is well known in the field of XRPD that while relative peak heights in spectra are depending on a number of factors, such as sample preparation and instrument geometry, peak positions are relatively insensitive to experimental details. Form I may also be characterized by DSC substantially as set forth in FIG. 2A and/or by the TGA set forth in FIG. 3A. In some embodiments, crystalline free base Form I may be characterized by one or more of an XRPD substantially as depicted in FIG. 1A, DSC substantially as set forth in FIG. 2A, and TGA as set forth in FIG. 3A.

However, it is preferred herein, that the composition for use according to the invention comprises one or more salts of D-BHB. The salts may be amorphous or crystalline salts and may exist in any stoichiometric form.

In a particular embodiment the invention relates to the composition for use according to the invention or the method according to the invention, wherein the one or more salts of D-BHB is selected from a sodium salt, a calcium salt, and/or a magnesium salt.

That is, the composition according to the invention may comprise at least one of a sodium salt, a calcium salt and a magnesium salt of D-BHB. In certain embodiments, the composition comprises at least two of a sodium salt, a calcium salt and a magnesium salt of D-BHB. In certain embodiments, the composition comprises a sodium salt, a calcium salt and a magnesium salt of D-BHB, i.e., the composition comprises a mixture of these three salts.

The sodium salt, calcium salt, and magnesium salt of D-BHB may be present in the composition in any suitable molar ratio. In certain embodiments, the sodium salt may be present at a lower molar ratio compared to the magnesium salt and calcium salt. For example, the molar ratio between (i) the sodium salt and (ii) the calcium salt and/or the magnesium salt may range from 1:1 to 1:10, such as 1:1, 1:2, 1:3, 1:4, 1:5, 1:6, 1:7, 1:8, 1:9 or 1:10. In some embodiments, the sodium salt, calcium salt, and magnesium salt of D-BHB may be present at a molar ratio of about 1:2:2 (Na:Ca:Mg).

Additionally, the composition may further comprise the free form of D-BHB in addition to the sodium salt, the calcium salt, and/or the magnesium salt of D-BHB. The free form of D-BHB can be mixed with the D-BHB salts at any suitable molar ratio. In certain embodiments, the molar ratio between the D-BHB salts and D-BHB in its free form ranges from 1:10 to 10:1, preferably 1:5 to 5:1, more preferably 1:2 to 2:1, even more preferably 1:1.5 to 1.5:1, most preferably about 1:1. Furthermore, in certain embodiments, the composition comprising the free form of D-BHB and the salt mixture may further comprise nicotinamide riboside.

In certain embodiments, the composition for use according to the invention may comprise one or more sodium salts of D-BHB. For example, the composition for use according to the invention may comprise one or more of D-BHB crystalline sodium salt Forms A, B and/or C, as characterized herein.

D-BHB crystalline sodium salt Form A can be characterized by XRPD pattern, obtained as set forth in the Examples, having peaks at 11.82, 17.02, 17.58, 20.34, and 20.83±0.2 2θ using Cu Kα radiation. Sodium salt Form A can be further characterized by having an XRPD pattern having additional peaks at 7.23, 14.39, 24.50, 29.54, and 30.17±0.2° 2θ using Cu Kα radiation. Sodium salt Form A can be further characterized by having an XRPD pattern having additional peaks at 12.22, 22.23, 22.60, 30.63, 33.35, and 37.28±0.2° 2θ using Cu Kα radiation. In some embodiments, sodium salt Form A has an XRPD pattern substantially as shown in FIG. 9A.

D-BHB crystalline sodium salt Form A may also be characterized by DSC substantially as set forth in FIG. 9B and/or by the TGA set forth in FIG. 9C. In some embodiments, D-BHB crystalline sodium salt Form A may be characterized by one or more of an XRPD substantially as depicted in FIG. 9A, DSC substantially as set forth in FIG. 9B, and TGA as set forth in FIG. 9C.

D-BHB crystalline sodium salt Form B can be characterized by XRPD pattern, obtained as set forth in the Examples, having peaks at 8.98, 11.49, 16.95, 17.53, and 19.38±0.2° 2θ using Cu Kα radiation. Sodium salt Form B can be further characterized by having an XRPD pattern having additional peaks at 7.17, 9.67, 12.16, 20.10, and 26.83±0.2° 2θ using Cu Kα radiation. Sodium salt Form B can be further characterized by having an XRPD pattern having additional peaks at 6.47, 14.02, 14.35, 18.35, 21.00, and 23.08±0.2° 2θ using Cu Kα radiation. In some embodiments, sodium salt Form B has an XRPD pattern substantially as shown in FIG. 10A.

D-BHB crystalline sodium salt Form B may also be characterized by DSC substantially as set forth in FIG. 10B and/or by the TGA set forth in FIG. 10C. In some embodiments, D-BHB crystalline sodium salt Form B may be characterized by one or more of an XRPD substantially as depicted in FIG. 10A, DSC substantially as set forth in FIG. 10B, and TGA as set forth in FIG. 10C.

D-BHB crystalline sodium salt Form C can be characterized by XRPD pattern, obtained as set forth in the Examples, having peaks at 6.40, 7.07, 12.11, 14.08, and 17.05±0.2° 2θ using Cu Kα radiation. Sodium salt Form C can be further characterized by having an XRPD pattern having additional peaks at 19.16, 22.57, 27.79, 28.75, and 30.11±0.2° 2θ using Cu Kα radiation. Sodium salt Form C can be further characterized by having an XRPD pattern having additional peaks at 11.98, 30.76, 33.12, 33.63, and 37.08±0.2° 2θ using Cu Kα radiation. In some embodiments, sodium salt Form C has an XRPD pattern substantially as shown in FIG. 29A.

D-BHB crystalline sodium salt Form C may also be characterized by DSC substantially as set forth in FIG. 29B and/or by the TGA set forth in FIG. 29C. In some embodiments, D-BHB crystalline sodium salt Form C may be characterized by one or more of an XRPD substantially as depicted in FIG. 29A, DSC substantially as set forth in FIG. 29B, and TGA as set forth in FIG. 29C.

Alternatively or in addition, the composition for use according to the invention may comprise one or more L-arginine salts of D-BHB. For example, the composition for use according to the invention may comprise one or more of D-BHB crystalline L-arginine salt Forms A and/or B, as characterized herein.

D-BHB crystalline L-arginine salt Form A can be characterized by XRPD pattern, obtained as set forth in the Examples, having peaks at 5.28, 7.28, 9.11, 14.99, and 15.82±0.2° 2θ using Cu Kα radiation. L-arginine salt Form A can be further characterized by having an XRPD pattern having additional peaks at 17.00, 17.76, 18.32, 19.32, and 24.10±0.2° 2θ using Cu Kα radiation. L-arginine salt Form A can be further characterized by having an XRPD pattern having additional peaks at 10.81, 11.08, 11.27, 11.75, and 26.96±0.2° 2θ using Cu Kα radiation. In some embodiments, L-arginine salt Form A has an XRPD pattern substantially as shown in FIG. 14A.

D-BHB crystalline L-arginine salt Form A may also be characterized by DSC substantially as set forth in FIG. 14B and/or by the TGA set forth in FIG. 14C. In some embodiments, D-BHB crystalline L-arginine salt Form A may be characterized by one or more of an XRPD substantially as depicted in FIG. 14A, DSC substantially as set forth in FIG. 14B, and TGA as set forth in FIG. 14C.

D-BHB crystalline L-arginine salt Form B can be characterized by XRPD pattern, obtained as set forth in the Examples, having peaks at 7.30, 9.94, 11.28, 14.56, and 15.83±0.2° 2θ using Cu Kα radiation. L-arginine salt Form B can be further characterized by having an XRPD pattern having additional peaks at 17.27, 18.33, 19.91, 21.90, and 23.49±0.2° 2θ using Cu Kα radiation. L-arginine salt Form B can be further characterized by having an XRPD pattern having additional peaks at 24.12, 26.45, 26.99, 28.56, 33.56, and 34.16±0.2° 2θ using Cu Kα radiation. In some embodiments, L-arginine salt Form B has an XRPD pattern substantially as shown in FIG. 30A.

D-BHB crystalline L-arginine salt Form B may also be characterized by DSC substantially as set forth in FIG. 30B and/or by the TGA set forth in FIG. 30C). In some embodiments, D-BHB crystalline L-arginine salt Form B may be characterized by one or more of an XRPD substantially as depicted in FIG. 30A, DSC substantially as set forth in FIG. 30B, and TGA as set forth in FIG. 30C.

Alternatively, or in addition, the composition for use according to the invention may comprise one or more magnesium salts of D-BHB. For example, the composition for use according to the invention may comprise one or more of D-BHB crystalline magnesium salt Forms A and/or B, as characterized herein.

D-BHB crystalline magnesium salt Form A can be characterized by XRPD pattern, obtained as set forth in the Examples, having peaks at 5.53, 7.80, 9.61, 11.95, and 13.15±0.2° 2θ using Cu Kα radiation. Magnesium salt Form A can be further characterized by having an XRPD pattern having additional peaks at 6.11, 11.02, 11.27, 15.61, and 18.98±0.2° 2θ using Cu Kα radiation. Magnesium salt Form A can be further characterized by having an XRPD pattern having additional peaks at 19.45, 21.13, 21.88, 23.70, and 25.99±0.2° 2θ using Cu Kα radiation. In some embodiments, magnesium salt Form A has an XRPD pattern substantially as shown in FIG. 7A.

D-BHB crystalline magnesium salt Form A may also be characterized by DSC substantially as set forth in FIG. 7B and/or by the TGA set forth in FIG. 7C. In some embodiments, D-BHB crystalline magnesium salt Form A may be characterized by one or more of an XRPD substantially as depicted in FIG. 7A, DSC substantially as set forth in FIG. 7B, and TGA as set forth in FIG. 7C.

D-BHB crystalline magnesium salt Form B can be characterized by XRPD pattern, obtained as set forth in the Examples, having peaks at 6.12, 8.31, 9.60, 11.87, and 13.45±0.2° 2θ using Cu Kα radiation. Magnesium salt Form B can be further characterized by having an XRPD pattern having additional peaks at 11.49, 12.23, 14.09, 14.31, and 18.46±0.2°20 using Cu Kα radiation. Magnesium salt Form B can be further characterized by having an XRPD pattern having additional peaks at 17.01, 19.61, 23.70, 24.62, 24.94, and 29.02 and 18.46±0.2° 2θ using Cu Kα radiation. In some embodiments, magnesium salt Form B has an XRPD pattern substantially as shown in FIG. 8A.

D-BHB crystalline magnesium salt Form B may also be characterized by DSC substantially as set forth in FIG. 8B and/or by the TGA set forth in FIG. 8C. In some embodiments, D-BHB crystalline magnesium salt Form B may be characterized by one or more of an XRPD substantially as depicted in FIG. 8A, DSC substantially as set forth in FIG. 8B, and TGA as set forth in FIG. 8C.

Alternatively or in addition, the composition for use according to the invention may comprise one or more L-lysine salts of D-BHB. For example, the composition for use according to the invention may comprise D-BHB crystalline L-lysine salt Form A, as characterized herein.

D-BHB crystalline L-lysine salt Form A can be characterized by XRPD pattern, obtained as set forth in the Examples, having peaks at 6.92, 9.16, 12.62, 23.29, and 25.91±0.2° 2θ using Cu Kα radiation. L-lysine salt Form A can be further characterized by having an XRPD pattern having additional peaks at 10.21, 26.93, 27.64, 28.45, and 29.87±0.2° 2θ using Cu Kα radiation. L-lysine salt Form A can be further characterized by having an XRPD pattern having additional peaks at 17.87, 18.27, 19.73, 24.58, and 25.02±0.2° 2θ using Cu Kα radiation. In some embodiments, L-lysine salt Form A has an XRPD pattern substantially as shown in FIG. 31A.

D-BHB crystalline L-lysine salt Form A may also be characterized by DSC substantially as set forth in FIG. 31B and/or by the TGA set forth in FIG. 31C. In some embodiments, D-BHB crystalline L-lysine salt Form A may be characterized by one or more of an XRPD substantially as depicted in FIG. 31A, DSC substantially as set forth in FIG. 31B, and TGA as set forth in FIG. 31C.

Alternatively or in addition, the composition for use according to the invention may comprise one or more erbumine salts of D-BHB. For example, the composition for use according to the invention may comprise one or more of D-BHB crystalline erbumine salt Forms A and/or B, as characterized herein.

D-BHB crystalline erbumine salt Form A can be characterized by XRPD pattern, obtained as set forth in the Examples, having peaks at 10.04, 10.77, 13.69, 14.71, and 18.24±0.2° 2θ using Cu Kα radiation. Erbumine salt Form A can be further characterized by having an XRPD pattern having additional peaks at 16.08, 16.95, 17.42, 19.37, and 20.07±0.2° 2θ using Cu Kα radiation. Erbumine salt Form A can be further characterized by having an XRPD pattern having additional peaks at 22.47, 24.35, 25.60, 26.05 and 28.36±0.2° 2θ using Cu Kα radiation. In some embodiments, erbumine salt Form A has an XRPD pattern substantially as shown in FIG. 25A.

D-BHB crystalline erbumine salt Form A may also be characterized by DSC substantially as set forth in FIG. 25B and/or by the TGA set forth in FIG. 25C. In some embodiments, D-BHB crystalline erbumine salt Form A may be characterized by one or more of an XRPD substantially as depicted in FIG. 25A, DSC substantially as set forth in FIG. 25B, and TGA as set forth in FIG. 25C.

D-BHB crystalline erbumine salt Form B can be characterized by XRPD pattern, obtained as set forth in the Examples, having peaks at 12.42, 16.07, 16.69, 17.62, and 18.35±0.2° 2θ using Cu Kα radiation. Erbumine salt Form B can be further characterized by having an XRPD pattern having additional peaks at 8.82, 20.71, 22.22, 23.95, and 27.27±0.2° 2θ using Cu Kα radiation. Erbumine salt Form B can be further characterized by having an XRPD pattern having additional peaks at 19.76, 21.22, 23.49, 28.46, 31.36, and 38.21±0.2° 2θ using Cu Kα radiation. In some embodiments, D-BHB erbumine salt Form B has an XRPD pattern substantially as shown in FIG. 26B.

D-BHB crystalline erbumine salt Form B may also be characterized by DSC substantially as set forth in FIG. 26C and/or by the TGA set forth in FIG. 26D. In some embodiments, D-BHB crystalline erbumine salt Form B may be characterized by one or more of an XRPD substantially as depicted in FIG. 26B, DSC substantially as set forth in FIG. 26C, and TGA as set forth in FIG. 26D.

In a particular embodiment, the invention relates to the composition for use or the method according to the invention, wherein the composition comprises one or more salts of D-β-hydroxybutyrate (D-BHB) selected from a sodium salt, an L-arginine salt, an-L-lysine salt and/or an erbumine salt. In particular, any of the salt forms of a sodium salt, an L-arginine salt, an-L-lysine salt and/or an erbumine salt disclosed herein above may be comprised in the composition for use. In the appended experimental examples, the L-arginine salt Form B, the L-lysine salt Form A, and the erbumine salt Form B showed good crystallinity, simple thermal behavior, reasonable stoichiometry, and good reproducibility. Moreover, the sodium salt Form C was identified as the most stable polymorph of the sodium salt identified so far.

Thus, in a particular embodiment, the invention relates to the composition for use according to the invention or the method according to the invention, wherein the composition comprises one or more salts of D-β-hydroxybutyrate (D-BHB) selected from sodium salt Form C, L-arginine salt Form B, L-lysine salt Form A and/or erbumine salt Form B.

Herein, the inventors identified sodium salts and L-arginine salts of D-BHB as particularly attractive for the treatment or dietary management of LC-FAOD; for the treatment, dietary management, prevention or amelioration of one or more symptoms associated with an LC-FAOD; or for restoring or promoting normative cellular energetics. Thus, in a particular embodiment, the invention relates to the composition for use according to the invention or the method according to the invention, wherein the composition comprises a sodium salt of D-BHB (Na-D-BHB) and an L-arginine salt of D-BHB (L-Arg-D-BHB).

The sodium salt of D-BHB may be any sodium salt, including amorphous Na-D-BHB salts and crystalline Na-D-BHB salts. The sodium salt of D-BHB may exist in various stoichiometric forms, including sesqui sodium salts, monosodium salts, and disodium salts. In a particular embodiment, the sodium salt of D-β-hydroxybutyrate (Na-D-BHB) is a crystalline sodium salt, preferably a crystalline sesqui-sodium salt. Herein, three distinct crystalline salt forms of Na-D-BHB have been identified, referred to as salt forms A, B, and C, as described elsewhere herein. The Na-D-BHB may be present in hydrated form or as an anhydrate.

In a particular embodiment, the invention relates to the composition for use according to the invention or the method according to the invention, wherein the sodium salt of D-BHB (Na-D-BHB) comprises salt form C, as defined herein. In particular, salt form C of Na-D-BHB was identified herein as the most stable sodium salt of D-BHB.

Accordingly, the composition for use according to the invention preferably comprises a crystalline D-BHB sodium salt having an XRPD pattern comprising peaks at 6.40, 7.07, 12.11, 14.08, and 17.05±0.2° 2θ using Cu Kα radiation.

In certain embodiments, the crystalline D-BHB sodium salt Form C may further exhibit one or more of the following characteristics:
(a) the XRPD pattern further comprises peaks at 19.16, 22.57, 27.79, 28.75, and 30.11±0.2° 2θ using Cu Kα radiation;
(b) the XRPD pattern further comprises peaks at 11.98, 30.76, 33.12, 33.63, and 37.08±2θ using Cu Kα radiation;
(c) an XRPD pattern substantially as shown in FIG. 29A;
(d) XRPD peaks substantially as listed in Table 20;
(e) a DSC profile substantially as shown in FIG. 29B; and/or
(f) a TGA profile substantially as shown in FIG. 29C.

Preferably, at least 70%, at least 75%, at least 80%, at least 85%, at least 90%, at least 95%, at least 98% or at least 99% of the Na-D-BHB in the composition for use according to the invention is present in salt form C.

The L-arginine salt of D-BHB that is to be combined with the Na-D-BHB may be any L-arginine salt, including amorphous L-Arg-D-BHB salts and crystalline L-Arg-D-BHB salts. The L-arginine salt of D-BHB may exist in various stoichiometric forms, including mono L-arginine salts, sesqui L-arginine salts, and di L-arginine salts. In a particular embodiment, the L-arginine salt of D-β-hydroxybutyrate (L-Arg-D-BHB) is a crystalline L-arginine salt, preferably a crystalline mono L-arginine salt. Herein, two distinct crystalline salt forms of L-Arg-D-BHB have been identified, referred to as salt forms A and B, as described elsewhere herein. The L-Arg-D-BHB may be present in hydrated form or as an anhydrate.

In a particular embodiment, the invention relates to the composition for use according to the invention or the method according to the invention, wherein the L-arginine salt of D-BHB (L-Arg-D-BHB) comprises salt form B, as defined herein. In particular, the D-BHB L-arginine salt Form B was identified herein as the most stable of all tested salt forms (see Example 11). Accordingly, the composition for use according to the invention preferably comprises a crystalline D-BHB L-arginine salt having an XRPD pattern comprising peaks at 7.30, 9.94, 11.28, 14.56, and 15.83±0.2° 2θ using Cu Kα radiation.

In certain embodiments, the crystalline D-BHB L-arginine salt Form B may further exhibit one or more of the following characteristics:
(a) the XRPD pattern further comprises peaks at 17.27, 18.33, 19.91, 21.90, and 23.49±0.2° 2θ using Cu Kα radiation;
(b) the XRPD pattern further comprises peaks at 24.12, 26.45, 26.99, 28.56, 33.56, and 34.16±0.2° 2θ using Cu Kα radiation;
(c) an XRPD pattern substantially as shown in FIG. 30A;
(d) XRPD peaks substantially as listed in Table 25;
(e) a DSC profile substantially as shown in FIG. 30B; and/or
(f) a TGA profile substantially as shown in FIG. 30C.

Preferably, at least 70%, at least 75%, at least 80%, at least 85%, at least 90%, at least 95%, at least 98% or at least 99% of the L-Arg-D-BHB in the composition for use according to the invention is present in salt form B.

The sodium salt of D-BHB (Na-D-BHB) and the L-arginine salt of D-BHB (L-Arg-D-BHB) may be mixed at a defined molar ratio. In a particular embodiment, the invention relates to the composition for use according to the invention or the method according to the invention, wherein the molar ratio between Na-D-BHB and L-Arg-D-BHB is between 1:10 and 10:1, preferably between 1:5 and 5:1, more preferably between 1:2 and 2:1, most preferably wherein the molar ratio between Na-D-BHB and L-Arg-D-BHB is about 1:1.

In certain embodiments, the molar ratio between Na-D-BHB and L-Arg-D-BHB in the composition for use may range from about 1:10 to about 10:1, about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, about 1:1.5 to about 1.5:1, and about 1:1. In a particularly preferred embodiment, the molar ratio between Na-D-BHB and L-Arg-D-BHB in the composition for use is about 1:1.

In certain embodiments, the composition for use according to the invention comprises crystalline sodium salt Form C of D-BHB and crystalline L-arginine salt Form B of D-BHB at a molar ratio ranging from about 1:10 to about 10:1, about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, about 1:1.5 to about 1.5:1, and about 1:1. In a particularly preferred embodiment, the composition for use according to the invention comprises crystalline sodium salt Form C of D-BHB and crystalline L-arginine salt Form B of D-BHB at a molar ratio of about 1:1.

The term “molar ratio” refers to the ratio of the number of moles of one component to the number of moles of another component in a mixture or compound. It is a quantitative expression used to describe the relative proportions of different substances in a chemical composition. For example, in the context of the salt forms of D-BHB, a composition might include a sodium salt of D-β-hydroxybutyrate (Na-D-BHB) and an L-arginine salt of D-β-hydroxybutyrate (L-Arg-D-BHB). If the molar ratio between Na-D-BHB and L-Arg-D-BHB is specified as 1:1, this indicates that there is one mole of Na-D-BHB for every one mole of L-Arg-D-BHB in the composition.

In a particular embodiment, the invention relates to the composition for use according to the invention or the method according to the invention, wherein the composition is free or essentially free of L-β-hydroxybutyrate (L-BHB). That is, the D-BHB and/or the D-BHB crystalline salts comprised in the composition are greater than about 95% enantiomerically pure, greater than about 98% enantiomerically pure, or greater than about 99% enantiomerically pure. “Enantiomerically pure” as used herein refers to the percentage of one enantiomer versus the other enantiomer, e.g. the percentage of D-BHD versus L-BHB in a preparation of D-BHB. It is understood that enantiomeric pairs (i.e. the R and S structural configuration of a compound with a chiral atom using absolute configuration convention, or D or L structural configuration of a compound with a chiral atom using the convention in relation to L- and D-glyceraldehyde as represented in Fischer projections) may be produced as a mixture having different percentages of the respective enantiomers. In the context of the invention, D-BHB as starting material to generate the composition according to the invention is greater than about 95% D-BHB and has less than about 5% L-DHB, greater than about 98% D-BHB and less than about 2% L-BHB, greater than about 99% D-BHB and less than about 1% L-BHB, or greater than about 99.5% D-BHB and less than about 0.5% L-BHB. A crystalline D-BHB having greater than 95% enantiomeric purity is understood as being comprised of greater than 95% D-BHB and less than 5% L-BHB. A crystalline D-BHB having greater than 98% enantiomeric purity is understood as being comprised of greater than 98% D-BHB and less than 2% L-BHB. A crystalline D-BHB having greater than 99% enantiomeric purity is understood as being comprised of greater than 99% D-BHB and less than 1% L-BHB. A crystalline D-BHB having greater than 99.5% enantiomeric purity is understood as being comprised of greater than 99.5% D-BHB and less than 0.5% L-BHB.

Preferably, the D-BHB used as starting material for the composition according to the invention is produced enzymatically. Enzymatic production methods are advantageous because they yield enantiomerically pure D-BHB.

For example, salts of D-BHB may be synthesized as follows: Ethyl Acetoacetate may be enzymatically converted to Ethyl (R)-hydroxybutanoate using a ketone reductase (step 1) (Moore et al, 2007, Accounts of Chemical Research, 40 (12), 1412-1419) leading after hydrolysis to the formation of enantiomerically pure D-BHB (Step 2) which can be used to produce either a Sodium or L-Arginine salt (step 3).

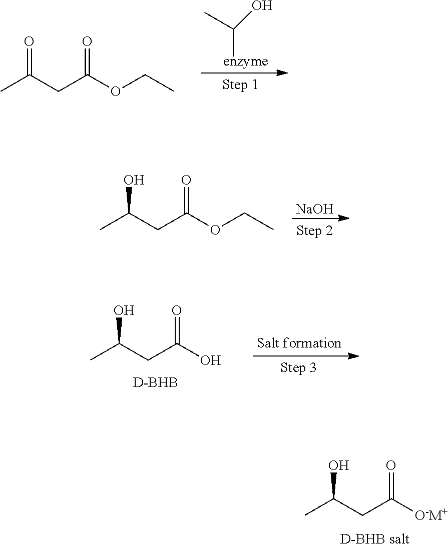

Accordingly, a composition is considered essentially free of L-BHB if the D-BHB and/or the D-BHB crystalline salts comprised in the composition are greater than about 95% enantiomerically pure, greater than about 98% enantiomerically pure, greater than about 99% enantiomerically pure, or greater than about 99.5% enantiomerically pure.

The composition for use according to the invention may be any type of composition, preferably any type of composition that is suitable for administration to a mammalian subject.

In certain embodiments, the composition according to the invention is a pharmaceutical composition comprising a pharmaceutically acceptable carrier. A “pharmaceutical composition” is a formulation that includes one or more active ingredients, i.e., D-BHB, combined with carriers or excipients that are suitable for administration to a mammalian subject. These carriers or excipients are typically inert substances that facilitate the delivery, absorption, and/or stability of the active ingredients. The pharmaceutical composition is designed to provide a therapeutic effect and is manufactured following stringent regulatory standards to ensure safety, efficacy, and quality.

Excipients that may be used in the composition according to the invention include, without limitation, diluents, fillers, flow aids/glidants, lubricants and/or flavoring agents.

Diluents are inert substances added to a formulation to increase its volume or weight, making it easier to handle and administer. Common diluents include lactose, cellulose, and starch.

Fillers are substances used to bulk up formulations, especially in tablets and capsules, to achieve the desired size and shape. Fillers can also help improve the consistency and stability of the formulation. Examples include microcrystalline cellulose and mannitol.

Flow Aids/Glidants are agents added to powders to improve their flow properties, preventing clumping and ensuring uniformity during the manufacturing process. Common glidants include silicon dioxide and talc.

Lubricants are substances that reduce friction between particles and machinery during the manufacturing process, facilitating the compression of tablets and the filling of capsules. Examples include magnesium stearate and stearic acid.

Flavoring Agents are compounds added to formulations to improve their taste, making them more palatable for patients. Flavoring agents can be natural or artificial and are commonly used in oral medications.

Pharmaceutical compositions comprising D-BHB are particularly well suited for the prevention and/or treatment of LC-FAOD.

In a particular embodiment, the invention relates to the composition according to the invention, wherein the composition is a nutritional composition. The term “nutritional composition” as used herein refers to a formulation designed to provide essential nutrients, such as vitamins, minerals, amino acids, and other dietary components, to support the overall health and well-being of a mammalian subject. Nutritional composition comprises, without limitation, food products, food supplements, functional beverage products, nutritional supplements, dietary supplements, over-the-counter (OCT) supplements, medical foods, Enteral Formula for Special Medical Use, Food for Specified Health Uses, Food for Special Medical Purposes (FSMP), Food for Special Dietary Use (FSDU), and a Medical Foods.

Nutritional compositions are intended to complement the diet, address specific nutritional deficiencies, enhance physical performance, or support specific health conditions. They are formulated to be safe, palatable, and effective in delivering the intended nutritional benefits.

The nutritional composition comprising D-BHB may be, or may be comprised in, a nutritional product. The term nutritional product typically refers to the final, market-ready item that comprises the nutritional composition according to the invention. In certain embodiments, nutritional composition comprising D-BHB may be, or may be comprised in, a complete or incomplete nutritional product.

As used herein, the term “incomplete nutritional product” refers to preferably nutritional products that do not contain sufficient levels of macronutrients (protein, fats and carbohydrates) or micronutrients to be sufficient to be a sole source of nutrition for the subject which consumes the nutritional product or the subject to which the nutritional product is being administered. As used herein, the “complete nutritional product” refers to nutritional products that contains sufficient levels of macronutrients (protein, fats and carbohydrates) or micronutrients to be sufficient to be a sole source of nutrition for the subject which consumes the nutritional product or the subject to which the nutritional product is being administered.

That is, the nutritional composition according to the invention, or the nutritional product comprising the same, may comprise one or more macronutrients and/or micronutrients. Macronutrients that may be present include proteins, carbohydrates, and fats, which are essential for providing energy and supporting bodily functions. Micronutrients that may be included are vitamins (such as vitamins A, C, D, E, and K, and B-complex vitamins) and minerals (such as calcium, magnesium, potassium, iron, zinc, and selenium), which are crucial for various metabolic processes and maintaining overall health. Additionally, the nutritional composition or product may contain free amino acids, fibers, nucleic acids, nucleotides, fruits, vegetables, emulsifiers, botanicals, acidifying agents, alkalinizing agents, buffering agents, and other ingredients that contribute to its nutritional and functional properties.

In certain embodiments, the nutritional composition is a food for special medical purpose (FSMP) and preferably comprises at least one macronutrient selected from the group consisting of a protein, a carbohydrate, a lipid, and combinations thereof. The term “food for special medical purpose (FSMP),” as used herein, refers to a category of foods that are specially formulated and intended for the dietary management of individuals with specific medical conditions, diseases, or disorders. FSMPs are designed to meet the distinctive nutritional requirements that cannot be achieved by normal diet alone. They are used under medical supervision and are tailored to support the nutritional needs of patients with conditions such as metabolic disorders, malabsorption syndromes, or chronic illnesses.

Nutritional compositions comprising D-BHB are particularly well suited for the dietary management of LC-FAOD.

In certain embodiments, the composition, in particular the pharmaceutical or nutritional composition, for use according to the invention comprises Na-D-BHB and L-Arg-D-BHB, preferably at a molar ratio of about 1:1, more preferably wherein Na-D-BHB is present in salt form C and L-Arg-D-BHB is present in salt form B, and further comprises one or more of microcrystalline cellulose, lactose (anhydrous), mannitol, magnesium stearate, and/or stearic acid, at a suitable concentration.

The composition for use according to the invention, including the pharmaceutical or nutritional composition according to the invention, may be formulated in any suitable way. That is, the composition may be prepared in various forms such as tablets, capsules, powders, granules, liquids, suspensions, emulsions, or reconstitutable powders. These formulations can be designed for different routes of administration, including oral, parenteral, transdermal, or inhalation, depending on the specific needs and preferences of the patient. The choice of formulation will depend on factors such as the stability of the active ingredients, the desired release profile, ease of administration, and patient compliance.

In certain embodiments, the composition for use according to the invention, including the pharmaceutical or nutritional composition according to the invention, is formulated as a powder, preferably a reconstitutable powder. The term “powder” refers to a dry, particulate form of the composition that can be easily measured and administered. Powders can be consumed directly or mixed with food or beverages. The term “reconstitutable powder” refers to a specific type of powder that is designed to be mixed with a liquid, such as water, to form a solution or suspension that is ready for consumption. Reconstitutable powders offer the advantage of convenient storage and transport, as well as the ability to customize the concentration and volume of the final product to meet individual patient needs.

The powder, in particular the reconstitutable powder, may be packaged in any suitable way. It can be provided in bulk containers, such as jars or canisters, which allow for multiple servings to be measured out as needed. Alternatively, it can be packaged in single-dose sachets or packets, which offer the convenience of pre-measured, individual servings that are easy to use and ensure accurate dosing.

The composition for use according to the invention, in particular the pharmaceutical or nutritional composition according to the invention, may be formulated for a specific route of administration. This includes, but is not limited to, oral administration, where the composition can be ingested in the form of tablets, capsules, powders, or liquids; parenteral administration, which involves injection or infusion of solutions or suspensions directly into the bloodstream or tissues; transdermal administration, where the composition is delivered through the skin via patches or gels; and inhalation, where the composition is administered as an aerosol or dry powder for respiratory uptake.

In a particular embodiment, the composition for use according to the invention, in particular the pharmaceutical or nutritional composition for use according to the invention, is formulated for enteral administration. Compositions that are formulated for enteral administration are designed to be delivered into the gastrointestinal tract. This can be achieved through oral ingestion (eating and drinking) or via feeding tubes, such as nasogastric, gastrostomy, or jejunostomy tubes. Enteral administration via feeding tubes may be beneficial for patients who are unable to consume food orally or require precise nutritional support.

In certain embodiments, the composition for use according to the invention, in particular the pharmaceutical or nutritional composition for use according to the invention, is formulated as a reconstitutable powder for enteral administration. This reconstitutable powder can be mixed with a suitable liquid, such as water, to create a solution or suspension that can be ingested orally or administered via a feeding tube.

In certain embodiments, the composition for use according to the invention, in particular the pharmaceutical or nutritional composition according to the invention, is formulated as a reconstitutable powder for gastric administration, in particular nasogastric administration or administration by gastrostomy. Again, the reconstitutable powder is mixed with a suitable liquid, such as water, to create a solution or suspension that can be administered gastrically via a feeding tube.

Compositions suitable for gastric administration are particularly suitable for pediatric subjects, since infants and young children often have difficulty swallowing or are unwilling to swallow sufficient amounts of a composition.

Alternatively, the composition for use according to the invention, in particular the pharmaceutical composition for use according to the invention, may be formulated for parenteral administration. Parenteral administration refers to delivering the composition by routes other than the digestive tract, typically through injection or infusion. This can include intravenous (IV), intramuscular (IM), subcutaneous (SC), or intraperitoneal (IP) routes. Formulating the composition for parenteral administration, in particular intravenous administration, can be advantageous for patients who require rapid onset of action, have difficulty with oral administration, or need precise control over dosage and bioavailability. The parenteral formulation may include appropriate excipients, stabilizers, and buffers to ensure the stability and efficacy of the active ingredients, such as D-BHB and its salts. Additionally, the formulation must be sterile and pyrogen-free to meet the stringent requirements for injectable products.

Accordingly, in a particular embodiment, the invention relates to the composition for use according to the invention or the method according to the invention, wherein the composition is administered enterally, preferably orally or gastrically, including nasogastrically or by gastrostomy.

In a particular embodiment, the invention relates to the composition for use according to the invention or the method according to the invention, wherein the composition is administered parentally, preferably intravenously.

The composition for use according to the invention may be administered in a pharmaceutically-effective amount to achieve the desired effect, i.e., to restore or promote normative cellular energetics and/or to treat or prevent symptoms associated with LC-FAOD. As used herein, the term “pharmaceutically-effective amount” means an amount sufficient to prevent, treat, or manage one or more symptoms of LC-FAOD.

It is preferred herein that an active dose of 25 to 1000 mg D-BHB per kg body weight per day (25 to 1000 mg/kg/d) is administered to a subject. The term “active dose” refers to the amount of the active ingredient, D-β-hydroxybutyrate (D-BHB), that is administered to a subject to achieve the desired effect. For example, for a subject weighing 70 kg, the active dose would range from 1,750 mg (25 mg/kg×70 kg) to 70,000 mg (1000 mg/kg×70 kg) of D-BHB per day.

In certain embodiments, the composition for use according to the invention is administered, without limitation, at an active dose of 25 mg D-BHB per kg per day, 35 mg D-BHB per kg per day, 50 mg D-BHB per kg per day, 75 mg D-BHB per kg per day, 100 mg D-BHB per kg per day, 150 mg D-BHB per kg per day, 200 mg D-BHB per kg per day, 250 mg D-BHB per kg per day, 300 mg D-BHB per kg per day, 400 mg D-BHB per kg per day, 500 mg D-BHB per kg per day, 600 mg D-BHB per kg per day, 700 mg D-BHB per kg per day, 800 mg D-BHB per kg per day, 900 mg D-BHB per kg per day, or 1000 mg D-BHB per kg per day.

Alternatively, the composition for use according to the invention may be administered to provide the equivalent of 5 g D-BHB in a single dose or up to 5 g D-BHB in a single dose, 6 g D-BHB in a single dose or up to 6 g D-BHB in a single dose, 7 g D-BHB in a single dose or up to 7 g D-BHB in a single dose, 8 g D-BHB in a single dose or up to 8 g D-BHB in a single dose, 9 g D-BHB in a single dose or up to 9 g D-BHB in a single dose, 10 g D-BHB in a single dose or up to 10 g D-BHB in a single dose, 11 g D-BHB in a single dose or up to 11 g D-BHB in a single dose, 12 g D-BHB in a single dose or up to 12 g D-BHB in a single dose, 13 g D-BHB in a single dose or up to 13 g D-BHB in a single dose, 14 g D-BHB in a single dose or up to 14 g D-BHB in a single dose, 15 g D-BHB in a single dose or up to 15 g D-BHB in a single dose, 16 g D-BHB in a single dose or up to 16 g D-BHB in a single dose, 17 g D-BHB in a single dose or up to 17 g D-BHB in a single dose, 18 g D-BHB in a single dose or up to 18 g D-BHB in a single dose, 19 g D-BHB in a single dose or up to 19 g D-BHB in a single dose, or 20 g D-BHB in a single dose or up to 20 g D-BHB in a single dose.

The composition for use according to the invention may be administered to provide the equivalent of 60 g D-BHB per day or up to 60 g D-BHB per day, 45 g D-BHB per day or up to 45 g D-BHB per day, 48 g D-BHB per day or up to 48 g D-BHB per day, 40 g D-BHB per day or up to 40 g D-BHB per day, 36 g D-BHB per day or up to 36 g D-BHB per day, 30 g D-BHB per day or up to 30 g D-BHB per day, 20 g D-BHB per day or up to 20 g D-BHB per day, 10 g D-BHB per day or up to 10 g D-BHB per day.

The composition for use according to the invention may be administered in multiple dosing regimens to achieve the desired effect. In certain embodiments, the composition may be administered as a single dose per day. Alternatively, the composition may be administered in two doses per day, three doses per day, or four doses per day, depending on the specific needs of the subject and the severity of the condition being treated. However, it is preferred herein that multiple doses of D-BHB are administered per day to provide a constant supply of energy.

For convenience, the composition for use according to the invention may be packaged as single doses. These single-dose packages can ensure accurate dosing and ease of administration, particularly for patients who require precise amounts of the composition. The single doses can be provided in various forms, such as single-dose sachets or packets.

Such a single-dose package may comprise an amount of the composition that corresponds to about 5 g, about 10 g, about 12 g, about 15 g, about 20 g, about 25 g, about 30 g, about 36 g, about 40 g, about 45 g, about 48 g, about 50 g, about 55 g, or about 60 g of D-BHB. It is understood by those skilled in the art that the total weight of the composition will depend on the specific form of D-BHB used and the quantity of other ingredients included in the composition.

It is preferred that the composition for use according to the invention be administered daily, or even multiple times per day, such as two, three, or four times per day, to provide a constant supply of D-BHB. Furthermore, it is recommended that the composition be administered continuously, i.e., for an indefinite number of days. This is particularly important for the treatment LC-FAOD, a genetic disease that cannot be cured, where a constant supply of an alternative energy source is required to prevent, ameliorate or manage the symptoms effectively. Thus, in a particular embodiment, the invention relates to the composition for use according to the invention or the method according to the invention, wherein the composition is administered daily, preferably for an indefinite number of days.

The dose of D-BHB that is administered to a subject may be adjusted during the course of the treatment to achieve optimal therapeutic outcomes. During the course of treatment, the dose may be increased or adjusted based on the patient's response to the therapy. For example, if an initial dose is well-tolerated and the patient shows improvement, the dose may be gradually increased to enhance therapeutic efficacy. Conversely, if adverse effects are observed, the dose may be reduced or adjusted accordingly. This flexible dosing regimen allows for personalized treatment plans that can be tailored to meet the unique needs of each patient, ensuring the best possible outcomes in managing the symptoms of LC-FAODs. Accordingly, in a particular embodiment, the invention relates to the composition for use according to the invention or the method according to the invention, wherein the active dose is adjusted, preferably increased, during the prevention, treatment or dietary management.

The composition for use according to the invention may be administered alone or in combination with one or more additional therapeutic agents. These additional agents can be selected based on the specific needs of the patient and the nature of the condition being treated. For example, in the case of LC-FAOD, the composition may be combined with other medications that support mitochondrial function, enhance energy metabolism, or alleviate specific symptoms associated with the disease.

In certain embodiments, the composition for use according to the invention may be administered in combination with medium chain triglycerides. The term “medium chain triglycerides” as used herein refers to triglycerides that contain fatty acids with a chain length of 6 to 12 carbon atoms. These medium chain triglycerides can provide a quick and efficient source of energy that can be metabolized independently of the defective long-chain fatty acid oxidation pathways. This helps to maintain energy balance and reduce the risk of metabolic crises in affected individuals. Accordingly, in a particular embodiment, the invention relates to the composition for use according to the invention or the method according to the invention, wherein the composition is co-administered with a medium-chain triglyceride, in particular wherein the medium-chain triglyceride is triheptanoin. In certain embodiments, the medium-chain triglyceride is administered in the form of an oil, i.e., an MCT oil.

In certain embodiments, the composition for use according to the invention may further comprise carnitine. In certain embodiments, the composition according to the invention, such as the pharmaceutical or nutritional composition according to the invention, may comprise carnitine as a further ingredient.

In certain embodiments, the composition for use according to the invention may be administered as part of a high calorie carbohydrate drink. In certain embodiments, the composition for use according to the invention may be a nutritional composition that is rich in carbohydrates, such as an FSMP. Thus, without limitation, the nutritional composition may be an FSMP comprising D-BHB and, as a further ingredient, carbohydrates and/or carnitine.

Further provided herein are compositions comprising salts of D-BHB. That is, in a particular embodiment, the invention relates to a composition comprising two or more salts of D-β-hydroxybutyrate (D-BHB), wherein at least one of the two or more salts is selected from a sodium salt, an L-arginine salt, an-L-lysine salt and/or an erbumine salt. Sodium salts, L-arginine salts, L-lysine salts and erbumine salts of D-BHB have been identified herein as particularly stable and are thus preferred for the use in the composition according to the invention. These salts may be combined with each other in any possible combination. Moreover, each of the sodium salt, L-arginine salt, L-lysine salt and/or erbumine salt may be combined with one or more additional forms of D-BHB, including other salt forms of D-BHB.

In a particular embodiment, the invention relates to the composition according to the invention, wherein the composition comprises at least one amino acid salt of D-BHB and one mineral salt of D-BHB, preferably wherein the mineral salt of D-BHB is a sodium salt of D-BHB (Na-D-BHB). That is, the composition may comprise as a first salt an amino acid salt of D-BHB and as a second salt a mineral salt of D-BHB.

Compositions comprising a mixture of mineral salts of D-BHB and amino acid salts of D-BHB offer significant advantages over those containing exclusively mineral salts of D-BHB. Specifically, the intake of large doses of minerals such as sodium or magnesium has been associated with adverse effects and toxicities. By replacing a portion of the mineral salts of D-BHB with amino acid salts of D-BHB, the risk of these toxicities and adverse events is reduced. This approach is particularly beneficial when the composition is administered daily over an extended period, as it helps to avoid the complications associated with high mineral intake.

Amino acid salts of D-BHB may comprise, without limitation, arginine salts, lysine salts, leucine salts, isoleucine salts, histidine salts, ornithine salts, citrulline salts, glutamine salts, and creatine salt. Preferably, the amino acid salt is a salt of an L-amino acid. Mineral salts of D-BHB may comprise, without limitation, lithium salts, sodium salts, potassium salts, magnesium salts, calcium salts, zinc salts, iron salts (as iron II and/or iron III), chromium salts, manganese salts, cobalt salts, copper salts, molybdenum salts, and selenium salts.

However, in preferred embodiments, the mineral salt of D-BHB is a sodium salt of D-BHB (Na-D-BHB), such as any of the sodium salt forms disclosed herein, and the sodium salt of D-BHB is combined with an amino acid salt of D-BHB. In certain embodiments, the sodium salt of D-BHB is combined with an amino acid salt of D-BHB. In certain embodiments, the sodium salt of D-BHB is combined with an L-arginine salt of D-BHB and/or an L-lysine salt of D-BHB.

In a particular embodiment, the invention relates to the composition according to the invention, wherein the composition comprises a sodium salt of D-BHB (Na-D-BHB) and an L-arginine salt of D-BHB (L-Arg-D-BHB).

The sodium salt of D-BHB may be any sodium salt, including amorphous Na-D-BHB salts and crystalline Na-D-BHB salts. The sodium salt of D-BHB may exist in various stoichiometric forms, Including sesqui sodium salts, monosodium salts, and disodium salts. In a particular embodiment, the sodium salt of D-β-hydroxybutyrate (Na-D-BHB) is a crystalline sodium salt, preferably a crystalline sesqui-sodium salt. Herein, three distinct crystalline salt forms of Na-D-BHB have been identified, referred to as salt forms A, B, and C, as described elsewhere herein. The Na-D-BHB may be present in hydrated form or as an anhydrate.

Besides being the most stable salt identified in the appended examples, L-arginine salts of D-BHB offer additional advantages. L-arginine is a precursor of nitric oxide, which is associated with various beneficial health effects. For example, Ikawa et al. (Curr Opin Clin Nutr Metab Care, 2019, 23(1): 17-22) reported that systematic administration of oral and intravenous L-arginine is therapeutically beneficial and clinically useful for patients with mitochondrial myopathy, encephalopathy, lactic acidosis, and stroke-like episodes (MELAS). In an earlier study by Arakawa et al. (Circ J. 2010, 74(12): 2702-11), it was further reported that L-arginine can enhance TCA-cycle metabolism and can be used as a treatment for patients with mitochondrial cardiomyopathy. Notably, cardiomyopathy is one of the symptoms of LC-FAOD, a disease linked to reduced TCA-cycle metabolism. Moreover, L-arginine has been reported to directly impact the metabolic fitness and survival capacity of T cells (Cell, 2016, 167(3): 829-842.e13). Thus, without wishing to be bound by theory, including L-arginine in the composition according to the invention, preferably in the form of L-arginine-D-BHB salts, may further improve mitochondrial function and help overcome or at least mitigate the LC-FAOD-associated energy deficit.

In a particular embodiment, the invention relates to the composition according to the invention, wherein the sodium salt of D-BHB (Na-D-BHB) comprises salt form C, as defined herein. In particular, salt form C of Na-D-BHB was identified herein as the most stable sodium salt of D-BHB. Accordingly, the composition for use according to the invention preferably comprises a crystalline D-BHB sodium salt having an XRPD pattern comprising peaks at 6.40, 7.07, 12.11, 14.08, and 17.05±0.2° 2θ using Cu Kα radiation.

In certain embodiments, the crystalline D-BHB sodium salt Form C may further exhibit one or more of the following characteristics:
(a) the XRPD pattern further comprises peaks at 19.16, 22.57, 27.79, 28.75, and 30.11±0.2° 2θ using Cu Kα radiation;
(b) the XRPD pattern further comprises peaks at 11.98, 30.76, 33.12, 33.63, and 37.08±0.2° 2θ using Cu Kα radiation;
(c) an XRPD pattern substantially as shown in FIG. 29A;
(d) XRPD peaks substantially as listed in Table 20;
(e) a DSC profile substantially as shown in FIG. 29B; and/or
(f) a TGA profile substantially as shown in FIG. 29C.

Preferably, at least 70%, at least 75%, at least 80%, at least 85%, at least 90%, at least 95%, at least 98% or at least 99% of the Na-D-BHB in the composition according to the invention is present in salt form C. In certain embodiments, all of the Na-D-BHB in the composition according to the invention is present in salt form C.

The L-arginine salt of D-BHB may be any L-arginine salt, including amorphous L-Arg-D-BHB salts and crystalline L-Arg-D-BHB salts. The L-arginine salt of D-BHB may exist in various stoichiometric forms, including mono L-arginine salts, sesqui L-arginine salts, and di L-arginine salts. In a particular embodiment, the L-arginine salt of D-β-hydroxybutyrate (L-Arg-D-BHB) is a crystalline L-arginine salt, preferably a crystalline mono L-arginine salt. Herein, two distinct crystalline salt forms of L-Arg-D-BHB have been identified, referred to as salt forms A and B, as described elsewhere herein. The L-Arg-D-BHB may be present in hydrated form or as an anhydrate.

In a particular embodiment, the invention relates to the composition according to the invention, wherein the L-arginine salt of D-BHB (L-Arg-D-BHB) comprises salt form B, as defined herein.

D-BHB L-arginine salt Form B was identified herein as the most stable of all tested salt forms (see Example 11).

Accordingly, the composition for use according to the invention preferably comprises a crystalline D-BHB L-arginine salt having an XRPD pattern comprising peaks at 7.30, 9.94, 11.28, 14.56, and 15.83±0.2° 2θ using Cu Kα radiation.

In certain embodiments, the crystalline D-BHB L-arginine salt Form B may further exhibit one or more of the following characteristics:
(a) the XRPD pattern further comprises peaks at 17.27, 18.33, 19.91, 21.90, and 23.49±0.2° 2θ using Cu Kα radiation;
(b) the XRPD pattern further comprises peaks at 24.12, 26.45, 26.99, 28.56, 33.56, and 34.16±0.2° 2θ using Cu Kα radiation;
(c) an XRPD pattern substantially as shown in FIG. 30A;
(d) XRPD peaks substantially as listed in Table 25;
(e) a DSC profile substantially as shown in FIG. 30B; and/or
(f) a TGA profile substantially as shown in FIG. 30C.

Preferably, at least 70%, at least 75%, at least 80%, at least 85%, at least 90%, at least 95%, at least 98% or at least 99% of the L-Arg-D-BHB in the composition according to the invention is present in salt form B. In certain embodiments, all of the L-Arg-D-BHB in the composition according to the invention is present in salt form B.

The sodium salt of D-BHB and the L-arginine salt of D-BHB are preferably mixed at a defined molar ratio. Thus, in a particular embodiment, the invention relates to the composition according to the invention, wherein the molar ratio between Na-D-BHB and L-Arg-D-BHB is between 1:10 and 10:1, preferably between 1:5 and 5:1, more preferably between 1:2 and 2:1, most preferably wherein the molar ratio between Na-D-BHB and L-Arg-D-BHB is about 1:1. In certain embodiments, the molar ratio between Na-D-BHB and L-Arg-D-BHB in the composition according to the invention may range from about 1:10 to about 10:1, about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, about 1:1.5 to about 1.5:1, and about 1:1. In a particularly preferred embodiment, the molar ratio between Na-D-BHB and L-Arg-D-BHB in the composition according to the invention is about 1:1.

In particularly preferred embodiments, the composition according to the invention comprises crystalline sodium salt Form C of D-BHB and crystalline L-arginine salt Form B of D-BHB at a defined molar ratio ranging from about 1:10 to about 10:1, about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, about 1:1.5 to about 1.5:1, and about 1:1. In a particularly preferred embodiment, the composition according to the invention comprises crystalline sodium salt Form C of D-BHB and crystalline L-arginine salt Form B of D-BHB at a molar ratio of about 1:1.

In another preferred embodiment, the composition according to the invention comprises two or more salts of D-BHB selected from a sodium salt, a calcium salt, and/or a magnesium salt. That is the composition according to the invention may comprise a mixture of a sodium salt and a calcium salt of D-BHB, a mixture of a sodium salt and a magnesium salt of D-BHB, or a mixture of a magnesium salt and a calcium salt of D-BHB. However, it is preferred herein that the composition according to the invention comprises all three of a sodium salt, a calcium salt, and a magnesium salt of D-BHB. The sodium salt, the calcium salt and/or the magnesium salts of D-BHB may be amorphous salts or crystalline salts, including any of the crystalline salt forms provided herein.

The three salts may be present in the composition according to the invention in any suitable molar ratio. However, it is preferred that the calcium salt and the magnesium salt of D-BHB are present in a higher molar ratio than the sodium salt. Specifically, the molar ratio between (i) the sodium salt and (ii) the calcium salt and/or the magnesium salt may range from 1:1 to 1:10, such as 1:1, 1:2, 1:3, 1:4, 1:5, 1:6, 1:7, 1:8, 1:9 or 1:10. Preferably, the molar ratio between the sodium salt, the calcium salt, and the magnesium salt is about 1:2:2 (Na:Ca:Mg). That is, a molar ratio of 1:2:2 means that for every one mole of the sodium salt of D-BHB, there are two moles of the calcium salt of D-BHB and two moles of the magnesium salt of D-BHB present in the composition.

The composition comprising the two or more salts of D-BHB selected from a sodium salt, a calcium salt, and/or a magnesium salt may further comprise D-BHB in its free form. The free form of D-BHB refers to the pure, unbound molecule of D-BHB without any additional ions, chemical groups, or modifications attached. In its free form, D-BHB exists as a simple, protonated organic acid. The free form of D-BHB can also be present as an anhydrate, meaning it is in a dehydrated state and free of water content. Anhydrous D-BHB can be reconstituted with water or other suitable solvents when needed for administration, where precise dosing and stability are important.

Including the free form of D-BHB in the composition is advantageous, as it helps to reduce the concentration of minerals in the formulation. As explained elsewhere herein, high doses of minerals, such as magnesium or sodium, can be toxic and/or result in adverse events, particularly when the composition is administered over long periods of time. Therefore, incorporating D-BHB in its free form can mitigate the risk of toxicities or adverse events associated with excessive mineral intake.

The salts of D-BHB, in particular the sodium salt, the calcium salt, and the magnesium salt and the free form of D-BHB may be mixed at any suitable ratio. In a particular embodiment, the invention relates to the composition according to the invention, wherein the molar ratio between the D-BHB salts and D-BHB in its free form ranges from 1:10 to 10:1, preferably 1:5 to 5:1, more preferably 1:2 to 2:1, even more preferably 1:1.5 to 1.5:1, most preferably about 1:1.

In a particular embodiment, the composition according to the invention comprises D-BHB salts and D-BHB in its free form in a molar ratio of about 1:1, wherein the salts of D-BHB consist of a sodium salt, a calcium salt, and a magnesium salt of D-BHB in a molar ratio of about 1:2:2.

The composition according to the invention, such as any of the compositions disclosed herein, may further comprise nicotinamide riboside. In particular, composition comprising salts of D-BHB, preferably the sodium salt, the calcium salt, and/or the magnesium salt of D-BHB and, optionally, the D-BHB in free form, may further comprise nicotinamide riboside. Thus, in a particular embodiment, the invention relates to the composition according to the invention, wherein the composition further comprises nicotinamide riboside. Nicotinamide Riboside (NR) is a naturally occurring form of vitamin B3 (niacin) and a precursor to nicotinamide adenine dinucleotide (NAD+), a vital coenzyme involved in numerous metabolic processes within the body. NR is a pyridine-nucleoside form of vitamin B3, which means it consists of a nicotinamide (a form of niacin) molecule attached to a ribose sugar. The nicotinamide riboside may be present in the composition at a concentration of about 0.5% (w/w) to about 10% (w/w), preferably about 1% (w/w) to about 5% (w/w), more preferably about 2% (w/w) to about 4.5% (w/w).

The composition according to the invention, in particular the compositions disclosed herein above, are preferably free of L-β-hydroxybutyrate (L-BHB). That is, in a particular embodiment, the invention relates to the composition according to the invention, wherein the composition is free or essentially free of L-β-hydroxybutyrate (L-BHB), as defined elsewhere herein.

The composition according to the invention may be formulated as defined elsewhere herein. Specifically, the composition may be prepared in any suitable form. However, it is preferred that the composition is formulated as a powder, and more preferably as a reconstitutable powder.

Moreover, the composition according to the invention may be any type of composition. Preferably, the composition is suitable for administration to a mammalian subject, more preferably a human subject. In certain embodiments, the composition may be a nutritional composition as defined elsewhere herein. Specifically, the nutritional composition may be a food for special medical purposes (FSMP), preferably comprising at least one macronutrient selected from the group consisting of a protein, a carbohydrate, a lipid, and combinations thereof. In a particularly preferred embodiment, the FSMP comprises a carbohydrate and can be reconstituted to obtain a high-calorie carbohydrate drink comprising D-BHB.

Alternatively, the composition may be a pharmaceutical composition further comprising a pharmaceutically acceptable carrier, as defined elsewhere herein.

The composition according to the invention, in particular the pharmaceutical composition according to the invention, may further comprise a diluent, a filler, a flow aid/glidant, a lubricant and/or a flavoring agent, as described elsewhere herein.

In certain embodiments, the composition, in particular the pharmaceutical composition according to the invention, comprises Na-D-BHB and L-Arg-D-BHB, preferably at a molar ratio of about 1:1, more preferably wherein Na-D-BHB is present in salt form C and L-Arg-D-BHB is present in salt form B, and further comprises one or more of microcrystalline cellulose, lactose (anhydrous), mannitol, magnesium stearate, and/or stearic acid, at a suitable concentration.

The compositions disclosed herein, including the nutritional and pharmaceutical compositions according to the invention, may comprise additional therapeutically active compounds. Specifically, these compositions may include one or more compounds suitable for the treatment or management of and/or prevention of symptoms associated with LC-FAOD.

In certain embodiments, the composition according to the invention may comprise L-arginine. As detailed elsewhere herein, L-arginine is associated with various physiological effects, such as enhancing TCA-cycle metabolism and improving mitochondrial function. These effects are particularly relevant for managing LC-FAOD symptoms, where energy production is compromised. Therefore, incorporating L-arginine in the composition may enhance or augment the therapeutic benefits of D-BHB.

The L-arginine may be comprised in the composition according to the invention in any form. In certain embodiments, the L-arginine is present in the form of a salt. It is preferred that the composition comprises an L-arginine salt of D-BHB, more preferably salt Form B of L-Arg-D-BHB, as defined elsewhere herein.

In certain embodiments, the composition according to the invention may comprise medium-chain triglycerides as an additional active ingredient. As discussed elsewhere herein, the medium-chain triglyceride triheptanoin has been approved for the treatment of LC-FAOD. Therefore, it is plausible that the combination of D-BHB with medium-chain triglycerides, particularly triheptanoin, would be especially suitable for the treatment of LC-FAOD.

Thus, in certain embodiments, the invention relates to a composition comprising D-BHB and a medium-chain triglyceride, particularly triheptanoin. In certain embodiments, the composition is a nutritional or pharmaceutical formulation comprising D-BHB and a medium-chain triglyceride, preferably triheptanoin. In certain embodiments, the composition is a nutritional or pharmaceutical formulation comprising an L-arginine salt of D-BHB and a medium-chain triglyceride, preferably triheptanoin. In certain embodiments, the composition is a nutritional or pharmaceutical formulation comprising an L-arginine salt of D-BHB (preferably salt Form B) and a sodium salt of D-BHB (preferably salt Form C) and a medium-chain triglyceride, preferably triheptanoin. In certain embodiments, the composition is a nutritional or pharmaceutical formulation comprising salt Form B of L-Arg-D-BHB and salt Form C of Na-D-BHB, preferably at a molar ratio of about 1:1, and a medium-chain triglyceride, preferably triheptanoin. The skilled person is capable of identifying a suitable concentration of the medium-chain triglyceride, particularly triheptanoin, that can be used in combination with D-BHB.

In other embodiments, the composition according to the invention, particularly the pharmaceutical or nutritional composition, may comprise nicotinamide riboside (NR). Nicotinamide riboside (NR) offers potential as an active ingredient due to its ability to elevate NAD+ levels in the body, which is essential for various metabolic pathways. It has been shown to be effective in treating cardiovascular, neurodegenerative, and metabolic disorders (see Mehmel et al., Nutrients, 2020 May 31; 12 (6): 1616). In certain embodiments, the composition according to the invention, particularly the pharmaceutical or nutritional composition, comprises NR and one or more salts of D-BHB. In certain embodiments, the composition comprises NR and an arginine salt of D-BHB, particularly Form B of L-Arg-D-BHB. In certain embodiments, the composition comprises NR, an arginine salt of D-BHB (preferably salt Form B of L-Arg-D-BHB), and a sodium salt of D-BHB (preferably salt Form C of Na-D-BHB). In certain embodiments, the composition comprises NR and a mixture of salt Form B of L-Arg-D-BHB and salt Form C of Na-D-BHB at a molar ratio of about 1:1. In certain embodiments, the composition comprises NR and one or more of a sodium salt, a magnesium salt, and/or a calcium salt of D-BHB, as defined in more detail elsewhere herein. The nicotinamide riboside may be present in any of the composition disclosed herein above at a concentration of about 0.5% (w/w) to about 10% (w/w), preferably about 1% (w/w) to about 5% (w/w), more preferably about 2% (w/w) to about 4.5% (w/w).

The composition, in particular the pharmaceutical or nutritional composition, according to the invention may be formulated for any suitable route of administration, as described elsewhere herein. Thus, in a particular embodiment, the invention relates to the composition according to the invention, wherein the composition is formulated for enteral administration, preferably oral administration or gastric administration, including nasogastric administration or administration by gastrostomy, or for parental administration, preferably intravenous administration.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. All ranges are inclusive of the endpoints of the range. For example, an amount between 1 and 10 includes both 1 and 10.

As used in this disclosure and the appended claims, the singular forms “a,” “an” and “the” include plural referents unless the context clearly dictates otherwise.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions and methods disclosed herein may lack any element that is not specifically disclosed herein. Thus, a disclosure of an embodiment using the term “comprising” includes a disclosure of embodiments “consisting essentially of” and “consisting of” the components or steps identified.

The terms “at least one of” and “and/or” used respectively in the context of “at least one of X and Y” and “X and/or Y” should be interpreted as “X without Y,” or “Y without X,” or “both X and Y.” Where used herein, the terms “example” and “such as,” particularly when followed by a listing of terms, are merely exemplary and illustrative and should not be deemed to be exclusive or comprehensive.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### BRIEF DESCRIPTION OF DRAWINGS

Examples of the invention will now be described in detail with reference to the accompanying drawings:

FIGS. 1A-1B: X-ray powder diffraction (“XRPD”) pattern of free base crystalline forms of D-BHB obtained from condition 1 (A) (XRPD Method 2) and condition 2 (B) (XRPD Method 2).

FIGS. 2A-2B: Differential scanning calorimetry (“DSC”) thermograph of free base crystalline forms of D-BHB obtained from condition 1 (A) and condition 2 (B).

FIGS. 3A-3B: Thermogravimetric analysis (“TGA”) trace of free base crystalline forms of D-BHB obtained from condition 1 (A) and condition 2 (B).

FIGS. 4A-4B: ¹H-NMR spectrum of free base crystalline forms of D-BHB obtained from condition 1 (A) and condition 2 (B).

FIGS. 5A-5G: XRPD (Method 2) of (A) sodium salt screening; (B) magnesium salt screening; (C) L-arginine salt screening; (D) L-lysine salt screening; (E) erbumine salt screening; (F) betaine salt screening; and (G) re-slurry screening of L-arginine salt screening.

FIG. 6: XRPD (Method 2) of crystalline magnesium salt forms A and B.

FIGS. 7A-7D: D-BHB magnesium salt Form A (FR03684-3-RC5D-re-EA) (A) XRPD (Method 2) (B) DSC (C) TGA and (D) ¹H-NMR spectrum.

FIGS. 8A-8D: D-BHB magnesium salt Form B (FR03684-3-RC5E-re-ACN) (A) XRPD (Method 2) (B) DSC (C) TGA and (D) ¹H-NMR spectrum.

FIGS. 9A-9D: D-BHB sodium salt Form A (FR03684-3-RC2F-THF) (A) XRPD (Method 2) (B) DSC (C) TGA and (D) ¹H-NMR spectrum.

FIGS. 10A-10D: D-BHB sodium salt Form B (FR03684-3-RC2C-Acetone) (A) XRPD (Method 2) (B) DSC (C) TGA and (D) ¹H-NMR spectrum.

FIGS. 11A-11D: D-BHB sodium salt Form C (FR03684-3-RC14F-THF) (A) XRPD (Method 2) (B) DSC (C) TGA and (D) ¹H-NMR spectrum.

FIGS. 12A-12D: D-BHB L-lysine salt Form A (FR03684-3-RC7B-EtOH) (A) XRPD (Method 2) (B) DSC (C) TGA and (D) ¹H-NMR spectrum.

FIGS. 13A-13D: D-BHB erbumine salt Form A (FR03684-3-RC9D-EA) (A) XRPD (Method 2) (B) DSC (C) TGA and (D) ¹H-NMR spectrum.

FIGS. 14A-14D: D-BHB L-arginine salt Form A (FR03684-3-RC6D-EA) (A) XRPD (Method 2) (B) DSC (C) TGA and (D) ¹H-NMR spectrum.

FIGS. 15A-15D: D-BHB L-arginine salt Form B (FR03684-3-RC6H-acetone-water-95-5) (A) XRPD (Method 2) (B) DSC (C) TGA and (D) ¹H-NMR spectrum.

FIGS. 16A-16B: D-BHB sodium salt Form C XRPD (Method 2) (A) FR03684-SU1-NaOH-1.5-THF and (B) FR03684-SU9-NaOH-1.5-THF.

FIGS. 17A-17D: D-BHB sodium salt Form C (FR03684-SU9-NaOH-1.5-THF) (A) XRPD (Method 2) (B) DSC (C) TGA and (D) ¹H-NMR spectrum.

FIGS. 18A-18B: D-BHB L-arginine salt Form B (FR03684-SU2-L-arginine-acetone-water-95-5-re) (A) overlay of XRPD (Method 2) of scale up Trial land (B) ¹H-NMR spectrum.

FIGS. 19A-19D: D-BHB L-arginine salt Form B (FR03684-SU2-L-arginine-acetone-water-95-5-re) (A) XRPD (Method 2) (B) DSC (C) TGA and (D) ¹H-NMR spectrum.

FIGS. 20A-20B: D-BHB L-arginine salt Form B (FR03684-SU5-L-arginine-acetone-water-95-5) (A) overlay of XRPD (Method 2) of scale up Trial 2 and (B) ¹H-NMR spectrum.

FIGS. 21A-21B: D-BHB L-lysine salt Form A FR03684-SU3-L-lysine-EtOH-re (A) overlay XRPD (Method 2) from scale up and (B) ¹H-NMR spectrum.

FIGS. 22A-22D: D-BHB L-lysine salt Form A (FR03684-SU3-L-lysine-EtOH-re) (A) XRPD (Method 2) (B) DSC (C) TGA and (D) ¹H-NMR spectrum.

FIGS. 23A-23B: D-BHB L-lysine salt Form A (FR03684-SU6-L-lysine-EtOH) (A) XRPD (Method 2) overlay of Trial 2 and (B) ¹H-NMR spectrum.

FIG. 24: Overlay of XRPD (Method 2) of D-BHB erbumine salt Form A and Form B from scale-up.

FIGS. 25A-25D: D-BHB erbumine salt Form A (FR03684-SU4-erbumine-EA) (A) XRPD (Method 2) (B) DSC (C) TGA and (D) ¹H-NMR spectrum.

FIGS. 26A-26E: (A) Overlay of XRPD (Method 3) of D-BHB erbumine salt Form A and Form B from scale-up of process to obtain form B; D-BHB erbumine salt Form B (FR03684-SU8-erbumine-ACN-water-95-5) (B) XRPD (Method 1) (C) DSC (D) TGA and (E) ¹H-NMR spectrum.

FIGS. 27A-271: Mini-polymorph screening-suspensions equilibrated at 25° C. for 1 week: (A) to (C) D-BHB sodium salt Form C (FR03684-SU1-NaOH-1.5-THF) as starting material-XRPD overlay; (D) and (E) D-BHB L-arginine salt Form C (FR03684-SU2-L-arginine-acetone-water-95-5) as starting material-XRPD overlay; (F) and (G) D-BHB L-lysine salt Form A (FR03684-SU3-L-lysine-EtOH-re) as starting material-XRPD overly; (H) and (I) D-BHB erbumine salt Form A (FR03684-SU4-erbumine-EA) as starting material-XRPD overly. XRPD Method 4 for all except (C) which was Method 2.

FIGS. 28A-28D: Bulk Stability XRPD (Method 2) (A) L-arginine salt Form B (FR03684-SU5-L-arginine-acetone-water-95-5); (B) D-BHB Erbumine salt Form B (FR03684-SU8-erbumine-ACN-water-95-5); (C) D-BHB sodium salt Form C (FR03684-SU1-NaOH-1.5-THF); (D) D-BHB L-lysine salt form A (FR03684-SU6-L-lysine-EtOH).

FIGS. 29A-29D: D-BHB sodium salt Form C (FR03684-SU1-NaOH-1.5-THF) (A) XRPD (Method 1) (B) DSC (C) TGA and (D) ¹H-NMR spectrum.

FIGS. 30A-30D: D-BHB L-arginine salt Form B (FR03684-SU5-L-arginine-acetone-water-95-5) (A) XRPD (Method 1) (B) DSC (C) TGA and (D) ¹H-NMR spectrum.

FIGS. 31A-31D: D-BHB L-lysine salt Form A (FR03684-SU6-L-lysine-EtOH) (A) XRPD (Method 1) (B) DSC (C) TGA and (D) ¹H-NMR spectrum.

FIG. 32: Basal respiration of all KO cell lines expressed as % of wild-type parental cell line. Results of 6 independent experiments are expressed as mean±SEM (*: p<0.05; **: p<0.01, ***: p<0.001 vs. Wild type control cell line).

FIG. 33: Maximal respiration in response to D-BHB treatment (1 or 3 mM) or vehicle. Since basal respiration is quite heterogenous between WT and KO lines (FIG. 32), data have been normalized for each genotype and expressed as % of untreated control cell lime. Results of 3 to 6 independent experiments are expressed as mean±SEM.

### EXAMPLES

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

Characterization of the crystalline forms was carried out by the following methods.

TABLE 1
X-Ray Powder Diffractometer (XRPD)

| | |
|---|---|
| Instrument | Bruker D8 Advance |

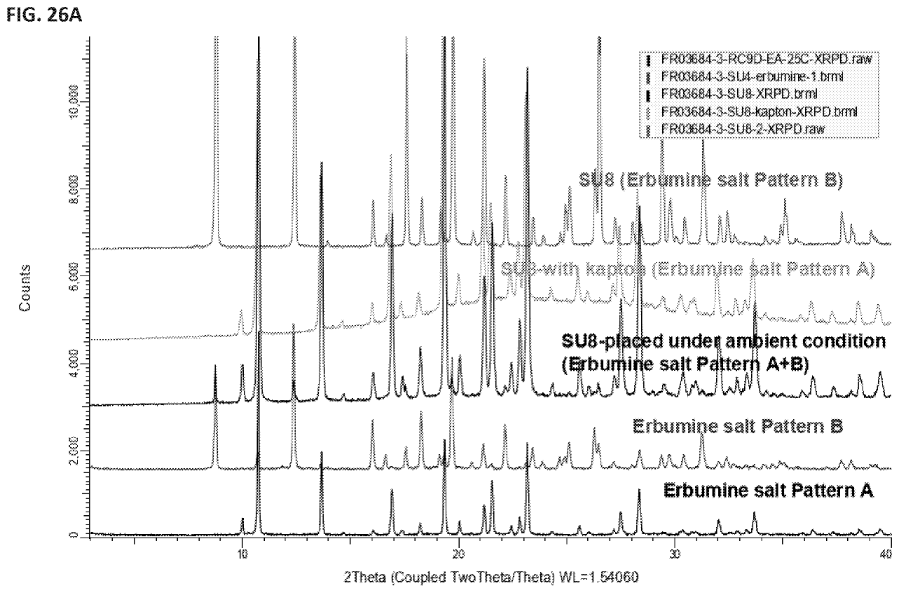

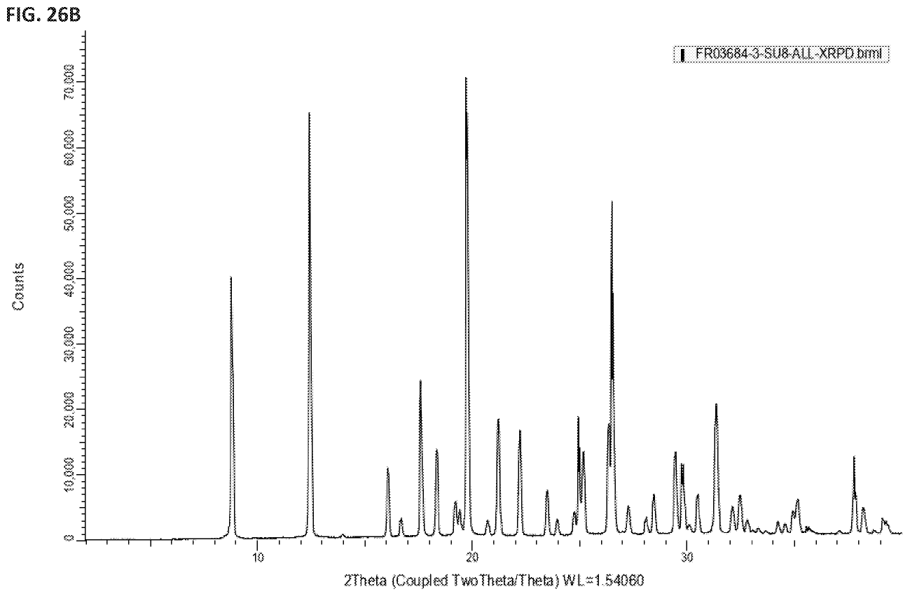

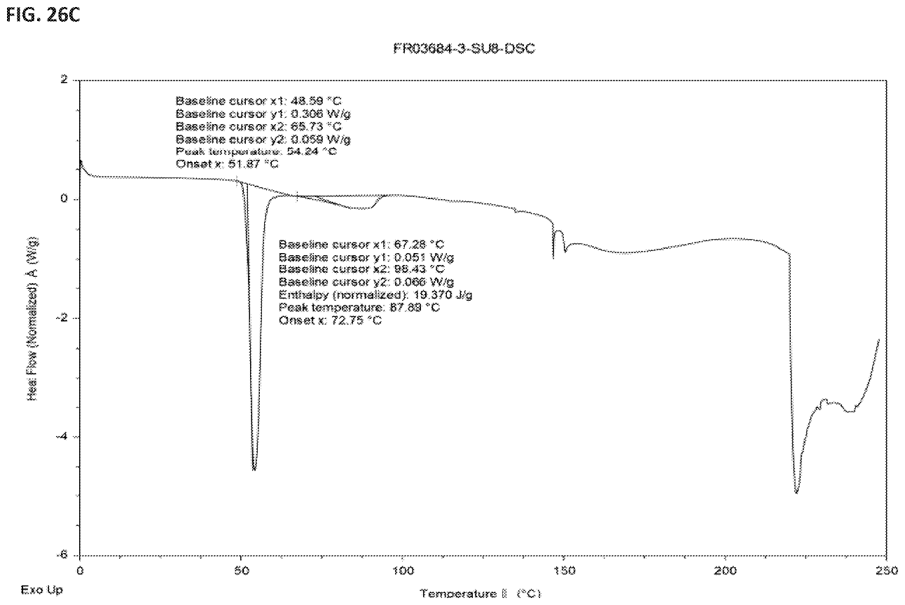

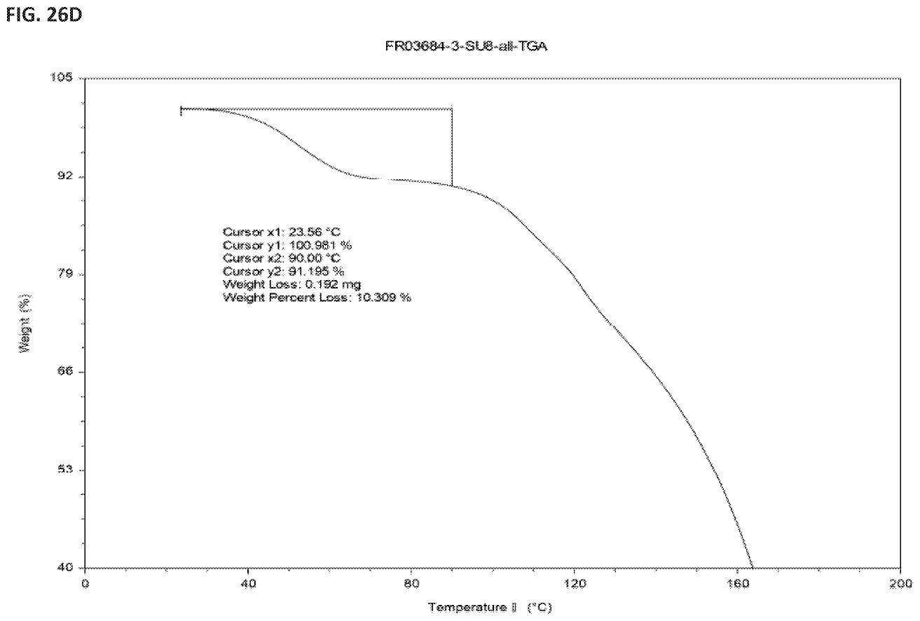

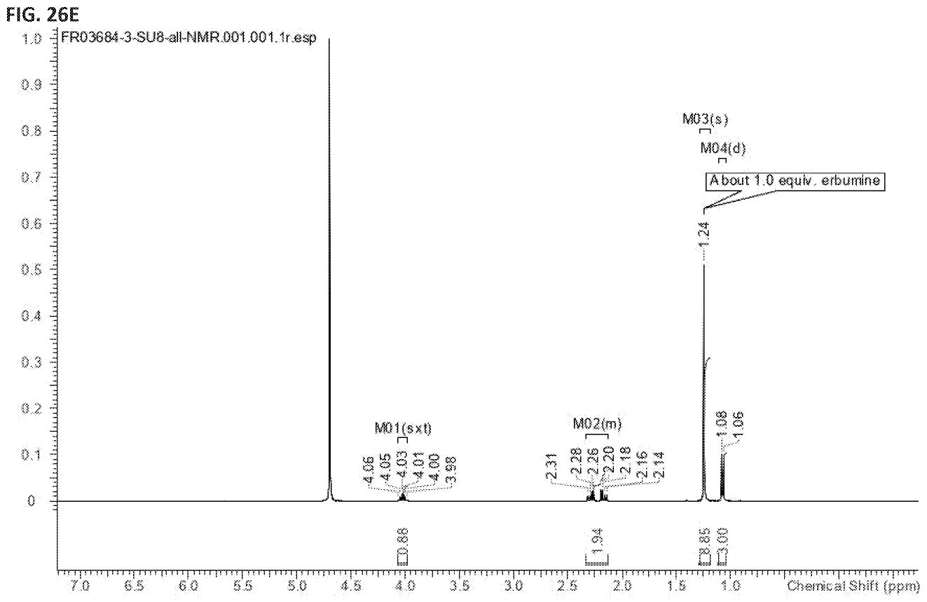

XRPD method 1 - Used for XRPD shown in FIGS. 26B, 29A-31D

| | |
|---|---|
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 10.0 mm by sample length; |
| | Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.4 second per step |
| Scan range | 2° to 40° |
| Sample rotation speed | 15 rpm |
| Sample holder | Flat monocrystalline silicon |

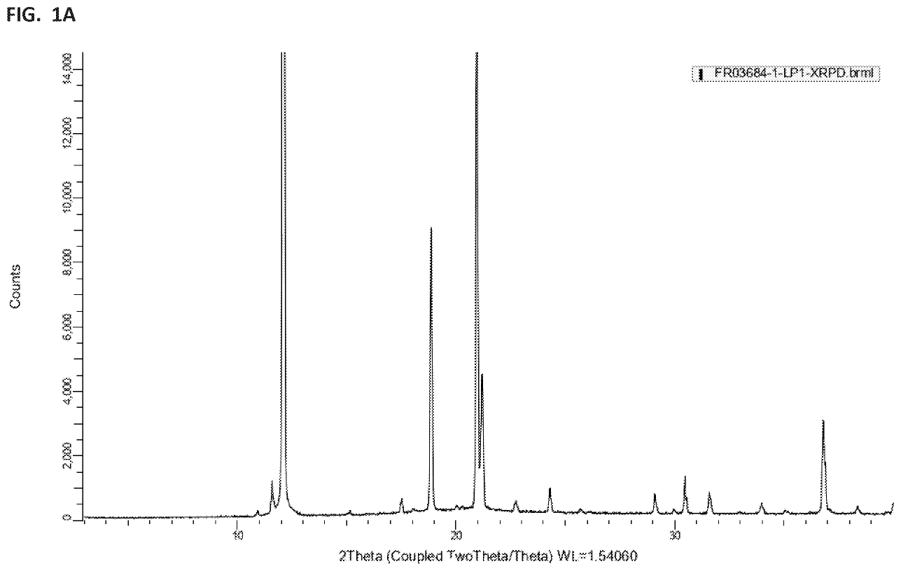

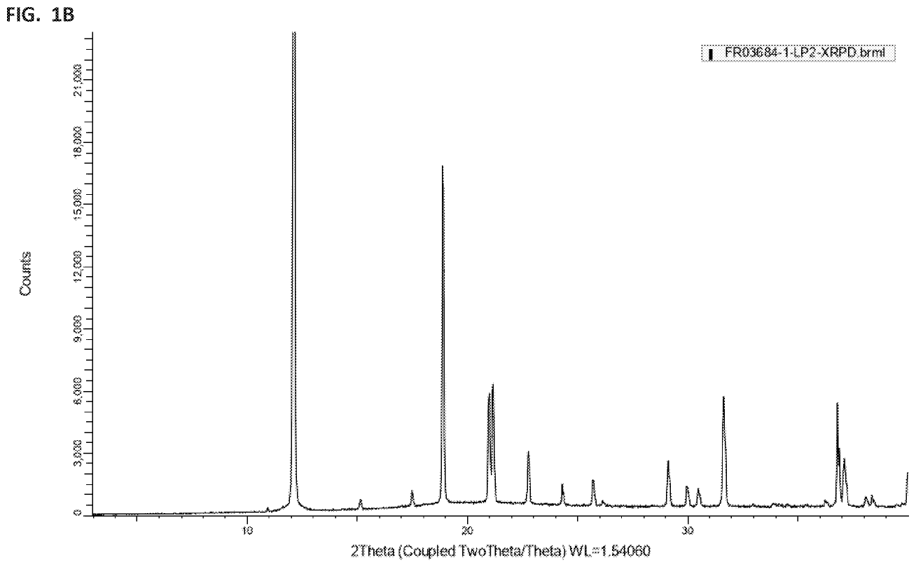

XRPD method 2 - Used for XRPD shown in FIGS. 1B, 5A-25D, 27C, and 28A-28D

| | |
|---|---|
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 10.0 mm by sample length; |
| | Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Flat monocrystalline silicon |

XRPD method 3 - Used for XRPD shown in FIG. 26A

| | |
|---|---|
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 10.0 mm by sample length; |
| | Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Flat monocrystalline silicon; covered by Kapton film |

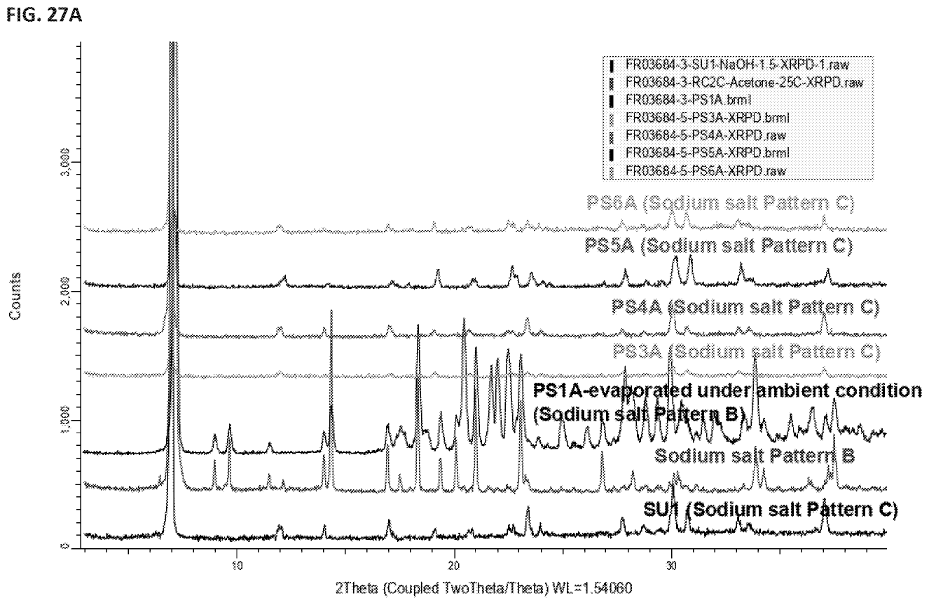

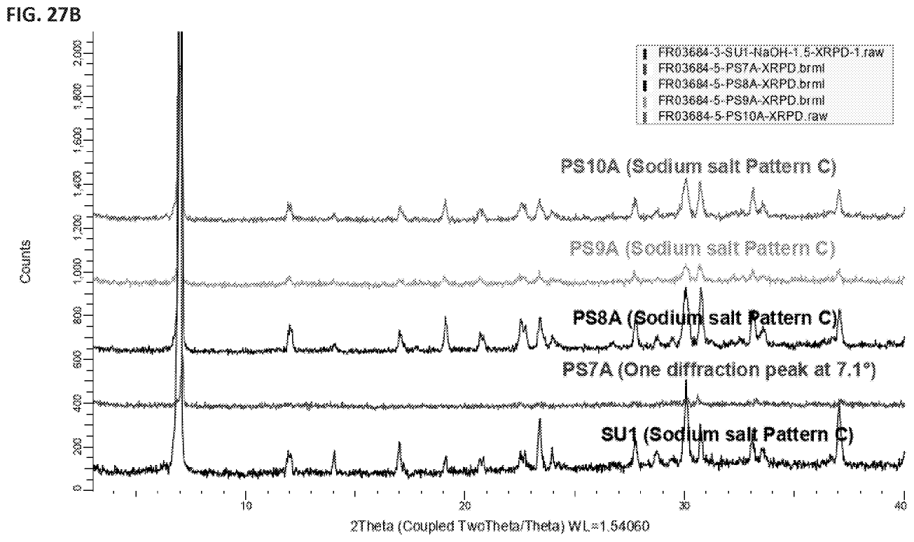

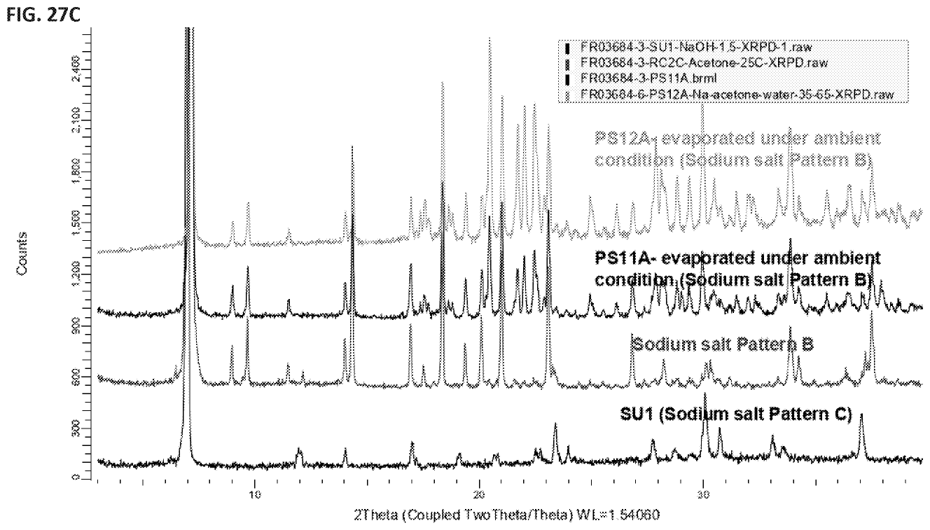

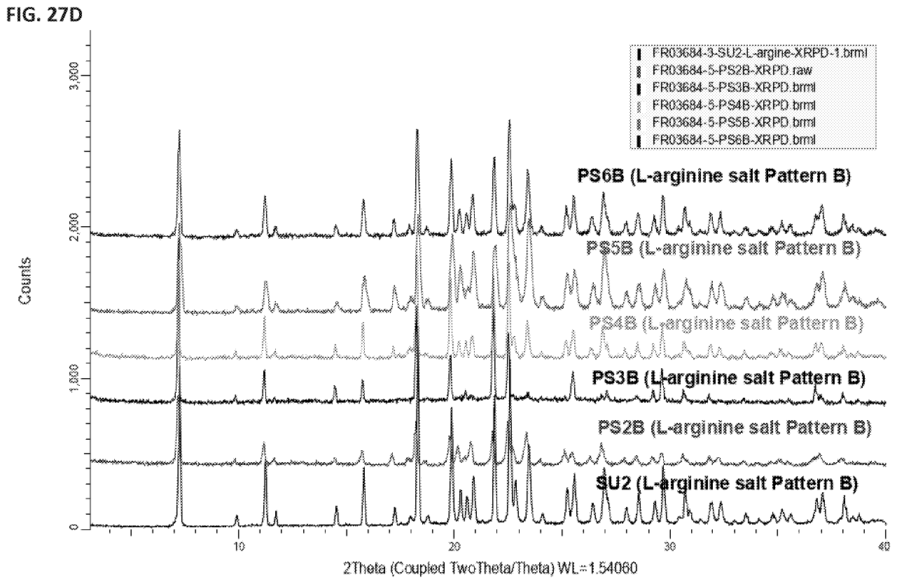

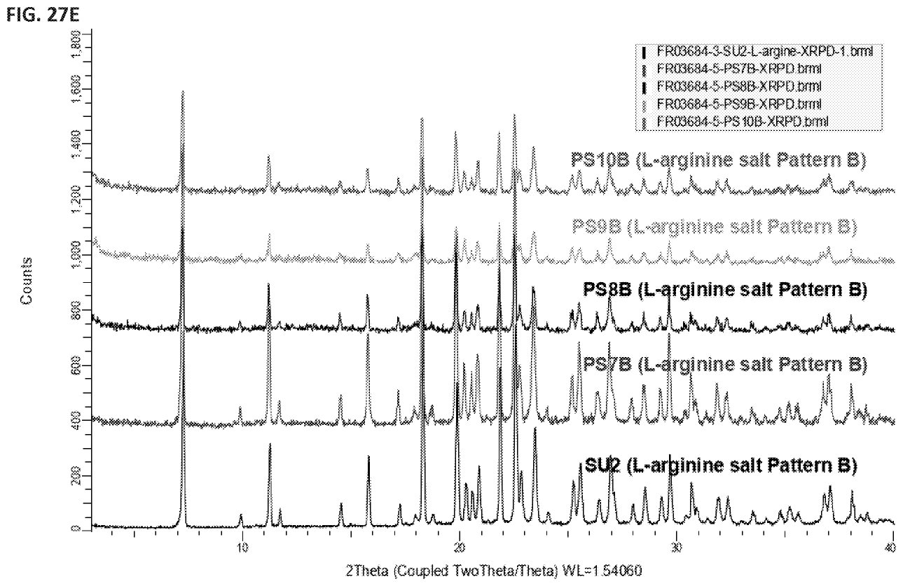

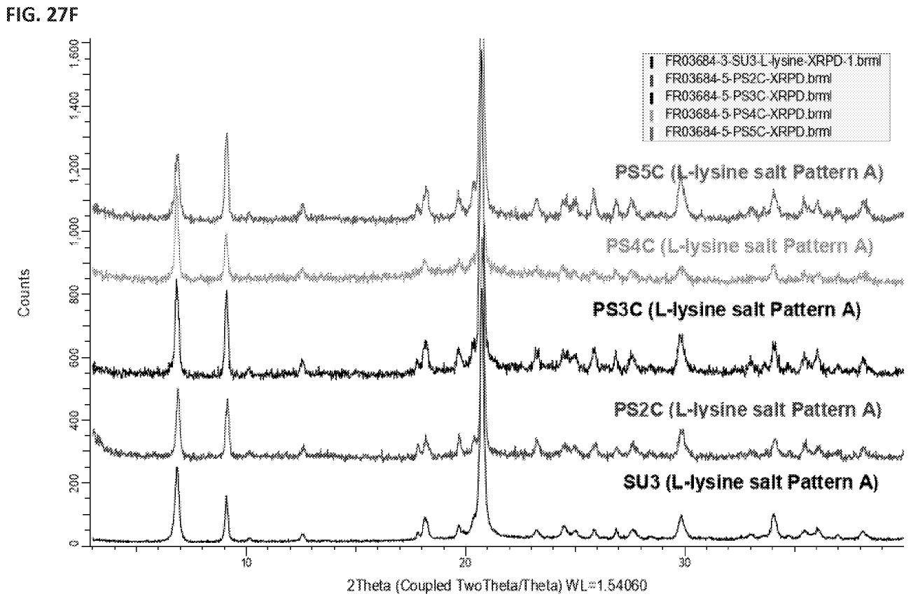

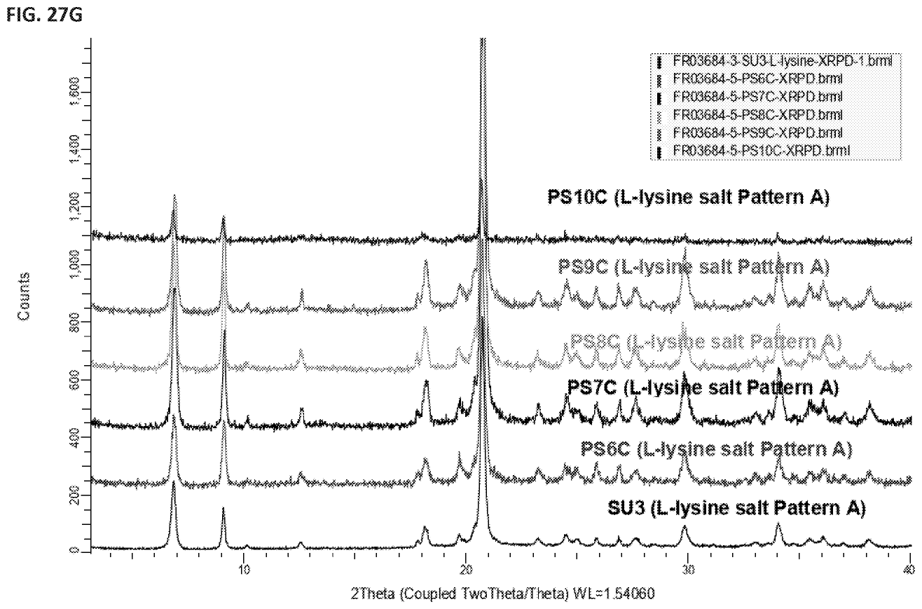

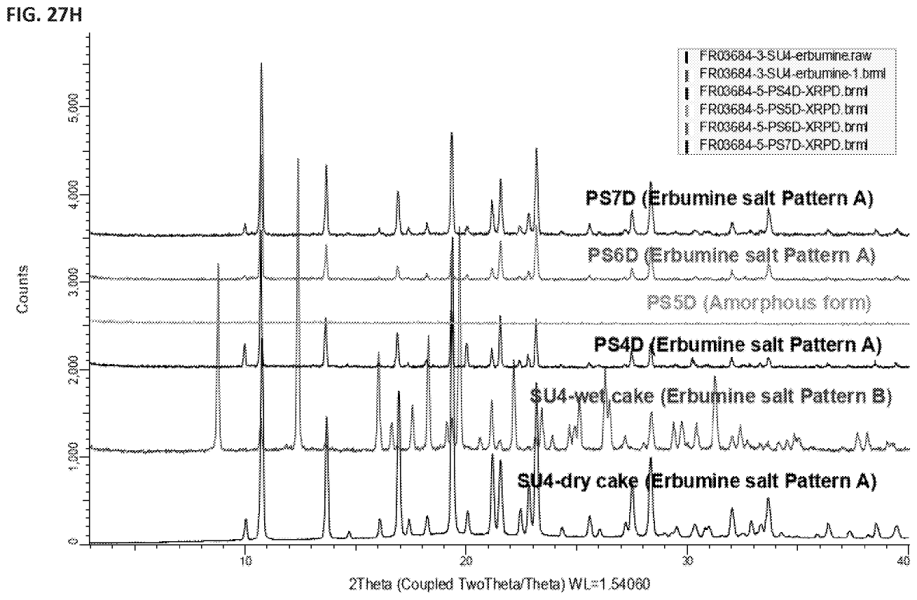

XRPD method 4 - Used for XRPD shown in FIGS. 27A-27B and FIGS. 27D-27I

| | |
|---|---|
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 10.0 mm by sample length; |
| | Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.06 second per step |
| Scan range | 3° to 40° |
| Sample rotation speed | 15 rpm |
| Sample holder | Flat monocrystalline silicon |

Differential Scanning Calorimeter (DSC)

| | |
|---|---|
| Instrument | TA Instruments Discovery 2500 |
| Sample pan | Tzero pan and Tzero hermetic lid |
| Temperature range | ~30° C. to 250° C. |
| Heating rate | 10° C./min |
| Nitrogen flow | 50 mL/min |
| Sample mass | ~0.5-5 mg |

Thermogravimetric Analyzer (TGA)

| | |
|---|---|
| Instrument | TA Instruments Discovery 5500 |
| Sample pan | Aluminum, closed |
| Start temperature | Ambient condition (below 35° C.) |
| Final temperature | 300° C. or abort next segment if weight <80% (w/w) (Weight |
| | loss of the compound is more than 20% (w/w).) |
| Heating rate | 10° C./min |
| Nitrogen flow | Balance 10 mL/min; |
| | sample chamber 25 mL/min |
| Sample mass | ~2-10 mg |

Dynamic Vapor Sorption (DVS)
Method 1 - Results shown in TABLES 40 and 41

| | |
|---|---|
| Instrument | ProUmid SPSx-1μ Advance |
| Total gas flow | Max. 4,000 mL/min |
| Oven temperature | 25° C. |
| Solvent | Water |
| Method | Cycle: 40-0-95-0-40% RH |
| | Stage Step: 10% RH |
| | Equilibrium: 240 min for each step |
| Sample mass | ~5-50 mg |

Method 2 - Results shown in TABLES 42 and 43

| | |
|---|---|
| Instrument | SMS Intrinsic |
| Total gas flow | 200 sccm |
| Oven temperature | 25° C. |
| Solvent | Water |
| Method | Cycle: 40-0-95-0-40% RH |
| | Stage Step: 10% RH |
| | Equilibrium: 240 min for each step |
| Sample mass | ~5-50 mg |

Nuclear Magnetic Resonance Spectrometer (NMR)

| | |
|---|---|
| Instrument | Bruker Avance-AV 400M |
| Frequency | 400 MHz |
| Probe | 5 mm PABBO BB/19F-1H/D Z-GRD Z108618/0406 |
| Number of scan | 8 |
| Temperature | 297.6 K |
| Relaxation delay | 1 second |

Karl Fischer Titration (KF)
Method

| | |
|---|---|
| Instrument | Mettler Toledo Coulometric KF Titrator C30 |
| Method | Coulometric |
| Sample mass | ~5-30 mg |

Ion chromatograph (IC)

| | |
|---|---|
| Instrument | Metrohm 940 professional IC |

Method 1 for stoichiometry

| | |
|---|---|
| Sample center | 889 IC |
| Detector | Conductivity detector |
| Eluent (anion) | 3.2 mmol/L Na₂CO₃ + 1.0 mmol/L NaHCO₃ |
| Eluent (cation) | 1.7 mmol/L HNO₃ + 0.7 mmol/L pyridinecarboxylic acid |
| Suppressor solutions | 2% H₃PO₄ |
| Column | Anion A SUPP 5-150 or Cation Column C4-150 |
| Column temperature | 30° C. |
| Flow rate | 0.7 mL/min (anion) or 0.9 mL/min (cation) |
| Diluent | Water |
| Injection volume: | 20 μL |

Method 2 for chemical purity, stoichiometry and solubility

| | |
|---|---|
| Sample center | 889 IC |
| Detector | Conductivity detector |
| Eluent (organic acid) | 0.5 mmol/L H₂SO₄ |
| Eluent (cation) | 1.7 mmol/L HNO₃ + 0.7 mmol/L dipicolinic acid |
| Suppressor solutions | 100 mM LiCl |
| Column | Acid-250 or Cation Column C4-150 |
| Column temperature | 40° C. |
| Flow rate | 0.5 mL/min (organic acid) or 0.9 mL/min (cation) |
| Diluent | Water |
| Injection volume | 100 μL (organic acid) or 10 μL (cation) |

### Example 1: D-BHB Crystalline Free Base Form (Form I)

Trial 1: 50 ml of the 40 wt % of D-BHB aqueous solution was added into a 500 ml glass bottle. Release specifications for D-BHB is greater than 95% D enantiomer (i.e. greater than 95% enantiomeric purity of the D enantiomer). This aqueous solution was treated by lyophilization. Sugar like crystals were obtained after 3 days. Obtained sugar like crystals were very hygroscopic and deliquesced after exposure to ambient condition (20-25° C./40-70% RH) within 30 minutes. Therefore, obtained sugar like crystals were dried under vacuum at 25° C. for 2 hours to remove surface moisture. 22.7 g of D-BHB free Form I was obtained. Obtained sugar like crystals were kept in a closed container. Referred to herein as sample: FR03684-1-LP1 (with XRPD shown in FIG. 1A with the following peaks in TABLE 2).

TABLE 2
FR03684-LP1

| | | | Net | Gross | Rel. |
|---|---|---|---|---|---|
| Index | Angle | d Value | Intensity | Intensity | Intensity |
| 1 | 10.938° | 8.08266 Å | 124.881 | 254.526 | 0.2% |
| 2 | 11.607° | 7.61803 Å | 724.011 | 888.303 | 1.3% |
| 3 | 12.113° | 7.30060 Å | 57733.3 | 57906.8 | 100.0% |
| 4 | 15.164° | 5.83788 Å | 114.168 | 273.282 | 0.2% |
| 5 | 17.530° | 5.05503 Å | 299.346 | 541.311 | 0.5% |
| 6 | 18.052° | 4.90998 Å | 56.0858 | 321.327 | 0.1% |
| 7 | 18.880° | 4.69650 Å | 6088.99 | 6389.02 | 10.5% |
| 8 | 20.049° | 4.42523 Å | 87.1356 | 418.417 | 0.2% |
| 9 | 20.270° | 4.37744 Å | 54.9704 | 393.695 | 0.1% |
| 10 | 20.962° | 4.23457 Å | 11967.7 | 12311.7 | 20.7% |
| 11 | 21.202° | 4.18718 Å | 3161.15 | 3500.66 | 5.5% |
| 12 | 22.749° | 3.90569 Å | 235.802 | 505.579 | 0.4% |
| 13 | 24.307° | 3.65882 Å | 575.102 | 823.387 | 1.0% |
| 14 | 25.698° | 3.46380 Å | 115.583 | 340.018 | 0.2% |
| 15 | 26.147° | 3.40542 Å | 35.0589 | 250.815 | 0.1% |
| 16 | 29.104° | 3.06578 Å | 530.918 | 737.657 | 0.9% |
| 17 | 29.953° | 2.98080 Å | 109.714 | 317.825 | 0.2% |
| 18 | 30.481° | 2.93035 Å | 1001.19 | 1215.82 | 1.7% |
| 19 | 31.604° | 2.82869 Å | 518.626 | 720.915 | 0.9% |
| 20 | 32.963° | 2.71513 Å | 50.2831 | 245.815 | 0.1% |
| 21 | 33.984° | 2.63584 Å | 276.808 | 475.550 | 0.5% |
| 22 | 35.067° | 2.55692 Å | 105.819 | 300.659 | 0.2% |
| 23 | 36.808° | 2.43984 Å | 2562.00 | 2776.71 | 4.4% |
| 24 | 38.377° | 2.34365 Å | 200.782 | 396.392 | 0.3% |

Trial 2: 50 ml of the 40 wt % of D-BHB aqueous solution was added into a 500 ml glass bottle. This aqueous solution was treated by lyophilization. Sugar like crystals were obtained after 6 days. Obtained sugar like crystals were very hygroscopic and deliquesced after exposure to ambient condition (20-25° C./40-70% RH) within 30 minutes. Obtained sugar like crystals were dried under vacuum at 25° C. for 1 day to remove surface moisture. 21.0 g of D-BHB free Form I was obtained. Obtained sugar like crystals were kept in a closed container. Referred to herein as sample: FR03684-1-LP2 (with XRPD shown in FIG. 1B with the following peaks in TABLE 3).

TABLE 3
FR03684-1-LP2

| | | | Net | Gross | Rel. |
|---|---|---|---|---|---|
| Index | Angle | d Value | Intensity | Intensity | Intensity |
| 1 | 10.937° | 8.08324 Å | 120.201 | 293.287 | 0.2% |
| 2 | 12.111° | 7.30215 Å | 67517.8 | 67761.7 | 100.0% |
| 3 | 15.153° | 5.84219 Å | 322.466 | 616.771 | 0.5% |
| 4 | 17.498° | 5.06424 Å | 531.232 | 973.303 | 0.8% |
| 5 | 18.882° | 4.69609 Å | 14136.8 | 14711.5 | 20.9% |
| 6 | 20.992° | 4.22864 Å | 3851.73 | 4461.38 | 5.7% |
| 7 | 21.157° | 4.19601 Å | 4784.87 | 5390.72 | 7.1% |
| 8 | 22.779° | 3.90065 Å | 1989.52 | 2537.35 | 2.9% |
| 9 | 24.313° | 3.65790 Å | 850.397 | 1331.28 | 1.3% |
| 10 | 25.717° | 3.46134 Å | 1049.81 | 1510.48 | 1.6% |
| 11 | 26.146° | 3.40555 Å | 223.193 | 674.104 | 0.3% |
| 12 | 29.126° | 3.06354 Å | 1993.23 | 2421.90 | 3.0% |
| 13 | 29.982° | 2.97792 Å | 877.087 | 1306.55 | 1.3% |
| 14 | 30.496° | 2.92891 Å | 716.945 | 1148.30 | 1.1% |
| 15 | 31.635° | 2.82600 Å | 4840.75 | 5280.26 | 7.2% |
| 16 | 32.983° | 2.71356 Å | 90.7581 | 508.048 | 0.1% |
| 17 | 33.858° | 2.64541 Å | 154.411 | 546.248 | 0.2% |
| 18 | 34.155° | 2.62308 Å | 129.786 | 533.020 | 0.2% |
| 19 | 34.538° | 2.59486 Å | 107.947 | 518.006 | 0.2% |
| 20 | 35.387° | 2.53448 Å | 55.6666 | 480.171 | 0.1% |
| 21 | 36.258° | 2.47557 Å | 273.528 | 716.315 | 0.4% |
| 22 | 36.805° | 2.44004 Å | 4703.89 | 5148.54 | 7.0% |
| 23 | 37.127° | 2.41961 Å | 1864.21 | 2301.50 | 2.8% |
| 24 | 38.102° | 2.35990 Å | 425.724 | 842.432 | 0.6% |
| 25 | 38.382° | 2.34336 Å | 537.158 | 952.878 | 0.8% |
| 26 | 39.504° | 2.27935 Å | 81.1735 | 514.234 | 0.1% |
| 27 | 39.738° | 2.26647 Å | 126.371 | 605.326 | 0.2% |

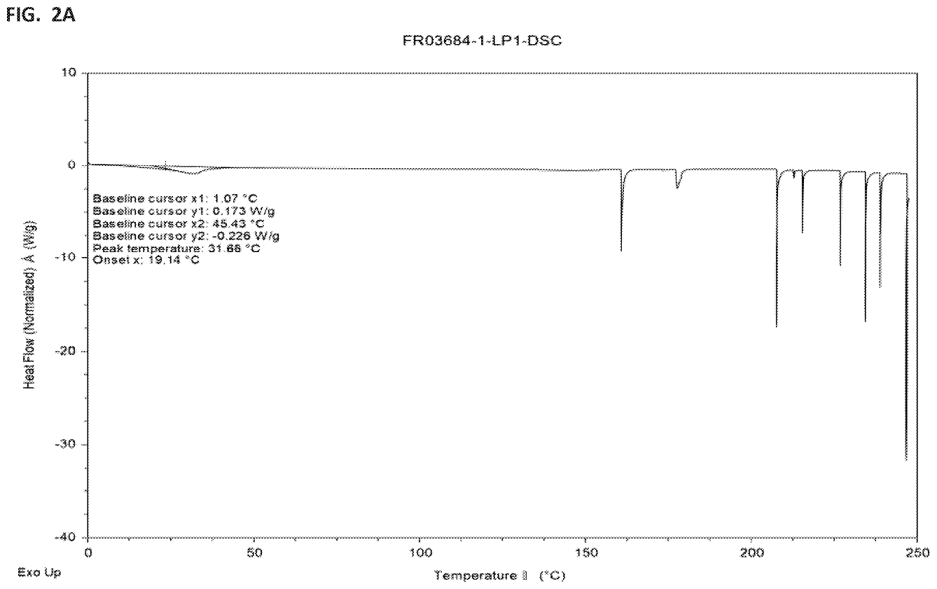

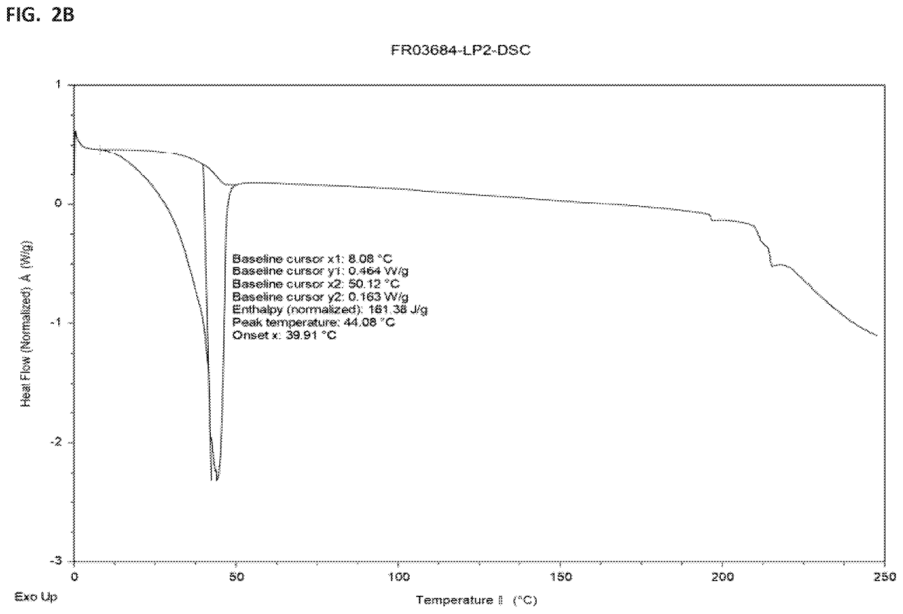

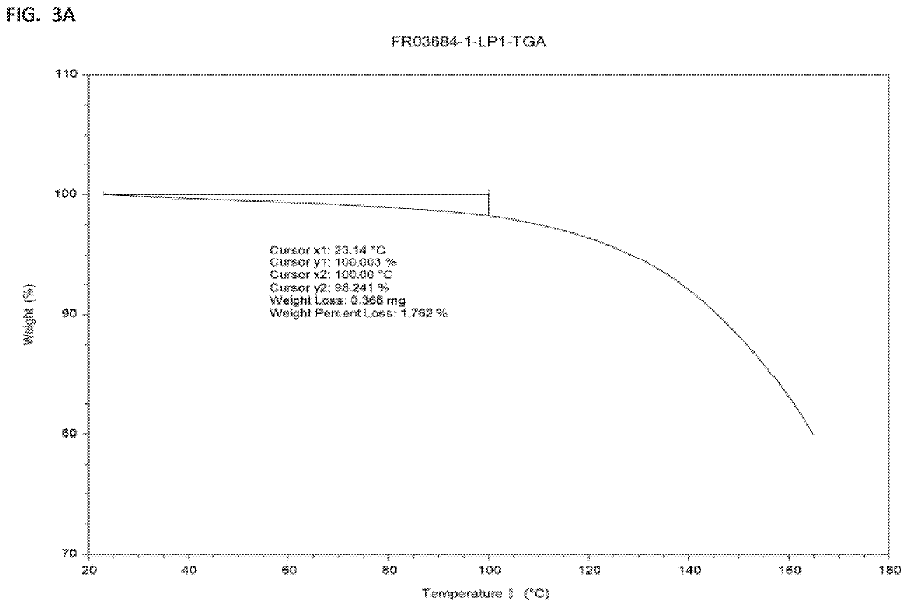

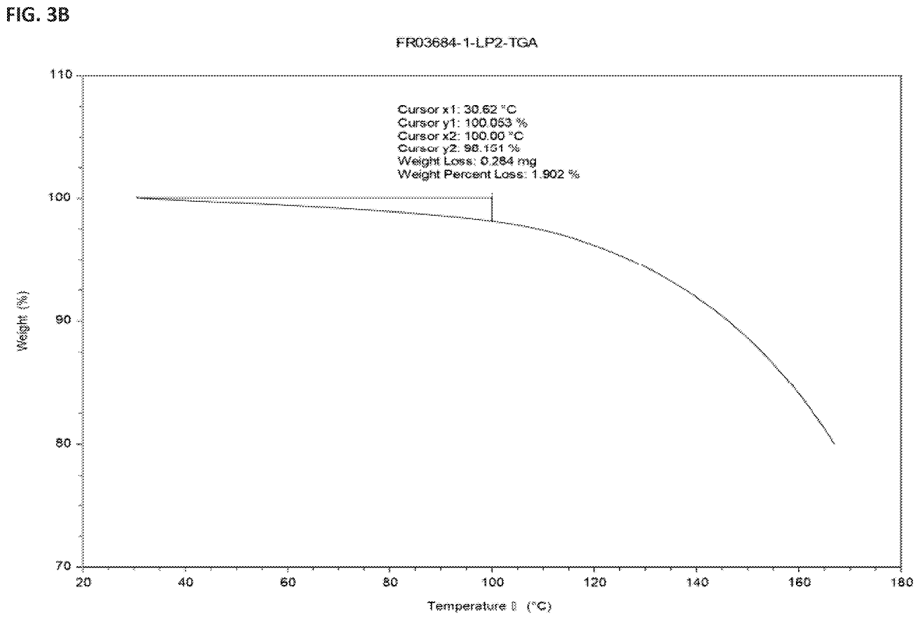

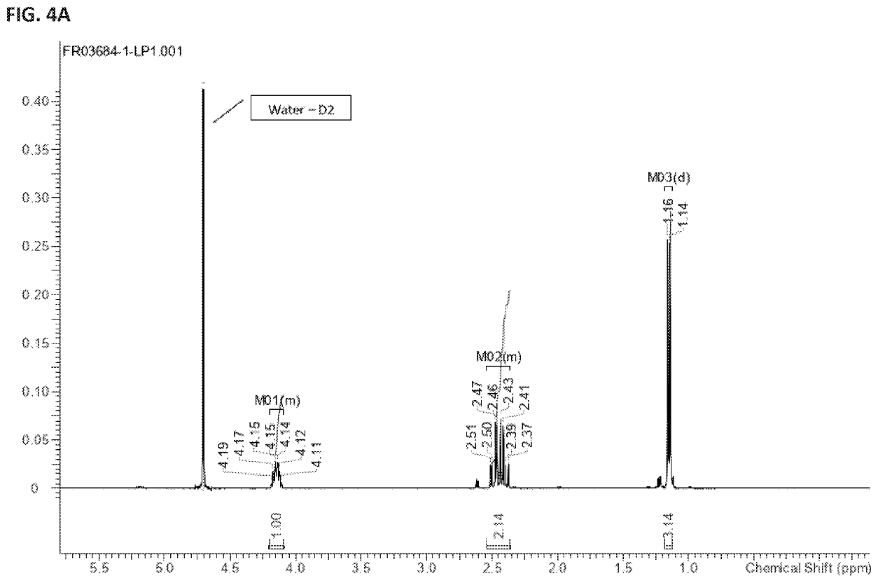

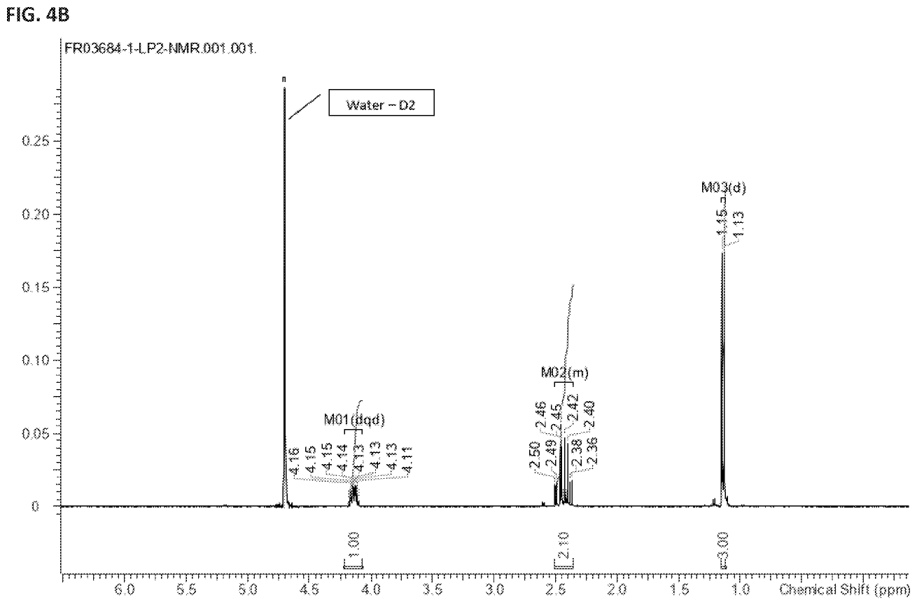

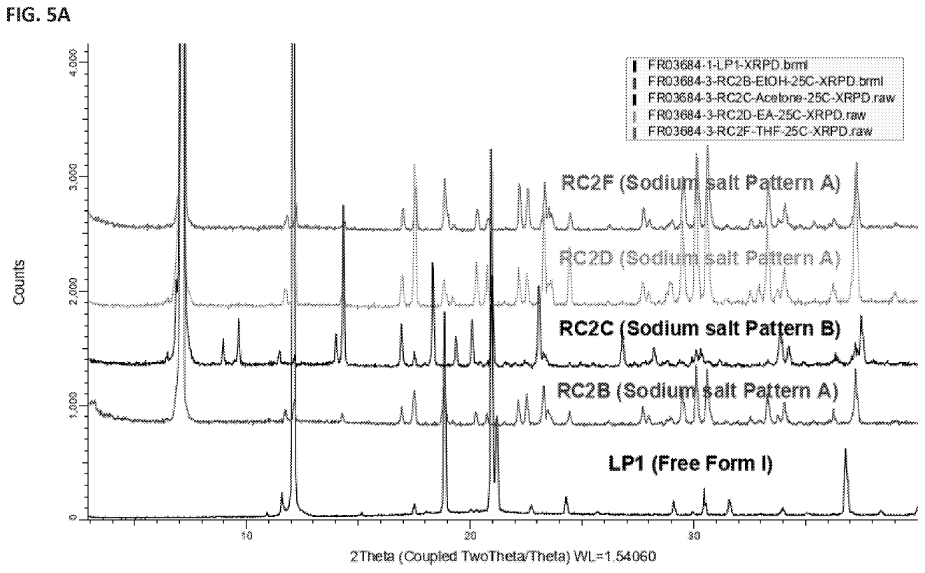

TABLE 4
D-BHB free Form I

| | | Result | |
|---|---|---|---|
| Parameter | Method | FR03684-1-LP1 | FR03684-1-LP2 |
| X-ray diffraction | METHOD 2 | FIG. 1A | FIG. 1B |
| | XRPD, 3-40° (2 | | |
| | theta) | | |
| Melting onset and | DSC, 10° C./min | FIG. 2A: Dehydration | FIG. 2B: Dehydration |
| enthalpy | | from about 1° C., no | from about 8° C., no |
| | | melting point before | melting point before |
| | | decomposition | decomposition |
| Thermogravimetry | TGA, 10° C./min | FIG. 3A: 1.8% @ 100° C. | FIG. 3B: 1.9% @ 100° C. |
| Residual solvent(s) | ¹H-NMR (D₂O- | FIG. 4A: Undetected | FIG. 4B: Undetected |
| | d₂) | | |
| Water content | Karl Fisher | 2.2% water by weight | 1.2% water by weight |

### Example 2: Salt Screening

The following counter-ions were selected for screening.

TABLE 5

| Counter ions | pKa(s)* | M.W. | Class | Theoretical chemical structure of the salt form |
|---|---|---|---|---|
| NaOH | ca.14 | 40.0 | I | 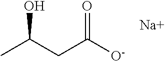 |
| KOH | ca.14 | 56.1 | I | 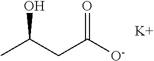 |
| Ca(OH)₂ | 12.6 | 74.1 | I | 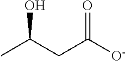 |
| | | | | 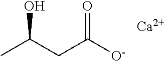 |
| Mg (OH)₂ | 11.4 | 58.3 | I | 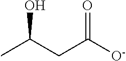 |
| | | | | 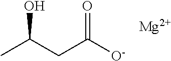 |
| L-Arginine | 13.2 | 174.2 | I | 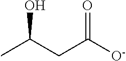 |
| | | | | 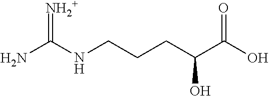 [SMILES: NC(=[NH2+])NCCC[C@H](O)C(=O)O] |
| L-Lysine | 10.8 | 146.2 | I | 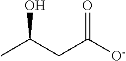 |
| | | | | 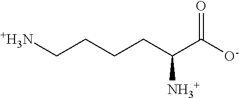 |
| Methylglucamine | 8.0 | 195.2 | I | 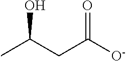 |
| | | | | 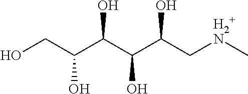 |
| Erbumine | 10.7 | 73.1 | I | 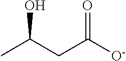 |
| | | | | 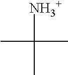 |
| NH₃ | 9.3 | 17 | I | 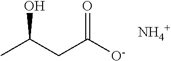 |
| TRIS | 8.1 | 121.1 | II | 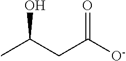 |
| | | | | 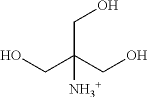 |
| Betaine | 12.2 | 117.2 | II | 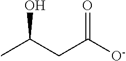 |
| | | | | 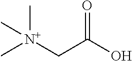 |

About 30 mg of the D-BHB free Form I (FR03684-1-LP1) and 1 or 0.5 equivalents of counter ions were added into 0.1-1.2 mL of screening solvents (water, ethanol, acetone, ethyl acetate (EA), acetonitrile (ACN) or tetrahydrofuran (THF)) in a 2 ml glass vial. Obtained mixtures were stirred at 25° C. for at least 48 hours. Obtained suspensions were filtered through a 0.45 μm nylon membrane filter by centrifugation at 14,000 rpm. After being dried at 50° C. under vacuum for 2h, solids were analyzed by XRPD.

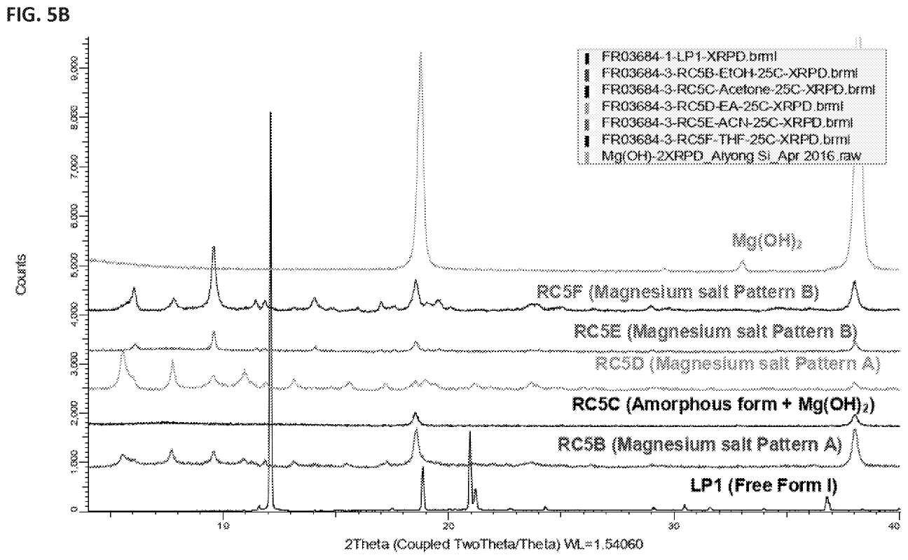

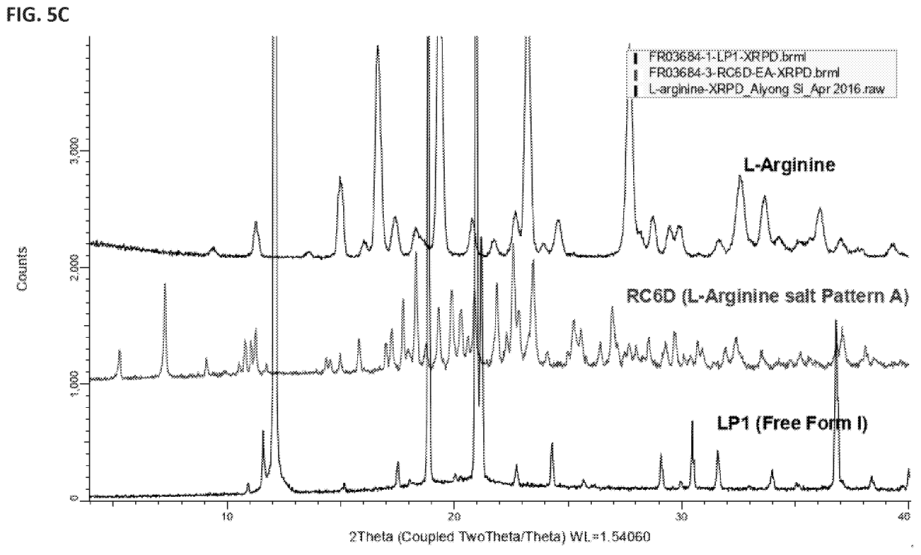

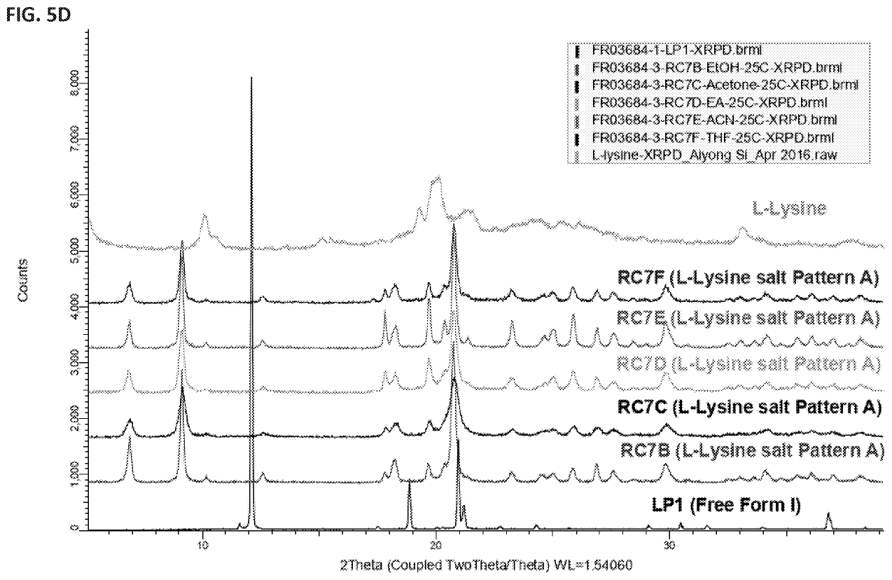

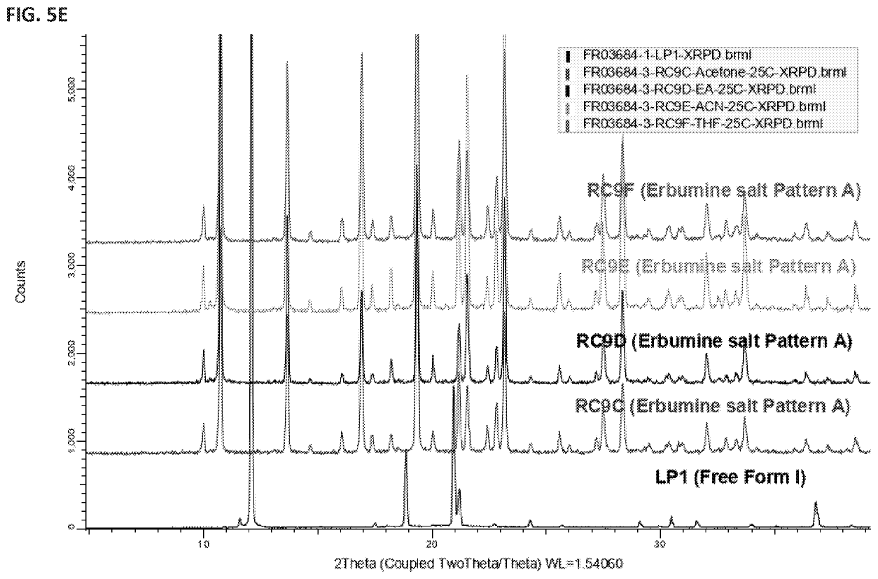

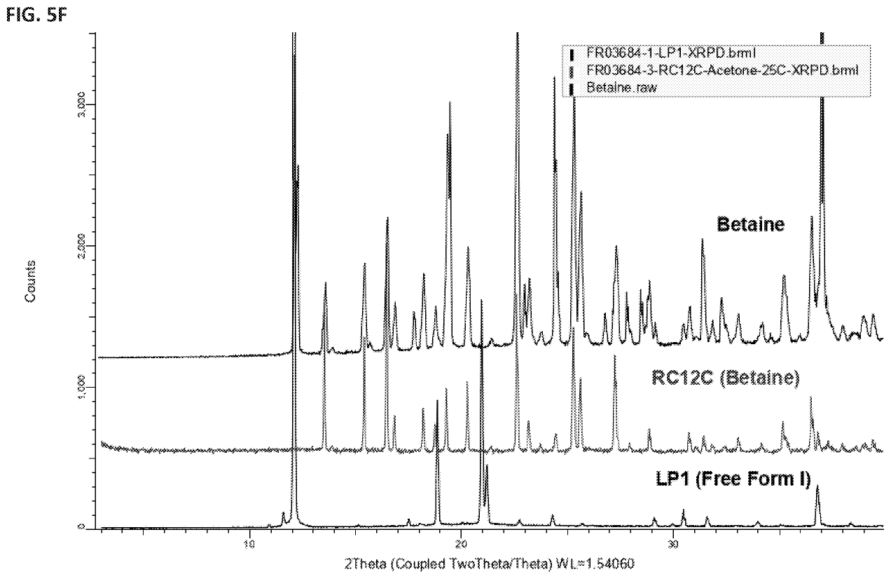

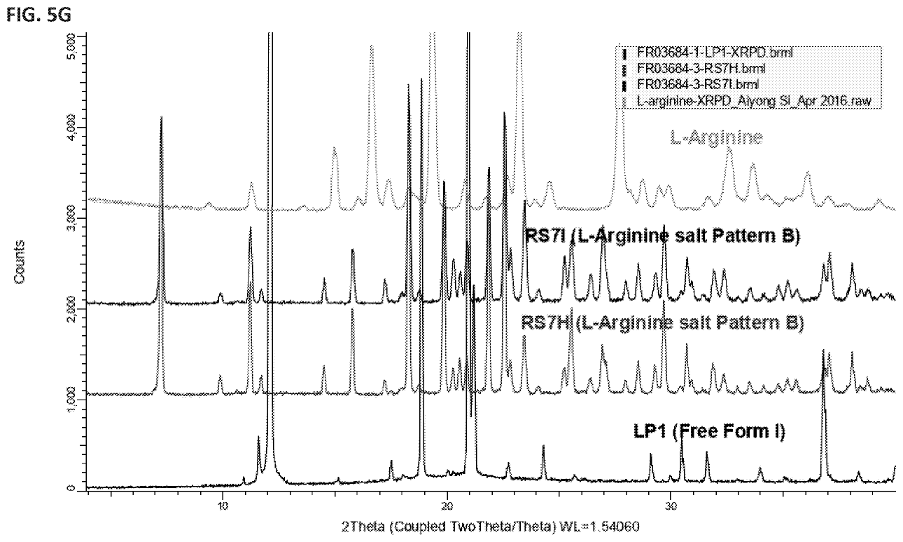

Crystalline salt forms including sodium salt Form A, sodium salt Form B, physical mixtures of magnesium salt Form A and Mg(OH)₂, physical mixtures of magnesium salt Form B and Mg(OH)₂, L-arginine salt Form A, L-lysine salt Form A and erbumine salt Form A were obtained after equilibration in water, EtOH, acetone, EA, ACN and THF. Screening with Ca(OH)₂ only led to gel. XRPD by Method 2 shown in FIG. 5.

TABLE 6

| | | A | B | C | D | E | F |
|---|---|---|---|---|---|---|---|
| | Counter ions | Water | Ethanol | Acetone | EA | ACN | THF |
| RC1 | N/A | // | Clear | Clear | Clear | Clear | Clear |
| | | | solution | solution | solution | solution | solution |
| RC2 | NaOH | // | Sodium | Sodium | Sodium | Gel | Sodium |
| | equiv.) | | salt Form | salt Form | salt Form | | salt Form |
| | | | A | B | A | | A |
| RC3 | KOH | // | Clear | Gel | Gel | Gel | Gel |
| | (1.0 equiv.) | | solution | | | | |
| RC4 | Ca(OH)₂ | Gel | Gel | Gel | Gel | Ge | Gel |
| | (0.5 equiv.) | | | | | | |
| RC5 | Mg(OH)₂ | After 3 | Magnesium | Amorphous | Magnesium | Magnesium | Magnesium |
| | (0.5 equiv.) | days: | salt | form + | salt | salt | salt |
| | FIGS. 5B, 5C | Amorphous | Form A + | Mg(OH)₂ | Form A + | Form B + | Form B + |
| | | form | Mg(OH)₂ | | Mg(OH)₂ | Mg(OH)₂ | Mg(OH)₂ |
| | | After 1 | | | | | |
| | | week: | | | | | |
| | | Amorphous | | | | | |
| | | form | | | | | |
| RC6 | L-Arginine | // | Gel | Gel | After 4 | Gel | Gel |
| | (1.0 equiv.) | | | | days: | | |
| | | | | | Gel | | |
| | | | | | After 1 | | |
| | | | | | week: | | |
| | | | | | L-Arginine | | |
| | | | | | salt Form | | |
| | | | | | A | | |
| RC7 | L-Lysine | // | L-Lysine | L-Lysine | L-Lysine | L-Lysine | L-Lysine |
| | (1.0 equiv.) | | salt Form | salt Form | salt Form | salt Form | salt Form |
| | | | A | A | A | A | A |
| RC8 | Methylglucamine | // | Clear | Gel | Gel | Gel | Gel |
| | (1.0 equiv.) | | solution | | | | |
| RC9 | Erbumine | // | Clear | Erbumine | Erbumine | Erbumine | Erbumine |
| | (1.0 equiv.) | | solution | salt Form | salt Form | salt Form | salt Form |
| | | | | A | A | A | A |
| RC10 | NH₃ | // | Clear | Clear | Clear | Clear | Oil |
| | (1.0 equiv.) | | solution | solution | solution | solution | |
| RC11 | TRIS | // | Clear | Gel | Gel | Gel | Gel |
| | (1.0 equiv.) | | solution | | | | |
| RC12 | Betaine | // | Clear | Amorphous | Gel | Hazy | Gel |
| | (1.0 equiv.) | | solution | form + | | suspension | |
| | | | | betaine | | | |

// = Note Carried Out.

Clear solutions obtain in slurry equilibration experiments were cooled to 5° C. to precipitate solids. Only clear solutions were obtained.

Clear solutions obtained from cooling experiments were further treated by addition of anti-solvent (one or more of heptane, methyl tert-butyl ether (MTBE), or toluene). Only gel, oil or clear solutions were obtained.

Clear solutions obtained from anti-solvent experiments were further treated by slow evaporation under ambient condition (20-30° C./20-70% RH). Only gel or oil was obtained.

Clear solutions obtained from cooling experiments were further treated by slow evaporation under ambient condition (20-30° C./20-70% RH). Only gel was obtained.

Gel samples obtained from slurry equilibration experiments were further treated by re-slurry at 25° C. for at least 48 hours by addition 0.1-0.5 ml of methanol (MeOH), isopropyl alcohol (IPA), MTBE, dichloromethane (DCM), methyl ethyl ketone (MEK), isopropyl acetate (IPAc), 2-Methyltetrahydrofuran (2-MeTHF), 1,4-dioxane, acetone/water (v:v=95:5), ACN/water (v:v=95:5), THF/water (v:v=95:5) and MeOH/water (v:v=95:5).

Obtained suspensions were filtered through a 0.45 μm nylon membrane filter by centrifugation at 14,000 rpm. After dried at 50° C. under vacuum for 2h, solids were analyzed by XRPD (Method 2). A new polymorph of arginine salt, assigned as L-arginine salt Form B, was obtained after re-slurry in acetone/water (v:v=95:5) and in ACN/water (v:v=95:5).

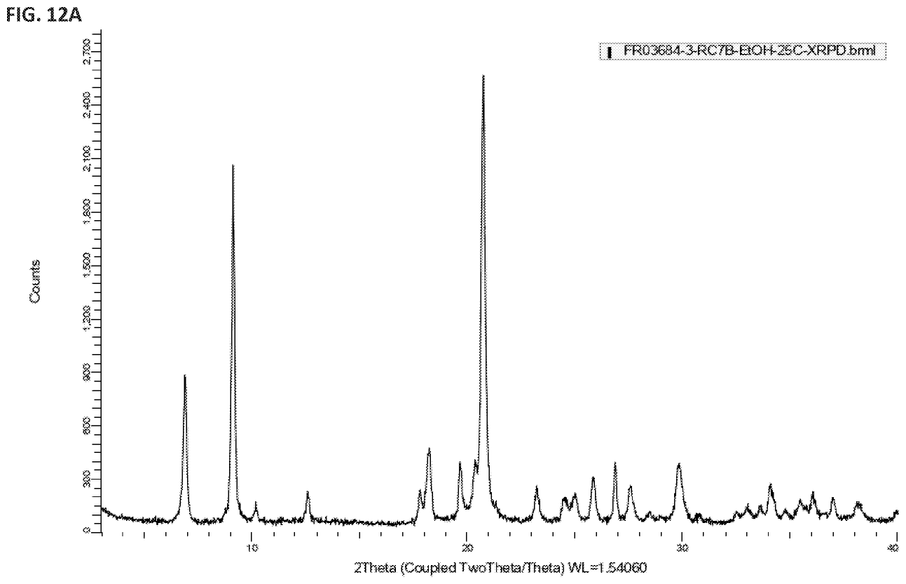

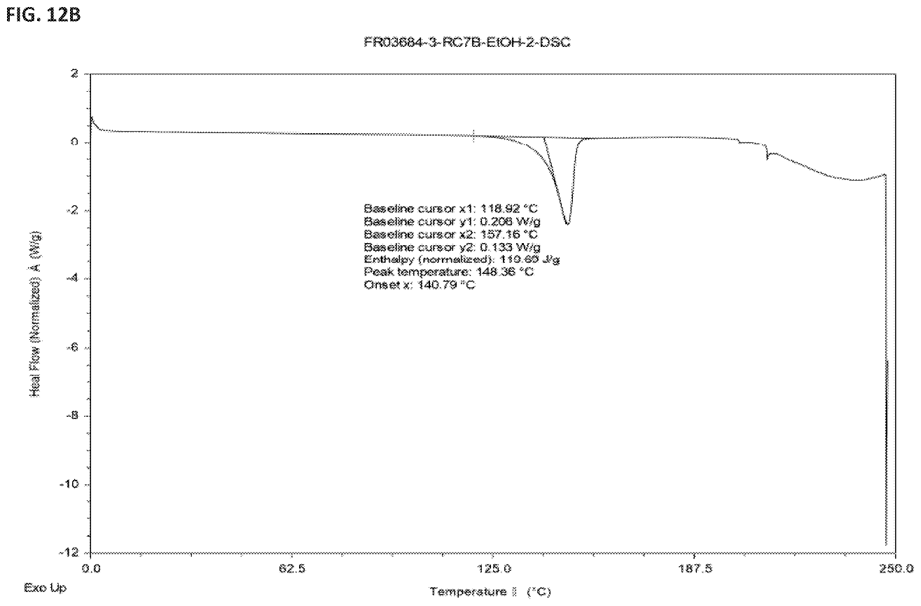

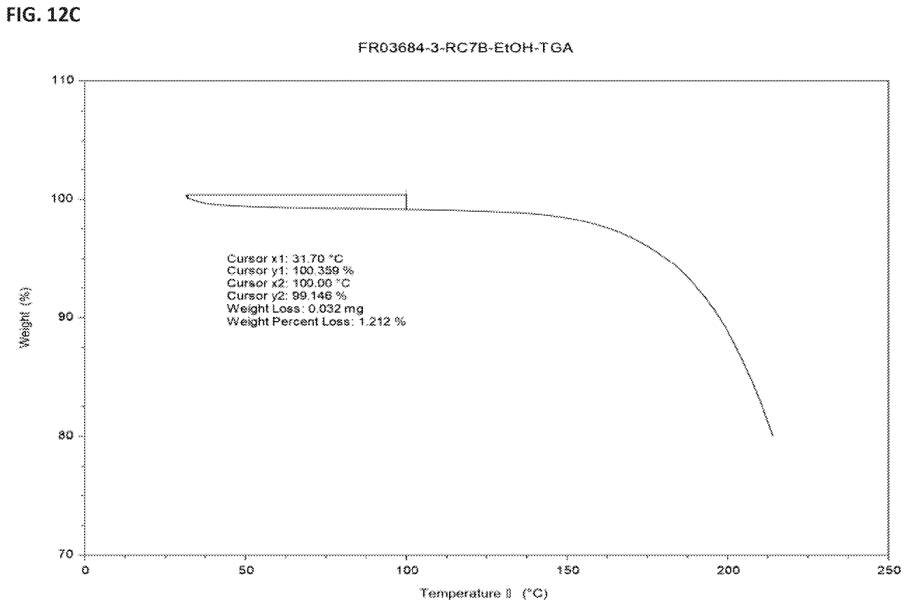

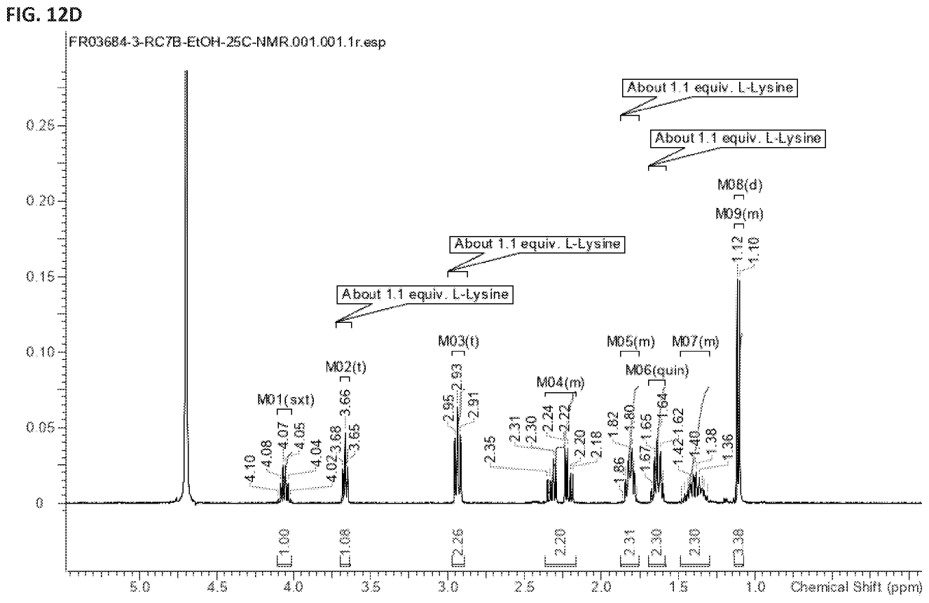

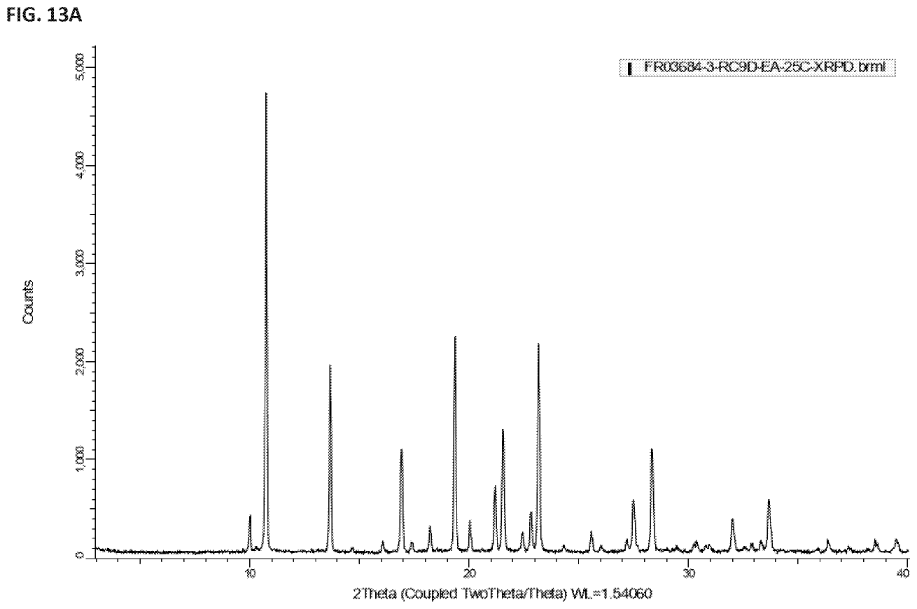

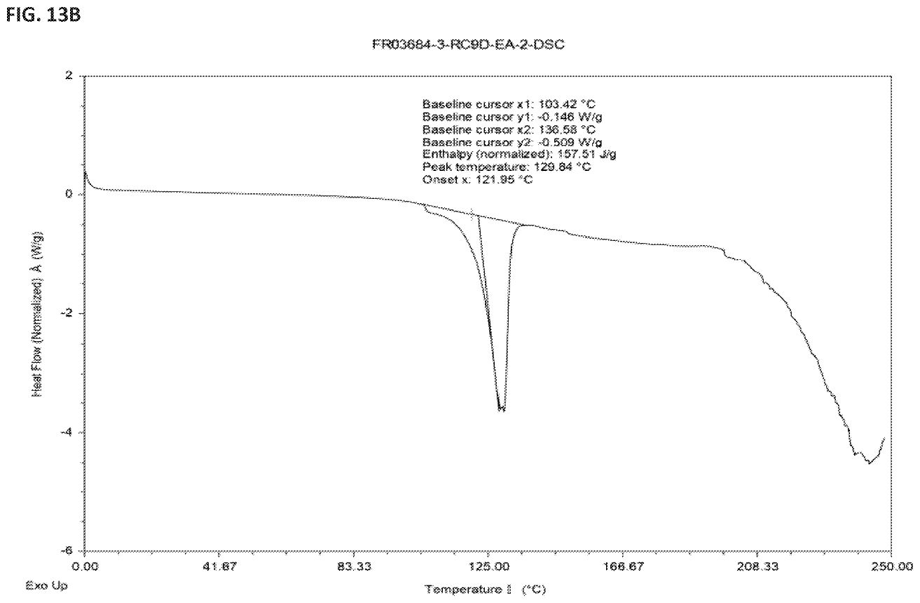

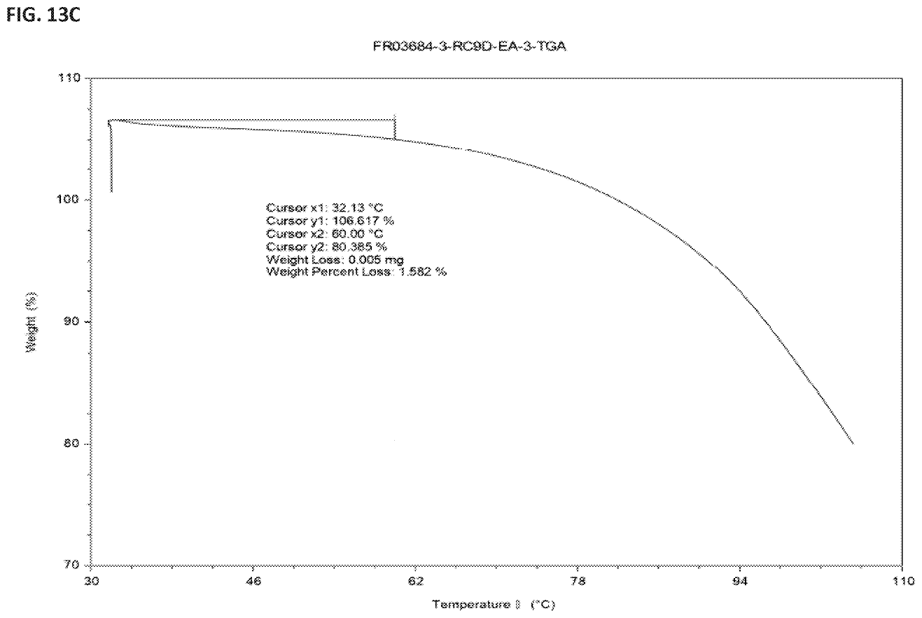

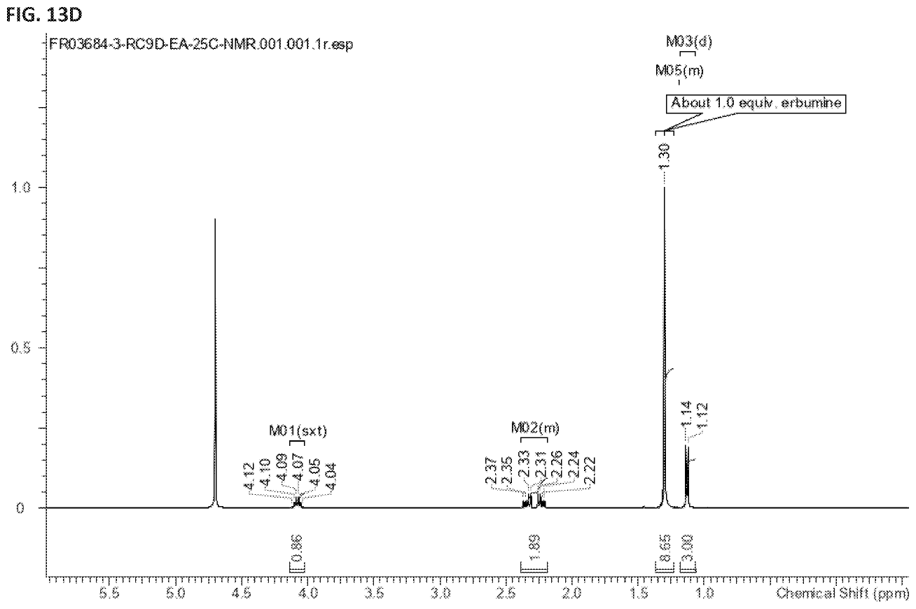

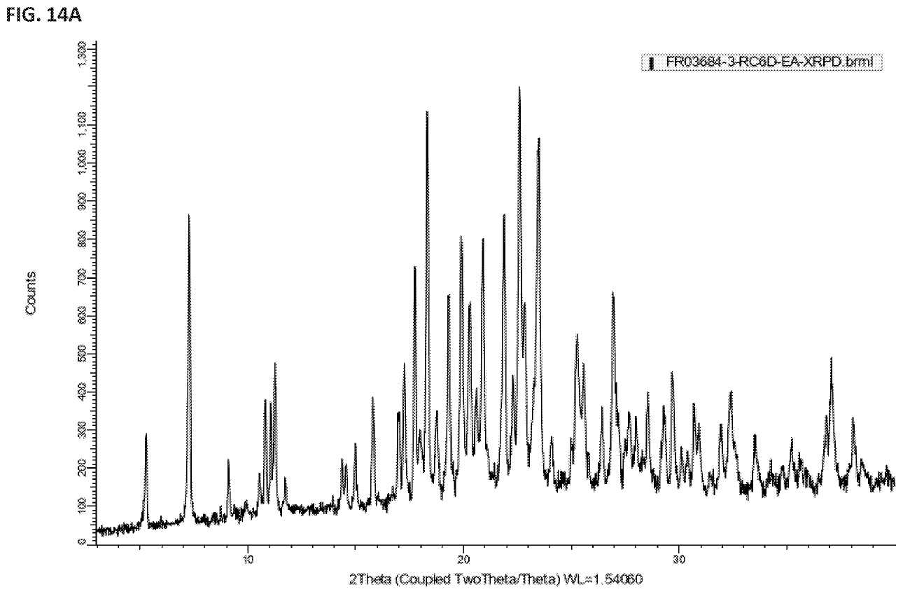

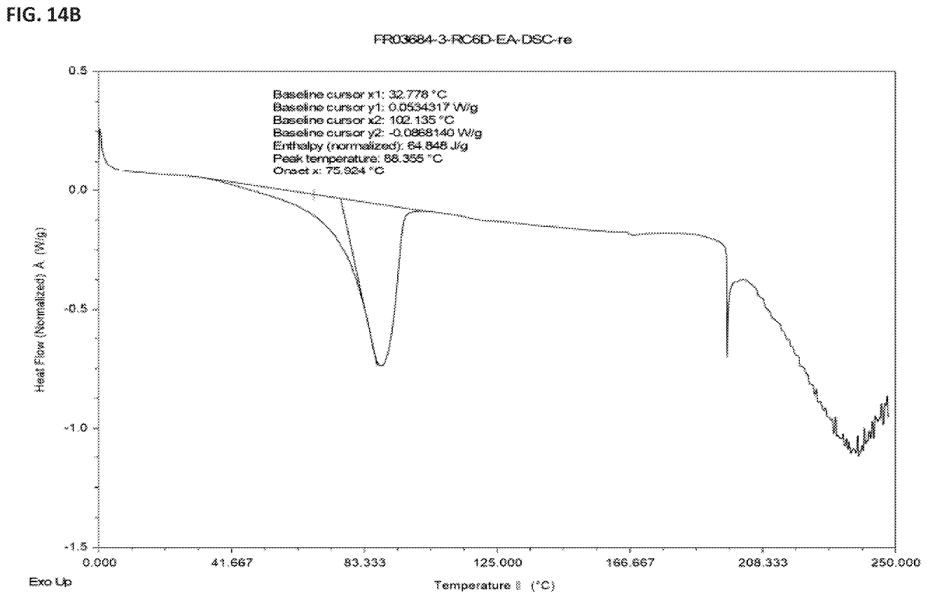

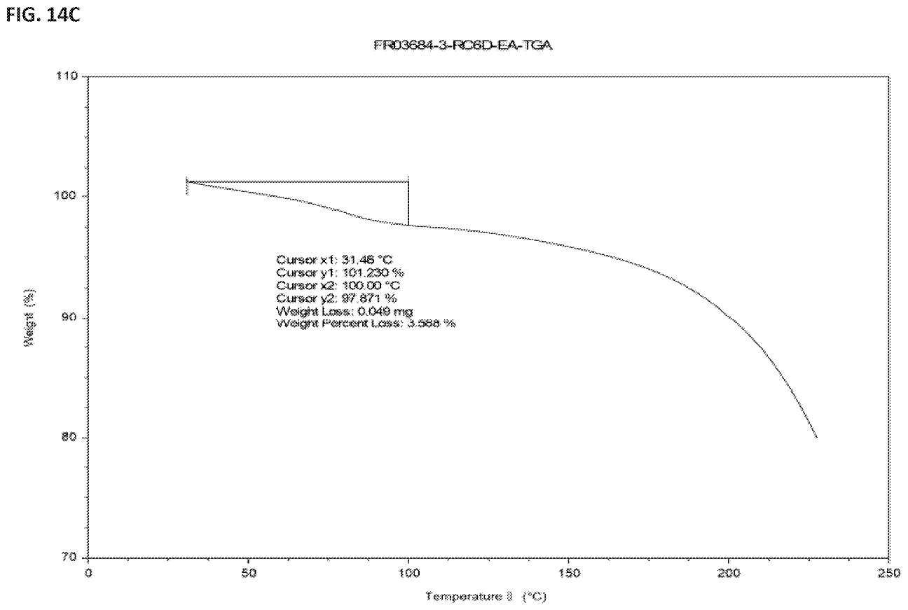

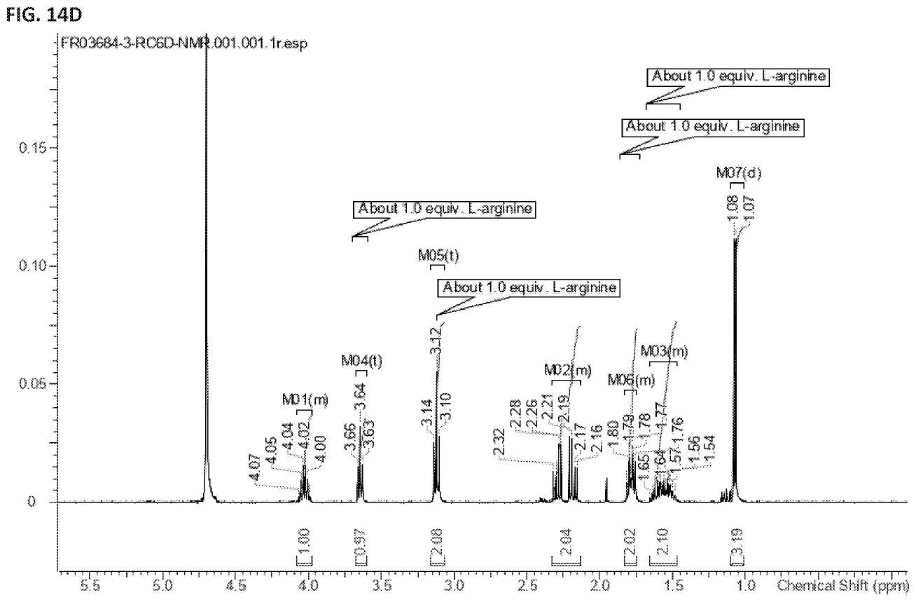

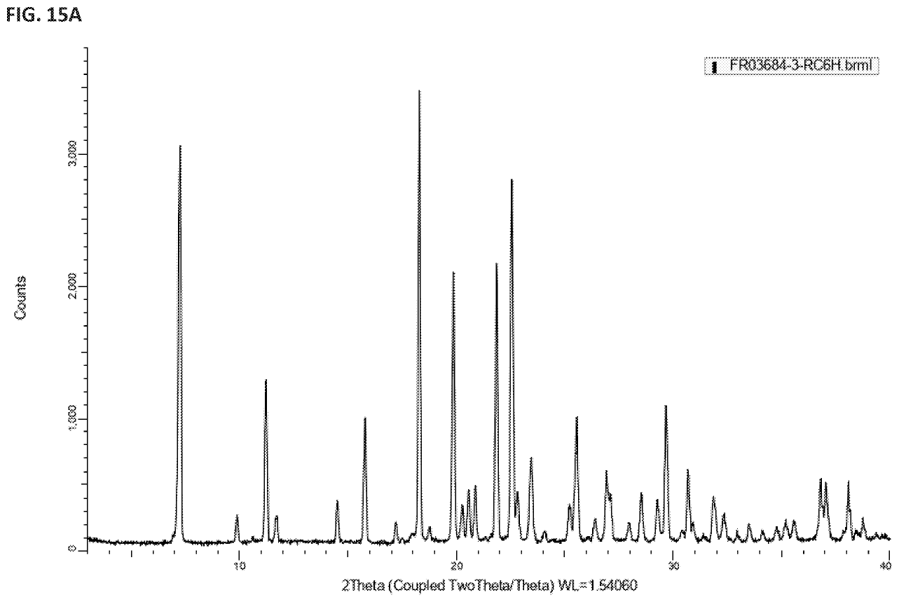

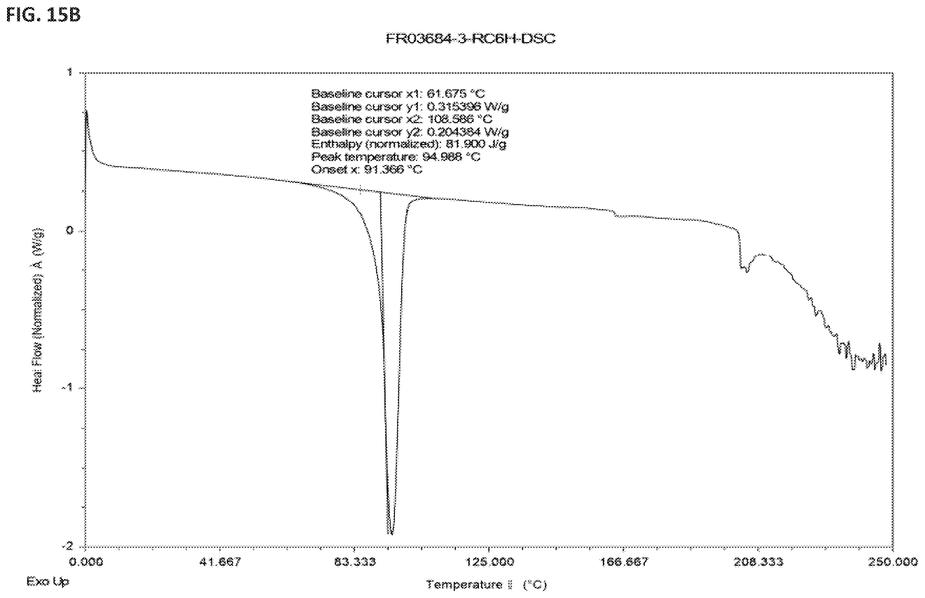

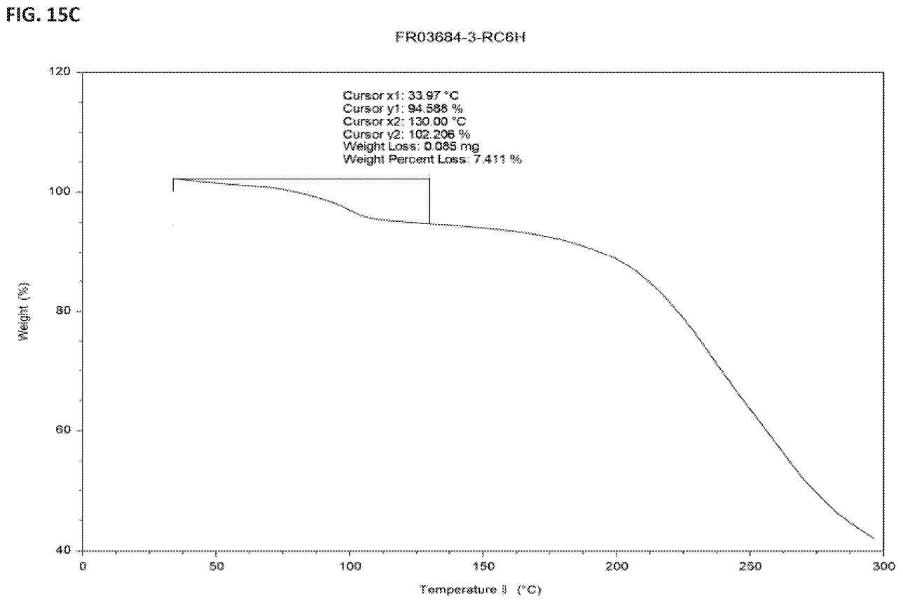

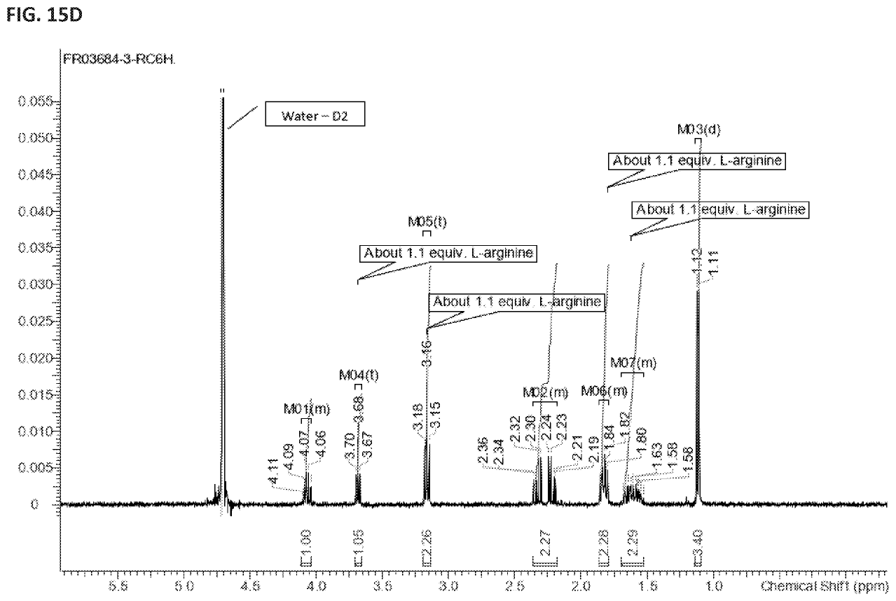

TABLE 7

| | L-Lysine | Erbumine | | L-Arginine |
|---|---|---|---|---|
| | salt | salt | L-Arginine | salt |
| | Form A, | Form A, | salt Form A, | Form B, |
| | anhydrate | anhydrate | hydrate | hydrate |
| Sample ID | FR03684-3- | FR03684-3- | FR03684-3- | FR03684-3- |
| | RC7B-EtOH | RC9D-EA | RC6D-EA | RC6H-acetone- |
| | | | | water-95-5 |
| Preparation | EtOH | EA | EA | Acetone/water |
| solvent | | | | (v:v = 95:5) |
| Crystallinity | High | High | Medium | High |
| (by XRPD | crystallinity | crystallinity | crystallinity | crystallinity |
| Method 2) | FIG. 12A | FIG. 13A | FIG. 14A | FIG. 15A |
| Melting | Melting | Melting Tₒₙₛₑₜ | Dehydration | Dehydration |
| onset (by | Tₒₙₛₑₜ | | | |
| DSC, ° C.) | @ 140.8° C. | @ 122.0° C.; | Tₒₙₛₑₜ @ | Tₒₙₛₑₜ @ |
| | FIG. 12B | Decomposition | 75.9° C.; | 91.4° C.; |
| | | upon melting | Dehydration | Dehydration |
| | | FIG. 13B | upon melting | upon melting |
| | | | FIG. 14B | FIG. 15B |
| Enthalpy (by | About | About | FIG. 14B | FIG. 15B |
| DSC, J/g) | 120 J/g | 158 J/g | | |
| | FIG. 12B | FIG. 13B | | |
| Weight | About 1.2% @ | About 1.6% @ | About 3.6% | About 7.4% @ |
| loss (by | 100° C. | 60° C. | @ 100° C. | 130° C. |
| TGA) | FIG. 12C | FIG. 13C | FIG. 14C | FIG. 15C |
| Stoichiometric | 1:1.1 | 1:1 | 1:1 | 1:1.1 |
| ratio | FIG. 12D | FIG. 13D | FIG. 14D | FIG. 15D |
| (by ¹H-NMR | | | | |
| or IC) | | | | |
| Residual | Undetected | Undetected | Undetected | Undetected |
| solvent | FIG. 12D | FIG. 13D | FIG. 14D | FIG. 15D |
| (by ¹H-NMR) | | | | |
| Water | // | // | 5.0% water | 5.8% water by |
| content (by | | | by weight | weight (1.0 |
| KF) for | | | (0.8 equiv. by | equiv. by |
| hydrate | | | molar ratio) | molar ratio) |

| | | | |
|---|---|---|---|
| Comments | — | — | Competitive equilibration |
| | | | between the L-arginine |
| | | | salt Form A and L-arginine |
| | | | salt Form B in EA at 25° C.: |
| | | | L-arginine salt Form |
| | | | B was obtained after 1 day. |

FIG. 12A shows the XRPD of D-BHB L-lysine salt Form A (FR03684-3-RC7B-EtOH) having peaks shown in TABLE 8.

TABLE 8
FR03684-3-RC7B-EtOH-25C

| | | | Net | Gross | |
|---|---|---|---|---|---|
| Index | Angle | d Value | Intensity | Intensity | Rel. Intensity |
| 1 | 6.901° | 12.79948 Å | 520.380 | 581.680 | 31.8% |
| 2 | 9.138° | 9.67037 Å | 1369.74 | 1436.89 | 83.6% |
| 3 | 10.182° | 8.68045 Å | 77.3529 | 142.893 | 4.7% |
| 4 | 12.603° | 7.01806 Å | 112.180 | 173.438 | 6.8% |
| 5 | 17.818° | 4.97405 Å | 117.468 | 178.255 | 7.2% |
| 6 | 18.245° | 4.85862 Å | 275.979 | 342.636 | 16.8% |
| 7 | 19.682° | 4.50701 Å | 210.294 | 288.486 | 12.8% |
| 8 | 20.376° | 4.35487 Å | 237.462 | 318.510 | 14.5% |
| 9 | 20.758° | 4.27558 Å | 1638.18 | 1718.71 | 100.0% |
| 10 | 23.268° | 3.81976 Å | 132.539 | 196.541 | 8.1% |
| 11 | 24.569° | 3.62037 Å | 72.9136 | 142.862 | 4.5% |
| 12 | 25.022° | 3.55585 Å | 100.676 | 173.032 | 6.1% |
| 13 | 25.870° | 3.44126 Å | 149.921 | 221.191 | 9.2% |
| 14 | 26.889° | 3.31309 Å | 241.093 | 313.212 | 14.7% |
| 15 | 27.586° | 3.23090 Å | 122.755 | 195.167 | 7.5% |
| 16 | 28.511° | 3.12818 Å | 34.2405 | 106.135 | 2.1% |
| 17 | 29.858° | 2.99000 Å | 200.775 | 273.617 | 12.3% |
| 18 | 30.782° | 2.90232 Å | 28.9599 | 94.7678 | 1.8% |
| 19 | 32.514° | 2.75161 Å | 34.6895 | 102.720 | 2.1% |
| 20 | 33.042° | 2.70881 Å | 64.3691 | 140.993 | 3.9% |
| 21 | 33.628° | 2.66295 Å | 47.1087 | 129.945 | 2.9% |
| 22 | 34.122° | 2.62556 Å | 123.704 | 209.083 | 7.6% |
| 23 | 34.776° | 2.57759 Å | 34.3996 | 121.545 | 2.1% |
| 24 | 35.475° | 2.52840 Å | 73.3416 | 164.557 | 4.5% |
| 25 | 36.086° | 2.48698 Å | 104.311 | 195.622 | 6.4% |
| 26 | 37.007° | 2.42721 Å | 79.6619 | 163.975 | 4.9% |
| 27 | 38.250° | 2.35113 Å | 58.4480 | 133.067 | 3.6% |

FIG. 13A shows the XRPD pattern of D-BHB erbumine salt Form A (FR03684-3-RC9D-EA) having peaks as shown in TABLE 9.

TABLE 9
FR03684-3-RC9D-EA

| Index | Angle | d Value | Net Intensity | Gross Intensity | Rel. Intesity |
|---|---|---|---|---|---|
| 1 | 10.018° | 8.82228 Å | 236.236 | 302.638 | 7.3% |
| 2 | 10.298° | 8.58329 Å | 24.4665 | 92.5738 | 0.8% |
| 3 | 10.762° | 8.21397 Å | 3237.94 | 3306.49 | 100% |
| 4 | 13.682° | 6.46710 Å | 1372.46 | 1436.96 | 42.4% |
| 5 | 14.687° | 6.02654 Å | 30.2357 | 85.5793 | 0.9% |
| 6 | 16.075° | 5.50907 Å | 79.4920 | 131.838 | 2.5% |
| 7 | 16.933° | 5.23187 Å | 725.893 | 783.576 | 22.4% |
| 8 | 17.405° | 5.09099 Å | 59.8622 | 119.520 | 1.8% |
| 9 | 18.237° | 4.86057 Å | 190.199 | 258.555 | 5.9% |
| 10 | 19.365° | 4.58008 Å | 1634.80 | 1706.95 | 50.5% |
| 11 | 20.057° | 4.42350 Å | 269.790 | 340.819 | 8.3% |
| 12 | 21.195° | 4.18842 Å | 507.696 | 581.069 | 15.7% |
| 13 | 21.560° | 4.11846 Å | 920.722 | 993.507 | 28-4% |
| 14 | 22.453° | 3.95664 Å | 132.986 | 204.967 | 4.1% |
| 15 | 22.836° | 3.89108 Å | 317.944 | 390.818 | 9.8% |
| 16 | 23.185° | 3.83327 Å | 1655.38 | 1727.30 | 51.1% |
| 17 | 24.334° | 3.65485 Å | 59.8396 | 123.452 | 1.8% |
| 18 | 25.601° | 3.47679 Å | 161.006 | 217.987 | 5.0% |
| 19 | 26.035° | 3.41977 Å | 56.0868 | 115.284 | 1.7% |
| 20 | 27.201° | 3.27574 Å | 97.9742 | 164.191 | 3.0% |
| 21 | 27.508° | 3.23989 Å | 422.263 | 489.888 | 13.0% |
| 22 | 28.355° | 3.14506 Å | 863.673 | 932.653 | 26.7% |
| 23 | 29.022° | 3.07427 Å | 15.9467 | 82.2430 | 0.5% |
| 24 | 29.315° | 3.04414 Å | 22.2069 | 85.7601 | 0.7% |
| 25 | 29.470° | 3.02852 Å | 60.5126 | 122.443 | 1.9% |
| 26 | 29.568° | 3.01866 Å | 18.4677 | 79.1965 | 0.6% |
| 27 | 29.835° | 2.99227 Å | 32.6357 | 91.6010 | 1.0% |
| 28 | 30.280° | 2.94935 Å | 55.1819 | 117.889 | 1.7% |
| 29 | 30.374° | 2.94038 Å | 78.6823 | 141.954 | 2.4% |
| 30 | 30.812° | 2.89963 Å | 51.6543 | 115.946 | 1.6% |
| 31 | 30.974° | 2.88477 Å | 71.0608 | 135.053 | 2.2% |
| 32 | 32.024° | 2.79261 Å | 296.165 | 369.667 | 9.1% |
| 33 | 32.573° | 2.74673 Å | 34.3847 | 111.853 | 1.1% |
| 34 | 32.904° | 2.71990 Å | 31.8102 | 109.654 | 1.9% |
| 35 | 33.325° | 2.68643 Å | 65.3848 | 141.857 | 1.0% |
| 36 | 33.684° | 2.65866 Å | 437.647 | 511.202 | 13.5% |
| 37 | 34.209° | 2.61902 Å | 26.1627 | 93.7126 | 0.8% |
| 38 | 36.384° | 2.46734 Å | 91.8363 | 152.003 | 2.8% |
| 39 | 37.317° | 2.40776 Å | 61.7713 | 125.497 | 1.9% |
| 40 | 38.533° | 2.33452 Å | 108.474 | 171.955 | 3.4% |
| 41 | 39.493° | 2.27994 Å | 111.904 | 175.589 | 3.5% |

FIG. 14A shows the XRPD of D-BHB L-arginine salt Form A (FR03684-3-RC6D-EA) having peaks as shown in TABLE 10.

TABLE 10
FR03684-3-RC6D-EA

| | | | Net | Gross | |
|---|---|---|---|---|---|
| Index | Angle | d Value | Intensity | Intensity | Rel. Intensity |
| 1 | 5.280° | 16.72266 Å | 149.883 | 195.988 | 20.3% |
| 2 | 7.282° | 12.13039 Å | 527.118 | 583.933 | 71.4% |
| 3 | 9.111° | 9.69814 Å | 79.9024 | 146.308 | 10.8% |
| 4 | 9.930° | 8.90025 Å | 21.9571 | 96.1724 | 3.0% |
| 5 | 10.553° | 8.37613 Å | 51.7468 | 132.893 | 7.0% |
| 6 | 10.811° | 8.17728 Å | 197.543 | 281.254 | 26.7% |
| 7 | 11.076° | 7.98212 Å | 185.588 | 271.463 | 25.1% |
| 8 | 11.269° | 7.84594 Å | 260.933 | 348.074 | 35.3% |
| 9 | 11.752° | 7.52397 Å | 39.6737 | 129.275 | 5.4% |
| 10 | 14.376° | 6.15621 Å | 87.8600 | 179.159 | 11.9% |
| 11 | 14.554° | 6.08118 Å | 75.3904 | 168.017 | 10.2% |
| 12 | 14.991° | 5.90508 Å | 128.012 | 222.970 | 17.3% |
| 13 | 15.815° | 5.59899 Å | 214.832 | 313.813 | 29.1% |
| 14 | 17.001° | 5.21110 Å | 146.916 | 266.363 | 19.9% |
| 15 | 17.256° | 5.13466 Å | 240.877 | 365.767 | 32.6% |
| 16 | 17.757° | 4.99085 Å | 424.882 | 559.151 | 57.5% |
| 17 | 17.978° | 4.92998 Å | 109.856 | 247.709 | 14.9% |
| 18 | 18.323° | 4.83804 Å | 707.347 | 850.113 | 95.8% |
| 19 | 18.774° | 4.72291 Å | 123.223 | 271.174 | 16.7% |
| 20 | 19.315° | 4.59178 Å | 364.929 | 519.760 | 49.4% |
| 21 | 19.902° | 4.45754 Å | 428.160 | 590.064 | 58.0% |
| 22 | 20.307° | 4.36970 Å | 316.495 | 481.877 | 42.8% |
| 23 | 20.616° | 4.30490 Å | 164.231 | 331.511 | 22.2% |
| 24 | 20.904° | 4.24615 Å | 442.316 | 610.769 | 59.9% |
| 25 | 21.889° | 4.05727 Å | 493.214 | 663.154 | 66.8% |
| 26 | 22.314° | 3.98087 Å | 195.963 | 365.968 | 26.5% |
| 27 | 22.604° | 3.93047 Å | 738.727 | 908.058 | 100.0% |
| 28 | 22.841° | 3.89016 Å | 321.940 | 490.285 | 43.6% |
| 29 | 23.475° | 3.78667 Å | 589.027 | 752.835 | 79.7% |
| 30 | 24.098° | 3.69010 Å | 94.5935 | 251.222 | 12.8% |
| 31 | 25.016° | 3.55676 Å | 76.9708 | 232.737 | 10.4% |
| 32 | 25.278° | 3.52043 Å | 256.140 | 413.309 | 34.7% |
| 33 | 25.578° | 3.47988 Å | 239.884 | 398.068 | 32.5% |
| 34 | 26.431° | 3.36941 Å | 153.198 | 314.182 | 20.7% |
| 35 | 26.957° | 3.30486 Å | 366.774 | 529.956 | 49.6% |
| 36 | 27.699° | 3.21805 Å | 129.874 | 292.905 | 17.6% |
| 37 | 28.009° | 3.18304 Å | 135.936 | 297.766 | 18.4% |
| 38 | 28.564° | 3.12250 Å | 183.373 | 341.407 | 24.8% |
| 39 | 29.298° | 3.04594 Å | 163.767 | 316.814 | 22.2% |
| 40 | 29.708° | 3.00479 Å | 218.093 | 370.296 | 29.5% |
| 41 | 30.120° | 2.96467 Å | 78.0114 | 228.198 | 10.6% |
| 42 | 30.407° | 2.93727 Å | 66.2784 | 214.358 | 9.0% |
| 43 | 30.768° | 2.90363 Å | 61.8369 | 206.460 | 8.4% |
| 44 | 30.917° | 2.89000 Å | 126.509 | 269.448 | 17.1% |
| 45 | 31.946° | 2.79925 Å | 83.6692 | 225.403 | 11.3% |
| 46 | 32.422° | 2.75916 Å | 140.162 | 282.459 | 19.0% |
| 47 | 33.529° | 2.67059 Å | 87.4170 | 225.812 | 11.8% |
| 48 | 34.292° | 2.61288 Å | 37.5582 | 177.108 | 5.1% |
| 49 | 34.798° | 2.57604 Å | 43.9456 | 186.991 | 5.9% |
| 50 | 35.235° | 2.54508 Å | 72.1142 | 216.754 | 9.8% |
| 51 | 35.641° | 2.51703 Å | 57.4126 | 202.348 | 7.8% |
| 52 | 36.839° | 2.43787 Å | 144.960 | 293.907 | 19.6% |
| 53 | 37.079° | 2.42262 Å | 260.028 | 410.978 | 35.2% |
| 54 | 38.095° | 2.36033 Å | 151.922 | 306.919 | 20.6% |
| 55 | 38.518° | 2.33536 Å | 37.0213 | 191.595 | 5.0% |
| 56 | 39.640° | 2.27182 Å | 49.6007 | 202.549 | 6.7% |

FIG. 15A shows the XRPD of D-BHB L-arginine salt Form B (FR03684-3-RC6H-acetone-water-95-5) having peaks as shown in TABLE 11.

TABLE 11
FR03684-3-RC6H-acetone-water-95-5

| | | | Net | Gross | |
|---|---|---|---|---|---|
| Index | Angle | d Value | Intensity | Intensity | Rel. Intensity |
| 1 | 7.253° | 12.17757 Å | 1970.18 | 2029.47 | 82.0% |
| 2 | 9.893° | 8.93362 Å | 149.595 | 211.312 | 6.2% |
| 3 | 10.617° | 8.32590 Å | 27.5226 | 92.4228 | 1.1% |
| 4 | 11.233° | 7.87069 Å | 831.979 | 900.700 | 34.6% |
| 5 | 11.707° | 7.55290 Å | 104.961 | 173.576 | 4.4% |
| 6 | 14.520° | 6.09552 Å | 212.380 | 275.481 | 8.8% |
| 7 | 15.797° | 5.60566 Å | 673.915 | 734.657 | 28.0% |
| 8 | 17.229° | 5.14261 Å | 103.718 | 163.787 | 4.3% |
| 9 | 17.512° | 5.06025 Å | 20.9727 | 85.6214 | 0.9% |
| 10 | 17.977° | 4.93043 Å | 33.3998 | 103.283 | 1.4% |
| 11 | 18.301° | 4.84367 Å | 2403.36 | 2475.38 | 100.0% |
| 12 | 18.771° | 4.72351 Å | 74.8745 | 147.805 | 3.1% |
| 13 | 19.867° | 4.46542 Å | 1460.81 | 1536.48 | 60.8% |
| 14 | 20.287° | 4.37397 Å | 179.567 | 258.593 | 7.5% |
| 15 | 20.581° | 4.31202 Å | 276.795 | 356.934 | 11.5% |
| 16 | 20.890° | 4.24900 Å | 295.532 | 375.741 | 12.3% |
| 17 | 21.619° | 4.10738 Å | 41.2573 | 120.144 | 1.7% |
| 18 | 21.861° | 4.06242 Å | 1547.59 | 1628.46 | 64.4% |
| 19 | 22.570° | 3.93633 Å | 2025.71 | 2108.42 | 84.3% |
| 20 | 22.834° | 3.89138 Å | 254.894 | 336.773 | 10.6% |
| 21 | 23.451° | 3.79043 Å | 445.482 | 522.218 | 18.5% |
| 22 | 24.077° | 3.69325 Å | 58.3383 | 128.451 | 2.4% |
| 23 | 25.234° | 3.52655 Å | 202.752 | 273.133 | 8.4% |
| 24 | 25.555° | 3.48289 Å | 654.664 | 728.222 | 27.2% |
| 25 | 26.120° | 3.40879 Å | 18.4813 | 94.6565 | 0.8% |
| 26 | 26.402° | 3.37303 Å | 114.168 | 192.581 | 4.8% |
| 27 | 26.949° | 3.30588 Å | 335.109 | 417.418 | 13.9% |
| 28 | 27.103° | 3.28744 Å | 223.826 | 306.599 | 9.3% |
| 29 | 27.995° | 3.18460 Å | 89.9143 | 169.799 | 3.7% |
| 30 | 28.539° | 3.12517 Å | 268.022 | 344.348 | 11.2% |
| 31 | 29.280° | 3.04777 Å | 250.611 | 328.382 | 10.4% |
| 32 | 29.683° | 3.00731 Å | 790.078 | 870.174 | 32.9% |
| 33 | 30.417° | 2.93634 Å | 48.7423 | 130.166 | 2.0% |
| 34 | 30.695° | 2.91039 Å | 440.485 | 522.310 | 18.3% |
| 35 | 30.902° | 2.89133 Å | 99.8403 | 181.372 | 4.2% |
| 36 | 31.406° | 2.84607 Å | 50.5617 | 129.264 | 2.1% |
| 37 | 31.880° | 2.80486 Å | 248.460 | 323.800 | 10.3% |
| 38 | 32.350° | 2.76517 Å | 153.255 | 225.250 | 6.4% |
| 39 | 32.606° | 2.74402 Å | 19.1302 | 88.2086 | 0.8% |
| 40 | 32.965° | 2.71500 Å | 76.0199 | 144.867 | 3.2% |
| 41 | 33.504° | 2.67249 Å | 108.180 | 180.990 | 4.5% |
| 42 | 34.123° | 2.62547 Å | 75.0143 | 148.510 | 3.1% |
| 43 | 34.789° | 2.57671 Å | 75.2529 | 152.833 | 3.1% |
| 44 | 35.202° | 2.54737 Å | 111.434 | 192.383 | 4.6% |
| 45 | 35.588° | 2.52068 Å | 106.726 | 188.998 | 4.4% |
| 46 | 36.811° | 2.43966 Å | 391.231 | 480.494 | 16.3% |
| 47 | 37.060° | 2.42382 Å | 363.142 | 452.777 | 15.1% |
| 48 | 38.090° | 2.36062 Å | 394.244 | 483.300 | 16.4% |
| 49 | 38.467° | 2.33838 Å | 35.2387 | 124.153 | 1.5% |
| 50 | 38.768° | 2.32091 Å | 130.679 | 218.281 | 5.4% |
| 51 | 39.373° | 2.28659 Å | 37.8548 | 125.350 | 1.6% |

### Example 3: Magnesium Salt Forms

Condition 1: To get pure magnesium salt polymorphs for characterization, obtained physical mixtures of crystalline magnesium salt and Mg(OH)₂ were used as seeds during screening.

About 30 mg of the D-BHB free Form I (FR03684-1-LP1) and 0.5 equiv. of Mg(OH)₂ were added into 0.1-0.3 mL of EtOH, EA, ACN and THE in a 2 ml glass vial. After stirring at 50° C. for 10 min, about 3 mg of physical mixtures of crystalline magnesium salt and Mg(OH)₂ were added into above suspension as seeds.

Obtained mixtures were stirred at 50° C. for 2 hours and then at 25° C. for at least 48 hours.

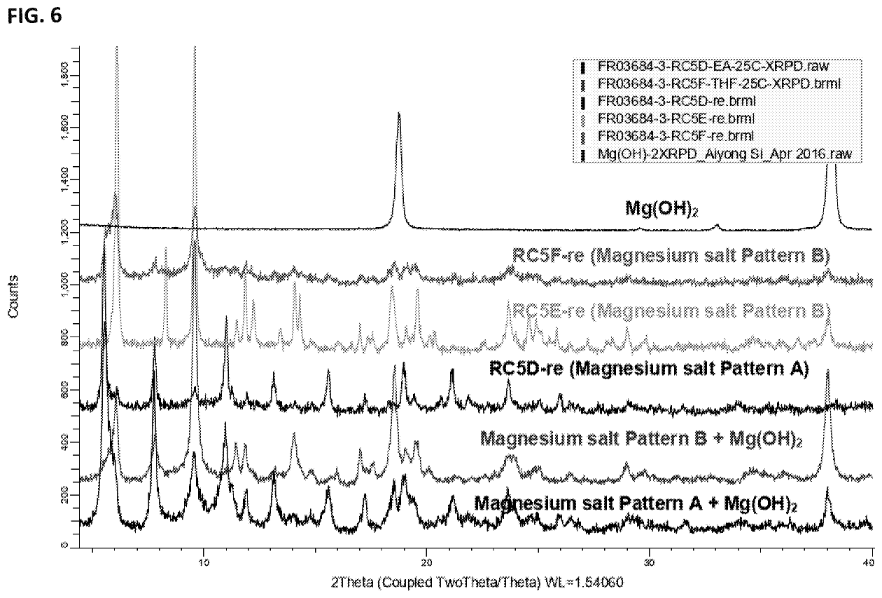

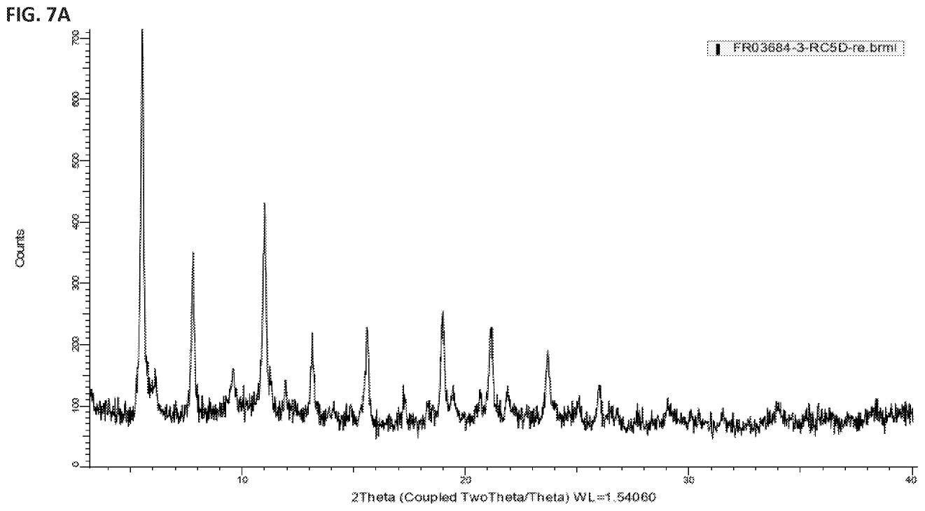

Hemi-magnesium salt Form A was obtained in EA. Hemi-magnesium salt Form B was obtained in ACN and in THF (FIG. 6).

TABLE 12

| Exp. | Counter | B | D | E | F |
|---|---|---|---|---|---|
| ID | ions | Ethanol | EA | ACN | THF |
| RC5- | Mg(OH)₂ | Hazy | Magnesium | Magnesium | Magnesium |
| re | (0.5 | suspension | salt Form A | salt Form B | salt Form B |
| | equiv.) | | FIG. 6 | FIG. 6 | FIG. 6 |

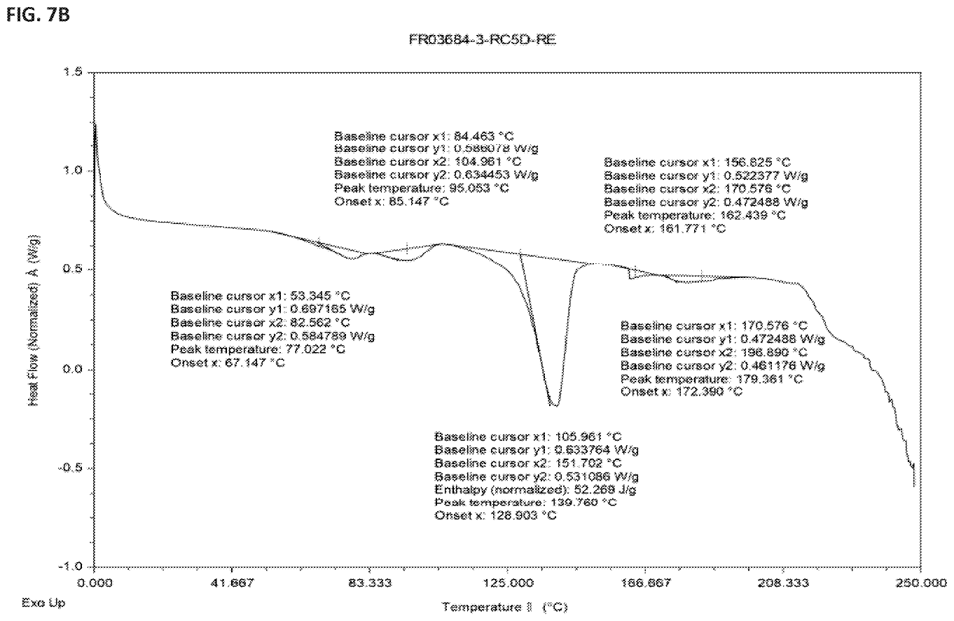

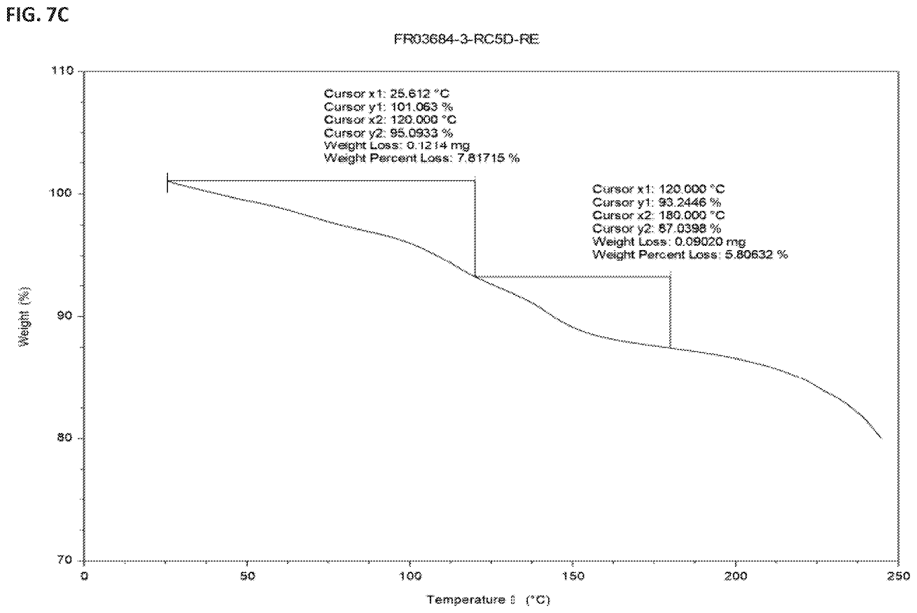

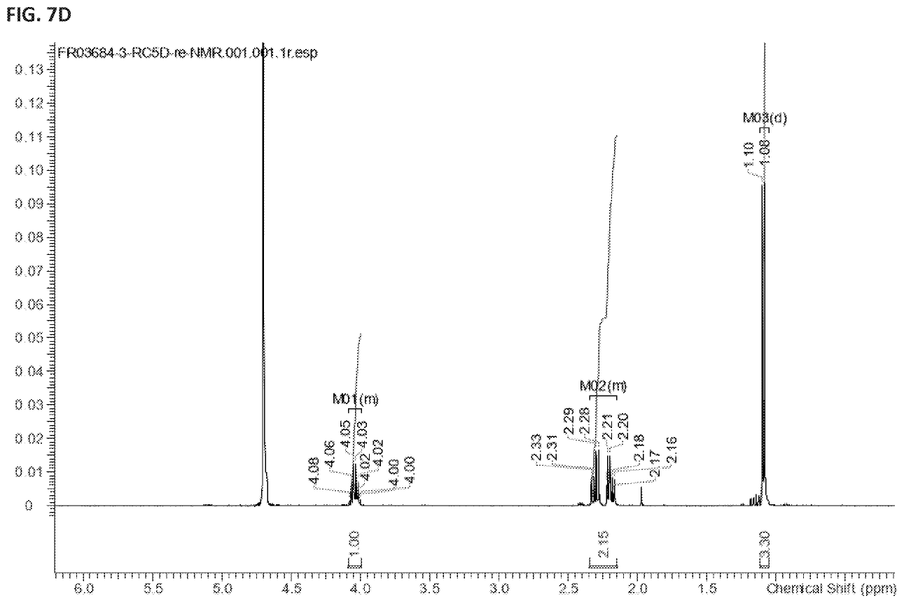

FIG. 7 shows D-BHB magnesium salt Form A (FR03684-3-RC5D-re-EA) with FIG. 7A showing the XRPD having peaks as shown in TABLE 13.

TABLE 13
FR03684-3-RC5D-re-EA

| | | | Net | Gross | |
|---|---|---|---|---|---|
| Index | Angle | d Value | Intensity | Intensity | Rel. Intensity |
| 1 | 5.535° | 15.95403 Å | 404.222 | 487.838 | 100.0% |
| 2 | 6.111° | 14.45141 Å | 47.3354 | 130.709 | 11.7% |
| 3 | 7.799° | 11.32675 Å | 175.003 | 258.611 | 43.3% |
| 4 | 9.605° | 9.20121 Å | 45.8208 | 138.226 | 11.3% |
| 5 | 11.017° | 8.02487 Å | 243.288 | 335.807 | 60.2% |
| 6 | 11.272° | 7.84358 Å | 39.6366 | 131.033 | 9.8% |
| 7 | 11.947° | 7.40172 Å | 40.6289 | 128.258 | 10.1% |
| 8 | 13.146° | 6.72954 Å | 107.481 | 188.637 | 26.6% |
| 9 | 14.864° | 5.95518 Å | 20.4580 | 92.6234 | 5.1% |
| 10 | 15.335° | 5.77344 Å | 15.3683 | 87.4989 | 3.8% |
| 11 | 15.605° | 5.67388 Å | 103.053 | 174.626 | 25.5% |
| 12 | 17.021° | 5.20508 Å | 27.3295 | 95.7886 | 6.8% |
| 13 | 17.243° | 5.13864 Å | 29.8079 | 98.8231 | 7.4% |
| 14 | 18.361° | 4.82804 Å | 25.4017 | 97.0853 | 6.3% |
| 15 | 18.540° | 4.78177 Å | 18.4416 | 91.5622 | 4.6% |
| 16 | 18.982° | 4.67143 Å | 119.897 | 195.818 | 29.7% |
| 17 | 19.450° | 4.56009 Å | 40.1638 | 117.911 | 9.9% |
| 18 | 20.708° | 4.28586 Å | 21.8418 | 102.533 | 5.4% |
| 19 | 21.130° | 4.20124 Å | 98.9649 | 180.334 | 24.5% |
| 20 | 21.881° | 4.05872 Å | 26.9361 | 107.156 | 6.7% |
| 21 | 23.549° | 3.77494 Å | 16.5220 | 93.0500 | 4.1% |
| 22 | 23.695° | 3.75200 Å | 79.6202 | 156.297 | 19.7% |
| 23 | 25.082° | 3.54757 Å | 25.9612 | 98.3522 | 6.4% |
| 24 | 25.989° | 3.42572 Å | 48.8960 | 117.052 | 12.1% |
| 25 | 26.419° | 3.37097 Å | 34.4814 | 102.247 | 8.5% |
| 26 | 26.806° | 3.32320 Å | 22.7861 | 89.3527 | 5.6% |
| 27 | 29.063° | 3.06997 Å | 30.6678 | 97.0416 | 7.6% |
| 28 | 30.072° | 2.96926 Å | 15.5136 | 78.8080 | 3.8% |
| 29 | 30.455° | 2.93280 Å | 29.7080 | 90.4035 | 7.3% |
| 30 | 31.520° | 2.83603 Å | 19.5942 | 77.7423 | 4.8% |
| 31 | 34.021° | 2.63308 Å | 22.1597 | 92.4162 | 5.5% |
| 32 | 38.450° | 2.33934 Å | 15.5469 | 90.5445 | 3.8% |

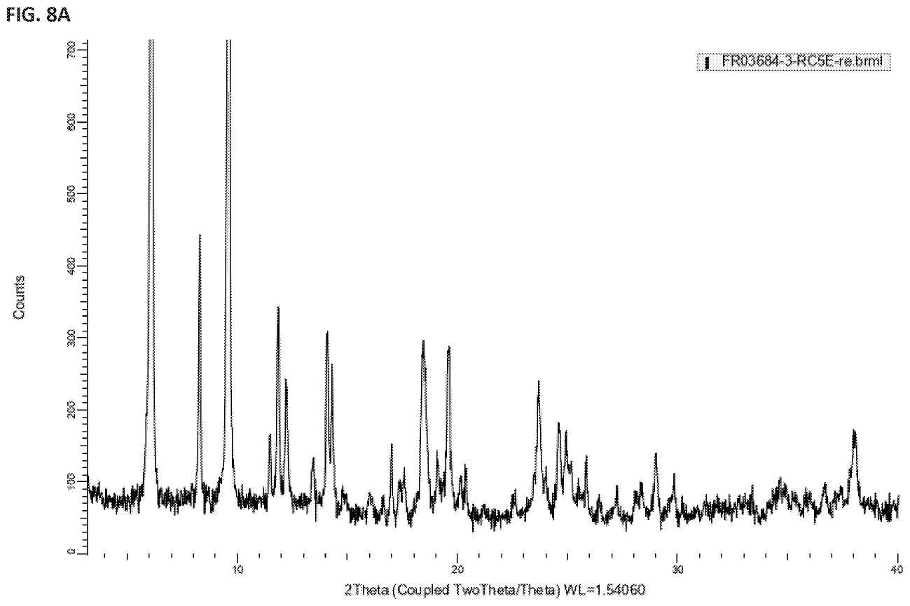

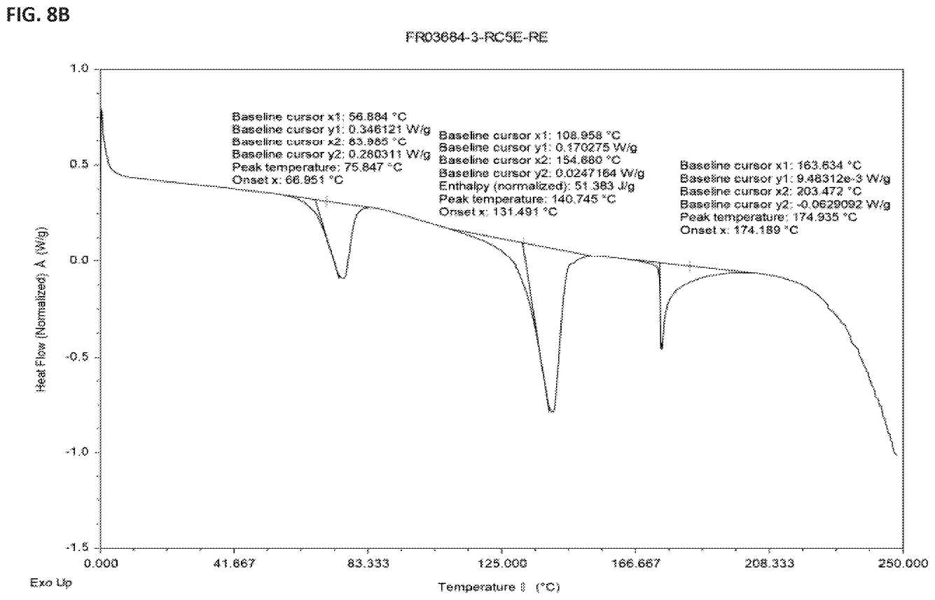

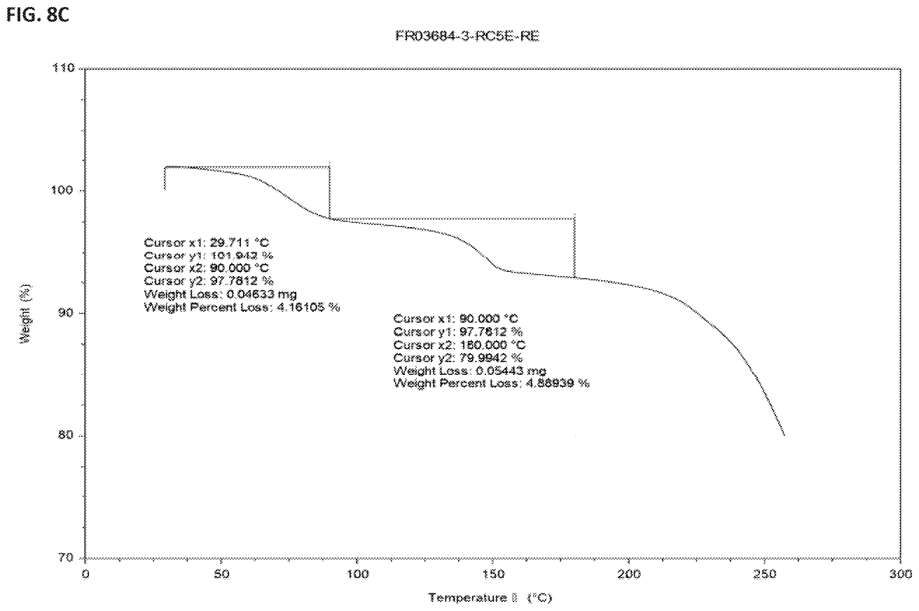

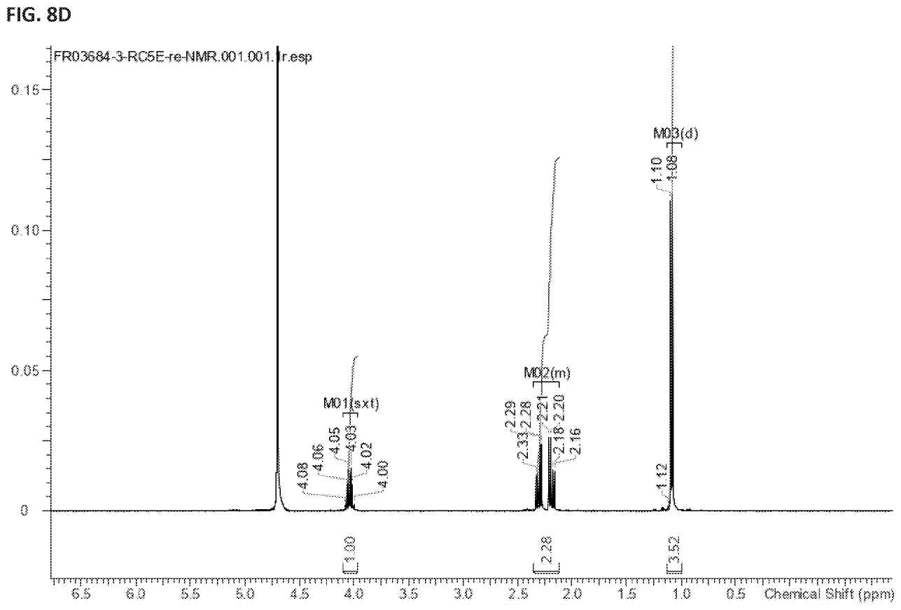

FIG. 8 shows D-BHB magnesium salt Form B (FR03684-3-RC5E-re-ACN) with FIG. 8A showing the XRPD having peaks as shown in TABLE 14.

TABLE 14
FR03684-3-RCSE-re-ACN

| | | | Net | Gross | |
|---|---|---|---|---|---|
| Index | Angle | d Value | Intensity | Intensity | Rel. Intensity |
| 1 | 6.115° | 14.44168 Å | 818.336 | 892.287 | 90.5% |
| 2 | 8.306° | 10.63635 Å | 252.936 | 321.424 | 28.0% |
| 3 | 8.666° | 10.19586 Å | 15.1044 | 81.1039 | 1.7% |
| 4 | 9.603° | 9.20269 Å | 904.001 | 973.279 | 100.0% |
| 5 | 11.493° | 7.69331 Å | 51.1348 | 124.278 | 5.7% |
| 6 | 11.865° | 7.45299 Å | 185.694 | 259.062 | 20.5% |
| 7 | 12.234° | 7.22893 Å | 103.043 | 175.609 | 11.4% |
| 8 | 13.445° | 6.58024 Å | 43.8614 | 112.606 | 4.9% |
| 9 | 14.085° | 6.28268 Å | 159.384 | 225.381 | 17.6% |
| 10 | 14.314° | 6.18254 Å | 145.670 | 209.936 | 16.1% |
| 11 | 16.029° | 5.52497 Å | 22.4205 | 72.9685 | 2.5% |
| 12 | 16.647° | 5.32122 Å | 23.1025 | 69.7972 | 2.6% |
| 13 | 17.013° | 5.20741 Å | 87.2689 | 134.203 | 9.7% |
| 14 | 17.391° | 5.09501 Å | 36.0137 | 85.1853 | 4.0% |
| 15 | 17.579° | 5.04101 Å | 47.7056 | 97.5896 | 5.3% |
| 16 | 18.459° | 4.80274 Å | 160.344 | 215.976 | 17.7% |
| 17 | 19.101° | 4.64263 Å | 42.0083 | 100.526 | 4.6% |
| 18 | 19.609° | 4.52364 Å | 141.057 | 199.666 | 15.6% |
| 19 | 20.149° | 4.40347 Å | 33.7349 | 90.3145 | 3.7% |
| 20 | 20.371° | 4.35612 Å | 43.5380 | 98.6550 | 4.8% |
| 21 | 23.699° | 3.75131 Å | 134.755 | 197.370 | 14.9% |
| 22 | 24.616° | 3.61360 Å | 74.9171 | 138.181 | 8.3% |
| 23 | 24.943° | 3.56691 Å | 85.7213 | 147.970 | 9.5% |
| 24 | 25.158° | 3.53694 Å | 43.7420 | 104.886 | 4.8% |
| 25 | 25.515° | 3.48831 Å | 38.2629 | 96.8105 | 4.2% |
| 26 | 25.849° | 3.44398 Å | 53.8097 | 109.059 | 6.0% |
| 27 | 26.457° | 3.36613 Å | 25.1784 | 75.1099 | 2.8% |
| 28 | 27.246° | 3.27050 Å | 37.5050 | 87.1215 | 4.1% |
| 29 | 28.349° | 3.14568 Å | 31.6555 | 84.6662 | 3.5% |
| 30 | 29.022° | 3.07428 Å | 61.0259 | 115.363 | 6.8% |
| 31 | 29.835° | 2.99227 Å | 27.8150 | 79.2154 | 3.1% |
| 32 | 33.395° | 2.68102 Å | 27.5223 | 79.7212 | 3.0% |
| 33 | 34.642° | 2.58728 Å | 33.2898 | 90.2827 | 3.7% |
| 34 | 35.852° | 2.50269 Å | 16.2603 | 75.9889 | 1.8% |
| 35 | 36.719° | 2.44554 Å | 18.8244 | 79.8748 | 2.1% |
| 36 | 37.422° | 2.40122 Å | 22.1298 | 85.1888 | 2.4% |
| 37 | 38.083° | 2.36104 Å | 62.1933 | 125.170 | 6.9% |

Condition 2: To get pure magnesium salt polymorphs for characterization, amorphous magnesium salt was also prepared for re-slurry.

About 500 mg of the D-BHB free Form I (ID FR03684-1-LP1) and 0.5 equiv. of Mg(OH)₂ were added into water in a 2 mL glass vial. Obtained mixtures were stirred at 25° C. for at least 48 hours. About 346 mg of amorphous magnesium salt was obtained.

Obtained amorphous hemi-magnesium salt was further treated by re-slurry at 25° C. for at least 48 hours in 0.1-0.3 ml of EtOH, EA, ACN and THF. About 3 mg of magnesium salt Form A and Mg(OH)₂ mixture seeds (FR03684-3-RC5B-EtOH and FR03684-3-RC5D-EA) was added into suspension RS9B and RS9D, respectively. About 3 mg of the magnesium salt Form B and Mg(OH)₂ mixture seeds (FR03684-3-RC5E-ACN and FR03684-3-RC5F-THF) was added into suspension RS9E and RS9F, respectively.

Obtained suspensions were filtered through a 0.45 μm nylon membrane filter by centrifugation at 14,000 rpm. After dried at 50° C. under vacuum for 2h, solids were analyzed by XRPD (Method 2). Based on results, only magnesium salt Form B was obtained after re-slurry in ACN.

Condition 3: To get pure magnesium salt polymorphs for characterization, EtOH/water (v:v=95:5), ACN/water (v:v=95:5) and THF/water (v:v=95:5) were also selected as screening solvents. About 150 mg of the D-BHB free Form I (sample ID FR03684-1-LP1) and 0.5 equiv. of Mg(OH)₂ were added into 0.1-0.5 mL of EtOH/water (v:v=95:5), ACN/water (v:v=95:5) and THF/water (v:v=95:5) solvent mixtures in a 2 mL glass vial. Obtained mixtures were stirred at 25° C. for at least 48 hours. Only emulsion was obtained.

TABLE 15

| | Hemi-Magnesium | Hemi-Magnesium |
|---|---|---|
| | salt Form A, | salt Form B, |
| | hydrate | hydrate |
| Sample ID | FR03684-3-RC5D-re-EA | FR03684-3-RC5E-re-ACN |
| Counter ion Class | I | I |
| Preparation solvent | EA | ACN |
| Crystallinity (by XRPD | Medium crystallinity | Medium crystallinity |
| Method 2) | FIG. 7A | FIG. 8A |
| Melting onset (by | Dehydration from | Dehydration from |
| DSC, ° C.) | about 53° C.; | about |
| | Endothermic event Tₒₙₛₑₜ @ | 57° C.; |
| | 85.1° C.; | Melting Tₒₙₛₑₜ @ 131.5° C.; |
| | Melting Tₒₙₛₑₜ @ 128.9° C.; | Endothermic event Tₒₙₛₑₜ |
| | Endothermic event Tₒₙₛₑₜ @ | @ 174.2° C. |
| | 161.8° C.; | FIG. 88 |
| | Endothermic event Tₒₙₛₑₜ @ | |
| | 172.4° C. | |
| | FIG. 7B | |
| Enthalpy (by DSC, J/g) | About 52 J/g - FIG. 7B | About 51 J/g - FIG. 8B |
| Weight loss (by TGA) | About 7.8% @ 120° C.; | About 4.2% @ 90° C.; |
| | About 5.8% @ 120° C.-180° C. | About 4.9% @ |
| | FIG. 7C | 90° C.-180° C. |
| | | FIG. 8C |
| Stoichiometric ratio | 1:0.6 | 1:0.6 |
| (by ¹H-NMR or IC) | | |
| Residual solvent | Undetected - FIG. 7D | Undetected - FIG. 8D |
| (by ¹H-NMR) | | |
| Water content | 9.1% water by weight (1.3 | 2.8% water by weight |
| (by KF) for hydrate | equiv. by molar ratio) | (0.4 equiv. by molar |
| | | ratio) |

| | |
|---|---|
| Comments | Competitive equilibration between |
| | the magnesium salt Form |
| | A and magnesium salt Form |
| | B in EA at 25° C. : |
| | A physical mixture of magnesium |
| | salt Form B (majority) and |
| | magnesium salt Form A was |
| | obtained after 3 days. |

### Example 4: Sodium Salt Crystalline Forms A, B, and C

The sodium salt Form A obtained from slurry equilibration in THE contained 1.5 equiv. of Na⁺, which is different from theoretical stoichiometry of the sodium salt. Therefore, screening with different ratios of NaOH were tried and crystalline sodium salt seeds were added during screening to optimize stoichiometry of sodium salt.

About 150 mg of the D-BHB free Form I (sample ID FR03684-1-LP1) and 1, 1.5 or 2 equiv. of NaOH were added into THE or acetone in a 2 ml glass vial. After stirring at 25° C. for 10 min, about 3 mg of the sodium salt Form A seeds (FR03684-3-RC2F-THF) were added into suspension RC13F, RC14F and RC2F-re, respectively. About 3 mg of the sodium salt Form B seeds (FR03684-3-RC2C-Acetone) were added into suspension RC2C-re. Obtained mixtures were stirred at 25° C. for at least 48 hours.

Sodium salt Form B was non-reproducible. When screening with 1 equiv. of NaOH in 0.1-0.5 ml of acetone, sodium salt Form A was obtained. IC showed D-BHB: Na⁺ is 1:1.6. When screening with 1.5 or 2 equiv. of NaOH in 0.2-0.5 mL of THF, a new polymorph of sodium salt, assigned as sodium salt Form C, was obtained. IC showed D-BHB: Na⁺ is 1:1.5.

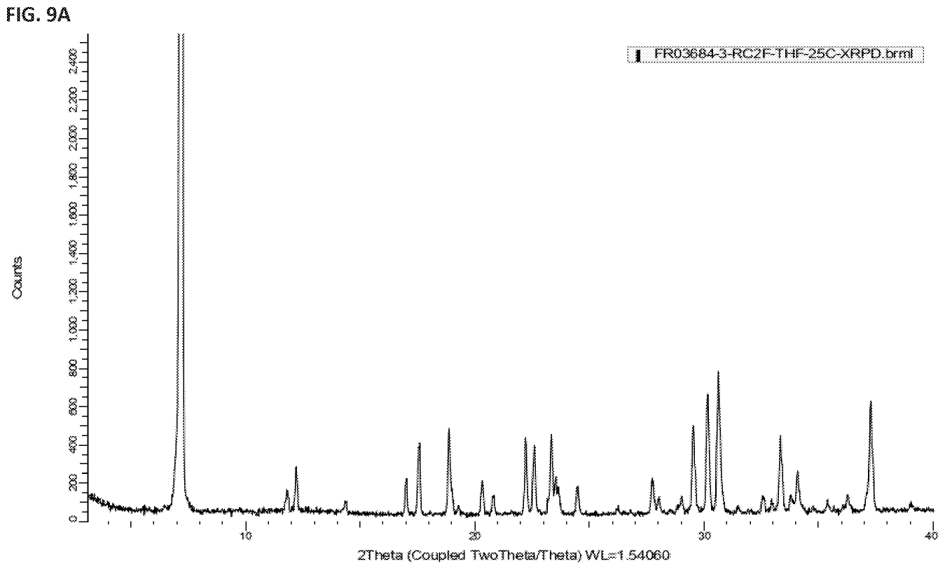

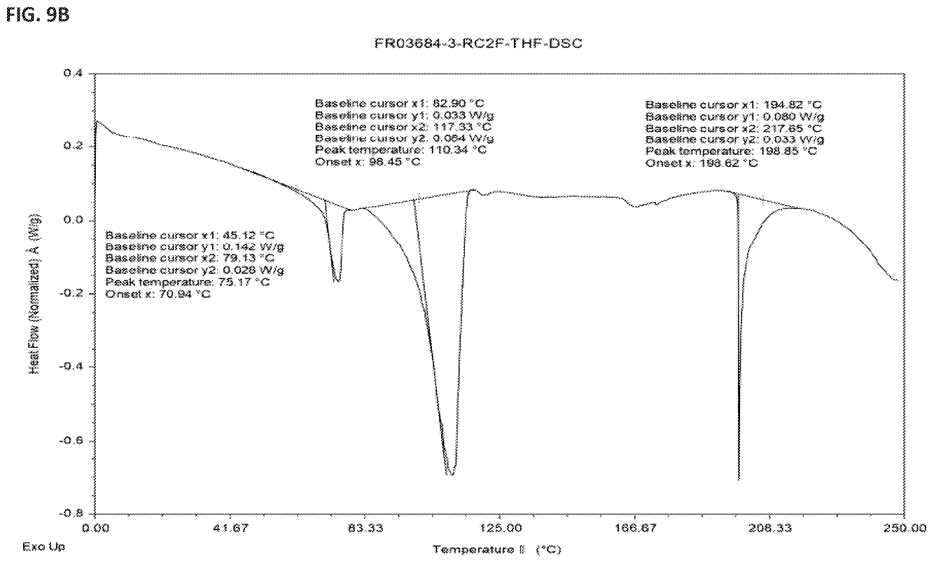

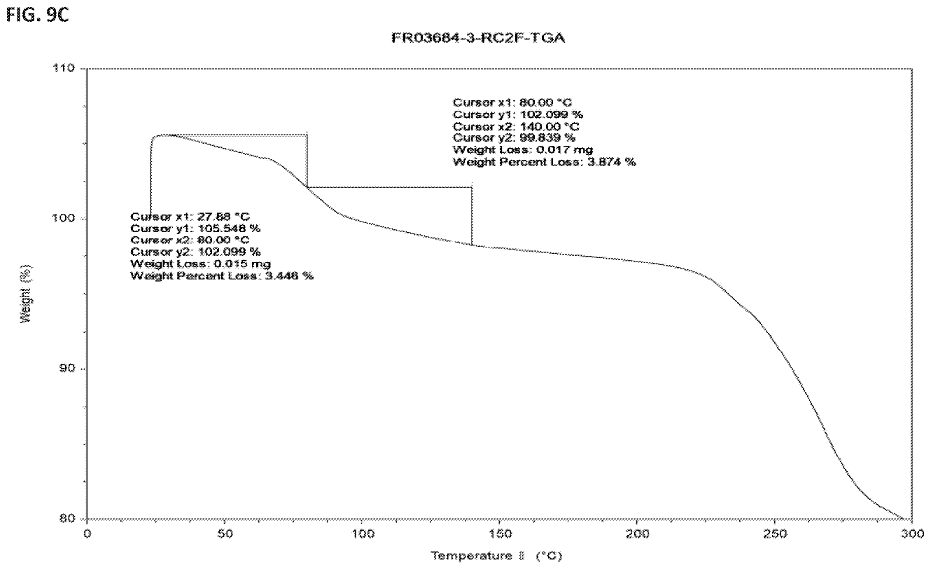

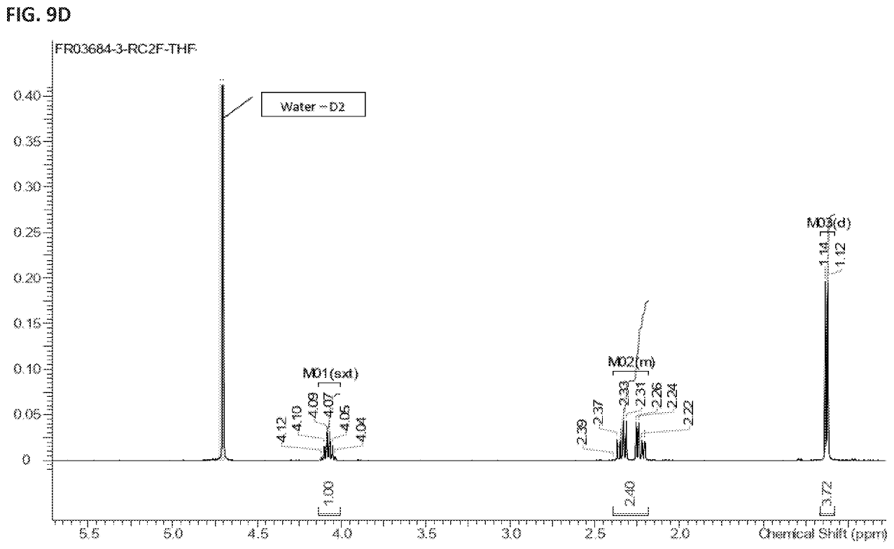

The XRPD of sodium salt Form A (FR03684-3-RC2F-THF) is shown in FIG. 9A having peaks as shown in TABLE 16.

TABLE 16
FR03684-3-RC2F-THF

| | | | Net | Gross | |
|---|---|---|---|---|---|
| Index | Angle | d Value | Intensity | Intensity | Rel. Intensity |
| 1 | 7.225° | 12.22606 Å | 6593.14 | 6651.50 | 100.0% |
| 2 | 11.820° | 7.48113 Å | 72.6678 | 131.238 | 1.1% |
| 3 | 12.217° | 7.23890 Å | 160.457 | 219.052 | 2.4% |
| 4 | 14.389° | 6.15055 Å | 29.7127 | 74.4219 | 0.5% |
| 5 | 17.018° | 5.20605 Å | 136.908 | 171.952 | 2.1% |
| 6 | 17.581° | 5.04059 Å | 233.120 | 268.987 | 3.5% |
| 7 | 18.053° | 4.90983 Å | 19.5886 | 54.7265 | 0.3% |
| 8 | 18.889° | 4.69429 Å | 327.490 | 365.689 | 5.0% |
| 9 | 19.296° | 4.59626 Å | 35.9500 | 71.2406 | 0.5% |
| 10 | 20.337° | 4.36315 Å | 125.535 | 163.014 | 1.9% |
| 11 | 20.827° | 4.26170 Å | 58.8296 | 97.4807 | 0.9% |
| 12 | 22.229° | 3.99597 Å | 308.561 | 349.263 | 4.7% |
| 13 | 22.595° | 3.93197 Å | 269.574 | 311.591 | 4.1% |
| 14 | 23.349° | 3.80676 Å | 319.040 | 362.351 | 4.8% |
| 15 | 23.538° | 3.77656 Å | 122.230 | 165.499 | 1.9% |
| 16 | 24.497° | 3.63090 Å | 108.628 | 147.301 | 1.6% |
| 17 | 26.264° | 3.39041 Å | 25.2331 | 65.4518 | 0.4% |
| 18 | 27.765° | 3.21046 Å | 143.411 | 187.804 | 2.2% |
| 19 | 28.027° | 3.18106 Å | 49.7682 | 95.0339 | 0.8% |
| 20 | 28.514° | 3.12784 Å | 18.7185 | 62.2387 | 0.3% |
| 21 | 29.036° | 3.07279 Å | 67.4665 | 115.840 | 1.0% |
| 22 | 29.544° | 3.02114 Å | 364.846 | 419.477 | 5.5% |
| 23 | 30.167° | 2.96014 Å | 483.718 | 541.659 | 7.3% |
| 24 | 30.633° | 2.91611 Å | 609.778 | 666.671 | 9.2% |
| 25 | 31.506° | 2.83730 Å | 18.1814 | 67.3141 | 0.3% |
| 26 | 32.589° | 2.74543 Å | 57.9073 | 109.506 | 0.9% |
| 27 | 32.977° | 2.71404 Å | 37.8259 | 95.5745 | 0.6% |
| 28 | 33.347° | 2.68476 Å | 337.605 | 398.387 | 5.1% |
| 29 | 33.804° | 2.64948 Å | 67.4595 | 128.180 | 1.0% |
| 30 | 34.092° | 2.62777 Å | 173.114 | 231.634 | 2.6% |
| 31 | 34.765° | 2.57842 Å | 29.4858 | 81.6653 | 0.4% |
| 32 | 35.400° | 2.53363 Å | 51.2446 | 102.767 | 0.8% |
| 33 | 36.091° | 2.48668 Å | 19.3650 | 74.2922 | 0.3% |
| 34 | 36.283° | 2.47392 Å | 76.3826 | 131.688 | 1.2% |
| 35 | 37.280° | 2.41004 Å | 511.760 | 567.072 | 7.8% |
| 36 | 39.069° | 2.30370 Å | 16.1800 | 75.9128 | 0.2 |

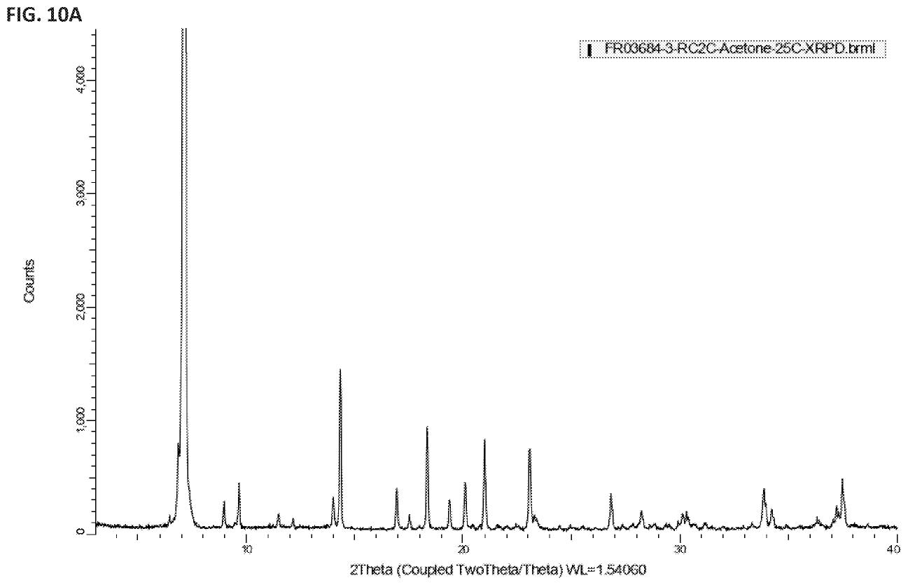

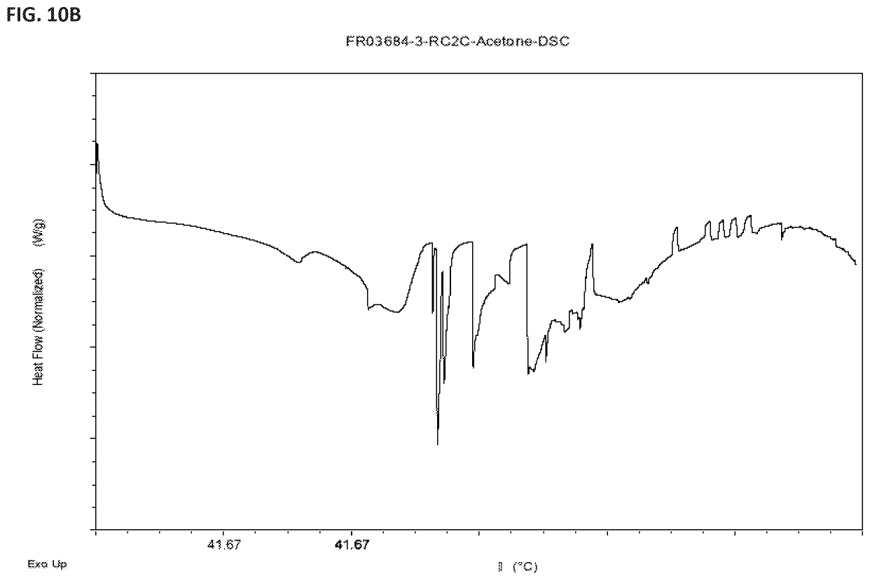

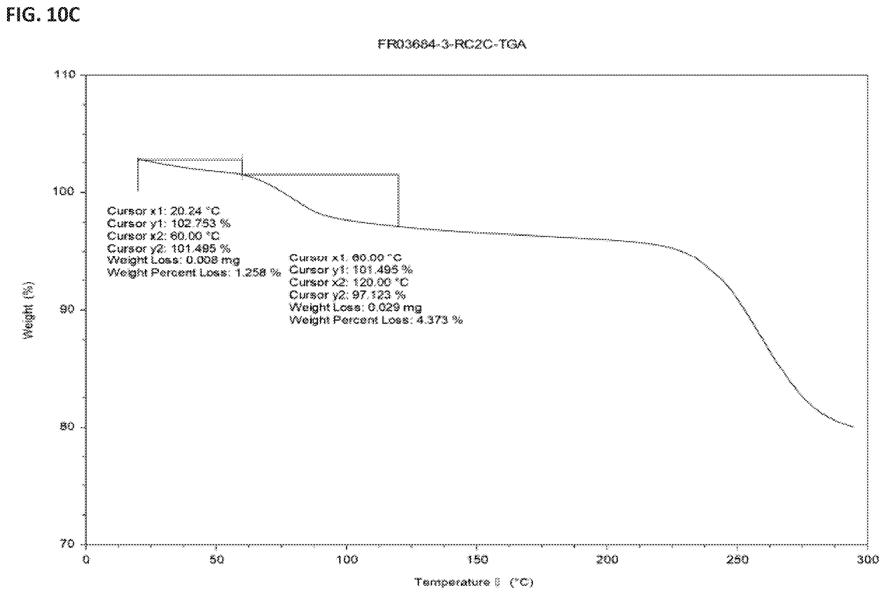

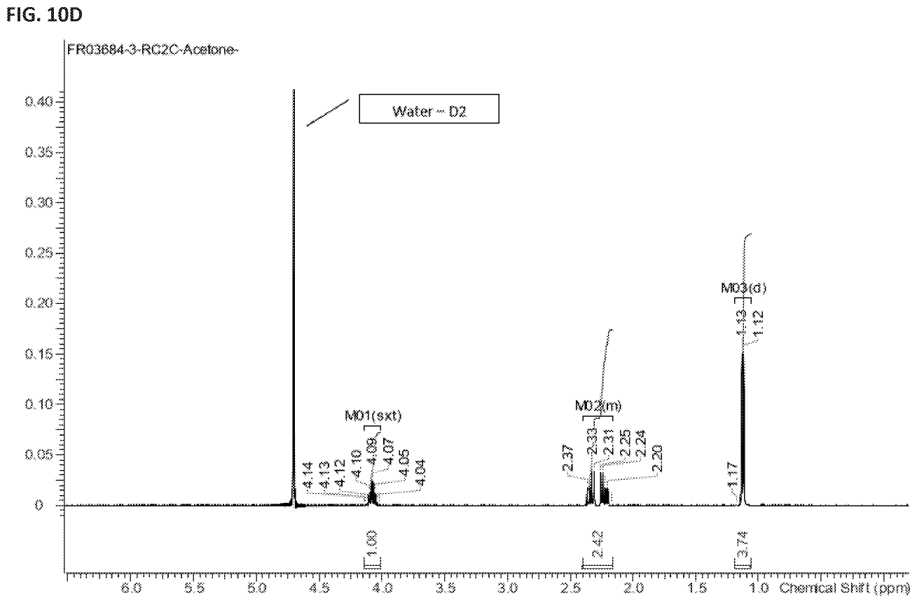

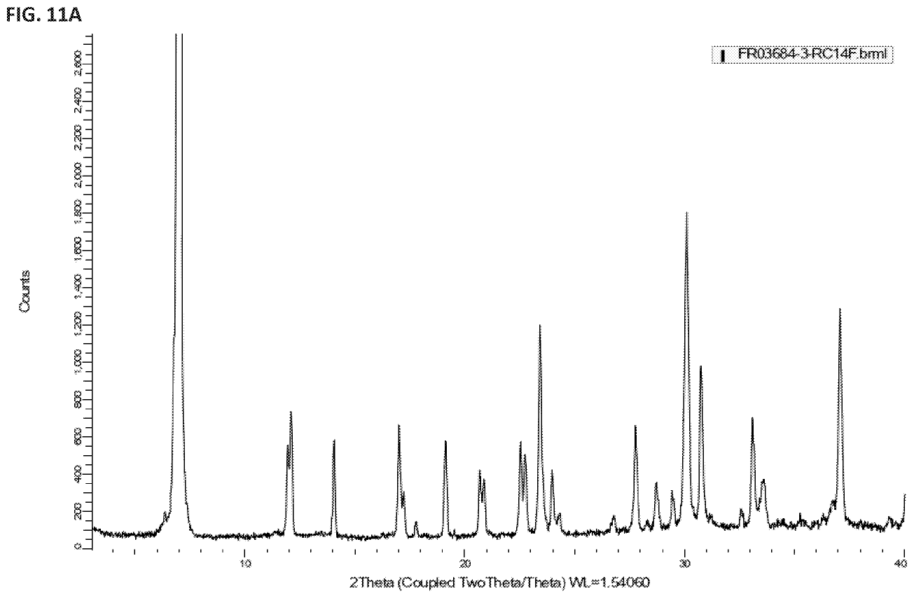

The XRPD of sodium salt Form B (FR03684-3-RC2C-Acetone) is shown in FIG. 10A having peaks as shown in TABLE 17.

TABLE 17
FR03684-3-RC2C-Acetone

| | | | Net | Gross | Rel. |
|---|---|---|---|---|---|
| Index | Angle | d Value | Intensity | Intensity | Intensity |
| 1 | 6.468° | 13.65397 Å | 47.9468 | 126.172 | 0.2% |
| 2 | 6.878° | 12.84105 Å | 442.517 | 523.331 | 1.5% |
| 3 | 7.169° | 12.32135 Å | 29117.0 | 29195.3 | 100.0% |
| 4 | 8.980° | 9.83936 Å | 146.029 | 206.430 | 0.5% |
| 5 | 9.501° | 9.30162 Å | 26.9967 | 86.3617 | 0.1% |
| 6 | 9.671° | 9.13834 Å | 278.716 | 338.425 | 1.0% |
| 7 | 11.487° | 7.69715 Å | 81.8618 | 147.093 | 0.3% |
| 8 | 12.158° | 7.27365 Å | 50.3291 | 110.641 | 0.2% |
| 9 | 14.018° | 6.31276 Å | 181.339 | 245.189 | 0.6% |
| 10 | 14.349° | 6.16760 Å | 1026.10 | 1086.24 | 3.5% |
| 11 | 16.948° | 5.22739 Å | 262.107 | 307.127 | 0.9% |
| 12 | 17.529° | 5.05527 Å | 87.4139 | 133.234 | 0.3% |
| 13 | 17.991° | 4.92668 Å | 20.8102 | 66.8364 | 0.1% |
| 14 | 18.349° | 4.83120 Å | 675.268 | 726.229 | 2.3% |
| 15 | 19.384° | 4.57553 Å | 191.061 | 236.896 | 0.7% |
| 16 | 20.103° | 4.41356 Å | 316.958 | 362.018 | 1.1% |
| 17 | 20.452° | 4.33889 Å | 28.8818 | 74.9416 | 0.1% |
| 18 | 20.813° | 4.26450 Å | 30.6969 | 77.7197 | 0.1% |
| 19 | 21.000° | 4.22689 Å | 634.967 | 681.652 | 2.2% |
| 20 | 21.576° | 4.11531 Å | 33.8322 | 76.8598 | 0.1% |
| 21 | 22.027° | 4.03205 Å | 20.6283 | 65.5674 | 0.1% |
| 22 | 22.450° | 3.95711 Å | 41.3225 | 89.7943 | 0.1% |
| 23 | 23.080° | 3.85047 Å | 565.345 | 616.190 | 1.9% |
| 24 | 23.296° | 3.81522 Å | 89.3168 | 139.807 | 0.3% |
| 25 | 23.384° | 3.80106 Å | 55.7765 | 105.569 | 0.2% |
| 26 | 24.456° | 3.63684 Å | 32.2282 | 71.0546 | 0.1% |
| 27 | 24.941° | 3.56723 Å | 32.5828 | 73.6181 | 0.1% |
| 28 | 25.480° | 3.49295 Å | 23.1009 | 64.5937 | 0.1% |
| 29 | 26.830° | 3.32020 Å | 253.938 | 299.615 | 0.9% |
| 30 | 27.363° | 3.25675 Å | 34.2164 | 80.3827 | 0.1% |
| 31 | 27.843° | 3.20171 Å | 29.9925 | 81.2518 | 0.1% |
| 32 | 28.237° | 3.15788 Å | 130.590 | 183.990 | 0.4% |
| 33 | 28.667° | 3.11151 Å | 14.9582 | 65.5574 | 0.1% |
| 34 | 28.834° | 3.09382 Å | 26.1612 | 75.8602 | 0.1% |
| 35 | 29.378° | 3.03778 Å | 33.9571 | 79.5461 | 0.1% |
| 36 | 29.513° | 3.02419 Å | 34.4373 | 78.8575 | 0.1% |
| 37 | 29.936° | 2.98241 Å | 60.8244 | 107.634 | 0.2% |
| 38 | 30.130° | 2.96365 Å | 108.123 | 158.974 | 0.4% |
| 39 | 30.313° | 2.94623 Å | 132.621 | 185.827 | 0.5% |
| 40 | 31.166° | 2.86751 Å | 36.8628 | 85.2109 | 0.1% |
| 41 | 33.335° | 2.68568 Å | 38.0485 | 92.5760 | 0.1% |
| 42 | 33.883° | 2.64349 Å | 276.558 | 337.818 | 0.9% |
| 43 | 34.254° | 2.61574 Å | 136.258 | 196.411 | 0.5% |
| 44 | 34.945° | 2.56552 Å | 25.0444 | 75.5781 | 0.1% |
| 45 | 36.356° | 2.46917 Å | 42.3645 | 103.566 | 0.1% |
| 46 | 36.429° | 2.46435 Å | 29.9337 | 91.0591 | 0.1% |
| 47 | 37.085° | 2.42229 Å | 47.3670 | 108.725 | 0.2% |
| 48 | 37.233° | 2.41300 Å | 163.498 | 228.219 | 0.6% |
| 49 | 37.504° | 2.39618 Å | 381.733 | 450.233 | 1.3% |
| 50 | 38.673° | 2.32637 Å | 26.3679 | 84.3676 | 0.1% |

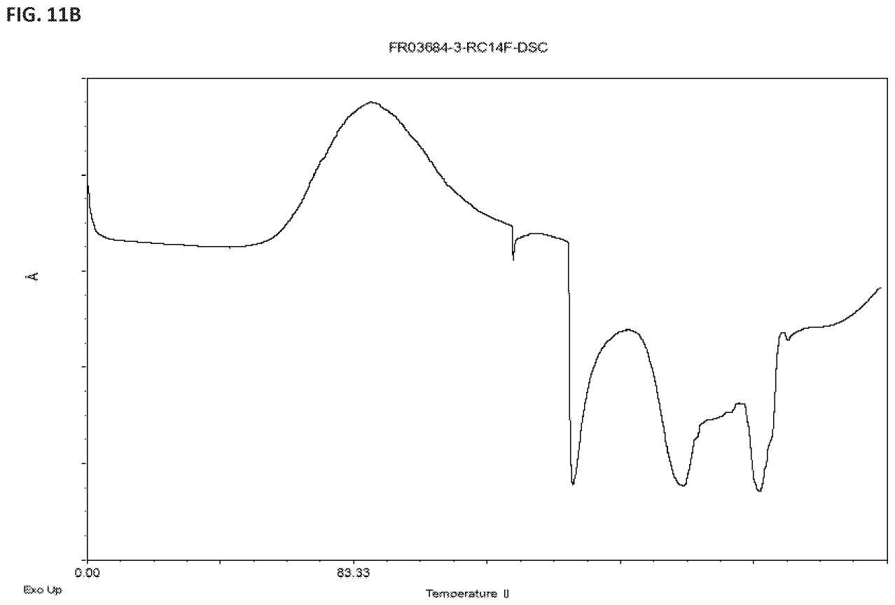

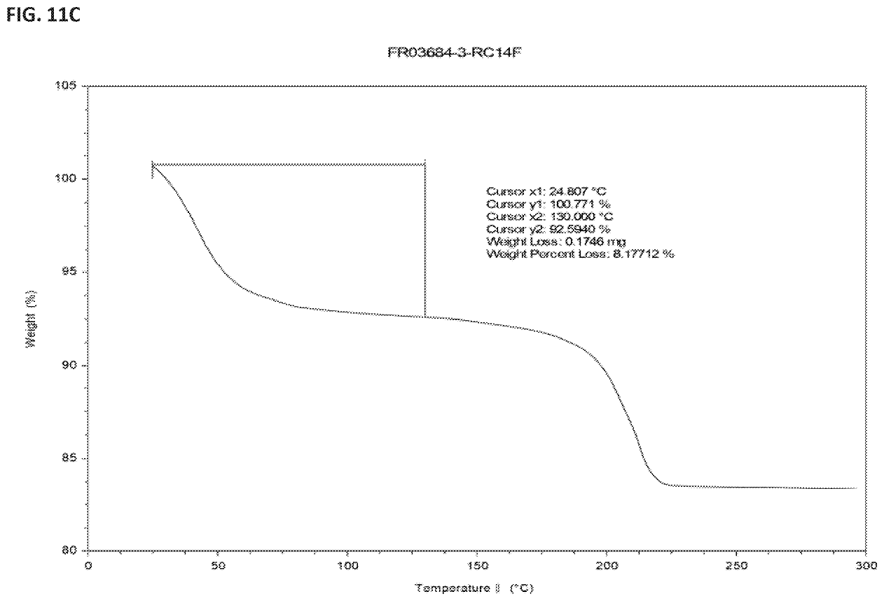

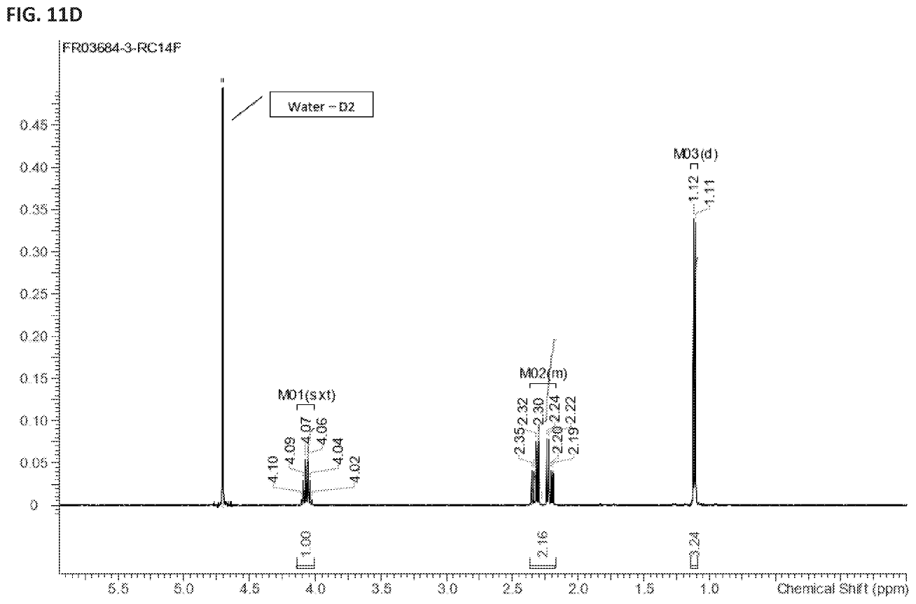

The XRPD of sodium salt form C (FR03684-3-RC14F-THF) is shown in FIG. 11A having peaks as shown in TABLE 18.

TABLE 18
FR03684-3-RC14F-THF

| | | | Net | Gross | Rel. |
|---|---|---|---|---|---|
| Index | Angle | d Value | Intensity | Intensity | Intensity |
| 1 | 6.360° | 13.88566 Å | 65.1244 | 153.366 | 0.2% |
| 2 | 7.040° | 12.54700 Å | 28231.4 | 28321.2 | 100.0% |
| 3 | 11.959° | 7.39476 Å | 317.100 | 399.171 | 1.1% |
| 4 | 12.101° | 7.30797 Å | 454.040 | 535.768 | 1.6% |
| 5 | 14.054° | 6.29667 Å | 333.863 | 409.252 | 1.2% |
| 6 | 16.294° | 5.43566 Å | 24.0532 | 87.5185 | 0.1% |
| 7 | 17.021° | 5.20510 Å | 414.792 | 486.202 | 1.5% |
| 8 | 17.227° | 5.14319 Å | 157.009 | 226.643 | 0.6% |
| 9 | 17.778° | 4.98519 Å | 50.8457 | 119.589 | 0.2% |
| 10 | 19.150° | 4.63094 Å | 344.903 | 415.476 | 1.2% |
| 11 | 19.538° | 4.53978 Å | 31.8866 | 101.712 | 0.1% |
| 12 | 20.724° | 4.28270 Å | 171.493 | 246.830 | 0.6% |
| 13 | 20.876° | 4.25169 Å | 217.641 | 293.193 | 0.8% |
| 14 | 22.558° | 3.93837 Å | 351.863 | 437.033 | 1.2% |
| 15 | 22.760° | 3.90398 Å | 300.321 | 390.552 | 1.1% |
| 16 | 23.434° | 3.79308 Å | 822.983 | 922.243 | 2.9% |
| 17 | 23.984° | 3.70739 Å | 263,626 | 361.291 | 0.9% |
| 18 | 24.338° | 3.65426 Å | 65.6908 | 155.694 | 0.2% |
| 19 | 26.800° | 3.32388 Å | 50,0653 | 146.572 | 0.2% |
| 20 | 27.777° | 3.20917 Å | 436.983 | 543.019 | 1.5% |
| 21 | 28.294° | 3.15165 Å | 26,6043 | 132.433 | 0.1% |
| 22 | 28.719° | 3.10600 Å | 190,490 | 300.570 | 0.7% |
| 23 | 29.471° | 3.02843 Å | 124.186 | 254.133 | 0.4% |
| 24 | 30.106° | 2.96601 Å | 1233.67 | 1382.29 | 4.4% |
| 25 | 30.759° | 2.90450 Å | 671.896 | 822.861 | 2.4% |
| 26 | 31.200° | 2.86441 Å | 36.9417 | 179.479 | 0.1% |
| 27 | 32.600° | 2.74452 Å | 68.3101 | 194.818 | 0.2% |
| 28 | 33.106° | 2.70375 Å | 460.216 | 593.152 | 1.6% |
| 29 | 33.616° | 2.66390 Å | 158.258 | 286.943 | 0.6% |
| 30 | 35.285° | 2.54156 Å | 54.3662 | 171.389 | 0.2% |
| 31 | 36.320° | 2.47153 Å | 46.4588 | 182.338 | 0.2% |
| 32 | 36.740° | 2.44420 Å | 87.2135 | 230.265 | 0.3% |
| 33 | 37.093° | 2.42177 Å | 949.082 | 1092.49 | 3.4% |
| 34 | 39.324° | 2.28935 Å | 48.6535 | 163.208 | 0.2% |

TABLE 19

| | Sesqui-sodium | Sesqui-sodium | Sesqui-sodium |
|---|---|---|---|
| | Salt Form A, | Salt Form B, | Salt Form |
| | hydrate | hydrate | C, hydrate |
| Sample ID | FR03684-3- | FR03684-3- | FR03684-3-RC14F- |
| | RC2F-THF | RC2C-Acetone | THF |
| | (Example 2) | (Example 2) | |
| Preparation | THF | Acetone | THF |
| solvent | | | |
| Crystallinity | High crystallinity | High | High crystallinity |
| (by XRPD | FIG. 9A | crystallinity | FIG. 11A |
| Method 2) | | FIG. 10A | |
| Melting onset | Dehydration from | Multiple | Multiple thermal |
| (by DSC, ° C.) | about 45° C.; | thermal events | events |
| | Endothermic event | FIG. 10B | FIG. 11B |
| | Tₒₙₛₑₜ @ 98.5° C.; | | |
| | Endothermic event | | |
| | Tₒₙₛₑₜ @ 198.6° C. | | |
| | FIG. 9B | | |
| Enthalpy (by | FIG. 9B | FIG. 10B | FIG. 11B |
| DSC, J/g) | | | |
| Weight loss (by | About 3.4% @ 80° C.; | About 1.3% @ | About 8.2% @ |
| TGA) | About 3.9% @ 80° C.- | 60° C.; | 130° C. |
| | 140° C. | About 4.4% @ | FIG. 11C |
| | FIG. 9C | 60° C.-120° C. | |
| | | FIG. 10C | |
| Stoichiometric | 1:1.6 | // | 1:1.5 |
| ratio | | | |
| (by ¹H-NMR or | | | |
| IC) | | | |
| Residual solvent | Undetected | Undetected | Undetected |
| (by ¹H-NMR) | FIG. 9D | FIG. 10D | FIG. 11D |
| Water content | 7.7% water by weight | // | 7.0% water by |
| (by KF) for | (0.7 equiv, by molar | | weight (0.6 equiv. |
| hydrate | ratio) | | by molar ratio) |
| Comments | Reproducible | Not | When 1.5 equiv. or |
| | When 1.0 equiv. NaOH | reproducible | 2.0 equiv. NaOH |
| | was added, sodium salt | When 1.0 equiv. | was added, sodium |
| | Form A was obtained | NaOH was | salt Form C was |
| | with addition of | added, sodium | obtained with |
| | sodium salt Form A | salt Form A with | addition of sodium |
| | seeds. | extra peaks was | salt Form A seeds. |
| | | obtained with | |
| | | addition of | |
| | | sodium salt | |
| | | Form B seeds. | |

// = Not carried out.

### Example 5: Scale Up of D-BHB Sesqui-Sodium Salt Form C

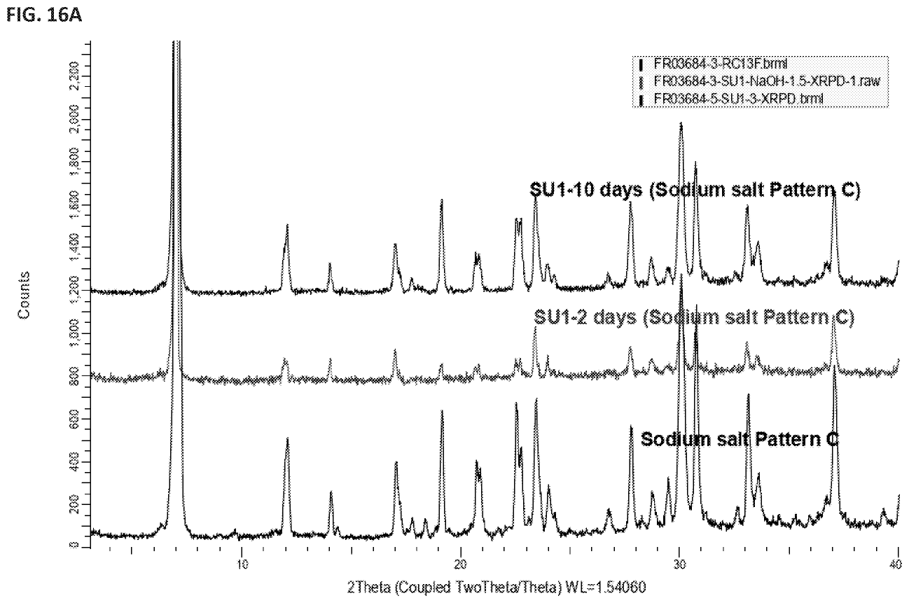

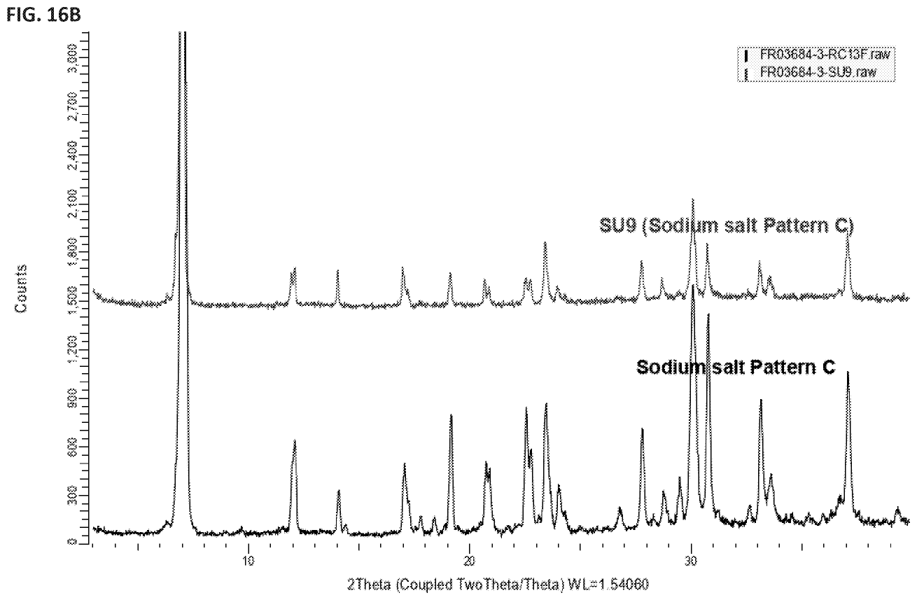

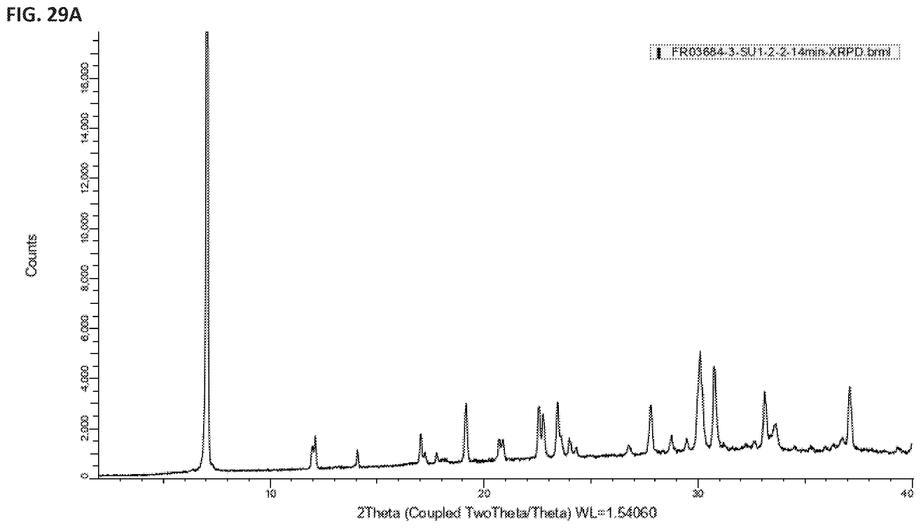

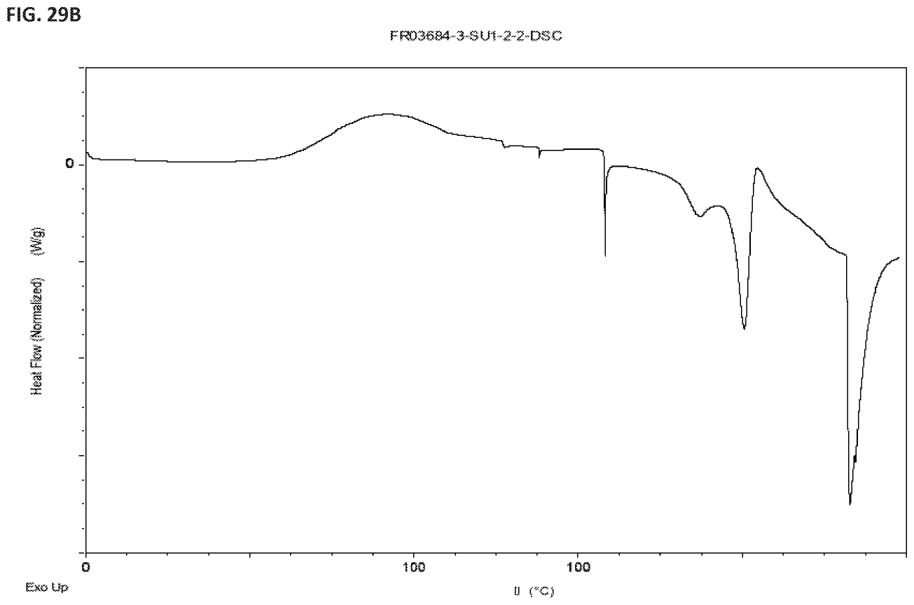

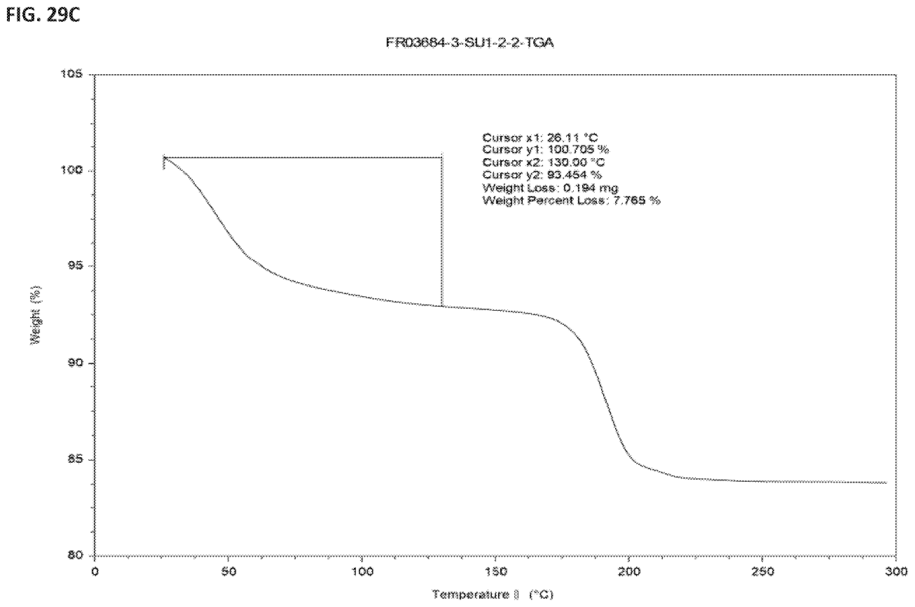

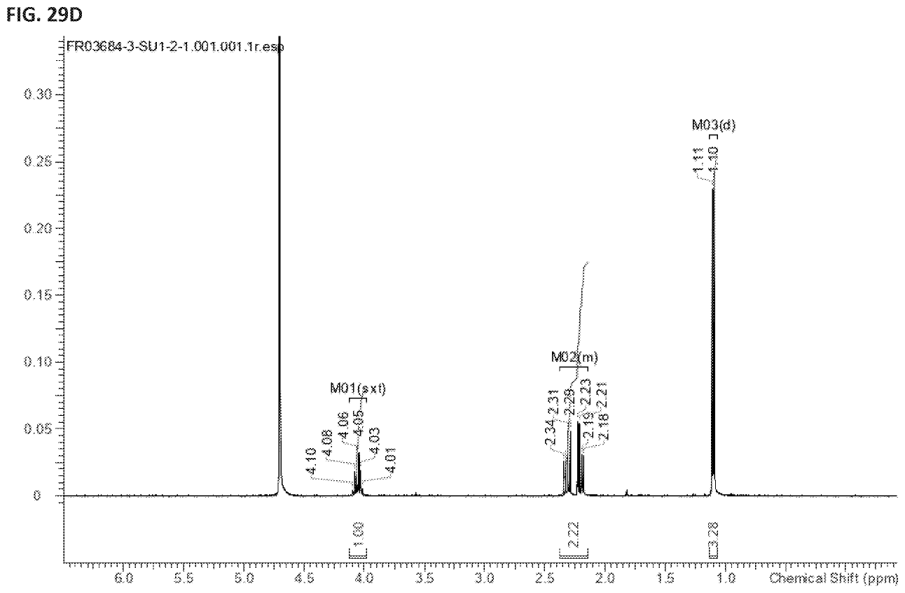

Trial 1: Sodium salt Form C (FR03684-SU1-NaOH-1.5-THF) was prepared as follows. 2.0 g of the D-BHB free Form I (FR03684-1-LP1) and 1.2 g of NaOH (~1.5 equivalent by molar ratio) were weighed into a 40 ml glass vial. 10 ml of THF was added into the vial under stirring at 25° C. for about 10 min. A suspension was obtained. About 1 mg of the sodium salt Form A seeds (FR03684-3-RC2D) was added into above suspension. The suspension was kept stirring at 25° C. for about 2 days. About 5 ml of suspension was taken out and centrifuged. Obtained THF saturated solution (~5 mL) was added back into the 40 ml glass vial. Obtained wet cake was dried at 50° C. under vacuum for about 2 hours. 585 mg of the sodium salt Form C (FIG. 16A-XRPD Method 2) was obtained. But crystallinity of the sodium salt Form C sample was a little low. To improve crystallinity of the sodium salt Form C, obtained dry cake was added into above-mentioned saturated solution. All of the suspension (10 mL) was kept stirring at 25° C. for another 8 days. About 1 ml of the suspension was taken out. The solid part was collected by filtered through a 0.45 μm nylon membrane and characterized by XRPD. Crystallinity of the sodium salt Form C improved (FIG. 16A—XRPD Method 2). Rest of suspension was collected by filtration and then dried at 50° C. under vacuum for about 2 hours. 1.9 g of the sodium salt Form C was obtained as an off-white solid in 59% yield. Characterization results are reported below in Example 14. FR03684-SU1-NaOH-1.5-THF was used for bulk stability study, solubility study and hygroscopicity reported below in Examples 11 to 13. The XRPD of D-BHB sodium salt Form C (FR03684-SU1-NaOH-1.5-THF) is shown in FIG. 29A and has peaks as shown in TABLE 20.

TABLE 20
FR03684-SU1-NaOH-1.5-THF

| | | | Net | Gross | |
|---|---|---|---|---|---|
| Index | Angle | d Value | Intensity | Intensity | Rel. Intensity |
| 1 | 6.396° | 13.80884 Å | 59.6373 | 327.049 | 0.3% |
| 2 | 7.069° | 12.49515 Å | 20929.6 | 21221.7 | 100.0% |
| 3 | 11.982° | 7.38033 Å | 610.247 | 995.189 | 2.9% |
| 4 | 12.113° | 7.30072 Å | 923.099 | 1310.54 | 4.4% |
| 5 | 14.082° | 6.28399 Å | 506.137 | 945.428 | 2.4% |
| 6 | 17.048° | 5.19682 Å | 844.052 | 1427.13 | 4.0% |
| 7 | 17.239° | 5.13979 Å | 330.536 | 922.731 | 1.6% |
| 8 | 17.798° | 4.97952 Å | 271.810 | 884.694 | 1.3% |
| 9 | 19.156° | 4.62946 Å | 1658.74 | 2311.21 | 7.9% |
| 10 | 20.716° | 4.28428 Å | 604.120 | 1316.31 | 2.9% |
| 11 | 20.874° | 4.25225 Å | 613.556 | 1332.81 | 2.9% |
| 12 | 22.568° | 3.93662 Å | 1533.90 | 2345.38 | 7.3% |
| 13 | 22.773° | 3.90178 Å | 1269.53 | 2092.92 | 6.1% |
| 14 | 23.449° | 3.79073 Å | 1610.32 | 2462.40 | 7.7% |
| 15 | 23.508° | 3.78135 Å | 471.902 | 1325.72 | 2.3% |
| 16 | 24.006° | 3.70398 Å | 557.559 | 1420.99 | 2.7% |
| 17 | 24.309° | 3.65853 Å | 244.573 | 1109.51 | 1.2% |
| 18 | 26.777° | 3.32662 Å | 300.397 | 1242.57 | 1.4% |
| 19 | 27.791° | 3.20757 Å | 1473.09 | 2477.18 | 7.0% |
| 20 | 28.356° | 3.14488 Å | 73.0076 | 1097.68 | 0.3% |
| 21 | 28.750° | 3.10271 Å | 488.943 | 1540.23 | 2.3% |
| 22 | 29.475° | 3.02803 Å | 422.004 | 1523.10 | 2.0% |
| 23 | 30.108° | 2.96582 Å | 2913.18 | 4042.36 | 13.9% |
| 24 | 30.760° | 2.90435 Å | 2804.71 | 3947.86 | 13.4% |
| 25 | 31.215° | 2.86311 Å | 165.431 | 1309.29 | 0.8% |
| 26 | 31.620° | 2.82733 Å | 96.4451 | 1245.76 | 0.5% |
| 27 | 32.228° | 2.77533 Å | 190.801 | 1357.68 | 0.9% |
| 28 | 32.615° | 2.74333 Å | 267.757 | 1438.89 | 1.3% |
| 29 | 33.121° | 2.70252 Å | 1889.33 | 3057.96 | 9.0% |
| 30 | 33.626° | 2.66313 Å | 669.114 | 1826.13 | 3.2% |
| 31 | 34.539° | 2.59477 Å | 120.624 | 1233.41 | 0.6% |
| 32 | 35.277° | 2.54214 Å | 166.332 | 1264.50 | 0.8% |
| 33 | 35.960° | 2.49541 Å | 135.461 | 1245.14 | 0.6% |
| 34 | 36.342° | 2.47005 Å | 204.511 | 1318.07 | 1.0% |
| 35 | 36.767° | 2.44250 Å | 368.471 | 1480.20 | 1.8% |
| 36 | 37.084° | 2.42232 Å | 2200.64 | 3306.80 | 10.5% |
| 37 | 39.347° | 2.28806 Å | 150.538 | 1178.68 | 0.7% |

Trail 2: Sodium salt Form C (FR03684-SU9-NaOH-1.5-THF) was prepared as follows. 2.0 g of the D-BHB free Form I (FR03684-1-LP2) and 1.2 g of NaOH (^{~}1.5 equivalent by molar ratio) were weighed into a 40 ml glass vial. 10 ml of THE was added into the vial under stirring at 25° C. for about 10 min. A suspension was obtained. About 1 mg of the sodium salt Form C seeds (FR03684-SU1-NaOH-1.5-THF as obtained from Trial 1) were added into above suspension. The suspension was kept stirring at 25° C. for about 4 days. Solids were collected by filtration and then dried at 50° C. under vacuum for about 2 hours. 2.6 g of the sodium salt Form C (FIG. 16B—XRPD Method 2) was obtained as an off-white solid in 81% yield. FR03684-SU9-NaOH-1.5-THF was used for solubility study in Example 12.

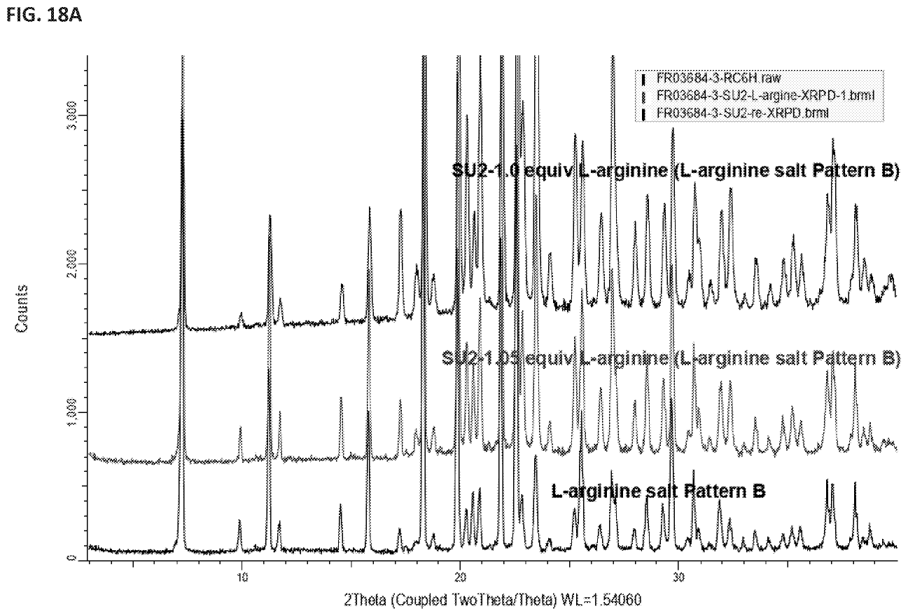

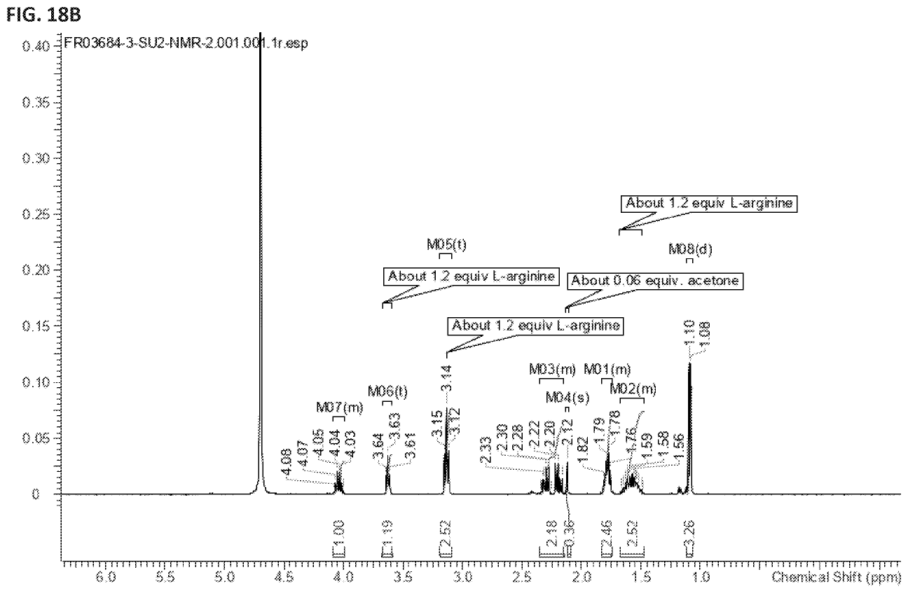

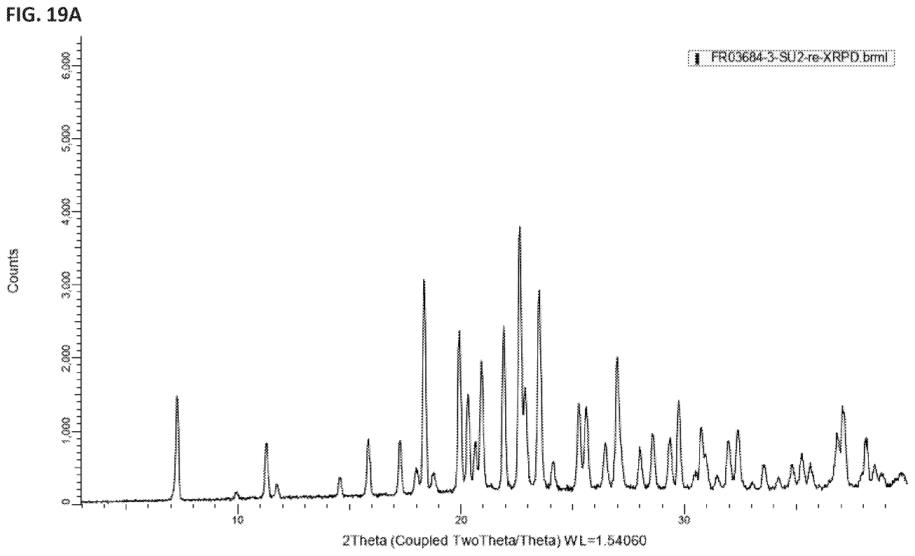

TABLE 21
D-BHB sodium salt Form C

| Sample ID | FR03684-SU9-NaOH-1.5-THF | |
|---|---|---|
| Parameter | Method | Result |
| X-ray diffraction | XRPD (Method 2), | High crystallinity, Form C |
| | 3-40° (2 theta) | FIG. 18A |
| Thermal events and | DSC, 10° C./min | Multiple thermal |
| enthalpy | | events, FIG. 18B |
| Thermogravimetry | TGA, 10° C./min | 10.0% @ 130° C., FIG. 18C |
| Residual solvent(s) | ¹H-NMR (D₂O-d₂) | Undetected, FIG. 18D |
| Stoichiometry | IC | 1:1.6 |
| (free form: | | |
| Na⁺) | | |
| Water content | Karl Fischer | 9.4% water by weight |
| | (coulometric) | (0.8 equiv. by molar ratio) |

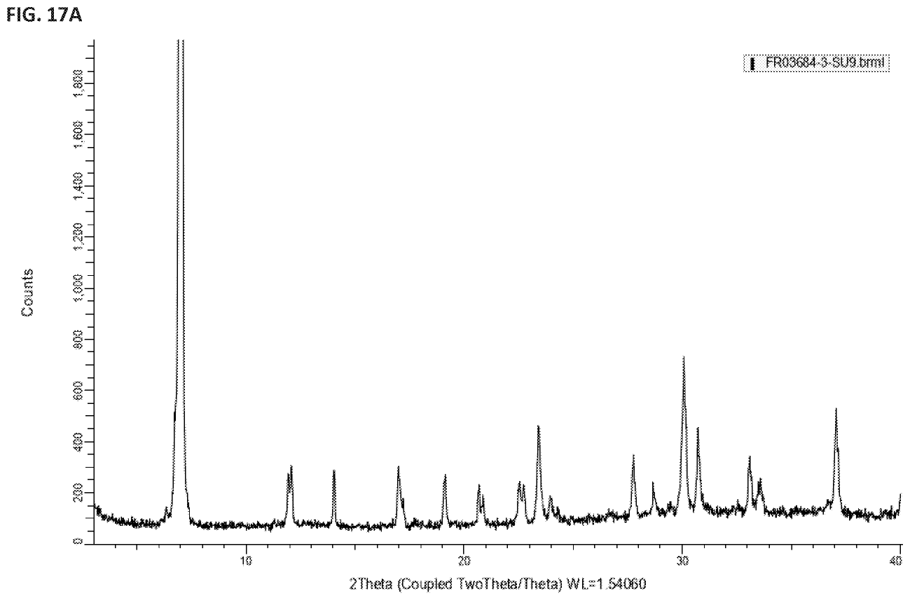

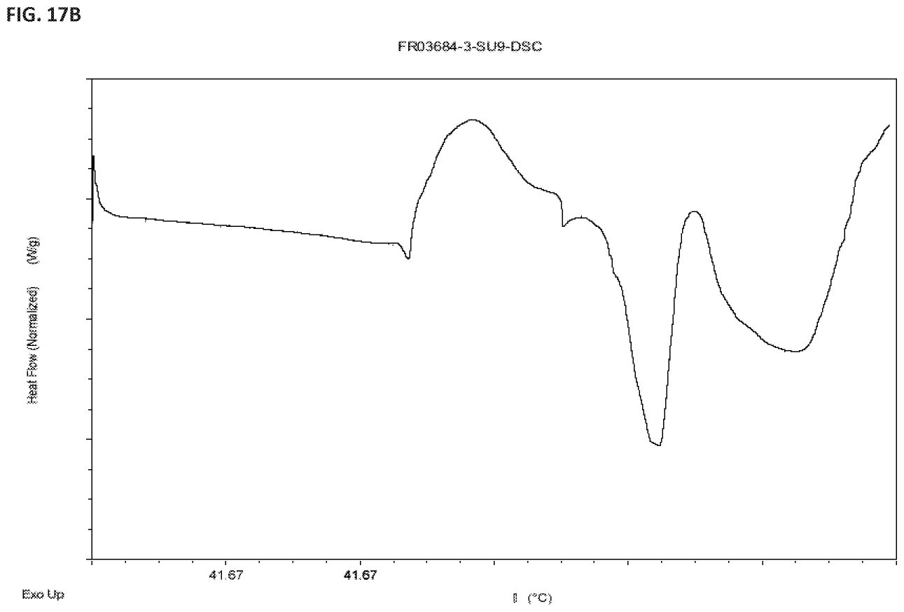

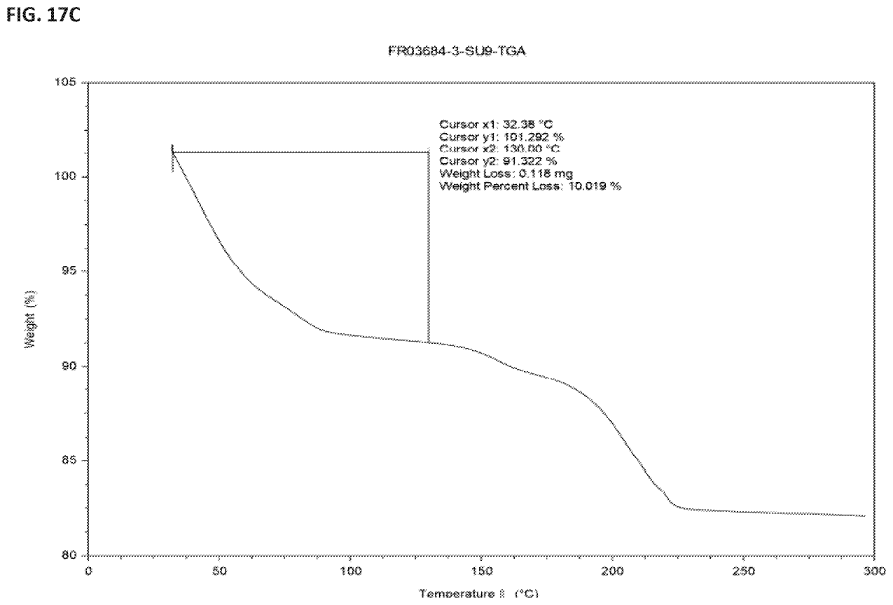

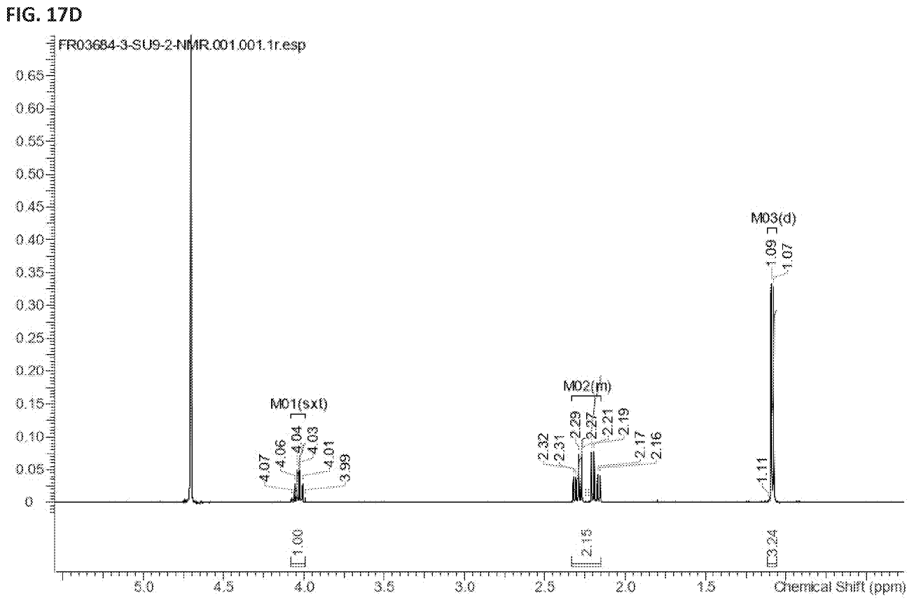

FIG. 17A shows the XRPD for D-BHB sodium salt Form C (FR03684-SU9-NaOH-1.5-THF) having peaks as shown in TABLE 22.

TABLE 22
FR03684-SU9-NaOH-1.5-THF

| | | | Net | Gross | Rel. |
|---|---|---|---|---|---|
| Index | Angle | d Value | Intensity | Intensity | Intensity |
| 1 | 5.213° | 16.94008 Å | 14.9496 | 84.9732 | 0.1% |
| 2 | 6.345° | 13.91876 Å | 38.3140 | 118.280 | 0.3% |
| 3 | 6.748° | 13.08855 Å | 249.465 | 334.262 | 1.7% |
| 4 | 7.025° | 12.57327 Å | 14374.5 | 14459.8 | 100.0% |
| 5 | 11.947° | 7.40165 Å | 128.849 | 205.159 | 0.9% |
| 6 | 12.090° | 7.31462 Å | 151.560 | 228.257 | 1.1% |
| 7 | 14.044° | 6.30088 Å | 158.288 | 231.859 | 1.1% |
| 8 | 16.998° | 5.21218 Å | 153.857 | 225.608 | 1.1% |
| 9 | 17.199° | 5.15173 Å | 53.7397 | 125.292 | 0.4% |
| 10 | 19.131° | 4.63555 Å | 138.723 | 210.845 | 1.0% |
| 11 | 20.683° | 4.29094 Å | 106.563 | 185.799 | 0.7% |
| 12 | 20.872° | 4.25268 Å | 73.8363 | 152.647 | 0.5% |
| 13 | 22.541° | 3.94137 Å | 108.718 | 192.428 | 0.8% |
| 14 | 22.737° | 3.90774 Å | 92.8506 | 179.210 | 0.6% |
| 15 | 23.427° | 3.79432 Å | 275.234 | 372.095 | 1.9% |
| 16 | 23.972° | 3.70923 Å | 56.4272 | 155.862 | 0.4% |
| 17 | 24.313° | 3.65796 Å | 39.8756 | 136.424 | 0.3% |
| 18 | 27.772° | 3.20973 Å | 176.593 | 288.069 | 1.2% |
| 19 | 28.696° | 3.10837 Å | 76.3143 | 197.395 | 0.5% |
| 20 | 30.092° | 2.96729 Å | 447.057 | 577.646 | 3.1% |
| 21 | 30.738° | 2.90642 Å | 254.581 | 387.942 | 1.8% |
| 22 | 33.100° | 2.70419 Å | 174.394 | 305.175 | 1.2% |
| 23 | 33.593° | 2.66561 Å | 75.9712 | 203.344 | 0.5% |
| 24 | 37.075° | 2.42291 Å | 316.342 | 441.438 | 2.2% |

### Example 6: Scale Up of D-BHB L-Arginine Salt Form B

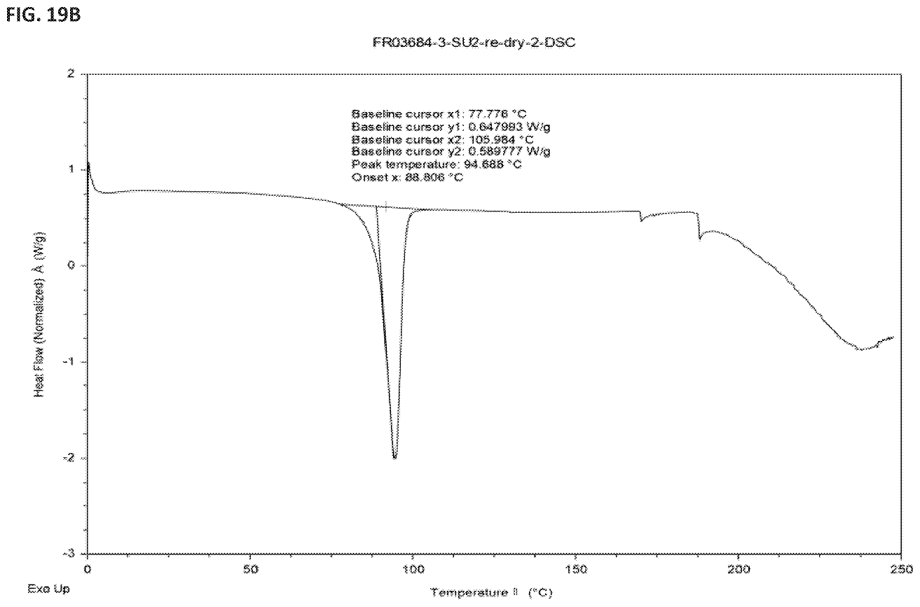

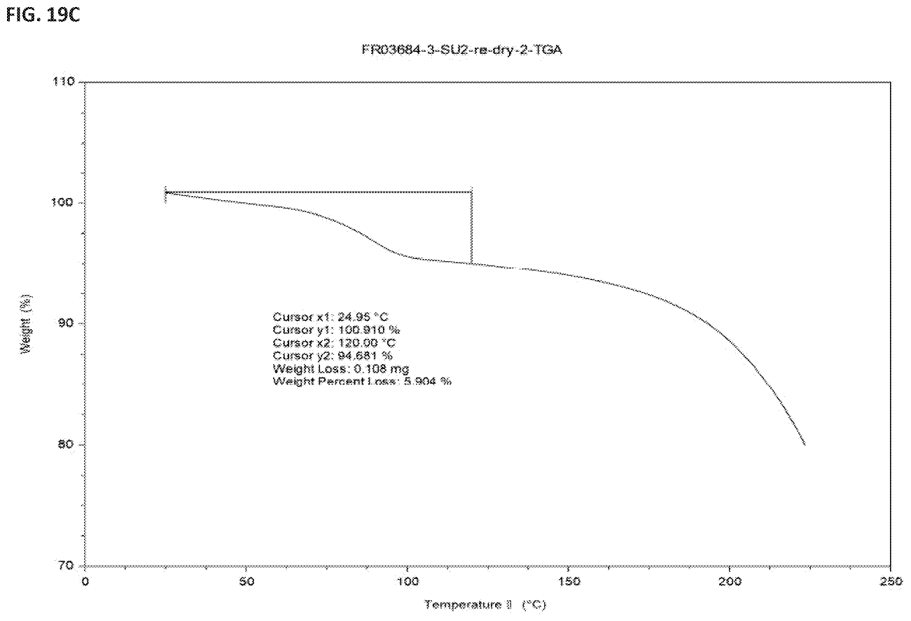

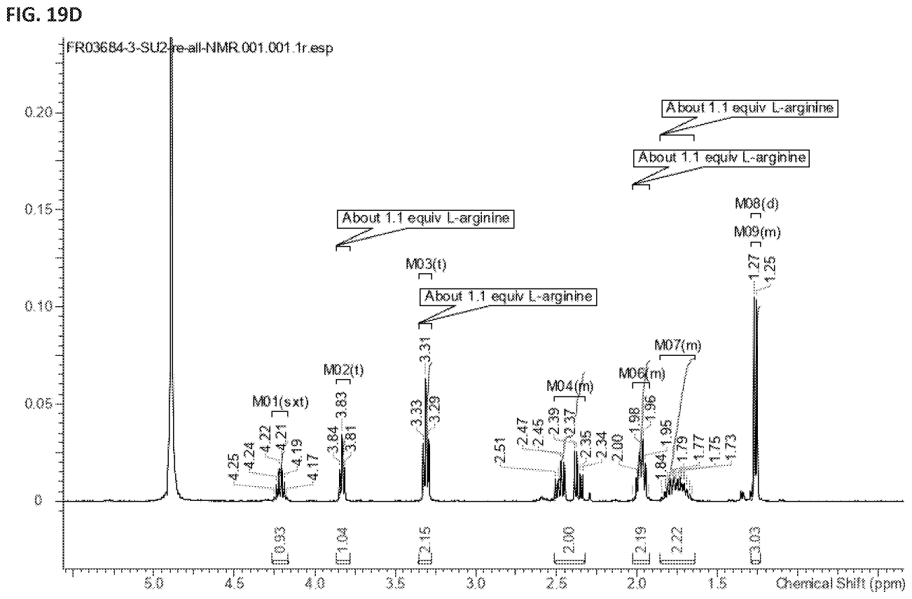

Trial 1: L-arginine salt Form B (FR03684-SU2-L-arginine-acetone-water-95-5-re) was prepared as follows. 2.0 g of the D-BHB free Form I (FR03684-1-LP1) and 3.5 g of L-arginine (~1.05 equivalent by molar ratio) were weighed into a 100 ml glass vial. 34 ml of acetone/water (v:v=95:5) was added into the vial under stirring at 50° C. for about 10 min. A gel-like material was obtained. About 3 mg of the L-arginine salt Form B seeds (ID FR03684-3-RC6H) was added. This gel-like material was stirred at 50° C. for about 2 hours. This gel-like material was cooled to 25° C. and kept stirring at 25° C. for about 1 day. The gel-like material gradually converted into a suspension. The suspension was kept stirring at 25° C. for another 1 day. Solids were collected by filtration and then dried at 50° C. under vacuum for about 2 hours. 4.8 g of the L-arginine salt Form B (FIG. 19A) was obtained (FR03684-3-SU2-L-arginine-acetone-water-95-5). The solid part showed a ratio of free form:L-arginine=1:1.2 (FIG. 19B).

In order to optimize the stoichiometry of L-arginine salt Form B, another 100 mg (~0.05 equivalent by molar ratio) of the D-BHB free Form I (FR03684-1-LP1) and 4.6 g of the L-arginine salt Form B (FR03684-3-SU2-L-arginine-acetone-water-95-5) were weighed into a 100 ml glass vial. 5 ml of acetone/water (v:v=95:5) saturated solution was added into the vial and kept stirring at 25° C. for about 2 days. Solids were collected by filtration and then dried at 50° C. under vacuum for about 2 hours. 3.2 g of the L-arginine salt Form B (FIG. 19A) was obtained as an off-white solid in 61% yield. FR03684-SU2-L-arginine-acetone-water-95-5-re was used for solubility study in Example 12.

TABLE 23
D-BHB L-arginine salt Form B

| | Sample ID | |
|---|---|---|
| | FR03684-SU2-L-arginine-acetone-water-95-5-re | |
| Parameter | Method | Result |
| X-ray diffraction | XRPD (Method 2), | High crystallinity, |
| | 3-40° (2 theta) | Form B, FIG. 19A |
| Thermal events and | DSC, 10° C./min | Dehydration from about |
| enthalpy | | 78° C., FIG. 19B |
| Thermogravimetry | TGA, 10° C./min | 5.9% @ 120° C. |
| | | FIG. 19C |
| Residual solvent(s) | ¹H-NMR (D₂O-d₂) | Undetected, FIG. 19D |
| Stoichiometry (free | ¹H-NMR (D₂O-d₂) | 1:1.1, FIG. 19D |
| form: L-arginine) | | |
| Water content | Karl Fischer | 5.5% water by weight |
| | (coulometric) | (1.0 equiv. by |
| | | molar ratio) |

FIG. 19A shows the XRPD for D-BHB L-arginine salt Form B (FR03684-SU2-L-arginine-acetone-water-95-5-re) having peaks as shown in TABLE 24.

TABLE 24
FR03684-SU2-L-arginine-acetone-water-95-5-re

| | | | Net | Gross | Rel. |
|---|---|---|---|---|---|
| Index | Angle | d Value | Intensity | Intensity | Intensity |
| 1 | 7.293° | 12.11083 Å | 891.537 | 946.237 | 35.4% |
| 2 | 9.964° | 8.87022 Å | 72.1331 | 139.353 | 2.9% |
| 3 | 11.290° | 7.83111 Å | 500.371 | 583.730 | 19.8% |
| 4 | 11.764° | 7.51642 Å | 122.749 | 209.589 | 4.9% |
| 5 | 14.586° | 6.06818 Å | 165.904 | 270.206 | 6.6% |
| 6 | 15.859° | 5.58389 Å | 502.386 | 616.197 | 19.9% |
| 7 | 17.268° | 5.13119 Å | 477.818 | 611.053 | 19.0% |
| 8 | 17.996° | 4.92512 Å | 235.095 | 387.080 | 9.3% |
| 9 | 18.351° | 4.83078 Å | 1977.46 | 2136.38 | 78.4% |
| 10 | 18.783° | 4.72057 Å | 161.870 | 327.144 | 6.4% |
| 11 | 19.931° | 4.45123 Å | 1472.82 | 1653.58 | 58.4% |
| 12 | 20.319° | 4.36711 Å | 890.558 | 1080.64 | 35.3% |
| 13 | 20.642° | 4.29941 Å | 414.792 | 611.226 | 16.5% |
| 14 | 20.923° | 4.24231 Å | 1198.90 | 1399.81 | 47.6% |
| 15 | 21.915° | 4.05243 Å | 1505.13 | 1716.76 | 59.7% |
| 16 | 22.630° | 3.92607 Å | 2521.19 | 2738.36 | 100.0% |
| 17 | 22.868° | 3.88571 Å | 923.402 | 1141.03 | 36.6% |
| 18 | 23.499° | 3.78279 Å | 1823.06 | 2038.51 | 72.3% |
| 19 | 24.129° | 3.68541 Å | 241.083 | 449.467 | 9.6% |
| 20 | 25.274° | 3.52099 Å | 829.501 | 1039.43 | 32.9% |
| 21 | 25.611° | 3.47546 Å | 774.334 | 990.208 | 30.7% |
| 22 | 26.466° | 3.36507 Å | 419.129 | 643.831 | 16.6% |
| 23 | 26.997° | 3.30002 Å | 1265.38 | 1491.03 | 50.2% |
| 24 | 28.016° | 3.18230 Å | 401.181 | 623.352 | 15.9% |
| 25 | 28.587° | 3.12004 Å | 514.814 | 738.670 | 20.4% |
| 26 | 29.362° | 3.03945 Å | 448.487 | 671.785 | 17.8% |
| 27 | 29.747° | 3.00099 Å | 876.854 | 1099.45 | 34.8% |
| 28 | 30.519° | 2.92681 Å | 90.2796 | 313.350 | 3.6% |
| 29 | 30.763° | 2.90413 Å | 561.016 | 786.240 | 22.3% |
| 30 | 30.882° | 2.89323 Å | 215.641 | 441.652 | 8.6% |
| 31 | 31.479° | 2.83964 Å | 125.009 | 352.319 | 5.0% |
| 32 | 31.968° | 2.79731 Å | 440.369 | 665.473 | 17.5% |
| 33 | 32.395° | 2.76145 Å | 551.909 | 772.691 | 21.9% |
| 34 | 33.040° | 2.70896 Å | 70.5273 | 284.068 | 2.8% |
| 35 | 33.565° | 2.66780 Å | 203.128 | 413.489 | 8.1% |
| 36 | 34.212° | 2.61880 Å | 107.596 | 322.096 | 4.3% |
| 37 | 34.811° | 2.57511 Å | 247.507 | 468.500 | 9.8% |
| 38 | 35.264° | 2.54309 Å | 290.718 | 513.691 | 11.5% |
| 39 | 35.643° | 2.51686 Å | 271.833 | 494.523 | 10.8% |
| 40 | 36.847° | 2.43734 Å | 534.735 | 771.279 | 21.2% |
| 41 | 37.120° | 2.42007 Å | 711.033 | 952.680 | 28.2% |
| 42 | 38.111° | 2.35938 Å | 560.210 | 814.956 | 22.2% |
| 43 | 38.520° | 2.33524 Å | 196.486 | 454.636 | 7.8% |
| 44 | 38.826° | 2.31754 Å | 146.557 | 405.909 | 5.8% |
| 45 | 39.726° | 2.26708 Å | 57.4195 | 332.555 | 2.3% |

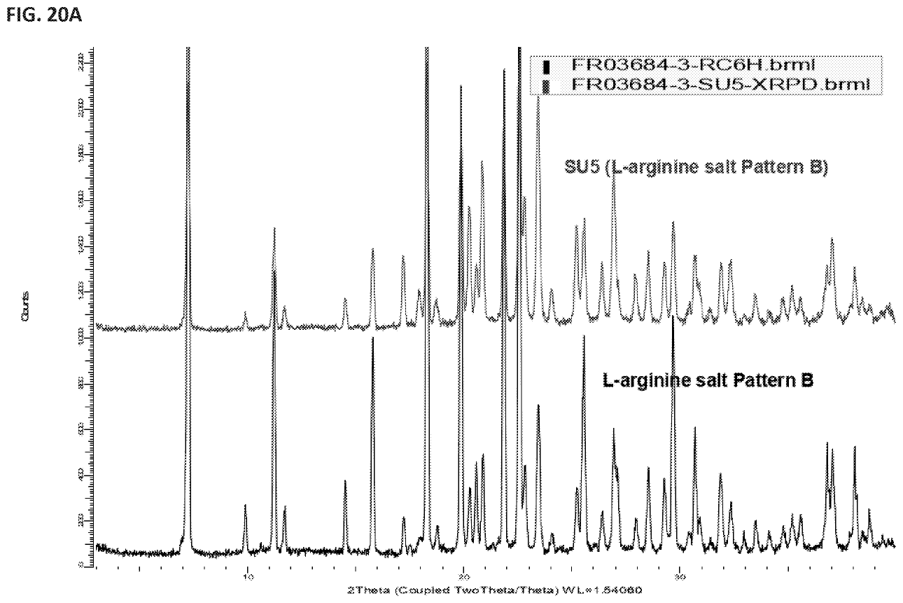

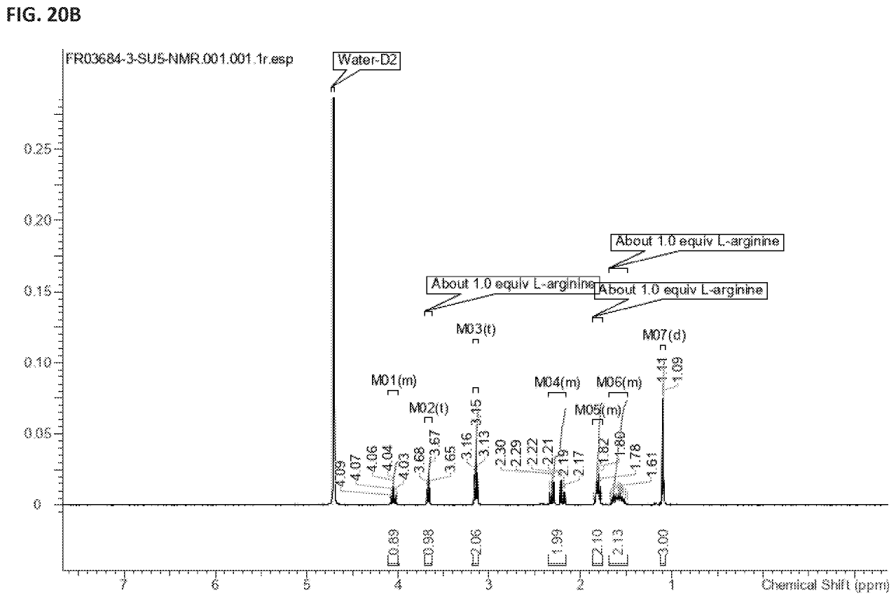

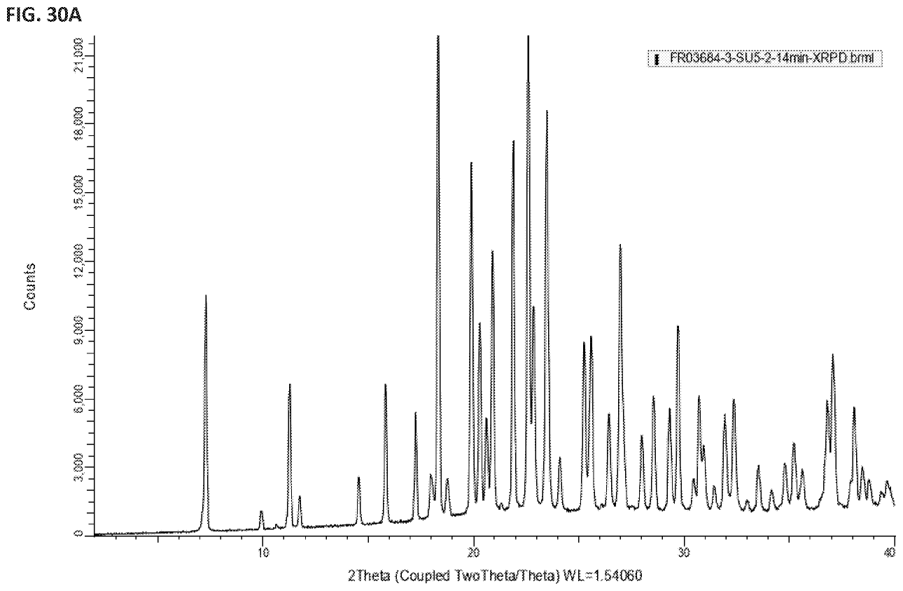

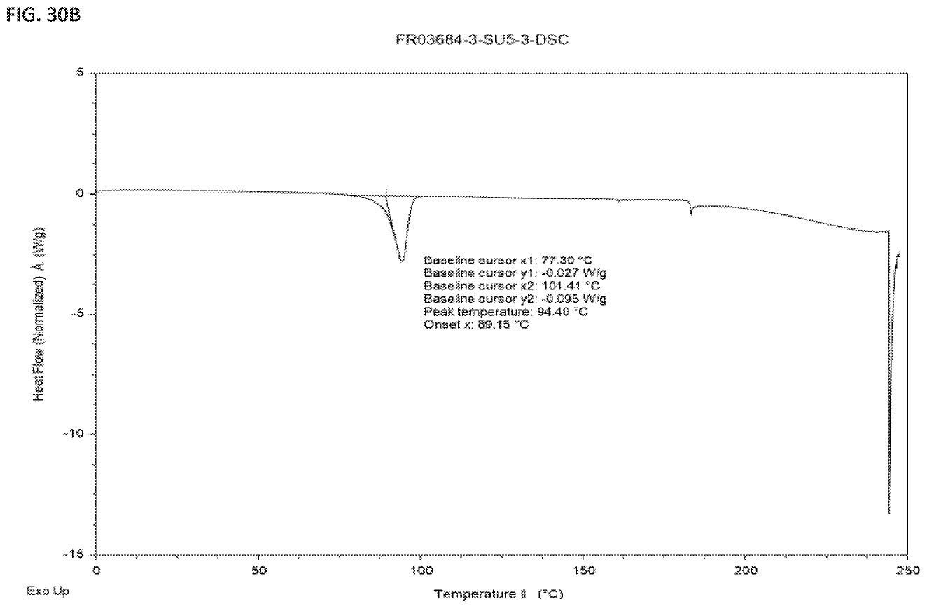

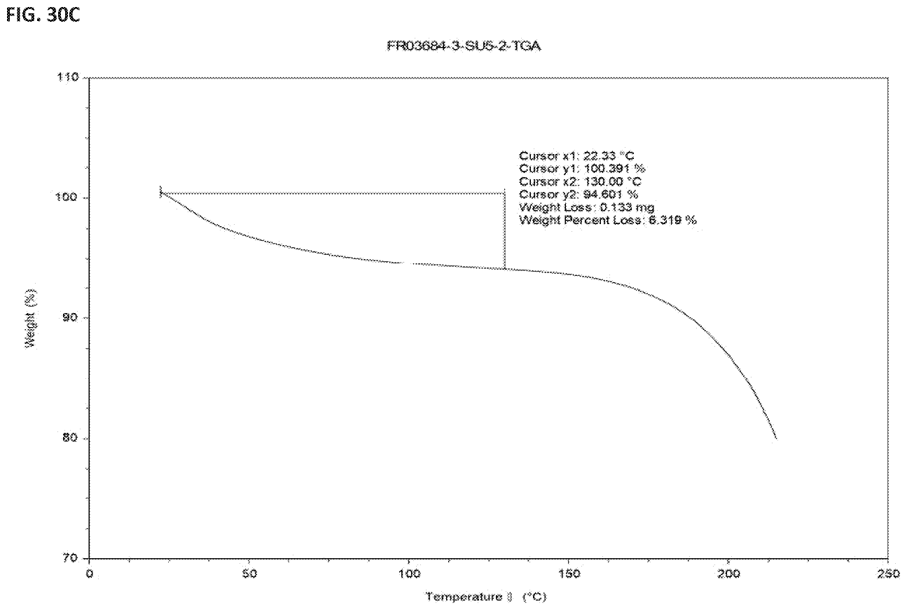

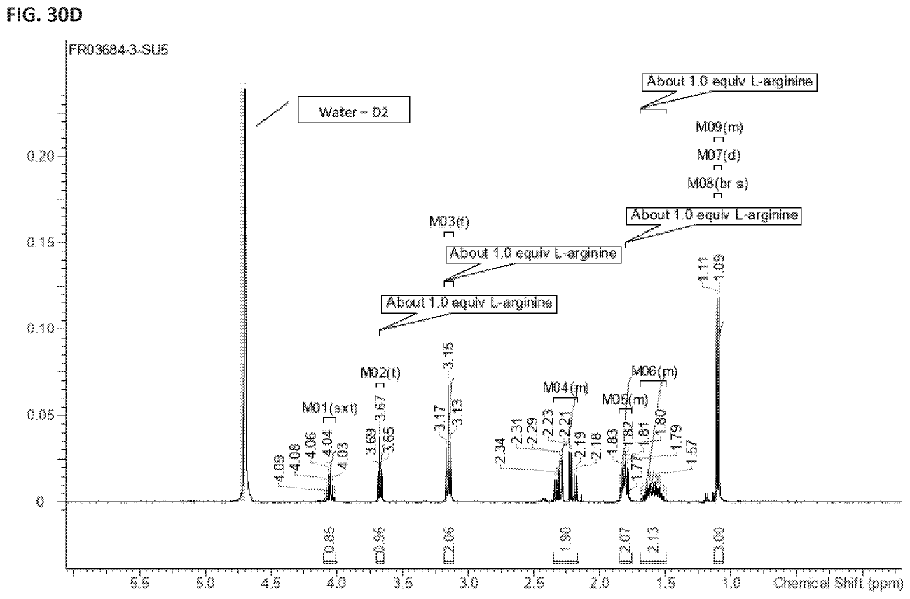

Trial 2: L-arginine salt Form B (FR03684-SU5-L-arginine-acetone-water-95-5) was prepared using the procedure below. 1.0 g of the D-BHB free Form I (FR03684-1-LP1) and 1.7 g of L-arginine (~1.0 equivalent by molar ratio) were weighed into a 40 ml glass vial. 15 ml of acetone/water (v:v=95:5) was added into the vial under stirring at 50° C. for about 10 min. A gel-like material was obtained. About 3 mg of the L-arginine salt Form B seeds (FR03684-3-RC6H) was added. This gel-like material was stirred at 50° C. for about 2 hours. This gel-like material was cooled to 25° C. and kept stirring at 25° C. for about 1 day. The gel-like material gradually converted into a suspension. The suspension was kept stirring at 25° C. for another 7 days. About 1 ml of the suspension was taken out. The solid part was collected by filtered through a 0.45 μm nylon membrane. The L-arginine salt Form B (FIG. 20A) was obtained. 1H-NMR showed that a ratio of free form:L-arginine was 1:1.0 (FIG. 20B). Solids were collected by filtration and then dried at 50° C. under vacuum for about 2 hours. 2.4 g of the L-arginine salt Form B was obtained as an off-white solid in 89% yield. Characterization results are reported in Example 14. FR03684-SU5-L-arginine-acetone-water-95-5 was used for bulk stability study, solubility study and hygroscopicity in Examples 11 to 13. The XRPD of D-BHB L-arginine salt Form B (FR03684-SU5-L-arginine-acetone-water-95-5) is shown in FIG. 30A and has peaks as shown in TABLE 25.

TABLE 25
FR03684-SU5-L-arginine-acetone-water-95-5

| | | | Net | Gross | Rel. |
|---|---|---|---|---|---|
| Index | Angle | d Value | Intensity | Intensity | Intensity |
| 1 | 7.294° | 12.11032 Å | 6716.63 | 6913.83 | 40.3% |
| 2 | 9.942° | 8.88951 Å | 525.292 | 808.894 | 3.2% |
| 3 | 10.647° | 8.30268 Å | 114.471 | 434.386 | 0.7% |
| 4 | 11.276° | 7.84061 Å | 4270.66 | 4622.98 | 25.6% |
| 5 | 11.752° | 7.52454 Å | 914.271 | 1282.82 | 5.5% |
| 6 | 14.564° | 6.07723 Å | 1400.87 | 1872.78 | 8.4% |
| 7 | 15.832° | 5.59303 Å | 4179.19 | 4726.99 | 25.1% |
| 8 | 17.266° | 5.13178 Å | 3237.70 | 3929.07 | 19.4% |
| 9 | 17.986° | 4.92803 Å | 1237.52 | 2023.85 | 7.4% |
| 10 | 18.334° | 4.83527 Å | 14735.2 | 15561.6 | 88.5% |
| 11 | 18.773° | 4.72308 Å | 1107.38 | 1979.00 | 6.6% |
| 12 | 19.912° | 4.45538 Å | 10549.9 | 11538.8 | 63.3% |
| 13 | 20.312° | 4.36862 Å | 5792.59 | 6817.26 | 34.8% |
| 14 | 20.620° | 4.30406 Å | 2997.78 | 4046.64 | 18.0% |
| 15 | 20.918° | 4.24333 Å | 8085.89 | 9155.34 | 48.5% |
| 16 | 21.336° | 4.16123 Å | 230.016 | 1323.60 | 1.4% |
| 17 | 21.897° | 4.05583 Å | 10944.8 | 12062.2 | 65.7% |
| 18 | 22.613° | 3.92902 Å | 16656.9 | 17790.3 | 100.0% |
| 19 | 22.867° | 3.88593 Å | 6260.02 | 7395.32 | 37.6% |
| 20 | 23.492° | 3.78392 Å | 12375.8 | 13507.0 | 74.3% |
| 21 | 24.116° | 3.68742 Å | 1682.79 | 2797.63 | 10.1% |
| 22 | 25.270° | 3.52154 Å | 5251.05 | 6386.88 | 31.5% |
| 23 | 25.599° | 3.47699 Å | 5402.26 | 6554.55 | 32.4% |
| 24 | 26.100° | 3.41137 Å | 176.415 | 1347.21 | 1.1% |
| 25 | 26.447° | 3.36742 Å | 3016.31 | 4195.29 | 18.1% |
| 26 | 26.990° | 3.30094 Å | 8361.14 | 9545.37 | 50.2% |
| 27 | 27.545° | 3.23567 Å | 68.0020 | 1248.06 | 0.4% |
| 28 | 28.009° | 3.18309 Å | 2453.49 | 3622.62 | 14.7% |
| 29 | 28.564° | 3.12248 Å | 3937.30 | 5084.51 | 23.6% |
| 30 | 29.331° | 3.04257 Å | 3240.09 | 4384.26 | 19.5% |
| 31 | 29.723° | 3.00329 Å | 6470.63 | 7633.20 | 38.8% |
| 32 | 30.469° | 2.93147 Å | 977.892 | 2162.13 | 5.9% |
| 33 | 30.722° | 2.90788 Å | 4214.30 | 5401.94 | 25.3% |
| 34 | 30.933° | 2.88849 Å | 2161.70 | 3350.63 | 13.0% |
| 35 | 31.451° | 2.84210 Å | 736.777 | 1922.96 | 4.4% |
| 36 | 31.950° | 2.79889 Å | 2979.86 | 4155.45 | 17.9% |
| 37 | 32.379° | 2.76275 Å | 3883.96 | 5044.17 | 23.3% |
| 38 | 33.020° | 2.71059 Å | 297.441 | 1424.86 | 1.8% |
| 39 | 33.559° | 2.66828 Å | 1377.01 | 2496.96 | 8.3% |
| 40 | 34.162° | 2.62250 Å | 692.180 | 1825.84 | 4.2% |
| 41 | 34.798° | 2.57605 Å | 1617.21 | 2782.00 | 9.7% |
| 42 | 35.226° | 2.54569 Å | 2376.02 | 3554.64 | 14.3% |
| 43 | 35.633° | 2.51754 Å | 1288.76 | 2475.19 | 7.7% |
| 44 | 36.728° | 2.44500 Å | 2045.18 | 3285.46 | 12.3% |
| 45 | 36.807° | 2.43994 Å | 3685.41 | 4931.97 | 22.1% |
| 46 | 37.083° | 2.42239 Å | 5737.22 | 7004.26 | 34.4% |
| 47 | 38.085° | 2.36093 Å | 3928.73 | 5246.63 | 23.6% |
| 48 | 38.080° | 2.36126 Å | 3945.37 | 5263.12 | 23.7% |
| 49 | 38.483° | 2.33742 Å | 1403.91 | 2730.87 | 8.4% |
| 50 | 38.803° | 2.31890 Å | 974.380 | 2305.02 | 5.8% |
| 51 | 39.430° | 2.28343 Å | 312.192 | 1640.73 | 1.9% |
| 52 | 39.644° | 2.27162 Å | 919.271 | 2244.29 | 5.5% |

### Example 7: Scale Up of D-BHB of L-Lysine Salt Form A

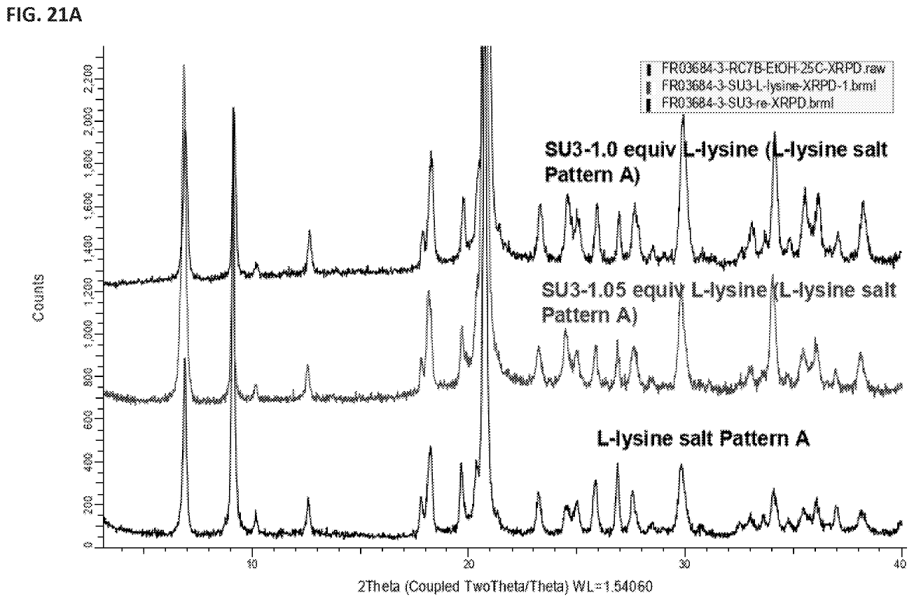

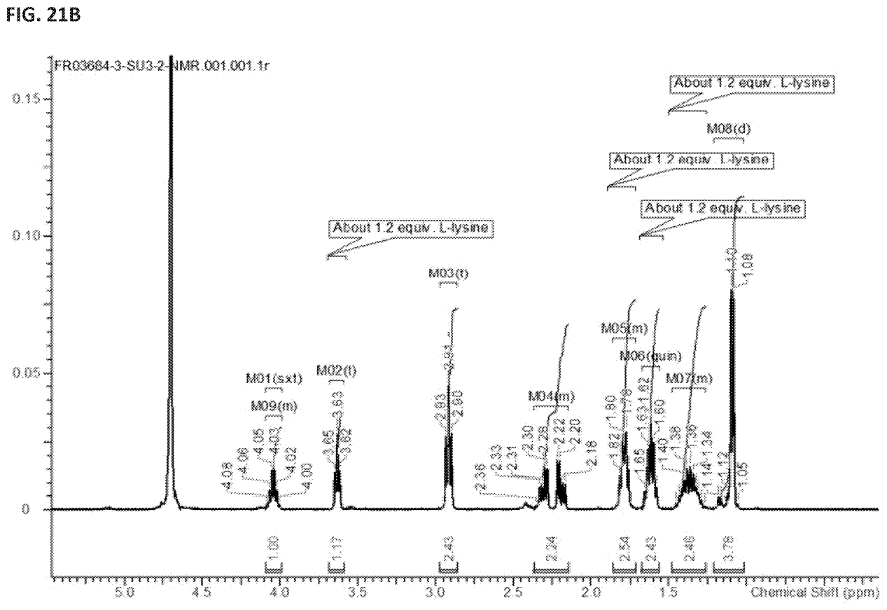

Trial 1: L-lysine salt Form A (FR03684-SU3-L-lysine-EtOH-re) was prepared as follows. 2.0 g of the D-BHB free Form I (FR03684-1-LP1) and 2.9 g of L-lysine (^{~}1.05 equivalent by molar ratio) were weighed into a 100 ml glass vial. 30 ml of EtOH was added into the vial under stirring at 25° C. for about 10 min. A suspension was obtained. About 3 mg of the L-lysine salt Form A seeds (FR03684-3-RC7B) was added into above suspension. The suspension was kept stirring at 25° C. for about 2 days. Solids were collected by filtration and then dried at 50° C. under vacuum for about 2 hours. 3.7 g of the L-lysine salt Form A (FIG. 21A) was obtained (FR03684-SU3-L-lysine-EtOH). The solid part showed a ratio of free form:L-lysine was 1:1.2 (FIG. 21B).

To optimize the stoichiometry of L-lysine salt Form A, another 100 mg (~0.05 equivalent by molar ratio) of the D-BHB free Form I (FR03684-1-LP1) and remaining 3.4 g of the L-lysine salt Form A (FR03684-3-SU3-L-lysine-EtOH) were weighed into a 100 ml glass vial. Another 5 ml of EtOH saturated solution was added into the vial and kept stirring at 25° C. for about 2 days. Solids were collected by filtration and then dried at 50° C. under vacuum for about 2 hours. 2.4 g of the L-lysine salt Form A (FIG. 21A) was obtained as an off-white solid in 54% yield. FR03684-SU3-L-lysine-EtOH-re was used for solubility study reported in Example 12.

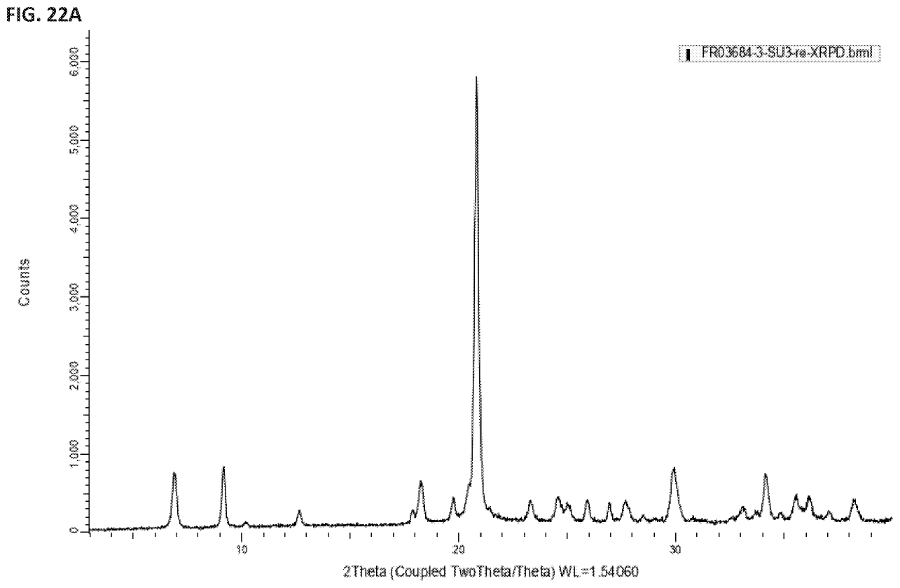

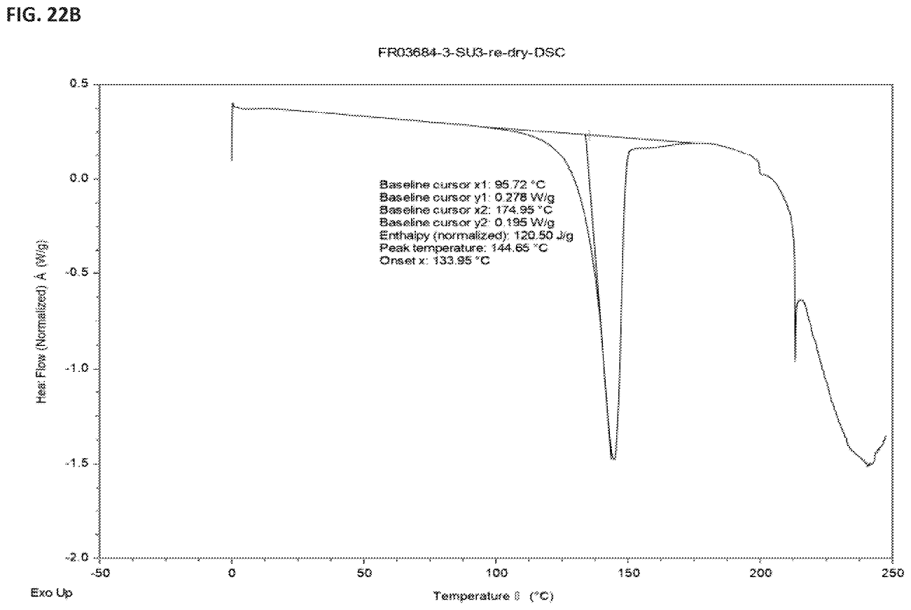

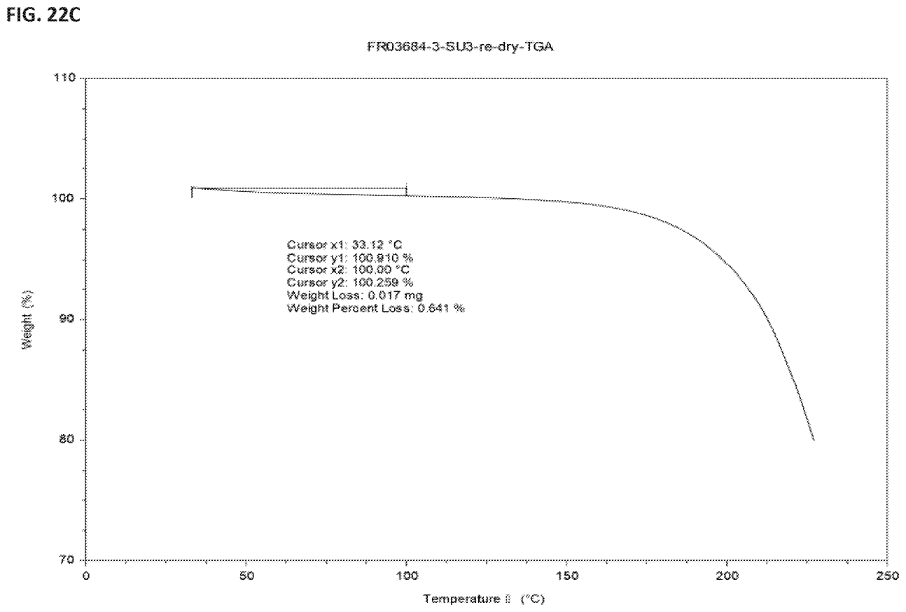

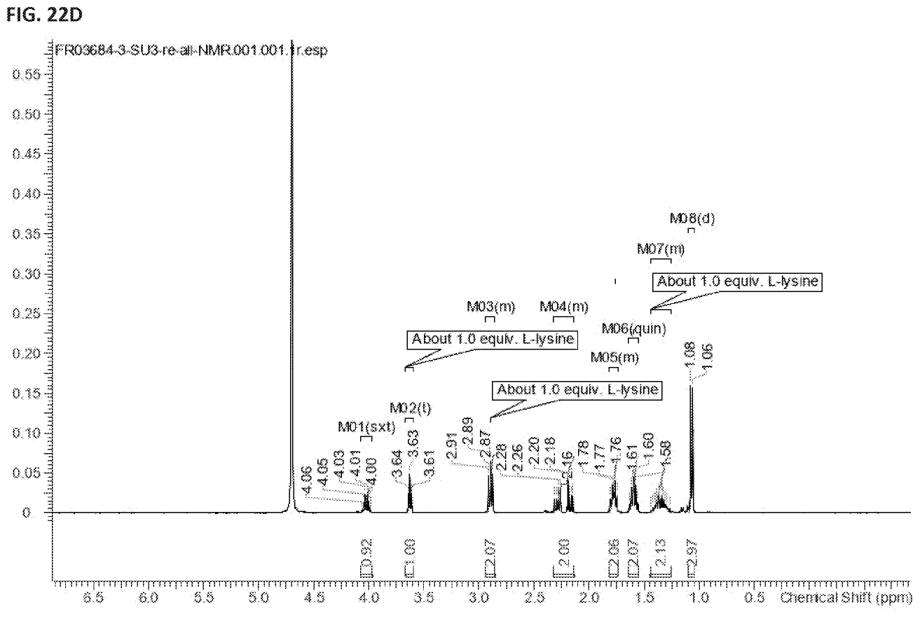

TABLE 26
D-BHB L-lysine salt Form A

| | Sample ID | |
|---|---|---|
| | FR03684-SU3-L-lysine-EtOH-re | |
| Parameter | Method | Result |
| X-ray diffraction | XRPD (Method 2), | High crystallinity, |
| | 3-40° (2 theta) | Form A, FIG. 22A |
| Thermal events | DSC, 10° C./min | Melting Tₒₙₛₑₜ @ 134° C., |
| and enthalpy | | enthalpy 121J/g, FIG. 22B |
| Thermogravimetry | TGA, 10° C./min | 0.6% @ 100° C., |
| | | FIG. 22C |
| Residual solvent(s) | ¹H-NMR (D₂O-d₂) | Undetected, FIG. 22D |
| Stoichiometry (free | ¹H-NMR (D₂O-d₂) | 1:1.0, FIG. 22D |
| form: L-lysine) | | |

FIG. 22A shows the XRPD for D-BHB L-lysine salt Form A (FR03684-SU3-L-lysine-EtOH-re) having peaks as shown in TABLE 27.

TABLE 27
FR03684-SU3-L-lysine-EtOH-re

| | | | Net | Gross | Rel. |
|---|---|---|---|---|---|
| Index | Angle | d Value | Intensity | Intensity | Intensity |
| 1 | 6.914° | 12.77429 Å | 444.076 | 505.867 | 11.5% |
| 2 | 9.181° | 9.62492 Å | 508.747 | 579.986 | 13.1% |
| 3 | 10.205° | 8.66079 Å | 25.2628 | 97.6419 | 0.7% |
| 4 | 12.669° | 6.98148 Å | 135.539 | 222.686 | 3.5% |
| 5 | 17.903° | 4.95052 Å | 98.8114 | 221.485 | 2.5% |
| 6 | 18.263° | 4.85380 Å | 360.911 | 493.260 | 9.3% |
| 7 | 19.784° | 4.48391 Å | 176.910 | 338.205 | 4.6% |
| 8 | 20.515° | 4.32574 Å | 290.907 | 462.201 | 7.5% |
| 9 | 20.840° | 4.25910 Å | 3878.22 | 4051.34 | 100.0% |
| 10 | 23.317° | 3.81191 Å | 173.128 | 330.574 | 4.5% |
| 11 | 24.629° | 3.61175 Å | 180.649 | 337.574 | 4.7% |
| 12 | 25.021° | 3.55607 Å | 149.286 | 306.989 | 3.8% |
| 13 | 25.943° | 3.43165 Å | 183.180 | 333.488 | 4.7% |
| 14 | 26.976° | 3.30261 Å | 145.470 | 289.488 | 3.8% |
| 15 | 27.701° | 3.21782 Å | 186.938 | 332.413 | 4.8% |
| 16 | 28.517° | 3.12751 Å | 54.1070 | 198.733 | 1.4% |
| 17 | 29.929° | 2.98309 Å | 412.271 | 558.216 | 10.6% |
| 18 | 30.795° | 2.90113 Å | 36.7394 | 173.892 | 0.9% |
| 19 | 32.635° | 2.74167 Å | 48.2160 | 192.988 | 1.2% |
| 20 | 33.096° | 2.70454 Å | 126.940 | 285.105 | 3.3% |
| 21 | 33.740° | 2.65436 Å | 29.6036 | 201.069 | 0.8% |
| 22 | 34.152° | 2.62326 Å | 392.462 | 569.115 | 10.1% |
| 23 | 34.848° | 2.57249 Å | 57.3374 | 236.882 | 1.5% |
| 24 | 35.556° | 2.52284 Å | 194.808 | 375.017 | 5.0% |
| 25 | 36.157° | 2.48225 Å | 174.040 | 350.648 | 4.5% |
| 26 | 37.071° | 2.42317 Å | 95.2603 | 255.866 | 2.5% |
| 27 | 38.244° | 2.35146 Å | 187.081 | 337.785 | 4.8% |

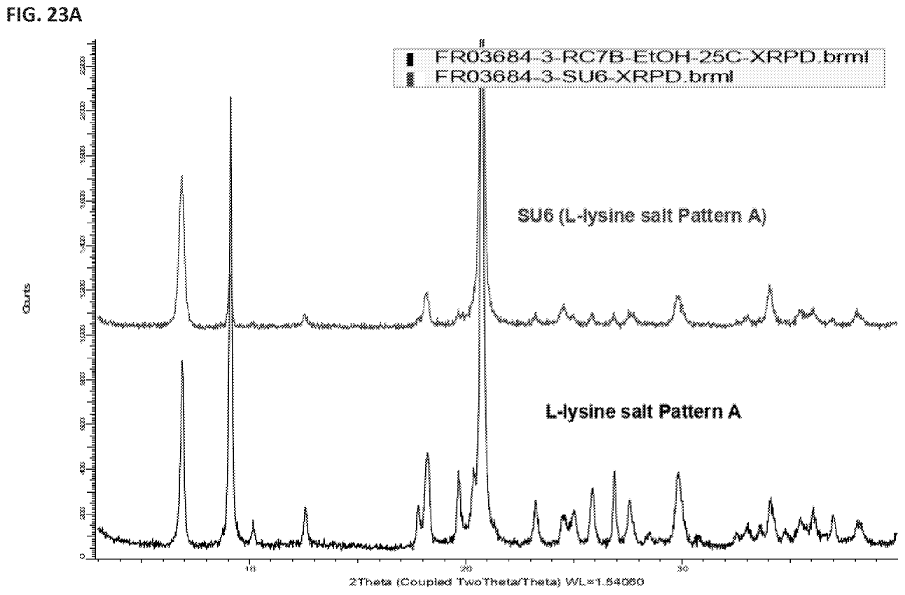

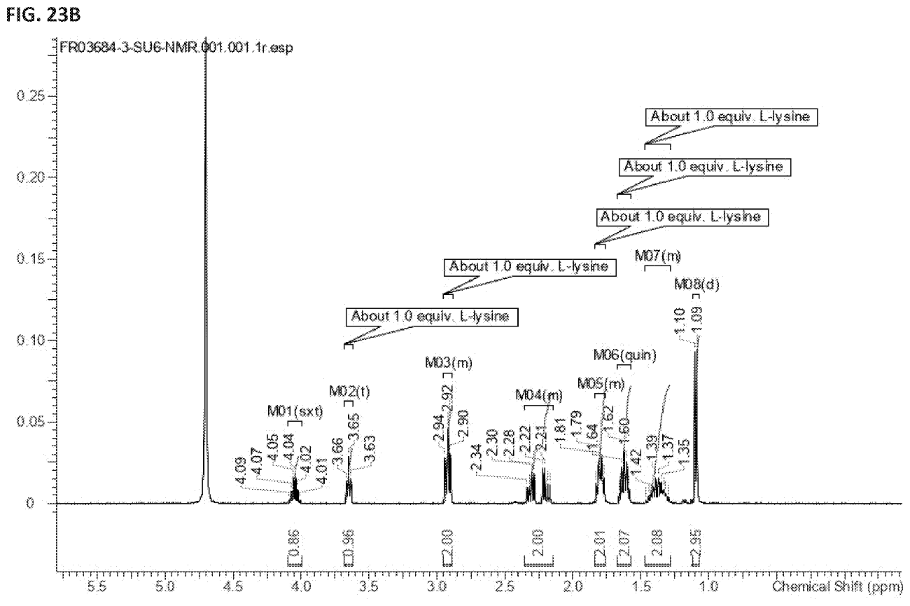

Trial 2: D-BHB L-lysine salt Form A (FR03684-SU6-L-lysine-EtOH) was prepared as follows. 1.0 g of the D-BHB free Form I (FR03684-1-LP1) and 1.4 g of L-lysine (^{~}1.0 equivalent by molar ratio) were weighed into a 100 ml glass vial. 30 ml of EtOH was added into the vial under stirring at 25° C. for about 10 min. A suspension was obtained. About 3 mg of the L-lysine salt Form A seeds (FR03684-3-RC7B) were added into above suspension. The suspension was kept stirring at 25° C. for about 7 days. About 1 ml of the suspension was taken out. The solid part was collected by filtered through a 0.45 μm nylon membrane. The L-lysine salt Form A (FIG. 23A) was obtained. ¹H-NMR showed a ratio of free form:L-lysine was 1:1.0 (FIG. 23B). Solids were collected by filtration and then dried at 50° C. under vacuum for about 2 hours. 1.6 g of the L-lysine salt Form A was obtained as an off-white solid in 67% yield. Characterization results are reported in Example 14. FR03684-SU6-L-lysine-EtOH was used for bulk stability study, solubility study and hygroscopicity reported in Examples 11 to 13.

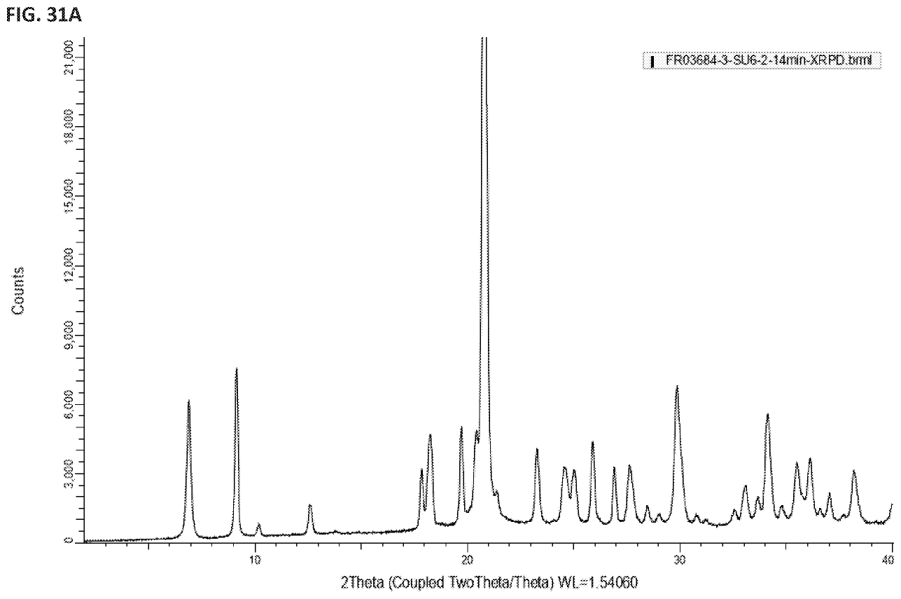

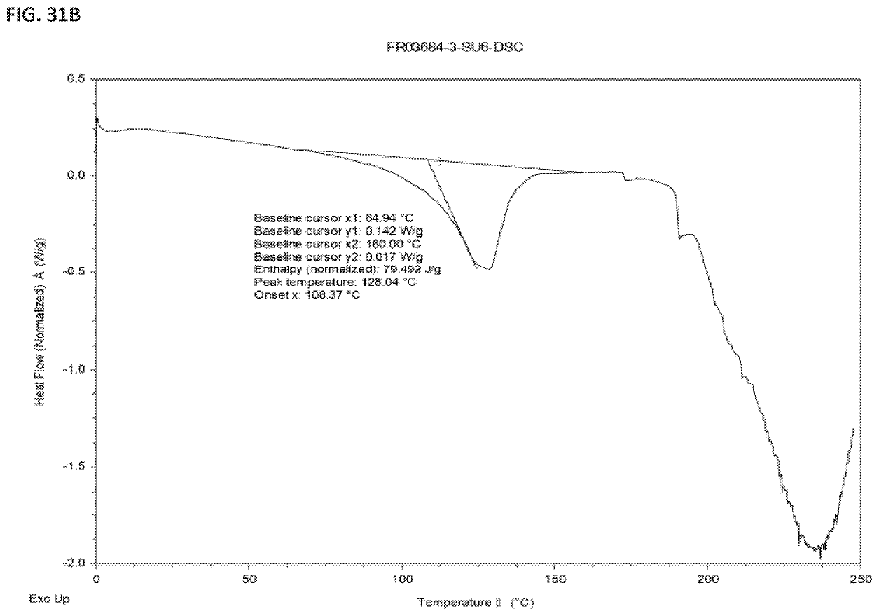

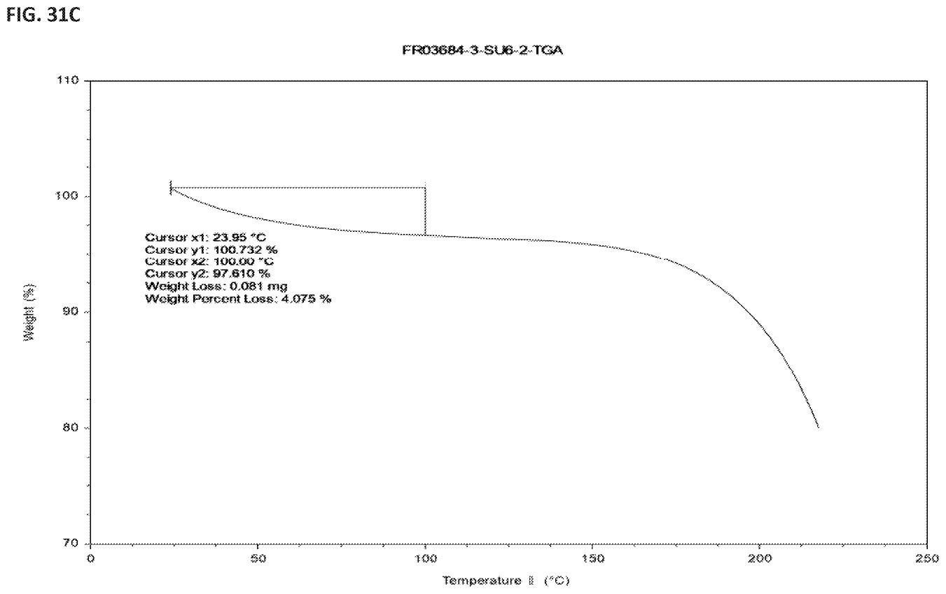

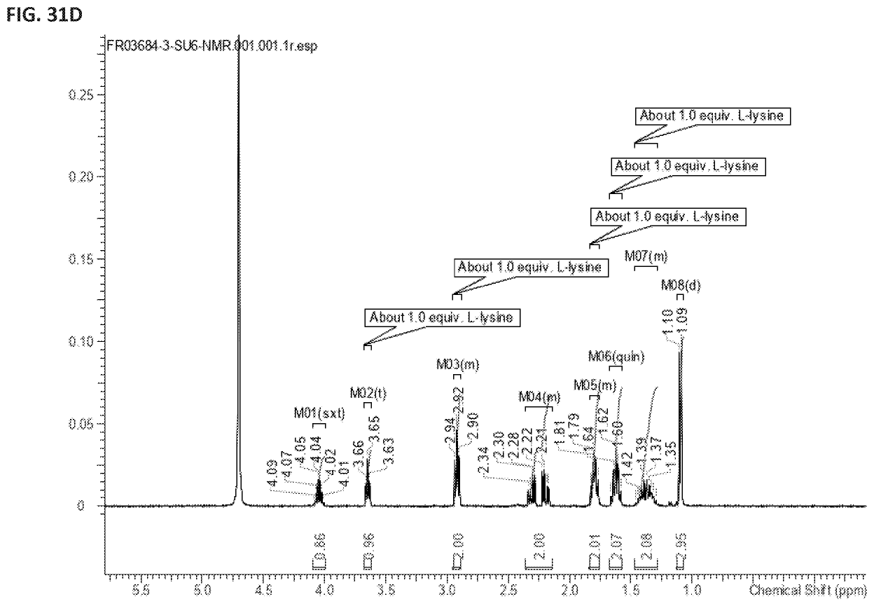

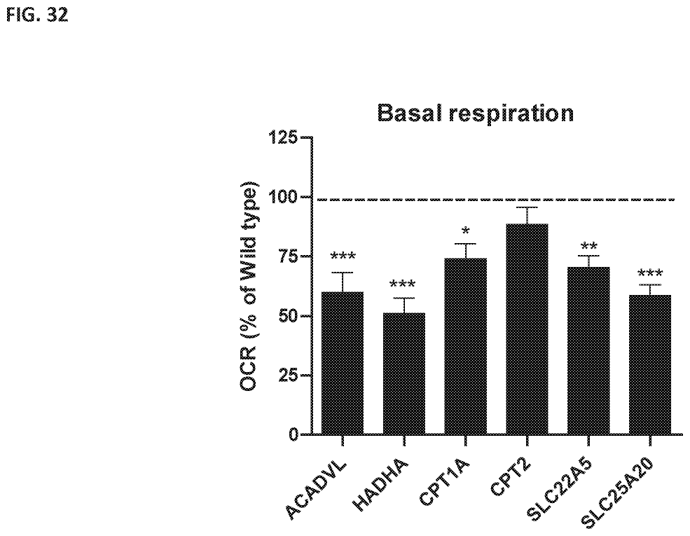

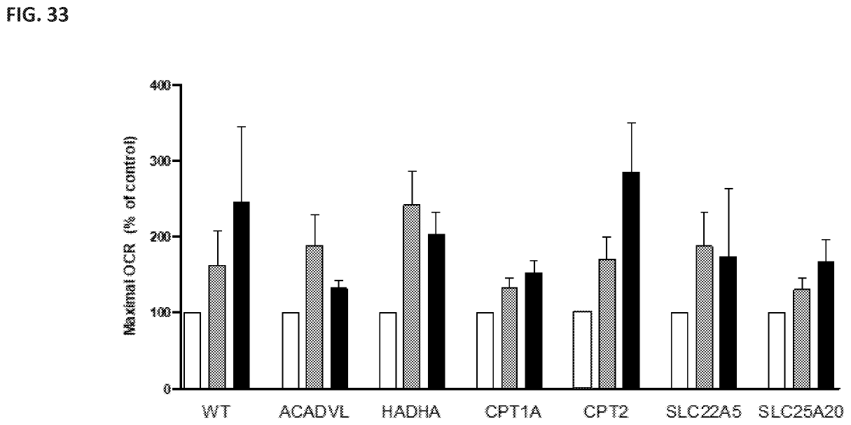

FIG. 31A shows the XPRD for D-BHB L-lysine salt Form A (FR03684-SU6-L-lysine-EtOH) having peaks as shown in TABLE 28.

TABLE 28
FR03684-SU6-L-lysine-EtOH

| | | | Net | Gross | Rel. |
|---|---|---|---|---|---|
| Index | Angle | d Value | Intensity | Intensity | Intensity |
| 1 | 6.918° | 12.76748 Å | 3849.79 | 4066.67 | 12.8% |
| 2 | 9.162° | 9.64455 Å | 4846.31 | 5125.60 | 16.1% |
| 3 | 10.211° | 8.65602 Å | 375.790 | 671.222 | 1.2% |
| 4 | 12.619° | 7.00943 Å | 817.007 | 1197.95 | 2.7% |
| 5 | 17.869° | 4.95989 Å | 1709.84 | 2326.44 | 5.7% |
| 6 | 18.274° | 4.85078 Å | 2669.94 | 3338.13 | 8.9% |
| 7 | 19.729° | 4.49619 Å | 2853.82 | 3669.32 | 9.5% |
| 8 | 20.443° | 4.34084 Å | 2693.09 | 3560.56 | 8.9% |
| 9 | 20.806° | 4.26592 Å | 30163.6 | 31049.2 | 100.0% |
| 10 | 21.390° | 4.15069 Å | 938.411 | 1841.39 | 3.1% |
| 11 | 23.289° | 3.81638 Å | 2158.19 | 3057.24 | 7.2% |
| 12 | 24.584° | 3.61817 Å | 1611.61 | 2518.65 | 5.3% |
| 13 | 25.024° | 3.55561 Å | 1513.47 | 2417.06 | 5.0% |
| 14 | 25.906° | 3.43646 Å | 2419.77 | 3291.60 | 8.0% |
| 15 | 26.926° | 3.30856 Å | 1700.39 | 2546.58 | 5.6% |
| 16 | 27.643° | 3.22438 Å | 1728.69 | 2594.88 | 5.7% |
| 17 | 28.453° | 3.13436 Å | 473.500 | 1349.08 | 1.6% |
| 18 | 29.013° | 3.07518 Å | 249.109 | 1135.97 | 0.8% |
| 19 | 29.868° | 2.98903 Å | 4049.80 | 4928.16 | 13.4% |
| 20 | 30.778° | 2.90275 Å | 255.016 | 1090.14 | 0.8% |
| 21 | 31.210° | 2.86355 Å | 166.948 | 969.171 | 0.6% |
| 22 | 32.568° | 2.74718 Å | 429.041 | 1275.61 | 1.4% |
| 23 | 33.089° | 2.70506 Å | 1033.46 | 1943.02 | 3.4% |
| 24 | 33.684° | 2.65866 Å | 616.720 | 1583.93 | 2.0% |
| 25 | 34.148° | 2.62356 Å | 2992.09 | 3993.82 | 9.9% |
| 26 | 34.828° | 2.57391 Å | 279.570 | 1315.32 | 0.9% |
| 27 | 35.503° | 2.52652 Å | 1741.33 | 2791.36 | 5.8% |
| 28 | 36.133° | 2.48389 Å | 1778.17 | 2824.01 | 5.9% |
| 29 | 36.604° | 2.45301 Å | 323.966 | 1355.63 | 1.1% |
| 30 | 37.040° | 2.42510 Å | 859.516 | 1869.59 | 2.8% |
| 31 | 37.731° | 2.38225 Å | 147.137 | 1117.04 | 0.5% |
| 32 | 38.180° | 2.35526 Å | 1593.85 | 2536.44 | 5.3% |

### Example 8: Scale Up of D-BHB Erbumine Salt Form A

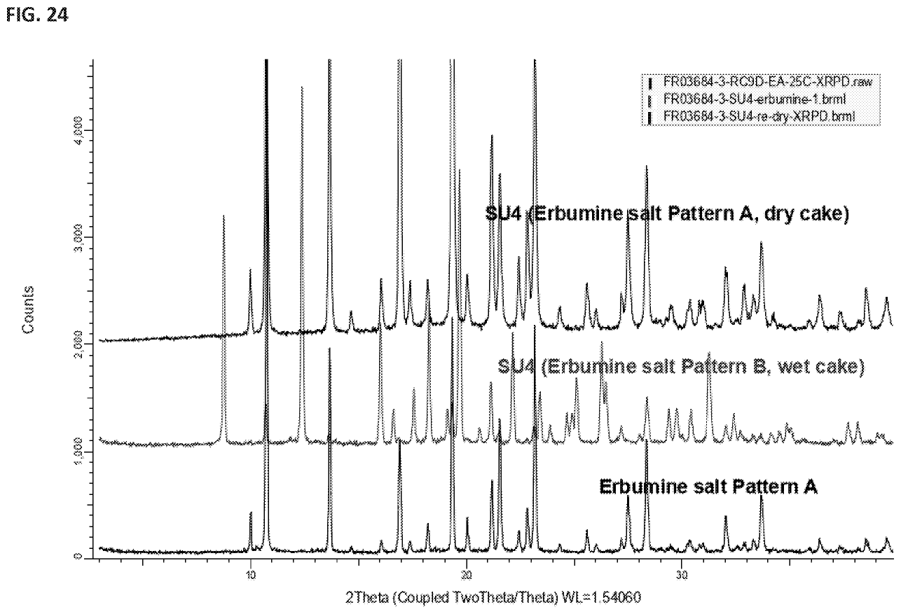

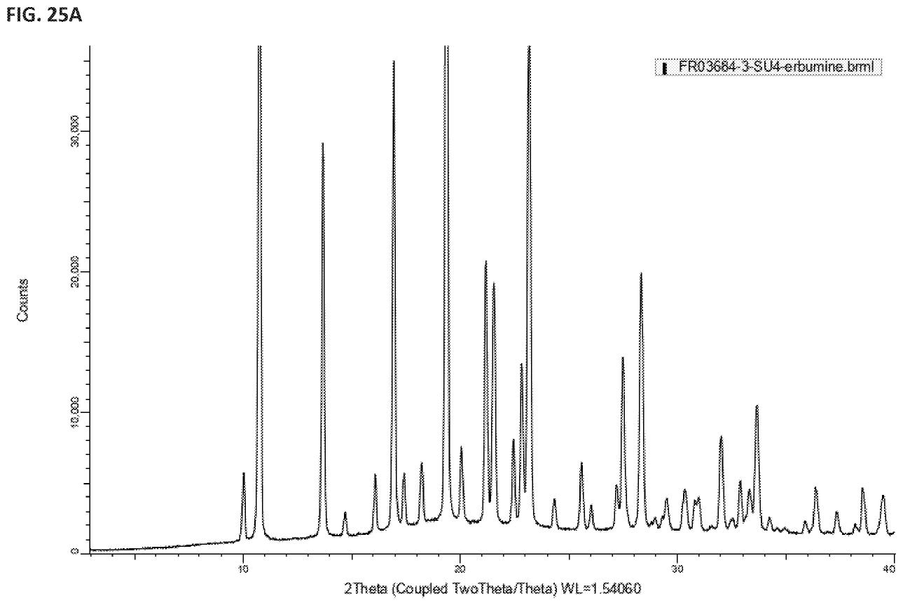

Erbumine salt Form A (FR03684-SU4-erbumine-EA) was prepared as follows. 2.0 g of the D-BHB free Form I (FR03684-1-LP1) and 1.5 g of erbumine (^{~}1.05 equivalent by molar ratio) were weighed into a 20 ml glass vial. 4.0 ml of EA was added into the vial under stirring at 25° C. for about 10 min. A suspension was obtained. About 3 mg of the erbumine salt Form A seeds (FR03684-3-RC9C) were added into above suspension. The suspension was kept stirring at 25° C. for about 2 days. About 2 ml of suspension was taken out and solid part (wet cake) was characterized by XRPD. A new crystalline form, assigned as erbumine salt Form B (FIG. 24) was obtained. After it was dried at 50° C. under vacuum for about 2 hours, the erbumine salt Form B converted to the erbumine salt Form A (FIG. 24). Rest of solids were collected by filtration and then dried at 50° C. under vacuum for about 2 hours. 2.5 g of the erbumine salt Form A was obtained as an off-white solid in 71% yield. FR03684-SU4-erbumine-EA was used for mini-polymorph screening reported in Example 10.

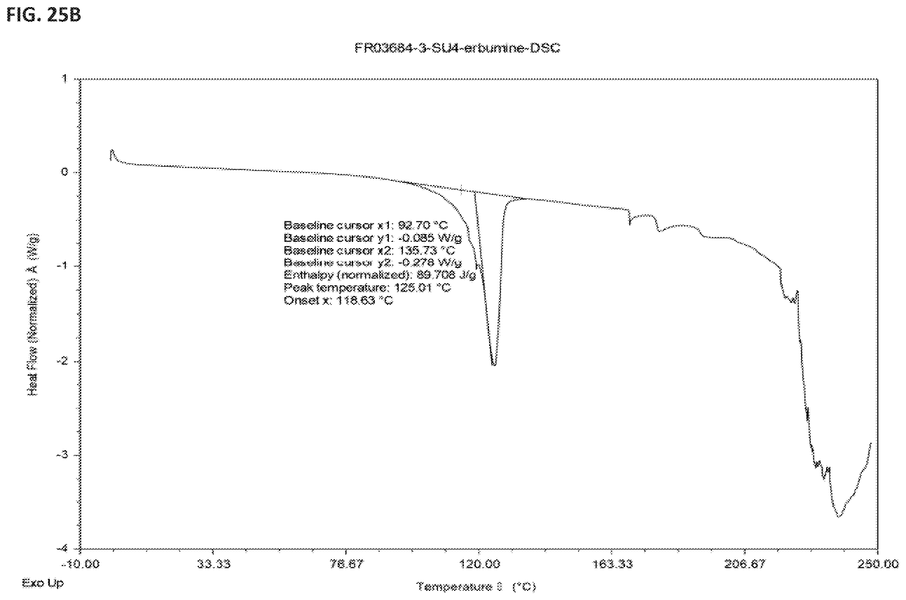

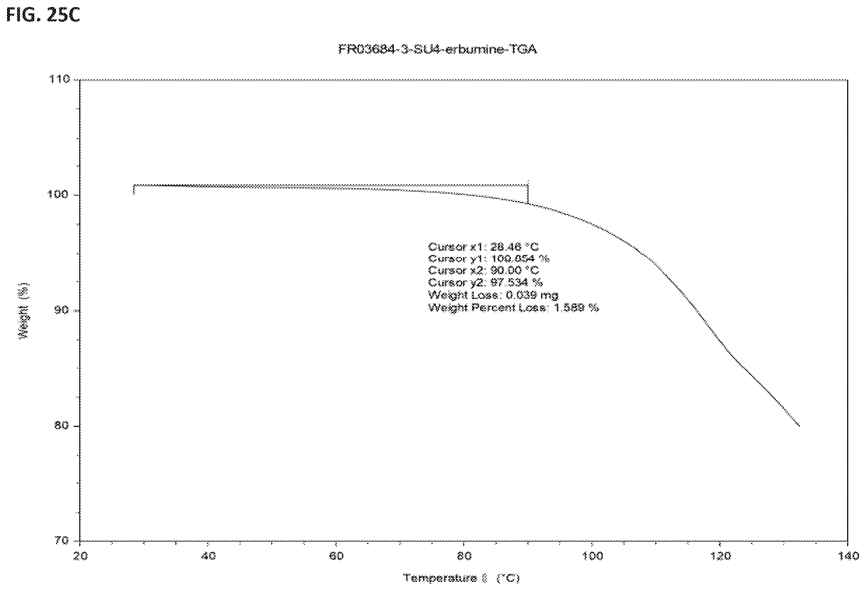

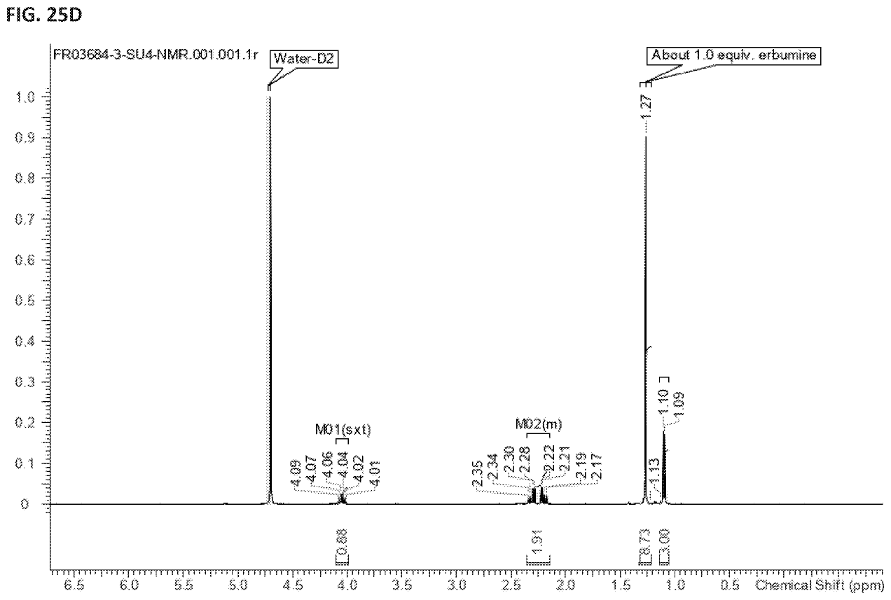

TABLE 29
D-BHB erbumine salt Form A

| | Sample ID | |
|---|---|---|
| | FR03684-SU4-erbumine-EA | |
| Parameter | Method | Result |
| X-ray diffraction | XRPD (Method 2), | High crystallinity, |
| | 3-40° (2 theta) | Form A, FIG. 25A |
| Thermal events and | DSC, 10° C./min | Melting Tₒₙₛₑₜ @ 118.6° C.; |
| enthalpy | | Decomposition upon melting, |
| | | FIG. 25B |
| Thermogravimetry | TGA, 10° C./min | About 1.6% @ 90° C., |
| | | FIG. 25C |
| Residual solvent(s) | ¹H-NMR (D₂O-d₂) | Undetected, FIG. 25D |
| Stoichiometry (free | ¹H-NMR (D₂O-d₂) | 1:1.0, FIG. 25D |
| form: erbumine) | | |

The XRPD for erbumine salt Form A (FR03684-SU4-erbumine-EA) is shown in FIG. 25A having peaks as shown in TABLE 30.

TABLE 30
FR03684-SU4-erbumine-EA

| | | | Net | Gross | Rel. |
|---|---|---|---|---|---|
| Index | Angle | d Value | Intensity | Intensity | Intensity |
| 1 | 10.039° | 8.80408 Å | 3243.41 | 4112.75 | 6.8% |
| 2 | 10.766° | 8.21077 Å | 30929.5 | 31863.2 | 64.9% |
| 3 | 13.688° | 6.46384 Å | 18988.4 | 20152.9 | 39.8% |
| 4 | 14.706° | 6.01889 Å | 1131.16 | 2384.28 | 2.4% |
| 5 | 16.084° | 5.50610 Å | 2975.07 | 4497.97 | 6.2% |
| 6 | 16.953° | 5.22567 Å | 22750.9 | 24476.0 | 47.7% |
| 7 | 17.420° | 5.08660 Å | 2602.96 | 4420.15 | 5.5% |
| 8 | 18.238° | 4.86050 Å | 3071.03 | 5021.55 | 6.4% |
| 9 | 19.374° | 4.57780 Å | 47659.3 | 49736.4 | 100.0% |
| 10 | 20.066° | 4.42148 Å | 3905.28 | 6026.01 | 8.2% |
| 11 | 21.197° | 4.18801 Å | 12839.0 | 14976.3 | 26.9% |
| 12 | 21.554° | 4.11955 Å | 11897.1 | 14025.6 | 25.0% |
| 13 | 22.466° | 3.95433 Å | 4129.23 | 6204.59 | 8.7% |
| 14 | 22.842° | 3.89015 Å | 8052.83 | 10093.5 | 16.9% |
| 15 | 23.183° | 3.83367 Å | 24177.4 | 26180.1 | 50.7% |
| 16 | 24.350° | 3.65254 Å | 1459.76 | 3286.91 | 3.1% |
| 17 | 25.597° | 3.47732 Å | 3450.65 | 5173.51 | 7.2% |
| 18 | 26.054° | 3.41730 Å | 1175.25 | 2887.96 | 2.5% |
| 19 | 27.224° | 3.27311 Å | 2172.46 | 3912.91 | 4.6% |
| 20 | 27.504° | 3.24041 Å | 8860.80 | 10614.3 | 18.6% |
| 21 | 28.351° | 3.14547 Å | 12894.6 | 14662.1 | 27.1% |
| 22 | 28.984° | 3.07820 Å | 620.032 | 2373.24 | 1.3% |
| 23 | 29.317° | 3.04398 Å | 688.176 | 2425.33 | 1.4% |
| 24 | 29.523° | 3.02322 Å | 1548.90 | 3273.19 | 3.2% |
| 25 | 30.355° | 2.94221 Å | 2045.68 | 3730.61 | 4.3% |
| 26 | 30.821° | 2.89878 Å | 1584.84 | 3256.90 | 3.3% |
| 27 | 30.976° | 2.88461 Å | 1794.04 | 3459.23 | 3.8% |
| 28 | 31.595° | 2.82954 Å | 236.243 | 1899.60 | 0.5% |
| 29 | 32.029° | 2.79219 Å | 4949.66 | 6628.11 | 10.4% |
| 30 | 32.542° | 2.74926 Å | 551.107 | 2234.56 | 1.2% |
| 31 | 32.901° | 2.72013 Å | 2755.44 | 4434.11 | 5.8% |
| 32 | 33.337° | 2.68551 Å | 2171.51 | 3835.15 | 4.6% |
| 33 | 33.677° | 2.65921 Å | 6481.81 | 8126.79 | 13.6% |
| 34 | 34.255° | 2.61566 Å | 728.421 | 2327.58 | 1.5% |
| 35 | 34.587° | 2.59129 Å | 262.209 | 1827.03 | 0.6% |
| 36 | 34.968° | 2.56395 Å | 234.363 | 1752.64 | 0.5% |
| 37 | 35.879° | 2.50089 Å | 683.956 | 2133.62 | 1.4% |
| 38 | 36.398° | 2.46641 Å | 2341.32 | 3787.16 | 4.9% |
| 39 | 37.354° | 2.40546 Å | 1180.33 | 2581.81 | 2.5% |
| 40 | 38.220° | 2.35291 Å | 519.652 | 1904.63 | 1.1% |
| 41 | 38.547° | 2.33370 Å | 2996.71 | 4385.76 | 6.3% |
| 42 | 39.478° | 2.28079 Å | 2051.89 | 3439.50 | 4.3% |

### Example 9: Scale Up of D-BHB Erbumine Salt Form B

Erbumine salt Form B (FR03684-SU8-erbumine-ACN-water-95-5) was prepared as follows. 2.0 g of the D-BHB free Form I (FR03684-1-LP2) and 1.5 g of erbumine (~1.05 equivalent by molar ratio) were weighed into a 20 mL glass vial. 10 mL of ACN was added into the vial under stirring at 25° C. for about 10 min. A suspension was obtained. About 1 mg of the erbumine salt Form B seeds (FR03684-3-PS8D) were added into above suspension. The suspension was kept stirring at 25° C. for about 1 day. About 0.1 ml of suspension was taken out and solid part was characterized by XRPD without Kapton film. A physical mixture of erbumine salt Form A and erbumine salt Form B was obtained after placed under ambient condition (20-25° C./30-60% RH) for about 30 min (FIG. 26A). Another 0.1 ml of suspension was taken out and solid part was characterized by XRPD with Kapton film. Erbumine salt Form A was obtained (FIG. 26A).

Considering that the erbumine salt Form B could be a hydrate, about 0.5 ml of water (5% water by volume) was added into above suspension and kept stirring at 25° C. for about 4 hours to obtained the erbumine salt Form B. Solids were collected by filtration, and placed under ambient condition (20-25° C./30-60% RH) for 1 day. 1.8 g of the erbumine salt Form B (FIG. 26A). was obtained as an off-white solid in 51% yield. Characterization results are reported in Example 14. FR03684-SU8-erbumine-ACN-water-95-5 was used for bulk stability study, solubility study and hygroscopcity reported in Examples 11 to 13. The XRPD for D-BHB erbumine salt Form B (FR03684-SU8-erbumine-ACN-water-95-5) is shown in FIG. 26B having peaks as shown in TABLE 31,

TABLE 31
FR03684-SU8-erbumine-ACN-water-95-5

| | | | Net | Gross | Rel. |
|---|---|---|---|---|---|
| Index | Angle | d Value | Intensity | Intensity | Intensity |
| 1 | 8.704° | 10.15112 Å | 2881.91 | 3132.93 | 6.1% |
| 2 | 8.819° | 10.01919 Å | 17854.1 | 18108.5 | 37.8% |
| 3 | 12.416° | 7.12351 Å | 47242.3 | 47631.0 | 100.0% |
| 4 | 13.971° | 6.33356 Å | 322.099 | 714.278 | 0.7% |
| 5 | 16.074° | 5.50942 Å | 7276.17 | 7768.78 | 15.4% |
| 6 | 16.689° | 5.30796 Å | 1819.65 | 2381.91 | 3.9% |
| 7 | 17.620° | 5.02953 Å | 8911.94 | 9572.89 | 18.9% |
| 8 | 18.345° | 4.83220 Å | 9185.88 | 9912.71 | 19.4% |
| 9 | 19.215° | 4.61528 Å | 3468.93 | 4249.65 | 7.3% |
| 10 | 19.409° | 4.56978 Å | 2671.71 | 3461.46 | 5.7% |
| 11 | 19.758° | 4.48979 Å | 37136.7 | 37937.8 | 78.6% |
| 12 | 20.712° | 4.28503 Å | 1510.22 | 2309.08 | 3.2% |
| 13 | 21.215° | 4.18453 Å | 12215.8 | 12994.0 | 25.9% |
| 14 | 22.223° | 3.99701 Å | 10952.8 | 11693.3 | 23.2% |
| 15 | 23.493° | 3.78369 Å | 4769.37 | 5512.15 | 10.1% |
| 16 | 23.952° | 3.71229 Å | 1773.77 | 2542.97 | 3.8% |
| 17 | 24.758° | 3.59320 Å | 2322.62 | 3167.55 | 4.9% |
| 18 | 24.994° | 3.55977 Å | 4696.50 | 5568.91 | 9.9% |
| 19 | 25.179° | 3.53409 Å | 8730.46 | 9622.26 | 18.5% |
| 20 | 26.356° | 3.37890 Å | 11815.9 | 12788.8 | 25.0% |
| 21 | 26.491° | 3.36200 Å | 45821.0 | 46798.5 | 97.0% |
| 22 | 27.273° | 3.26724 Å | 2879.27 | 3864.28 | 6.1% |
| 23 | 28.094° | 3.17361 Å | 1722.47 | 2722.09 | 3.6% |
| 24 | 28.462° | 3.13342 Å | 4177.92 | 5194.74 | 8.8% |
| 25 | 29.458° | 3.02977 Å | 8707.89 | 9783.13 | 18.4% |
| 26 | 29.818° | 2.99400 Å | 5875.30 | 6979.44 | 12.4% |
| 27 | 30.098° | 2.96678 Å | 918.472 | 2040.30 | 1.9% |
| 28 | 30.493° | 2.92918 Å | 4349.17 | 5488.92 | 9.2% |
| 29 | 31.357° | 2.85044 Å | 13921.9 | 15086.1 | 29.5% |
| 30 | 32.118° | 2.78463 Å | 2895.47 | 4057.87 | 6.1% |
| 31 | 32.469° | 2.75534 Å | 4494.83 | 5646.04 | 9.5% |
| 32 | 32.790° | 2.72904 Å | 1645.76 | 2780.97 | 3.5% |
| 33 | 33.308° | 2.68781 Å | 644.742 | 1742.68 | 1.4% |
| 34 | 33.649° | 2.66132 Å | 392.220 | 1458.21 | 0.8% |
| 35 | 34.227° | 2.61768 Å | 1372.78 | 2421.30 | 2.9% |
| 36 | 34.551° | 2.59388 Å | 1285.78 | 2339.77 | 2.7% |
| 37 | 34.916° | 2.56758 Å | 2983.04 | 4036.52 | 6.3% |
| 38 | 35.149° | 2.55115 Å | 3781.27 | 4830.73 | 8.0% |
| 39 | 35.671° | 2.51500 Å | 583.186 | 1613.18 | 1.2% |
| 40 | 37.083° | 2.42240 Å | 412.310 | 1424.24 | 0.9% |
| 41 | 37.768° | 2.38000 Å | 10349.4 | 11399.9 | 21.9% |
| 42 | 38.216° | 2.35316 Å | 3070.01 | 4132.22 | 6.5% |
| 43 | 38.725° | 2.32336 Å | 316.034 | 1378.64 | 0.7% |
| 44 | 39.159° | 2.29864 Å | 1162.99 | 2215.05 | 2.5% |
| 45 | 39.257° | 2.29309 Å | 1137.84 | 2186.10 | 2.4% |

### Example 10: Mini-Polymorph Screening

mg of each physical form was added to 50-200 μL solvent. Obtained suspensions were equilibrated at 25° C. for 1 week. Solids were isolated by centrifugation filtration, and wet cakes were analyzed by XRPD to determine crystal form change.

Clear solutions were evaporated under ambient condition (20-25° C./40-70% RH), and obtained wet cakes were analyzed by XRPD to determine crystal form change.

TABLE 32

| | | Physical Form | | | |
|---|---|---|---|---|---|
| | | | L-arginine | | |
| | | | salt Form B, | L-lysine salt | |
| | | Sodium salt | hydrate | Form A, | Erbumine |
| | | Form C, | FR03684-SU2- | anhydrate | salt Form A, |
| | | hydrate | L-arginine- | FR03684-SU3- | anhydrate |
| | | FR03684-SU1- | acetone- | L-lysine- | FR03684-SU4- |
| Exp. | Batch/ | NaOH-1.5-THF | water-95-5 | EtOH-re | erbumine-EA |
| ID | sample ID | XRPD | XRPD | XRPD | XRPD |
| PS1 | Water | Clear solution | Clear solution | Oil | Clear solution |
| | | was evaporated | was evaporated | | was evaporated |
| | | under ambient | under | | under ambient |
| | | condition (20- | ambient | | condition |
| | | 25° C./40- | condition | | (20-25° C./ |
| | | 70% RH). | (20-25° C./ | | 40-70% RH). Gel |
| | | Sodium Form B | 40-70% RH). Gel | | was obtained |
| | | was obtained | was obtained | | after 4 days. |
| | | after 7 days. | after 4 days. | | |
| | | FIG. 27A | | | |
| | | XRPD Method 4 | | | |
| PS2 | Methanol | Hazy | L-arginine | L-lysine salt | Clear solution |
| | | suspension | salt Form B | Form A | was evaporated |
| | | | FIG. 27D | FIG. 27F | under ambient |
| | | | XRPD Method 4 | XRPD Method 4 | condition |
| | | | | | (20-25° C./ |
| | | | | | 40-70% RH). Gel |
| | | | | | was obtained |
| | | | | | after 4 days. |
| PS3 | Ethanol | Sodium salt | L-arginine | L-lysine salt | Clear solution |
| | | Form C | salt Form B | Form A | was evaporated |
| | | FIG. 27A | FIG. 27D | FIG. 27F | under ambient |
| | | XRPD Method 4 | XRPD Method 4 | XRPD Method 4 | condition |
| | | | | | (20-25° C./ |
| | | | | | 40-70% RH). Gel |
| | | | | | was obtained |
| | | | | | after 4 days. |
| PS4 | Isopropanol | Sodium salt | L-arginine | L-lysine salt | Erbumine |
| | | Form C | salt Form B | Form A | salt Form A |
| | | FIG. 27A | FIG. 27D | FIG. 27F | FIG. 27H |
| | | XRPD Method 4 | XRPD Method 4 | XRPD Method 4 | XRPD |
| | | | | | Method 4 |
| PS5 | Acetone | Sodium salt | L-arginine | L-lysine salt | Amorphous |
| | | Form C | salt Form B | Form A | form, limited |
| | | FIG. 27A | FIG. 27D | FIG. 27F | sample |
| | | XRPD Method 4 | XRPD Method 4 | XRPD Method 4 | amount |
| PS6 | Methyl ethyl | Sodium salt | L-arginine | L-lysine salt | Erbumine |
| | ketone | Form C | salt Form B | Form A | salt Form A |
| | | FIG. 27A | FIG. 27D | FIG. 27G | FIG. 27H |
| | | XRPD Method 4 | XRPD Method 4 | XRPD Method 4 | XRPD Method 4 |
| PS7 | Ethyl acetate | One diffraction | L-arginine | L-lysine salt | Erbumine |
| | | peak at 7.1°, | salt Form B | Form A | salt Form A |
| | | limited sample | FIG. 27E | FIG. 27G | FIG. 27H |
| | | amount | XRPD Method 4 | XRPD Method 4 | XRPD Method 4 |
| | | FIG. 27B | | | |
| | | XRPD Method 4 | | | |
| PS8 | Acetonitrile | Sodium salt | L-arginine | L-lysine salt | Erbumine |
| | | Form C | salt Form B | Form A | salt Form B |
| | | FIG. 27B | FIG. 27E | FIG. 27G | FIG. 271 |
| | | XRPD Method 4 | XRPD Method 4 | XRPD Method 4 | XRPD Method 4 |
| PS9 | Tetrahydrofuran | Sodium salt | L-arginine | L-lysine salt | Erbumine |
| | | Form C | salt Form B | Form A | salt Form A |
| | | FIG. 27B | FIG. 27E | FIG. 27G | FIG. 271 |
| | | XRPD Method 4 | XRPD Method 4 | XRPD Method 4 | XRPD Method 4 |
| PS10 | Dichloromethane | Sodium salt | L-arginine | L-lysine salt | Erbumine |
| | | Form C | salt Form B | Form A | salt Form A |
| | | FIG. 27B | FIG. 27E | FIG. 27G | FIG. 271 |
| | | XRPD Method 4 | XRPD Method 4 | XRPD Method 4 | XRPD Method 4 |
| PS11 | IPA/water | Clear solution | Clear solution | Oil | Clear solution |
| | (v:v = 77:23) | was evaporated | was evaporated | | was evaporated |
| | a.w. = 0.9* | under ambient | under ambient | | under ambient |
| | | condition | condition | | condition |
| | | (20-25° C./ | (20-25° C./ | | (20-25° C./ |
| | | 40-70% RH). | 40-70% RH). Gel | | 40-70% RH). Gel |
| | | Sodium Form B | was obtained | | was obtained |
| | | was obtained | after 4 days. | | after 4 days. |
| | | after 7 days. | | | |
| | | FIG. 27C | | | |
| | | XRPD Method 2 | | | |
| PS12 | Acetone/water | Clear solution | Clear solution | Oil | Clear solution |
| | (v:v = 35:65) | was evaporated | was evaporated | | was evaporated |
| | a.w. = 0.9* | under ambient | under ambient | | under ambient |
| | | condition | condition | | condition |
| | | (20-25° C./ | (20-25° C./ | | (20-25° C./ |
| | | 40-70% RH). | 40-70% RH). Gel | | 40-70% RH). Gel |
| | | Sodium salt | was obtained | | was obtained |
| | | Form B was | after 4 days. | | after 4 days. |
| | | obtained after | | | |
| | | 4 days. | | | |
| | | FIG. 27C | | | |
| | | XRPD Method 2 | | | |

### Example 11: Bulk Stability

Salts were placed at 25° C./92.5% RH in an open container, at 25° C./60% RH in an open container, at 40° C./75% RH in an open container and at 60° C. in a closed container for 2 weeks. Samples after the stress were characterized by XRPD and IC and inspected for color change.

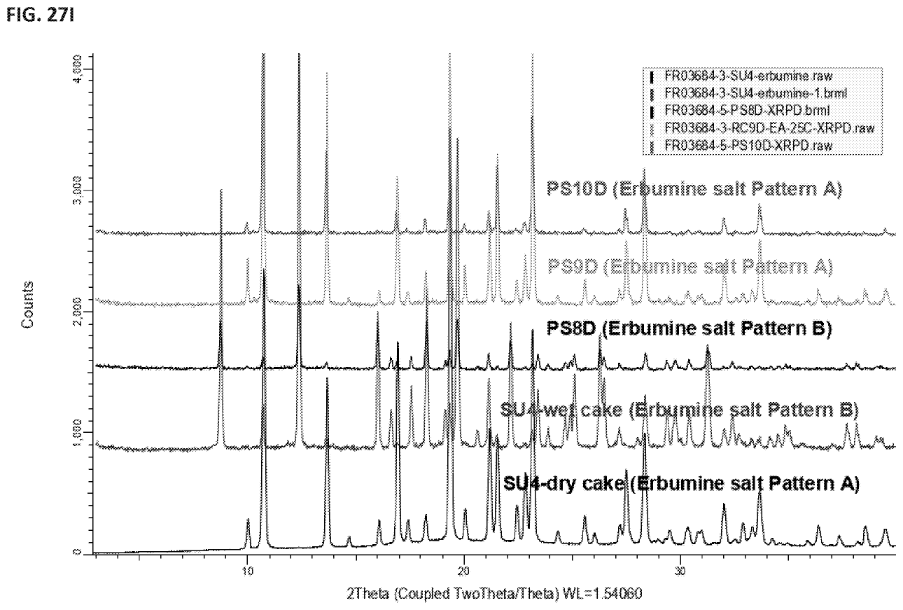

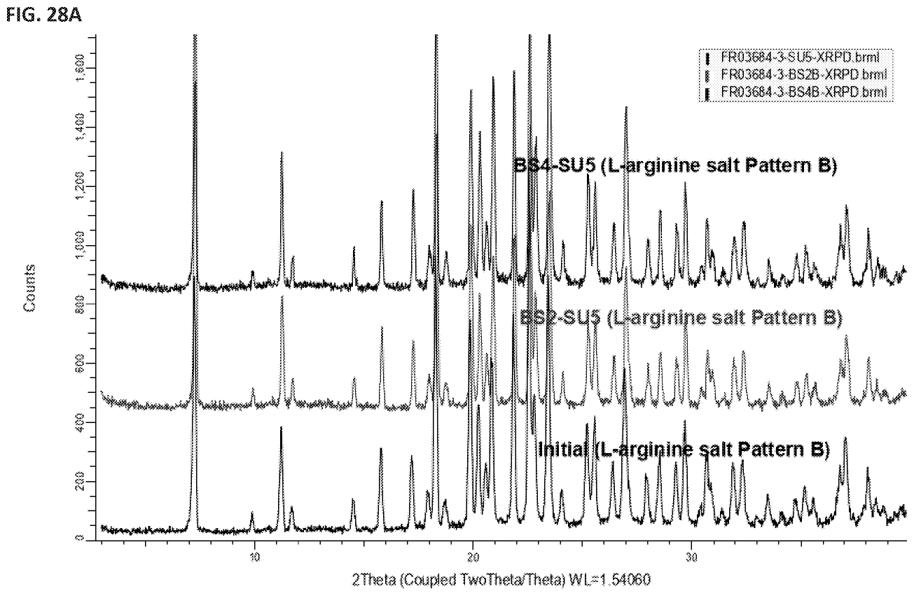

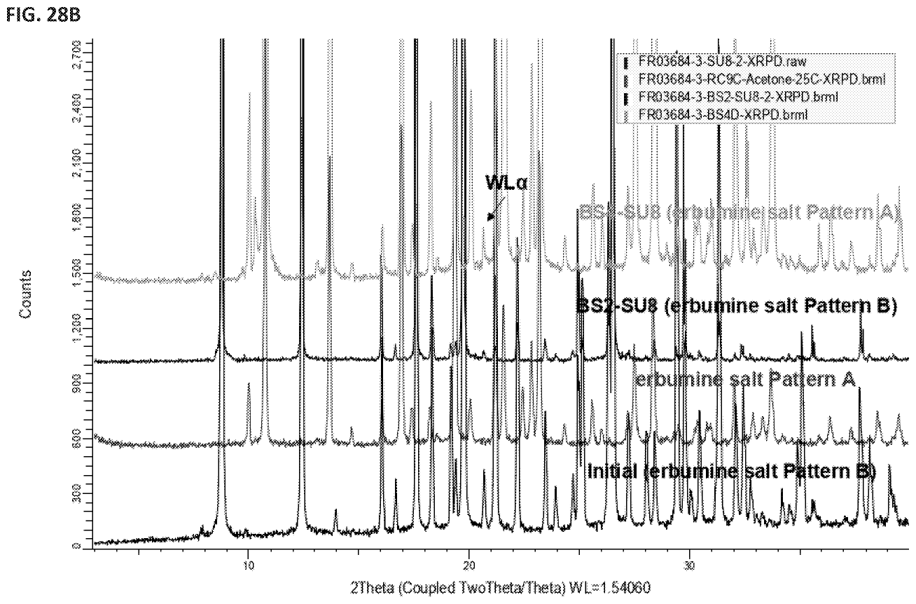

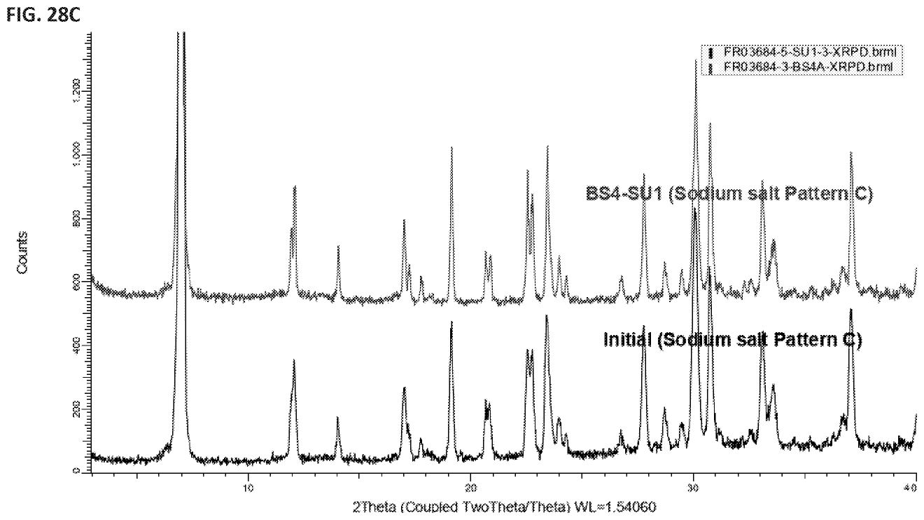

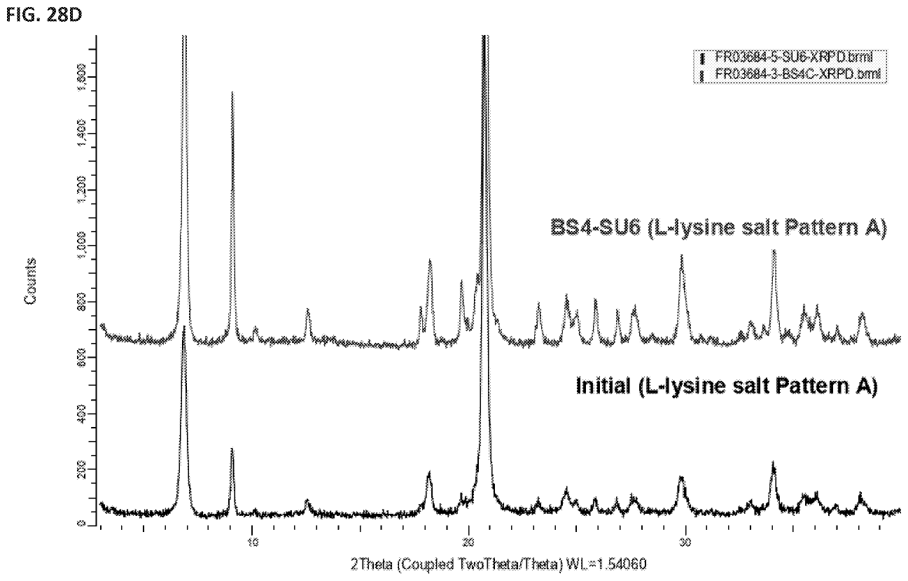

TABLE 33

| | | Physical Form | | | |
|---|---|---|---|---|---|
| | | | L-arginine salt | | |
| | | Sodium salt | Form B, | L-lysine salt | Erbumine salt |
| | | Form C, | hydrate | Form A, | Form B, |
| | | hydrate | FR03684-SU5- | anhydrate | hydrate |
| | | FR03684- | L-arginine- | FR03684-SU6- | FR03684-SU8- |
| | Batch/ | SU1-NaOH- | acetone- | L-lysine- | erbumine-ACN- |
| | sample ID | 1.5-THF | water-95-5 | EtOH | water-95-5 |
| Exp. ID | Initial color | Off-white | Off-white | Off-white | Off-white |
| BS1 | Solid state, 25° C./92.5% RH, open container, 2 weeks | | | | |
| | Bulk (XRPD) | Deliquesce | Deliquesce | Deliquesce | Deliquesce |
| BS2 | Solid state, 25° C./60% RH, open container, 2 weeks | | | | |
| | Bulk (XRPD) | Partially | Solids | Deliquesce | Solids Erbumine |
| | | deliquesce | L-arginine salt | | salt Form B, |
| | | | Form B | | XRPD test with |
| | | | FIG. 28A | | nickel plate |
| | | | XRPD Method 1 | | FIG. 28B |
| | | | | | XRPD Method 1 |
| BS3 | Solid state, 40° C./75% RH, open container, 2 weeks | | | | |
| | Bulk (XRPD) | Deliquesce | Deliquesce | Deliquesce | Deliquesce |
| BS4 | Solid state, 60° C., tight container, 2 weeks | | | | |
| | Bulk (XRPD) | Sodium salt | L-arginine salt | L-lysine salt | Erbumine salt |
| | | Form C | Form B | Form A | Form A |
| | | FIG. 28C | FIG. 28A | FIG. 28D | FIG. 28B |
| | | XRPD Method 1 | XRPD Method 1 | XRPD Method 1 | XRPD Method 1 |

All salts tested were chemically stable under these conditions.

No obvious impurity peaks were observed for the sodium salt Form C, the L-arginine salt Form B, the L-lysine salt Form A, or the erbumine salt Form B by IC.

The sesqui-sodium salt Form C and the L-lysine salt Form A were physically stable with no form change after stressed at 60° C. in a tight container over 2 weeks. However, each of these were partially deliquesced or deliquesced after stressed at 25° C./92.5% RH in an open container, at 25° C./60% RH in an open container, or at 40° C./75% RH in an open container over 2 weeks.

The L-arginine salt Form B and the erbumine salt Form B showed obvious advantage in physical stability at 25° C./60% RH. Each form remained as a solid and showed no physical change after placed at 25° C./60% RH in an open container over 2 weeks. The L-arginine salt Form B was also physically stable with no form change after stressed at 60° C. in a tight container over 2 weeks, while the erbumine salt Form B dehydrated and converted to the erbumine salt Form A after stressed at 60° C. in a tight container over 2 weeks. Both the L-arginine salt Form B and the erbumine salt Form B are not tolerant to high humidity. They deliquesced after stressed at 25° C./92.5% RH in an open container, or at 40° C./75% RH in an open container over 2 weeks.

### Example 12: Solubility

Solubility of the salts were measured in comparison of the D-BHB free Form I in seven aqueous pH buffers and bio-relevant fluids, including pH 1.2 HCl buffer, pH 4.5 acetate buffer (50 mM), pH 6.8 phosphate buffer (50 mM), water, pH 1.6 FaSSGF (Fasted State Simulated Gastric Fluid—Biorelevant), pH 6.5 FaSSIF-v1 (Fasted State Simulated Intestinal Fluid-Biorelevant), and pH 5.0 FeSSIF-v1 (Fed State Simulated Intestinal Fluid—Biorelevant), at 37° C. for 2 hours and 24 hours.

Trial 1: Accurate 1.01 mg of the D-BHB free Form I (FR03684-3-LP2), 1.43 mg of the sodium salt Form C (FR03684-SU1-NaOH-1.5-THF), 1.49 mg of the sodium salt Form C (FR03684-SU9-NaOH-1.5-THF), 3.04 mg of the L-arginine salt Form B FR03684-SU5-L-arginine-acetone-water-95-5), 2.58 mg of the L-lysine salt Form A (FR03684-SU6-L-lysine-EtOH) or 1.89 mg of the erbumine salt Form B (FR03684-SU8-erbumine-CAN-water-95-5) was weighed into a 2 ml glass vial, respectively. 0.5 ml of solubility medium (including pH 1.2 HCl buffer, pH 4.5 acetate buffer, pH 6.8 phosphate buffer water, FaSSGF, FaSSIF and FeSSIF) was added. The salt amount used are equivalent to 1 mg anhydrous free form.

Obtained clear solutions were stirred at 37° C. at 400 rpm and sampled at 2 hours and at 24 hours. Obtained clear solutions were analyzed by pH meter for pH value.

TABLE 34

| | Solubility at 37° C., target concentration 2 mg/mL (in free form), equilibration for 24 hours | | | | | | | | | | |
|---|---|---|---|---|---|---|---|---|---|---|---|
| | | Physical Form | | | | | | | | | |
| | | | | Sesqui-sodium | | L-arginine | | L-lysine | | Erbumine | |
| | | Free Form I, | | salt Form C, | | salt Form B, | | salt Form A, | | salt Form B, | |
| | | anhydrate | | hydrate | | hydrate | | anhydrate | | hydrate | |
| | | Batch/sample ID | | | | | | | | | |
| | | | | FR03684-SU1- | | | | | | | |
| | | | | NaOH-1.5-THF | | | | | | | |
| | | | | (ES1-ES3) | | FR03684-SU5- | | | | | |
| | | | | FR03684-SU9- | | L-arginine- | | | | FR03684-SU8- | |
| | | | | NaOH-1.5-THF | | acetone- | | FR03684-SU6- | | erbumine-ACN- | |
| | | FR03684-3-LP2 | | (ES4-ES7) | | water-95-5 | | L-lysine-EtOH | | water-95-5 | |
| | | Solubility media | | | | | | | | | |
| Exp. | | Solubility (pH) | | Solubility (pH) | | Solubility (pH) | | Solubility (pH) | | Solubility (pH) | |
| ID | | 2 h | 24 h | 2 h | 24 h | 2 h | 24 h | 2 h | 24 h | 2 h | 24 h |
| ES1 | pH 1.2 HCl | >2 | >2 | >2 | >2 | >2 | >2 | >2 | >2 | >2 | >2 |
| | buffer | | (1.3) | | (1.5) | | (1.3) | | (1.5) | | (1.5) |
| ES2 | pH 4.5 acetate | >2 | >2 | >2 | >2 | >2 | >2 | >2 | >2 | >2 | >2 |
| | buffer | | (4.2) | | (4.6) | | (4.7) | | (4.7) | | (4.8) |
| | (50 mM) | | | | | | | | | | |
| ES3 | pH 6.8 phosphate | >2 | >2 | >2 | >2 | >2 | >2 | >2 | >2 | >2 | >2 |
| | buffer | | (6.8) | | (6.9) | | (6.7) | | (6.7) | | (6.8) |
| | (50 mM) | | | | | | | | | | |
| ES4 | Water | >2 | >2 | >2 | >2 | >2 | >2 | >2 | >2 | >2 | >2 |
| | | | (3.5) | | (11.6) | | (7.2) | | (7.6) | | (8.0) |
| ES5 | FaSSGF, | >2 | >2 | >2 | >2 | >2 | >2 | >2 | >2 | >2 | >2 |
| | pH 1.6 | | (1.6) | | (4.6) | | (3.4) | | (3.4) | | (3.2) |
| ES6 | FaSSIF-v1, | >2 | >2 | >2 | >2 | >2 | >2 | >2 | >2 | >2 | >2 |
| | pH 6.5 | | (4.2) | | (7.2) | | (6.5) | | (6.5) | | (6.5) |
| ES7 | FeSSIF-v1, | >2 | >2 | >2 | >2 | >2 | >2 | >2 | >2 | >2 | >2 |
| | pH 5.0 | | (4.9) | | (5.3) | | (5.2) | | (5.1) | | (5.1) |

Trial 2: Accurate 5.06 mg of the free Form I (FR03684-3-LP2), 7.14 mg of the sodium salt Form C (FR03684-SU1-NaOH-1.5-THF), 7.47 mg of the sodium salt Form C (FR03684-SU9-NaOH-1.5-THF), 15.18 mg of the L-arginine salt Form B (FR03684-SU5-L-arginine-acetone-water-95-5), 12.89 mg of the L-lysine salt Form A (FR03684-SU6-L-lysine-EtOH), or 9.43 mg of the erbumine salt Form B (FR03684-SU8-erbumine-ACN-water-95-5) was weighed into a 2 ml glass vial, respectively. 0.5 ml of solubility medium (including pH 1.2 HCl buffer, pH 4.5 acetate buffer, pH 6.8 phosphate buffer, water, FaSSGF, FaSSIF and FeSSIF) was added. The salt amount used are equivalent to 5 mg anhydrous free form.

Obtained clear solutions were stirred at 37° C. at 400 rpm and sampled at 2 hours and at 24 hours. Obtained clear solutions were analyzed by pH meter for pH value.

TABLE 35

| | Solubility at 37° C., target concentration 10 mg/mL (in free form), equilibration for 24 hours | | | | | | | | | | |
|---|---|---|---|---|---|---|---|---|---|---|---|
| | | Physical Form | | | | | | | | | |
| | | | | Sesqui-sodium | | L-arginine | | L-lysine | | Erbumine | |
| | | Free Form I, | | salt Form C, | | salt Form B, | | salt Form A, | | salt Form B, | |
| | | anhydrate | | hydrate | | hydrate | | anhydrate | | hydrate | |
| | | Batch/sample ID | | | | | | | | | |
| | | | | FR03684-SU1- | | | | | | | |
| | | | | NaOH-1.5-THF | | | | | | | |
| | | | | (ES1-ES3) | | FR03684-SU5- | | | | | |
| | | | | FR03684-SU9- | | L-arginine- | | | | FR03684-SU8- | |
| | | | | NaOH-1.5-THF | | acetone- | | FR03684-SU6- | | erbumine-ACN- | |
| | | FR03684-3-LP2 | | (ES4-ES7) | | water-95-5 | | L-lysine-EtOH | | water-95-5 | |
| | | Solubility media | | | | | | | | | |
| Exp. | | Solubility (pH) | | Solubility (pH) | | Solubility (pH) | | Solubility (pH) | | Solubility (pH) | |
| ID | | 2 h | 24 h | 2 h | 24 h | 2 h | 24 h | 2 h | 24 h | 2 h | 24 h |
| ES1 | pH 1.2 HCl | >10 | >10 | >10 | >10 | >10 | >10 | >10 | >10 | >10 | >10 |
| | buffer | | (1.2) | | (1.3) | | (1.4) | | (1.5) | | (1.4) |
| ES2 | pH 4.5 acetate | >10 | >10 | >10 | >10 | >10 | >10 | >10 | >10 | >10 | >10 |
| | buffer | | (4.2) | | (4.6) | | (4.6) | | (4.7) | | (4.5) |
| | (50 mM) | | | | | | | | | | |
| ES3 | pH 6.8 phosphate | >10 | >10 | >10 | >10 | >10 | >10 | >10 | >10 | >10 | >10 |
| | buffer | | (6.8) | | (6.6) | | (6.6) | | (6.6) | | (6.8) |
| | (50 mM) | | | | | | | | | | |
| ES4 | Water | >10 | >10 | >10 | >10 | >10 | >10 | >10 | >10 | >10 | >10 |
| | | | (2.9) | | (12.4) | | (7.2) | | (7.0) | | (7.8) |
| ES5 | FaSSGF, | >10 | >10 | >10 | >10 | >10 | >10 | >10 | >10 | >10 | >10 |
| | pH 1.6 | | (1.3) | | (12.0) | | (5.0 | | (5.0) | | (5.0) |
| ES6 | FaSSIF-v1, | >10 | >10 | >10 | >10 | >10 | >10 | >10 | >10 | >10 | >10 |
| | pH 6.5 | | (3.6) | | (11.6) | | (6.4) | | (6.5) | | (6.5) |
| ES7 | FeSSIF-v1, | >10 | >10 | >10 | >10 | >10 | >10 | >10 | >10 | >10 | >10 |
| | pH 5.0 | | (4.4) | | (9.8) | | (5.2) | | (5.1) | | (5.2) |

Trial 3: Accurate 253.04 mg D-BHB free Form I (FR03684-3-LP2), 753.54 mg L-arginine salt Form B (sample ID FR03684-SU2-L-arginine-acetone-water-95-5-re), 603.66 mg L-lysine salt Form A (sample ID FR03684-SU3-L-lysine-EtOH-re), 321.16 mg sodium salt Form C (sample ID FR03684-SU1-NaOH-1.5-THF), or 377.25 mg of the erbumine salt Form B (sample ID FR03684-SU8-erbumine-ACN-water-95-5) was weighed into a 2 ml glass vial, respectively. 1 ml of pH 1.2 HCl buffer, pH 4.5 acetate buffer or pH 6.8 phosphate buffer was added in the D-BHB free Form I, L-arginine salt Form B and L-lysine salt Form A. The D-BHB free Form I, L-arginine salt Form Band L-lysine salt Form A amount used are equivalent to 250 mg anhydrous free Form I. 0.9 mL of pH 1.2 HCl buffer, pH 4.5 acetate buffer or pH 6.8 phosphate buffer was added in sodium salt Form C. The sodium salt amount used is equivalent to 225 mg anhydrous free form. 0.8 mL of pH 1.2 HCl buffer, pH 4.5 acetate buffer or pH 6.8 phosphate buffer was added in erbumine salt Form B. The erbumine salt Form B used is equivalent to 200 mg anhydrous free form.

Accurate 75.91 mg of the D-BHB free Form I (batch FR03684-3-LP2), 110.85 mg of the sodium salt Form C (sample ID FR03684-SU9-NaOH-1.5-THF), 227.76 mg of the L-arginine salt Form B (sample ID FR03684-SU5-L-arginine-acetone-water-95-5), 190.97 mg of the L-lysine salt Form A (sample ID FR03684-SU6-L-lysine-EtOH) or 235.77 mg of the erbumine salt Form B (sample ID FR03684-SU8-erbumine-ACN-water-95-5) was weighed into a 2 ml glass vial, respectively. 0.3 ml of water was added to the D-BHB free Form I, the sodium salt Form C, the L-arginine salt Form B, and the L-lysine salt Form A. The D-BHB free Form I, the sodium salt Form C, the L-arginine salt Form B, the L-lysine salt Form A amount used are equivalent to 75 mg anhydrous D-BHB free Form I. 0.5 ml of water was added to the erbumine salt Form B. The erbumine salt Form B amount used is equivalent to 125 mg anhydrous free form.

Obtained clear solutions/almost clear solutions were stirred at 37° C. at 400 rpm and sampled at 2 hours and at 24 hours. The samples were centrifuged at 37° C. at 14,000 rpm for 5 min. Supernatants were analyzed by IC and pH meter for solubility and pH value, respectively.

TABLE 36

| | Solubility at 37° C., target concentration 250 mg/mL (in free form), equilibration for 24 hours, LOQ: 0.103 mg/mL | | | | | | | | | | |
|---|---|---|---|---|---|---|---|---|---|---|---|
| | | Physical Form | | | | | | | | | |
| | | | | Sesqui-sodium | | L-arginine | | L-lysine | | Erbumine | |
| | | Free Form I, | | salt Form C, | | salt Form B, | | salt Form A, | | salt Form B, | |
| | | anhydrate | | hydrate | | hydrate | | anhydrate | | hydrate | |
| | | Batch/sample ID | | | | | | | | | |
| | | | | | | FR03684-SU2- | | | | | |
| | | | | | | L-arginine- | | | | | |
| | | | | | | acetone- | | | | | |
| | | | | | | water-95-5-re | | | | | |
| | | | | FR03684-SU1- | | (ES1-ES3) | | FR03684-SU3- | | | |
| | | | | NaOH-1.5-THF | | FR03684-SU5- | | L-lysine-EtOH-re | | | |
| | | | | (ES1-ES3) | | L-arginine- | | (ES1-ES3) | | | |
| | | | | FR03684-SU9- | | acetone- | | FR03684-SU6- | | FR03684-SU8- | |
| | | | | NaOH-1.5-THF | | water-95-5 | | L-lysine-EtOH | | erbumine-ACN- | |
| | | FR03684-3-LP2 | | (ES4) | | (ES4) | | (ES4) | | water-95-5 | |
| | | Solubility media | | | | | | | | | |
| Exp. | | Solubility (pH) | | Solubility (pH) | | Solubility (pH) | | Solubility (pH) | | Solubility (pH) | |
| ID | | 2 h | 24 h | 2 h | 24 h | 2 h | 24 h | 2 h | 24 h | 2 h | 24 h |
| ES1 | pH 1.2 HCl | >250 | >250 | 206.5 | 230.2 | 152.2 | 213.2 | 150.4 | 173.5 | 155.9 | 183.4 |
| | buffer | | (1.1) | | (1.5) | | (1.5) | | (1.3) | | (0.9) |
| ES2 | pH 4.5 acetate | >250 | >250 | 141.5 | 156.6 | 113.0 | 116.3 | 143.1 | 145.2 | 132.9 | 141.4 |
| | buffer | | (4.4) | | (4.7) | | (4.8) | | (4.8) | | (4.9) |
| | (50 mM) | | | | | | | | | | |
| ES3 | pH 6.8 phosphate | >250 | >250 | 151.4 | 157.0 | 84.8 | 102.7 | 138.7 | 144.5 | 124.3 | 140.9 |
| | buffer | | (6.7) | | (6.6) | | (7.0) | | (6.8) | | (7.2) |
| | (50 mM) | | | | | | | | | | |
| ES4 | Water | >250 | >250 | 240.2 | 243.1 | 178.6 | 189.9 | 183.5 | 205.3 | 201.3 | 229.5 |
| | | | (1.9) | | (13.4) | | (7.2) | | (7.3) | | (7.8) |

Both the D-BHB crystalline salts and the D-BHB free Form I demonstrated good solubility in selected aqueous media. They showed >10 mg/ml solubility in pH 1.6 FaSSGF, pH 6.5 FaSSIF-v1, and pH 5.0 FeSSIF-v1. When target concentration for solubility test was increased to 250 mg/mL, the D-BHB free Form I showed >250 mg/ml solubility in pH 1.2 HCl buffer, pH 4.5 acetate buffer, pH 6.8 phosphate buffer and water. The D-BHB salts showed 170-240 mg/ml solubility in pH 1.2 HCl buffer, 120-160 mg/ml solubility in pH 4.5 acetate buffer and 100-160 mg/mL in pH 6.8 phosphate buffer after 24 h.

### Example 13: Hygroscopicity

TABLE 37
Hygroscopicity by DVS at 25° C. - METHOD 1
Physical Form

| | Sesqui-sodium salt Form C, hydrate | | | | L-arginine salt Form B, hydrate | | | |
|---|---|---|---|---|---|---|---|---|
| | Batch no./sample ID | | | | | | | |
| | | | | | FR03684-SU5-L-arginine- | | | |
| Relative | FR03684-SU1-NaOH-1.5-THF | | | | acetone-water-95-5 | | | |
| humidity at | Desorp. | Sorp. | Desorp. | Sorp. | Desorp. | Sorp. | Desorp. | Sorp. |
| 25° C. | (%) | (%) | (%) | (%) | (%) | (%) | (%) | (%) |
| 0% | 0.0 | 0.0 | 40.3 | 40.3 | 0.0 | 0.0 | 0.8 | 0.8 |
| 10% | 0.4 | 0.1 | 42.7 | 42.4 | 0.4 | 0.4 | 1.6 | 0.7 |
| 20% | 0.6 | 0.5 | 44.2 | 43.7 | 0.9 | 0.9 | 2.8 | 1.1 |
| 30% | 1.0 | 0.8 | 46.1 | 45.4 | 1.5 | 1.4 | 4.6 | 2.8 |
| 40% | 2.2 | 2.4 | 49.3 | 48.4 | 1.9 | 1.8 | 8.0 | 7.1 |
| 50% | // | 66.5 | 93.9 | // | // | 2.5 | 13.6 | // |
| 60% | // | 95.8 | 119.7 | // | // | 3.4 | 21.5 | // |
| 70% | // | 137.6 | 155.0 | // | // | 5.9 | 33.4 | // |
| 80% | // | 195.6 | 213.0 | // | // | 52.5 | 52.6 | // |
| 90% | // | 323.5 | 384.2 | // | // | 105.5 | 104.8 | // |
| 95% | // | 473.4 | 473.4 | // | // | 196.8 | 196.8 | // |
| Hygroscopicity | Moderately hygroscopic below | | | | Moderately hygroscopic below | | | |
| | 40% RH, | | | | 70% RH, | | | |
| | Very hygroscopic and 471.0% water | | | | Very hygroscopic and 190.9% water | | | |
| | uptake from 40% RH to 95% RH | | | | uptake from 70% RH to 95% RH | | | |
| XRPD after DVS | Sodium salt Form B | | | | Amorphous form | | | |
| test | | | | | | | | |

“//” = Not carried out
Non-hygroscopic water uptake <0.2%
Slightly hygroscopic water uptake ≥0.2% but <2%
Moderately hygroscopic water uptake ≥2% but <15%
Very hygroscopic water uptake ≥15%
Water uptake = water sorption in a specific RH (80% to 95%) − water sorption in 40% RH
The criteria are modified from the European Pharmacopeia criteria about hygroscopicity

TABLE 38
Hygroscopicity by DVS at 25° C. - METHOD 2
Physical Form

| | L-lysine salt Form A, anhydrate | | | | Erbumine salt Form B, hydrate | | | |
|---|---|---|---|---|---|---|---|---|
| | Batch no./sample ID | | | | | | | |
| | | | | | FR03684-SU8-erbumine- | | | |
| Relative | FR03684-SU6-L-lysine-EtOH | | | | ACN-water-95-5 | | | |
| humidity at | Desorp. | Sorp. | Desorp. | Sorp. | Desorp. | Sorp. | Desorp. | Sorp. |
| 25° C. | (%) | (%) | (%) | (%) | (%) | (%) | (%) | (%) |
| 0% | 0.0 | 0.0 | 5.2 | 5.2 | 0.1 | 0.1 | 4.0 | 4.0 |
| 10% | 0.2 | 0.1 | 6.9 | 4.6 | 0.3 | 0.1 | 4.4 | 3.6 |
| 20% | 0.4 | 0.2 | 10.1 | 4.5 | 9.5 | 0.0 | 4.8 | 3.4 |
| 30% | 0.8 | 0.5 | 14.4 | 5.4 | 10.7 | 0.0 | 5.4 | 3.1 |
| 40% | 1.4 | 1.0 | 20.3 | 6.6 | 10.9 | 0.8 | 9.2 | 2.7 |
| 50% | // | 2.9 | 28.8 | // | // | 10.0 | 18.8 | // |
| 60% | // | 35.4 | 40.7 | // | // | 11.6 | 34.7 | // |
| 70% | // | 57.5 | 59.6 | // | // | 43.1 | 76.1 | // |
| 80% | // | 86.1 | 106.3 | // | // | 86.9 | 147.5 | // |
| 90% | // | 137.5 | 180.4 | // | // | 140.2 | 201.2 | // |
| 95% | // | 190.7 | 190.7 | // | // | 194.7 | 194.7 | // |

| Hygroscopicity | Moderately hygroscopic below | Moderately hygroscopic from 20% to |
|---|---|---|
| | 50% RH, | 60% RH, |
| | Very hygroscopic and 187.8% water | Very hygroscopic and 183.1% water |
| | uptake from 50% RH to 95% RH | uptake from 60% RH to 95% RH |
| XRPD after DVS | Low crystalline form | Erbumine salt Form B deliquesce to |
| test | | liquid after DVS test |

“//” = Not carried out
Non-hygroscopic water uptake <0.2%
Slightly hygroscopic water uptake ≥0.2% but <2%
Moderately hygroscopic water uptake ≥2% but <15%
Very hygroscopic water uptake ≥15%
Water uptake = water sorption in a specific RH (80% to 95%) − water sorption in 40% RH
The criteria are modified from the European Pharmacopeia criteria about hygroscopicity

Hygroscopicity of the D-BHB salts was evaluated by dynamic vapor sorption (DVS) test at 25° C. The L-arginine salt Form B showed advantage over the other D-BHB salts in hygroscopicity.

The L-arginine salt Form B is moderately hygroscopic below 70% RH. After the DVS test, the L-arginine salt Form B converted to an amorphous form.

The sesqui-sodium salt Form C and the L-lysine salt Form A became very hygroscopic at above 40% RH and 50% RH, respectively. Then they deliquesced in high humidity. After the DVS test, the sesqui-sodium salt Form C converted to the sesqui-sodium salt Form B and the L-lysine salt Form A converted to a low crystalline form

The erbumine salt Form B underwent dehydration below 20% RH and lost about 9.4% water. The water was absorbed back after the relative humidity (RH) was increased to 50% RH. Then the erbumine salt Form B became very hygroscopic in above 60% RH and deliquesced in high humidity. After the DVS test, and the erbumine salt Form B converted to a liquid state.

### Example 14—Chemical and Physiochemical Properties

Sodium salt Form C is a hydrate. Sample FR03684-SU1-NaOH-1.5-THF is of high crystallinity. DSC shows multiple thermal events. TGA shows about 7.8% weight loss at about 130° C. IC shows D-BHB: Na⁺ is 1:1.5. ¹H-NMR shows no detectable residual solvent. KF shows it contains about 6.7% water by weight, equivalent to 0.6 water molecule.

L-arginine salt Form B is a hydrate. Sample FR03684-SU5-L-arginine-acetone-water-95-5 is of high crystallinity. DSC shows a dehydration Tₒₙₛₑₜ of 89.2° C. Melting occurs upon dehydration. TGA shows about 6.3% weight loss at about 130° C. ¹H-NMR shows D-BHB: L-arginine is 1:1.0 and no detectable residual solvent. KF shows it contains about 6.2% water by weight, equivalent to 1.1 water molecule.

L-lysine salt Form A is an anhydrate. Sample FR03684-SU6-L-lysine-EtOH is of high crystallinity. DSC shows a melting Tₒₙₛₑₜ of 108.4° C. with an enthalpy of about 79 J/g. TGA shows about 4.1% weight loss at about 100° C. ¹H-NMR shows D-BHB: L-lysine is 1:1.0 and no detectable residual solvent.

Erbumine salt Form B is a hydrate. Sample FR03684-SU8-erbumine-ACN-water-95-5 is of high crystallinity. DSC shows a dehydration Tₒₙₛₑₜ of 51.9° C. and then a melting Tₒₙₛₑₜ of 72.8° C. TGA shows about 10.3% weight loss at about 90° C. ¹H-NMR shows D-BHB: erbumine is 1:1.0 and no detectable residual solvent. KF shows it contains about 9.7% water by weight, equivalent to 1.1 water molecule.

TABLE 39

| | Physical Form | | | | |
|---|---|---|---|---|---|
| | | | L-arginine salt | | Erbumine salt |
| | | | Form B, | | Form B, |
| | | Sodium salt | hydrate | L-lysine salt | hydrate |
| | | Form C, | FR03684-SU5- | Form A, | FR03684-SU8- |
| | | hydrate | L-arginine- | anhydrate | erbumine- |
| Batch no./ | Free Form I | FR03684-SU1- | acetone- | FR03684-SU6- | ACN-water- |
| sample ID | FR03684-1-LP2 | NaOH-1.5-THF | water-95-5 | L-lysine-EtOH | 95-5 |

Stoichiometry by ¹H-NMR or IC

| | | | | | |
|---|---|---|---|---|---|
| Free from: counter | N/A | 1:1.5 | 1:1.0 FIG. 30D | 1:1.0 FIG. 31D | 1:1.0 FIG. 26E |
| ion | | | | | |

Residual solvent(s) by ¹H-NMR

| | | | | | |
|---|---|---|---|---|---|
| Weight (%) | Undetected | Undetected | Undetected | Undetected | Undetected |
| | FIG. 4B | FIG. 29B | FIG. 30B | FIG. 31D | FIG. 26E |

Water content by Karl Fisher (for hydrate only)
Crystallinity by XRPD

| | | | | | |
|---|---|---|---|---|---|
| High/medium/low | High - | High - | High - | High - | High - |
| | FIG. 1B | FIG. 29A | FIG. 30A | FIG. 31A | FIG. 26B |

DSC, heating rate [10° C./min]

| | | | | | |
|---|---|---|---|---|---|
| Melting onset (° C.) | Evaporation of | Multiple | Dehydration | Melting Tₒₙₛₑₜ | Dehydration |
| | surface | thermal events | and melting | @ 108.4° C. | Tₒₙₛₑₜ @ |
| | moisture from | FIG. 29B | Tₒₙₛₑₜ @ 89.2° C. | FIG. 31B | 51.9° C.; |
| | about 8° C. | | FIG. 30B | | Melting Tₒₙₛₑₜ |
| | FIG. 3B | | | | @ 72.8° C. |
| | | | | | FIG. 26C |
| Melting enthalpy | No melting | FIG. 29B | FIG. 30B | About 79J/g | FIG. 26C |
| (J/g) | point before | | | FIG. 31B | |
| | decomposition | | | | |
| | FIG. 3B | | | | |

Themogravimetry, heating rate [10° C./min]

| | | | | | |
|---|---|---|---|---|---|
| Weight loss in (%) | About 1.9% @ | About 7.8% @ | About 6.3% @ | About 4.1% @ | About 10.3% @ |
| @ (° C.) | 100° C. | 130° C. | 130° C. | 100° C. | 90° C. |
| | FIG. 3C | FIG. 29C | FIG. 30C | FIG. 31C | FIG. 26D |

### Example 15—DVS Isotherm Plots

(1) DVS Method 1 was used to generate a DVS isotherm plot of D-BHB sodium salt Form C (FR03684-SU1-NaOH-1.5-THF), at 25° C.

TABLE 40
Sample name: FR03684-3-SU1-2-DVS
Batch no.:
Sample mass: 14.986 mg
Reference weight: Lowest net weight
Temperature: 25° C.

| Sorption | | Desorption | | Sorption | | Desorption | |
|---|---|---|---|---|---|---|---|
| cycle 1 | | cycle 1 | | cycle 2 | | cycle 2 | |
| RH read | dm | RH read | dm | RH read | dm | RH read | Dm |
| [%] | [%] | [%] | [%] | [%] | [%] | [%] | [%] |
| 0.7 | 0.03 | 40.0 | 2.20 | 0.6 | 40.30 | 95.0 | 473.42 |
| 10.0 | 0.13 | 30.0 | 1.02 | 10.0 | 42.39 | 90.0 | 384.19 |
| 20.0 | 0.49 | 20.0 | 0.64 | 20.0 | 43.71 | 80.0 | 212.95 |
| 30.0 | 0.83 | 10.1 | 0.41 | 30.0 | 45.38 | 70.0 | 154.98 |
| 40.0 | 2.37 | 0.7 | 0.03 | 40.0 | 48.43 | 60.1 | 119.69 |
| 50.0 | 66.52 | | | | | 50.0 | 93.85 |
| 60.0 | 95.78 | | | | | 40.0 | 49.29 |
| 69.9 | 137.56 | | | | | 30.1 | 46.07 |
| 79.9 | 195.60 | | | | | 20.1 | 44.21 |
| 90.0 | 323.46 | | | | | 10.1 | 42.69 |
| 95.0 | 473.42 | | | | | 0.6 | 40.30 |

(2) DVS Method 1 was used to generate a DVS isotherm plot of D-BHB L-arginine salt Form B (FR03684-SU5-L-arginine-acetone-water-95-5), at 25° C.

TABLE 41
Sample name: FR03684-3-SU5-DVS
Batch no.:
Sample mass: 8.678 mg
Reference weight: Lowest net weight
Temperature: 25° C.

| Sorption | | Desorption | | Sorption | | Desorption | |
|---|---|---|---|---|---|---|---|
| cycle 1 | | cycle 1 | | cycle 2 | | cycle 2 | |
| RH read | dm | RH read | dm | RH read | dm | RH read | dm |
| [%] | [%] | [%] | [%] | [%] | [%] | [%] | [%] |
| 0.7 | 0.00 | 40.0 | 1.89 | 0.6 | 0.81 | 95.0 | 196.83 |
| 10.0 | 0.40 | 30.0 | 1.47 | 10.0 | 0.65 | 90.0 | 104.82 |
| 20.0 | 0.89 | 20.0 | 0.92 | 20.0 | 1.05 | 80.0 | 52.63 |
| 30.0 | 1.36 | 10.1 | 0.44 | 30.0 | 2.79 | 70.0 | 33.38 |
| 40.0 | 1.75 | 0.7 | 0.00 | 40.0 | 7.12 | 60.1 | 21.45 |
| 50.0 | 2.51 | | | | | 50.0 | 13.60 |
| 59.9 | 3.36 | | | | | 40.0 | 8.02 |
| 69.9 | 5.91 | | | | | 30.0 | 4.59 |
| 79.9 | 52.50 | | | | | 20.1 | 2.79 |
| 90.0 | 105.54 | | | | | 10.1 | 1.59 |
| 95.0 | 196.83 | | | | | 0.6 | 0.81 |

(3) DVS Method 2 was used to generate a DVS isotherm plot of D-BHB L-lysine salt Form A (FR03684-SU6-L-lysine-EtOH), at 25° C.

TABLE 42
Meth: STA-SSD Lab DVS-05_40-0-95-0-40%
RH time 240 min _step 10%
Sample: FR03684-3-SU6-DVS
Temp: 25.2° C.
MRef: 9.7902 from Custom Mass

| | | | Change In | |
|---|---|---|---|---|
| | Target | | Mass (%) - ref | |
| | % P/Po | Sorption | Desorption | Hysteresis |
| Cycle 1 | 0.0 | 0.0 | 0.0 | |
| | 10.0 | 0.1 | 0.2 | 0.1 |
| | 20.0 | 0.2 | 0.4 | 0.2 |
| | 30.0 | 0.5 | 0.8 | 0.3 |
| | 40.0 | 1.0 | 1.4 | 0.4 |
| | 50.0 | 2.9 | | |
| | 60.0 | 35.4 | | |
| | 70.0 | 57.5 | | |
| | 80.0 | 86.1 | | |
| | 90.0 | 137.5 | | |
| | 95.0 | 190.7 | | |
| Cycle 2 | 0.0 | 5.2 | 5.2 | |
| | 10.0 | 4.6 | 6.9 | 2.3 |
| | 20.0 | 4.5 | 10.1 | 5.7 |
| | 30.0 | 5.4 | 14.4 | 9.0 |
| | 40.0 | 6.6 | 20.3 | 13.7 |
| | 50.0 | | 28.8 | |
| | 60.0 | | 40.7 | |
| | 70.0 | | 59.6 | |
| | 80.0 | | 106.3 | |
| | 90.0 | | 180.4 | |
| | 95.0 | | 190.7 | |

(4) DVS Method 2 was used to generate a DVS isotherm plot of D-BHB erbumine salt Form B (FR03684-SU8-erbumine-ACN-water-95-5), at 25° C.

TABLE 43
Meth: STA-SSD Lab DVS-06_40-0-95-0-40%
RH_Step 10%_time240 min
Sample: FR03684-3-SU8-2-DVS
Temp: 24.7° C.
MRef: 15.0475 from Custom Mass

| | | | Change In | |
|---|---|---|---|---|
| | Target | | Mass (%) - ref | |
| | % P/Po | Sorption | Desorption | Hysteresis |
| Cycle 1 | 0.0 | 0.1 | 0.1 | |
| | 10.0 | 0.1 | 0.3 | 0.2 |
| | 20.0 | 0.0 | 9.5 | 9.5 |
| | 30.0 | 0.0 | 10.7 | 10.7 |
| | 40.0 | 0.8 | 10.9 | 10.1 |
| | 50.0 | 10.0 | | |
| | 60.0 | 11.6 | | |
| | 70.0 | 43.1 | | |
| | 80.0 | 86.9 | | |
| | 90.0 | 140.2 | | |
| | 95.0 | 194.7 | | |
| Cycle 2 | 0.0 | 4.0 | 4.0 | |
| | 10.0 | 3.6 | 4.4 | 0.7 |
| | 20.0 | 3.4 | 4.8 | 1.5 |
| | 30.0 | 3.1 | 5.4 | 2.4 |
| | 40.0 | 2.7 | 9.2 | 6.5 |
| | 50.0 | | 18.8 | |
| | 60.0 | | 34.7 | |
| | 70.0 | | 76.1 | |
| | 80.0 | | 147.5 | |
| | 90.0 | | 201.2 | |
| | 95.0 | | 194.7 | |

### Example 16: Extended Stability Study

D-BHB sodium salt Form C, hydrate (C210608011-B/PJ06432-24-B-DRY) and D-BHB L-arginine salt Form B, hydrate (C210608011-FP/PJ06432-23-FP-DRY) (TABLE 45) were studied at 25° C.±2° C./60% RH±5% RH, 40° C.±2° C./75% RH±5% RH, 50° C.±2° C./75% RH±5% RH. The samples were tested at weeks 1, 2, 3, and 4. The bulk substance was studied in a container closure system that simulates packaging of bulk materials: approximately 1.0 g/package for each of the tests and time points, with the sample packed into double antistatic LDPE bag that was secured with a cable tie. 4 bags of desiccant were then put between two layers of antistatic LDPE bag. Each package (sample in bag with 4 bags of desiccant) into a foil bag that was heat sealed. The foil bag was then stored within a Fiber drum. The drums were then stored in a stability chamber at the conditions noted above with the temperature and humidity monitored and recorded. The tests were Appearance, Related substance by Ion Chromatography (IC), Potency, Coulometric water content determination according to Karl Fischer, XPRD, and Chiral purity.

D-BHB sodium salt Form C, hydrate (C210608011-B/PJ06432-24-B-DRY): Throughout the course of the four-week study, at each of the three temperature/relative humidity conditions, the compound: Maintained a constant appearance, without change: white solid; Maintained chiral purity, without change: 100.0%; and Maintained XPRD of Form C, without change.

Other parameters are shown in TABLE 44, in which the Conditions (abbreviated “Con”) are (A) 25° C.±2° C./60% RH±5% RH, (B) 40° C.±2° C./75% RH±5% RH, and (C) 50° C.±2° C./75% RH±5% RH. D-BHB L-arginine salt Form B, hydrate (C210608011-FP/PJ06432-23-FP-DRY) Throughout the course of the four-week study, at each of the three temperature/relative humidity conditions, the compound: Maintained a constant appearance, without change: white solid; Maintained chiral purity, without change: 100.0%; and Maintained XPRD of Form B, without change. Other parameters are shown in TABLE 45, in which the Conditions (abbreviated “Con”) are (A) 25° C.±2° C./60% RH±5% RH, (B) 40° C.±2° C./75% RH±5% RH, and (C) 50° C.±2° C./75% RH±5% RH.

TABLE 44
D-BHB sodium salt Form C, hydrate (C210608011-B/PJ06432-24-B-DRY)

| TEST | | Initial | Con | 1 Week | 2 Week | 3 Week | 4 Week |
|---|---|---|---|---|---|---|---|
| Related | Individual | RRT 1.61 = 0.77 | (A) | RRT 1.61 = 0.84 | RRT 1.62 = 0.77 | RRT 1.63 = 0.80 | RRT 1.16 = 0.09 |
| substances | impurity | RRT 2.44 = 1.7 | | RRT 2.43 = 1.5 | RRT 2.42 = 1.6 | RRT 2.40 = 1.7 | RRT 1.62 = 0.79 |
| (% | | | | | | | RRT 2.46 = 1.9 |
| area) | | | (B) | RRT 1.16 = 0.05 | RRT 1.16 = 0.05 | RRT 1.16 = 0.06 | RRT 1.16 = 0.20 |
| | | | | RRT 1.61 = 0.87 | RRT 1.62 = 0.78 | RRT 1.63 = 0.82 | RRT 1.62 = 0.84 |
| | | | | RRT 2.43 = 1.5 | RRT 2.42 = 1.6 | RRT 2.40 = 1.9 | RRT 2.46 = 1.8 |
| | | | (C) | RRT 1.16 = 0.06 | RRT 1.16 = 0.07 | RRT 1.16 = 0.13 | RRT 1.16 = 0.27 |
| | | | | RRT 1.61 = 0.84 | RRT 1.61 = 0.94 | RRT 1.63 = 1.1 | RRT 1.62 = 1.2 |
| | | | | RRT 2.43 = 1.5 | RRT 2.43 = 1.6 | RRT 2.40 = 1.8 | RRT 2.46 = 1.8 |
| | Total | 2.4 | (A) | 2.3 | 2.4 | 2.5 | 2.7 |
| | impurities | | (B) | 2.4 | 2.4 | 2.8 | 2.8 |
| | | | (C) | 2.4 | 2.6 | 3.0 | 3.2 |

| Potency | 95.7 | (A) | 94.7 | 93.1 | 92.3 | 90.8 |
|---|---|---|---|---|---|---|
| (% w/w) | | (B) | 92.2 | 90.3 | 89.5 | 89.6 |
| | | (C) | 91.2 | 89.2 | 89.1 | 88.4 |
| Water | 1.9 | (A) | 2.9 | 4.6 | 5.2 | 6.6 |
| Content by KF | | (B) | 5.5 | 7.4 | 7.9 | 7.7 |
| (% w/w) | | (C) | 6.5 | 8.4 | 8.1 | 8.6 |

TABLE 45
D-BHB L-arginine salt Form B, hydrate (C210608011-FP/PJ06432-23-FP-DRY)

| TEST | | Initial | Con | 1 Week | 2 Week | 3 Week | 4 Week |
|---|---|---|---|---|---|---|---|
| Related | Individual | RRT 1.63 = 0.05 | (A) | RRT 2.49 = 1.0 | RRT 2.48 = 1.2 | RRT 2.45 = 1.2 | RRT 1.65 = 0.06 |
| substances | impurity | RRT 2.49 = 1.1 | | | | | RRT 2.51 = 1.4 |
| (% area) | | | (B) | RRT 1.63 = 0.05 | RRT 2.49 = 1.1 | RRT 1.61 = 0.05 | RRT 1.65 = 0.06 |
| | | | | RRT 2.48 = 0.98 | | RRT 2.49 = 1.2 | RRT 2.51 = 1.4 |
| | | | (C) | RRT 1.63 = 0.05 | RRT 1.64 = 0.05 | RRT 1.61 = 0.05 | RRT 1.65 = 0.07 |
| | | | | RRT 2.48 = 1.0 | RRT 2.46 = 1.2 | RRT 2.49 = 1.4 | RRT 2.51 = 1.4 |
| | Total | 1.1 | (A) | 1.0 | 1.2 | 1.2 | 1.4 |
| | impurities | | (B) | 1.0 | 1.1 | 1.3 | 1.4 |
| | | | (C) | 1.1 | 1.2 | 1.5 | 1.4 |

| Potency | 92.1 | (A) | 92.8 | 92.1 | 91.9 | 91.8 |
|---|---|---|---|---|---|---|
| (% w/w) | | (B) | 92.8 | 92.0 | 92.1 | 92.0 |
| | | (C) | 93.1 | 92.5 | 92.4 | 92.1 |
| Water Content | 5.8 | (A) | 5.2 | 5.7 | 5.9 | 5.8 |
| by KF | | (B) | 5.1 | 5.9 | 5.6 | 5.6 |
| (% w/w) | | (C) | 4.8 | 5.3 | 5.2 | 5.5 |

1. A composition comprising D-β-hydroxybutyrate (D-BHB) for use in the treatment or dietary management of a Long-chain Fatty Acid Oxidation Deficiency (LC-FAOD).

2. The composition for use according to claim 1, wherein the treatment or dietary management is the prevention or amelioration of one or more symptoms associated with said LC-FAOD.

3. The composition for use according to claim 1, wherein the composition comprises one or more salts of D-β-hydroxybutyrate (D-BHB).

4. The composition for use according to claim 3, wherein the composition comprises a sodium salt of D-BHB (Na-D-BHB) and an L-arginine salt of D-BHB (L-Arg-D-BHB); wherein the sodium salt of D-BHB (Na-D-BHB) comprises salt form C and/or wherein the L-arginine salt of D-BHB (L-Arg-D-BHB) comprises salt form B.

5. The composition for use according to claim 4, wherein the molar ratio between the sodium salt of D-BHB (Na-D-BHB) and the L-arginine salt of D-BHB (L-Arg-D-BHB) is between 1:10 and 10:1.

6. The composition for use according to claim 1, wherein the composition is free or essentially free of L-β-hydroxybutyrate (L-BHB).

7. The composition for use according to claim 1, wherein the composition is formulated to be administered enterally or parentally.

8. A method for restoring or promoting normative cellular energetics by providing an alternative energy source and/or bypassing mitochondrial fatty acid oxidation, in a subject, comprising administering to the subject a composition comprising D-β-hydroxybutyrate (D-BHB).

9. A composition comprising two or more salts of D-β-hydroxybutyrate (D-BHB), wherein at least one of the two or more salts is selected from a sodium salt, an L-arginine salt, an-L-lysine salt and/or an erbumine salt.

10. The composition according to claim 9, wherein the composition comprises at least one amino acid salt of D-BHB and one mineral salt of D-BHB.

11. The composition according to claim 9, wherein the composition comprises a sodium salt of D-BHB (Na-D-BHB) and an L-arginine salt of D-BHB (L-Arg-D-BHB); and/or wherein the L-arginine salt of D-BHB (L-Arg-D-BHB) comprises salt form B.

12. The composition according to claim 11, wherein the molar ratio between the sodium salt of D-BHB (Na-D-BHB) and the L-arginine salt of D-BHB (L-Arg-D-BHB) is between 1:10 and 10:1.

13. The composition according to claim 9, wherein the two or more salts of D-BHB are selected from a sodium salt, a calcium salt, and/or a magnesium salt.

14. The composition according to claim 13, wherein the molar ratio between (i) the sodium salt and (ii) the calcium salt and/or the magnesium salt ranges from 1:1 to 1:10.

15. The composition according to claim 13, wherein the composition further comprises D-BHB in its free form, wherein the ratio between the D-BHB salts and D-BHB in its free form ranges from 1:10 to 10:1.

16. The composition according to claim 9, wherein the composition is free or essentially free of L-β-hydroxybutyrate (L-BHB).

17. The composition according to claim 9, wherein the composition is formulated as a powder; and/or wherein the composition is formulated for enteral administration or for parental administration.

18. The composition according to claim 9, wherein the composition is a nutritional composition, including as a food for special medical purpose (FSMP) and comprising at least one macronutrient selected from the group consisting of a protein, a carbohydrate, a lipid, and combinations thereof; or wherein the composition is a pharmaceutical composition further comprising a pharmaceutically acceptable carrier.

19. The composition according to claim 9, wherein the composition further comprises nicotinamide riboside, L-arginine and/or a medium-chain triglyceride.

20. The composition for use according to claim 1, wherein the LC-FAOD is caused by a mutation in one or more genes encoding the following mitochondrial enzymes: carnitine palmitoyl transferase I (CPT-I), carnitine palmitoyl transferase (CPT-II), very long-chain acyl-CoA dehydrogenase (VLCAD), long-chain 3-hydroxy-acyl-CoA dehydrogenase (LCHAD), carnitine acyl-carnitine translocase (CACT), trifunctional protein (TFP), and/or long-chain 3 ketoacyl-CoA thiolase (LCKAT).

21. The composition for use according to claim 3, wherein the one or more salts of D-β-hydroxybutyrate (D-BHB) is selected from:
a) a sodium salt, a calcium salt, and/or a magnesium salt; and/or
b) a sodium salt, an L-arginine salt, an-L-lysine salt and/or an erbumine salt.

22. The composition according to claim 13, wherein the two or more salts of D-BHB include all three of the sodium salt, the calcium salt, and the magnesium salt.

23. The composition according to claim 19, wherein the nicotinamide riboside, L-arginine and/or a medium-chain triglyceride includes triheptanoin.
